(19) United States
(12) Patent Application Publication
(10) Pub. No.: US 2025/0025551 A1
(43) Pub. Date: Jan. 23, 2025

(54) COMPOSITIONS AND METHODS FOR INDUCING AND ENHANCING AN IMMUNE RESPONSE

(71) Applicant: Board of Trustees of Michigan State University, East Lansing, MI (US)

(72) Inventors: Christopher Waters, East Lansing, MI (US); Andrea Amalfitano, East Lansing, MI (US)

(21) Appl. No.: 18/773,259

(22) Filed: Jul. 15, 2024

Related U.S. Application Data
(63) Continuation of application No. 18/384,544, filed on Oct. 27, 2023, now abandoned.
Application No. 18/384,544 is a continuation of application No. 18/075,809, filed on Dec. 6, 2022, now abandoned.
Application No. 18/075,809 is a continuation of application No. 17/727,019, filed on Apr. 22, 2022, now abandoned.
Application No. 17/727,019 is a continuation of application No. 15/760,726, filed on Mar. 16, 2018, now abandoned.
(60) Provisional application No. 62/219,387, filed on Sep. 16, 2015.

(51) Int. Cl. A61K 39/39; A61K 39/00; A61K 39/08; C12N 9/12

(52) U.S. Cl. CPC A61K 39/39; C12N 9/1241; A61K 39/08; A61K 2039/55511; A61K 2039/55561; A61K 2039/55594; C12N 2710/10343; C12N 2740/16234

(57) ABSTRACT

The present invention relates to compositions and methods for modulating immune responses using at least one cycli di-nucleotide synthetase enzyme gene. Such compositions may be combined with a number of other therapeutics which target modulating immune responses, as well as, treatments that include immune events.

### CROSS-REFERENCE TO RELATED APPLICATIONS

This application is a continuation of U.S. patent application Ser. No. 18/384,544, filed Oct. 27, 2023, which is a continuation of U.S. patent application Ser. No. 18/075,809, filed Dec. 6, 2022, which is a continuation of U.S. patent application Ser. No. 17/727,019, filed on Apr. 22, 2022, which is a continuation of U.S. patent application Ser. No. 15/760,726, filed on Mar. 16, 2018, which is a 371 National Stage Application of PCT/US16/52198, filed Sep. 16, 2016, which claims the benefit of priority to U.S. Provisional Application No. 62/219,387 filed 16 Sep. 2015, the entire contents of said application is incorporated herein in its entirety by this reference.

### STATEMENT OF RIGHTS

This invention was made with government support under AI057153 and AI105499 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Aug. 26, 2024, is named MSS-01305 SL.xml and is 218,000 bytes in size.

### BACKGROUND OF THE INVENTION

With a limited number of adjuvants approved for human administration, there is a pressing need for the development and testing of vaccine adjuvants that can improve the efficacy and maintain the safety profile of vaccines against resilient infectious diseases and cancers (Alving, C R et al. (2012) Curr Opin Immunol 24: 310-315). The addition of adjuvants to vaccine formulations can serve to significantly improve vaccine efficacy when using less immunogenic antigens (Vessely, C et al. (2009) Journal of pharmaceutical sciences 98: 2970-2993), to decrease vaccine toxicity by diminishing the need for higher vaccine dosages, or reduce the need for repeated boosting (Ahmed, S S et al. (2011) Science translational medicine 3: 93rv92).

Many significant cellular functions in bacteria, including regulation of motile/sessile phenotypes, virulence capabilities, and global gene expression are mediated by the second messenger bis-(3′-5′)-cyclic-dimeric-guanosine monophosphate (c-di-GMP) (Gomelsky, M (2012) J Bacteriol 194: 911-913). C-di-GMP is generated by diguanylate cyclase (DGC) enzymes combining two guanosine-5′-triphosphate (GTP) molecules (Krasteva, P V et al. (2012) Protein Sci 21: 929-948). In the mammalian cytosol, the presence of c-di-GMP molecules can be detected by nucleotide sensors, including absent in melanoma 2 (AIM2) (Jones, J W et al. (2010) Proc Natl Acad Sci USA 107: 9771-9776), the DEAD box-containing helicase (DDX41) (Parvatiyar, K et al. (2012) Nat Immunol 13: 1155-1161), and stimulator of interferon genes (STING), each of which directly binds to c-di-GMP, resulting in the increased expression of type I interferons (IFNs) and other innate immune responses (Burdette, D L and R. E. Vance (2013) Nat Immunol 14: 19-26; McWhirter, S M et al. (2009) J Exp Med 206: 1899-1911).

The direct administration of c-di-GMP has been shown to induce innate immune responses that can enhance protection of mice against challenges with Klebsiella pneumoniae (Karaolis, D K et al. (2007) Infect Immun 75: 4942-4950), Staphylococcus aureus (Brouillette, E et al. (2005) Antimicrob Compositions Chemother 49: 3109-3113), methicillin-resistant S. aureus (MRSA) (Hu, D L et al. (2009) Vaccine 27: 4867-4873), Bordetella pertussis (Elahi, S et al. (2014) PLoS One 9: e109778), Streptococcus pneumoniae (Yan, H et al. (2009) Biochem Biophys Res Commun 387: 581-584), and avian influenza A/H5N1 (Pedersen, G K et al. (2011) PLoS One 6: e26973; Svindland, S C et al. (2013) Influenza Other Respir Viruses 7: 1181-1193). Specifically, following intranasal challenge with B. pertussis in BALB/c mice, c-di-GMP induced production of cytokines such as IFN-γ, TNF-α, IL-6, and the chemokine MCP-1 in lung tissue (Elahi, S et al. (2014) PLoS One 9: e109778). Recently, the ability of c-di-GMP to cause robust induction of IFN-β has been shown to attenuate experimental autoimmune encephalitis (EAE) progression and onset through the induction of T regulatory (Treg) cells, which suppress helper/effector T cell responses (Huang, L et al. (2013) J Immunol 191: 3509-3513; Lemos, H et al. (2014) J Immunol 192: 5571-5578).

The ability of c-di-GMP to trigger mammalian inflammatory responses has recently been harnessed for potential use as a promising vaccine adjuvant (Karaolis, D K. et al. (2007) J Immunol 178: 2171-2181). Several studies suggest that inclusion of c-di-GMP in vaccine formulations can improve vaccine efficacy so as to provide immune protection against various bacterial infections (Elahi, S et al. (2014) PLoS One 9: e109778; Fatima, M et al. (2013) Poult Sci 92: 2644-2650), and cancers (Miyabe, H et al. (2014) J Control Release 184: 20-27; Chandra, D et al. (2014) Cancer Immunol Res 2: 901-910; Ohkuri, T et al. (2014) Cancer Immunol Res 2: 1199-1208). Local co-administration (intranasal and sublingual) of H5N1 virosomes and c-di-GMP to BALB/c mice resulted in strong H5N1-specific B cell and T cell adaptive immunity, but the intramuscular (i.m.) route of vaccination resulted in significantly less protection (Pedersen, G K et al. (2011) PLoS One 6: e26973). A liposome-based delivery system that improved c-di-GMP cell uptake in vivo resulted in IFN-β induction and enhanced tumor-specific cytotoxic T cell activity associated with regression of tumor growth in mice (Miyabe, H et al. (2014) J Control Release 184: 20-27). However, this study suggests that pure extracellular c-di-GMP does not efficiently enter target cells.

Because c-di-GMP activates a robust immune response, there has been ongoing focus on using c-di-GMP as an adjuvant to improve vaccine efficacy (Chen W X et al. (2010) Vaccine 28:3080-3085) and delivering or synthesizing c-di-GMP directly within host cells to stimulate innate immunity. Adjuvants are compounds administered alongside vaccine antigens for the purpose of enhancing the longevity, potency, or reducing the effective dose of the antigen without introducing toxic side effects. This is accomplished by stimulating the innate arm of the immune system, resulting in increased cytokine and chemokine production and upregulation of proinflammatory genes (Mosca F et al. (2008) Proc. Natl. Acad. Sci. U.S.A 105:10501-10506), which then enhances antigen recognition and response (Coffman R L et al. (2010) Immunity 33:492-503). The development of novel adjuvants may be critical to the success of vaccines targeting diseases for which vaccinations have previously failed such as Clostridium difficile, human immunodeficiency virus, malaria, and cancer. Despite the demand, currently there are few adjuvants approved for human use. The most commonly used adjuvants are aluminum-salt (Alum)-based; however, these adjuvants suffer drawbacks including local reactions to administration, inadequate T-cell responses, allergic IgE-type responses, and are ineffective with specific types of antigens (Gupta R K (1998) Adv. Drug Deliv. Rev. 32:155-172). Other less-commonly utilized adjuvants include oil and water emulsions, lipopolysacharide derivatives, self-assembling viral nanoparticles, and cholera toxin B subunit (Gupta R K (1998) Adv. Drug Deliv. Rev. 32:155-172). While each adjuvant offers different advantages and disadvantages, there is a large demand for novel adjuvants and compositions that can be paired with and improve vaccine antigens.

In addition to cyclic di-GMP, additional cyclic di-nucleotides have similarly been shown to bind to eukaryotic cytoplasmic receptors, such as STING, to stimulated a Type-I interferon response. Cyclic di-AMP (c-di-AMP) is an additional second messenger synthesized in bacteria by diadenylate cyclase (DAC) domain containing enzymes that has important roles in cell-wall and metabolic homeostatis (Commichau F. M. et. al. (2015) Mol Microbiol. (2):189-204). C-di-AMP is secreted by invasive bacterial pathogens such as Listeria monocytogenes and Chlamydia trachomatis to upregulate inflammatory responses via STING (Barker J R et. al. (2013) MBio. 4(3):e00018-13; Woodward J J et. al. (2010) Science 328(5986):1703-5). A third cyclic di-nucleotide, cyclic GMP-AMP (cGAMP), which is synthesized by both bacteria and eukaryotes in a different isomeric form, also activates STING-dependent inflammation. cGAMP was first shown to be synthesized by the enzyme DncV in the bacterial pathogen Vibrio cholerae (Davies B. W. et. al. (2012) Cell. 149(2):358-70) where it controls chemotaxis and intestinal colonization. cGAMP has not been widely studied, but a recent report indicates that it is important in exoelectrogenesis in Geobacter species (Nelson J. W. et. al. (2015) Proc Nat Acad Sci 112(17):5389-94). All bacterial cyclic di-nucleotides including c-di-GMP, c-di-AMP, and cGAMP exists as cyclic rings with two 3′-5′ phosphodiester linkages. Recently, the eukaryotic protein cGAS, which is well known to activate Type I interferon pathways in response to cytoplasmic DNA, was shown to synthesize cGAMP with a mixed ring linkage of 2′-5′ and 3′-5′ (cGAMP-ML) (Sun L. et. al. (2013) Science. 339(6121):786-91; Gao P. (2013) Cell. 153(5):1094-107). cGAMP-ML then binds to STING to induce inflammation. All of these cyclic di-nucleotides are capable of inducing Type I interferon responses in a STING-dependent manner (Yi G. et. al. (2013) PLoS One. 8(10): e77846).

### SUMMARY OF THE INVENTION

The present invention is based, at least in part, on a novel platform to produce cyclic di-nucleotides (e.g., c-di-GMP, c-di-AMP, cGAMP) inside host cells, as an adjuvant to exploit a host-pathogen interaction, that is useful in upregulating, initiating, enhancing, or stimulating an immune response to thereby treat conditions that would benefit from upregulating an immune response (e.g., pathogenic infections and cancers). In one aspect, provided herein are compositions of matter comprising a vector (e.g., any gene therapy vector, including but not limited to, all adenovirus serotypes, similar vectors derived from AAV, retroviruses, lentiviruses, and DNA based vectors, AdVCA0956, or AdVCA0848) having at least one cyclic di-nucleotide synthetase enzyme gene (e.g., DAC, DncV, Hypr-GGDEF, cGAS, DisA, DGCs, Vibrio cholerae DGCs, such as VCA0956 or VCA0848). Numerous embodiments are described herein that can be applied to any aspect of the present invention or embodiment thereof. For example, in one embodiment, pharmaceutical compositions, vaccines, and adjuvants comprising the vectors of the present invention, are provided. In another embodiment, co-administration of a combination vaccine comprising the vectors of the present invention and an extracellular antigen (Ag) (e.g., viral-associated antigen, bacterial-associated antigen, tumor-associated antigen, such as ovalbumin, Clostridium difficile-derived Toxin B or Toxin A, or HIV-1 derived Gag antigen), is provided. Any of the aforementioned compostions, when introducted in vitro and in vivo, markedly increases cycli di-nucleotide (e.g., c-di-GMP, c-di-AMP, cGAMP) levels and stimulates immune responses (e.g., the innate, adaptive, or humoral immune response).

One aspect of the invention relates to a vector comprising at least one cyclic di-nucleotide synthetase enzyme gene. In some embodiments, the vector is a gene-therapy vector. In another embodiment, the vector is selected from the group consisting of adenovirus, adeno-associated virus (AAV), retrovirus, and lentivirus. In yet another embodiment, the vector is a DNA-based vector. In some embodiments, the vector is an adenoviral vector. In another embodiment, the vector is a replication defective adenoviral vector. In yet another embodiment, the at least one cyclic di-nucleotide synthetase enzyme gene is derived from a bacterial, fungal, protozoal, viral, or pathogenic strain. In some embodiments, the at least one cyclic di-nucleotide synthetase enzyme gene is derived from a bacterial strain. In another embodiment, the bacterial strain is Vibrio cholerae. In some embodiments, the at least one cyclic di-nucleotide synthetase enzyme gene is selected from the group consisting of diadenylate cyclase (DAC), DncV, Hypr-GGDEF, DisA, cGAS, and diguanylate cyclase (DGC). In another embodiment, the at least one cyclic di-nucleotide synthetase enzyme gene is DGC. In yet another embodiment, the DGC comprises a sequence which is at least 80% identical to the sequences set forth in Table 1. In some embodiments, the DGC gene is VCA0956 gene. In another embodiment, the VCA0956 gene comprises a nucleotide sequence which is at least 80% identical to SEQ ID NO: 33. In yet another embodiment, the DGC gene is VCA0848 gene. In some embodiments, the VCA0848 gene comprises a nucleotide sequence which is at least 80% identical to SEQ ID NO: 68. In another embodiment, the vector comprises an adenovirus selected from non-human, human adenovirus serotype, or any adenovirus serotype developed as a gene transfer vector. In still another embodiment, the non-human adenovirus comprises an adenovirus selected from chimp, equine, bovine, mouse, chicken, pig, or dog. In some embodiments, the adenovirus is human adenovirus serotype 5. In some embodiments, the adenovirus has at least one mutation or deletion in at least one adenoviral gene. In another embodiment, the adenoviral gene is selected from the group consisting of E1A, E1B, E2A, E2B, E3, E4, L1, L2, L3, L4, and L5. In yet another embodiment, the adenovirus has a deletion in E1A, E1B, and E3, or combinations thereof. In some embodiments, the at least one cyclic di-nucleotide synthetase enzyme gene is operatively linked to a transcriptional and translational regulatory sequences.

Another aspect of the invention relates to a combination comprising any of the aforementioned vectors. In some embodiments, the combination comprises at least one therapeutic agent. In some embodiments, the agent is another vaccine, an immunemodulatory drug, a checkpoint inhibitor, or a small molecule inhibitor. In some embodiments, the combination further comprises a therapy for immune events. In some embodiments, the therapy is irradiation.

Yet another aspect of the invention relates to a pharmaceutical composition comprising any of the aforementioned vectors, and a pharmaceutically acceptable composition selected from the group consisting of excipients, diluents, and carriers. In some embodiments, the pharmaceutical composition comprises the vector at a purity of at least 75%.

Still another aspect of the invention relates to an adjuvant comprising any of the aforementioned vectors. Another aspect of the invention relates to a vaccine comprising any of the aforementioned vectors, any of the aforementioned pharmaceutical compositions, or any of the aforementioned adjuvants. In some embodiments, the vaccine further comprises an antigen. In some embodiments, the antigen is provide in a second adenoviral vector. In yet some embodiment, the antigen is immunogenic. In some embodiments, the antigen is an extracellular antigen. In still another embodiment, the antigen is a viral-associated antigen, pathogenic-associated antigen, protozoal-associated antigen, bacterial-associated antigen, fungal antigen, or tumor-associated antigen. In some embodiments, the antigen is selected from the group consisting of Ovalbumin (OVA)-specific, HIV-1-derived Gag Ag, Clostridium difficile-derived toxin B, and Clostridium difficile-derived toxin A.

Yet another aspect of the invention relates to a method of inducing or enhancing an immune response in a mammal, comprising: administering to the mammal a pharmaceutically effective amount of any of the aforementioned vaccines such that the immune response is enhanced or stimulated.

Another aspect of the invention relates to a method of treating a mammal having a condition that would benefit from upregulation of an immune response comprising administering to the subject a therapeutically effective amount of any of the aforementioned vaccines such that the condition that would benefit from upregulation of an immune response is treated. In some embodiments, the method further comprises administering one or more additional compositions or therapies that upregulates an immune response or treats the condition. In another embodiment, the one or more additional compositions or therapies is selected from the group consisting of anti-viral therapy, immunotherapy, chemotherapy, radiation, and surgery. In yet another embodiment, the condition that would benefit from upregulation of an immune response is selected from the group consisting of cancer, a viral infection, a bacterial infection, fungal infection, and a protozoan infection. In still another embodiment, the immune response is the innate immune response, adaptive immune response, or humoral immune response. In some embodiments, the vaccine increases or stimulates cyclic di-GMP (c-di-GMP), cyclic di-AMP (c-di-AMP), cyclic GMP-AMP (cGAMP), any cyclic di-nucleotide, or combinations therof, levels in said mammal. In another embodiment, the vaccine increases or stimulates the secretion of cytokines and chemokines. In yet another embodiment, the cytokines and chemokines are selected from the group consisting of IFN-β, IL-1α, IL-4, IL-6, IL12-p40, IFN-γ, G-CSF, Eotaxin, KC, MCP-1, MIP-1α, MIP-1β, and RANTES. In still another embodiment, the vaccine increases or stimulates an immune response selected from the group consisting of DC maturation, NK cell response, T-cell response, and B-cell response, or combination thereof. In some embodiments, the immune response increases the population of immunce cells selected from the group consisting of CD86+CD11c⁺CD11b-DCs, CD69⁺ NK1.1⁺ CD3⁻ NK cells, CD69⁺ CD19⁺ CD3⁻ B cells, CD69⁺ CD3⁺ CD8⁻ T cells, and CD69⁺ CD3⁺ CD8⁺ T cells, or combinations thereof. In another embodiment, the subject is a mammal.

In some embodiments, the mammal is an animal model of the condition. In yet another embodiment, the mammal is a human. In some embodiments, the mammal is an avian species. In still another embodiment, the avian species is G. gallus, or eggs derived therefrom. In some embodiments, the vaccine is administered intradermally, intramuscularly, intraperitoneally, intratumorally, peritumoroally, retroorbiatlly, or intravenously via injection. In another embodiment, the vaccine is administered concomitantly or conjointly. In yet another embodiment, the first vector comprising the cyclic di-nucleotide synthetase enzyme gene lowers the effective dose for the second vector comprising the antigen. In still another embodiment, the administration is repeated at least once. In some embodiments, the effective amount is from about 1×10⁶ vp to about 5×10¹¹ vp. In another embodiment, the effective amount is from about 1×10⁶ vp to about 5×10⁹ vp. In yet another embodiment, the effective amount is about 1×10⁶ vp, about 1×10⁷ vp, about 1×10⁸ vp, or about 5×10⁹ vp. In some embodiments, the effective amount is about 5×10⁹ vp. In another embodiment, the effective amount is about 1×10¹⁰, about 0.5×10¹¹, about 1×10¹¹, about 2×10¹¹, about 3×10¹¹, about 4×10¹¹, or about 5×10¹¹ viral particles (vp). In some embodiments, the effective amount is about 2×10¹¹ vp. In yet another embodiment, the effective amount is about 10 μg/mL, about 20 μg/mL, about 30 μg/mL, about 40 μg/mL, about 50 μg/mL, about 60 μg/mL, about 70 μg/mL, about 80 μg/mL, about 90 μg/mL, about 100 μg/mL, about 125 μg/mL, about 150 μg/mL, about 175 μg/mL, and 200 μg/mL. In some embodiments, the effective amount is about 100 μg/mL.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF FIGURES

FIG. 1 contains 2 panels, identified as panels A and B, depicting LC-MS/MS used to quantify c-di-GMP in HeLa cells. Panel A shows that HeLa cells were transfected with plasmid vectors containing the VCA0956 allele or the active site mutant allele, VCA0956*. Bars represent the mean of 5 independent cultures. Panel B shows c-di-GMP in HeLa cells cultured in T75 flasks and transfected with plasmid vectors containing the VCA0956 allele at 24 and 48 hours. Bars represent the mean of independent cell cultures (24 hours, N=3; 48 hours, N=2).

FIG. 2 depicts HeLa cells infected with 500 M.O.I. Ad5 vectors. Bars represent the mean of 3 independent cultures; error bars indicate standard deviation. bd indicates below detection.

FIG. 3 contains 2 panels, identified as panels A and B, depicting infection of Ad5-VCA0956 in a murine system. Panel A shows that after 24 hours qPCR was used to quantify Ad5 genomes in liver cells (black) or spleen cells (checkered). Data were normalized to internal GADPH control. Panel B depicts LC-MS/MS was used to quantify c-di-GMP extracted from the liver (black) or spleen (checkered). Bars represent the mean of 3 independent mouse samples; error bars indicate standard deviation. bd indicates below detection. Panel B depicts that in the presence of rIFNg, 72.9% of the cells was PE positive.

FIG. 4 contains 3 panels, identified as panels A, B and C, depicting qRT-PCR of mouse liver gene transcripts 24 hours after infection with Ad5 vectors. The data were normalized to internal GADPH control. Fold change indicates each value normalized to values measured from mock treated mice. Results are separated into liver gene expression increased by Ad5-VCA0956 (Panel A), decreased by Ad5-VCA0956 (Panel B), or unaffected by Ad5-VCA0956 (Panel C). Bars represent the mean of 3 independent mouse samples; error bars indicate standard deviation. Brackets indicate statistical significance, which was determined using a two-tailed Student's t-test (P<0.05).

FIG. 5 depicts IFN-β concentrations in the plasma of mice infected with Ad5 vectors. Mice were infected with either Ad5-Null (stripes), Ad5-VCA0956 (black), or Ad5-VCA0956* (grey). At 6 and 24 hours, IFN-β was quantified from plasma samples. Brackets indicate statistical significance, which was determined using a one-way ANOVA test combined with a Bonferroni posttest (**p<0.01). Bars indicate the mean of independent mouse plasma samples (n=2: Mock, Ad5-Null; n=3: Ad5-VCA0956, Ad5-VCA0956*) and error bars indicate standard deviation. bd indicates below detection.

FIG. 6 contains 12 panels, identified as panels A, B, C, D, E, F, G, H, I, J, K, and L, depicting plasma cytokine and chemokine levels in mice infected with Ad5 vectors. Mice were infected with either Ad5-Null (stripes), Ad5-VCA0956 (black), or Ad5-VCA0956* (grey). At 6 and 24 hours, cytokines and chemokines were quantified from plasma samples. Brackets indicate statistical significance, which was determined using a two-way ANOVA test combined with a Bonferroni posttest (*p<0.05; **p<0.01). Bars indicate the mean of independent mouse plasma samples (n=2: Mock, Ad5-Null; n=3: Ad5-VCA0956, Ad5-VCA0956*) and error bars indicate standard deviation. IL-1α (Panel (A)), IFN-γ (Panel (B)), MCP-1 (Panel (C)), IL-4 (Panel (D)), G-CSF (Panel (E)), MIP-1α (Panel (F)), IL-6 (Panel (G)), Eotaxin (Panel (H)), MIP-1β (Panel (I)), IL-12p40 (Panel (J)), KC (Panel (K)), and RANTES (Panel (L)).

FIG. 7 contains two panels, identified as panels A and B, depicting C. difficile TA-specific IgG from the plasma of mice I.M. vaccinated with (A) 1×10⁷ vp Ad5-TA and Ad5-VCA0956 or (B) 5×10⁹ vp Ad5-TA and Ad5-VCA0956 (both 14 d.p.i.) was quantified using an ELISA assay. The OD₄₅₀ was measured at various plasma dilutions. Each point represents the mean of 6 independent mouse plasma samples, and error bars indicate standard deviation.

FIG. 8 shows IFN-γ ELISPOT analysis of mice vaccinated with Ad5-TA and Ad5 vectors. Mice were administered (I.M.) varying doses of both Ad-TA and either Ad-VCA0956 (black) or Ad-VCA0956* (grey). After 14 days, splenocytes were ex vivo stimulated with a C. difficile specific peptide and the number of IFNγ secreting splenocytes was determined using ELISPOT. Each point represents an individual mouse. Lines indicate the mean of the replicates, and error bars indicate standard error. * indicates statistical significance using a two-way ANOVA test combined with a Bonferroni posttest (P<0.05).

FIG. 9 shows that active VCA0848 produces significant amounts of c-di-GMP in mice. Male 6-8 weeks old BALB/c WT mice were retro-orbitally i.v. injected with 2×10⁹ vps/mouse of AdVCA0848 (n=3); or 2×10¹¹ vps/mouse of AdVCA0848^{mut} (n=3) or AdVCA0848 (n=3). As a control not injected (naïves) mice (n=2) were included. At 24 hpi mice were sacrificed and liver samples were collected, and immediately snap frozen in liquid nitrogen. 20 mg of liver samples were used for c-di-GMP extraction as described in methods section. C-di-GMP production measurements were performed using liquid chromatography coupled with tandem mass spectrometry (LC-MS/MS). Bars represent mean±SD from different groups. Statistical analysis was completed using One Way ANOVA followed by a Student-Newman-Keuls post-hoc test. A value of p<0.05 was deemed statistically significant. “bd”, below detection.

FIG. 10 contains 6 panels, identified as panels A, B, C, D, E, and F, depicting that AdVCA0848 stimulates strong induction of IFN-β and activates innate and adaptive immune cells. Male 6-10 weeks old C57BL/6 WT mice (n=4) were i.v. injected (retro-orbitally) with 1×10¹⁰ vps/mouse of AdNull, AdVCA848, or not injected (naive) as control. At 6 hpi mice were sacrificed and spleens and blood samples were obtained. Panel A shows an ELISA-based assay to determine the amount of IFN-β produced in plasma (diluted 1:2) from naive, mice injected with AdNull, AdVCA0848. Splenocytes harvested and FACS analysis conducted as described in methods and materials. Effects of AdNull and AdVCA0848 (with representative results) on the activation of CD86⁺CD11c⁺CD11b− DCs (Panel B), CD69⁺ NK1.1⁺ CD3⁻ NK cells (Panel C), CD69⁺ CD19⁺ CD3⁻ B cells (Panel D), CD69⁺ CD3⁺ CD8⁻ T cells (Panel E), and CD69⁺ CD3⁺ CD8⁺ T cells (Panel F). Bars with the indicated colors represent mean±SD. Statistical analysis was completed using One Way ANOVA followed by a Student-Newman-Keuls post-hoc test. A value of p<0.05 was deemed statistically significant. The (**) and (***) denote significance over naïve animals p<0.05 and p<0.001, respectively.

FIG. 11 contains 4 panels, identified as panels A, B, C, and D, depicting that AdVCA0848 enhances OVA-specific adaptive T cell responses. Male 6-10 weeks old C57BL/6 mice (n=5) were injected with OVA alone, OVA+AdVCA0848, OVA+AdNull, or not injected as described in materials and methods. At 14 dpi, mice were sacrificed and splenocytes at 1×10⁶ cells/well were ex vivo stimulated with MHC class I-restricted OVA-derived peptide SIINFEKL, OVA protein, heat-inactivated Ad5 particles, or with only media (unstimulated). The ELISPOT assays for IFN-γ (Panels A and B) and IL-2 (Panels C and D) were performed. Bars with the indicated colors represent mean±SD for samples stimulated with the indicated stimulations. Results are representative of two independent experiments. Statistical analysis was completed using One Way ANOVA followed by a Student-Newman-Keuls post-hoc test. A value of p<0.05 was deemed statistically significant. The (**) and (***) denote significance over naïve animals p<0.05 and p<0.001, respectively.

FIG. 12 contains 4 panels, identified as panels A, B, C, and D, depicting that AdVCA0848 enhances OVA-specific adaptive B cell responses. Male 8-10 weeks old C57BL/6 mice (n=5) were injected with OVA+AdNull, OVA+AdVCA0848, or not injected (naïve) as described in materials and methods. Panels A and B show that at 6 dpi, mice were retro-orbitally bleeded to determine OVA and Ad5-specific B cell response by ELISA-based measurement for total IgG with the indicated plasma dilutions. Panels C and D shows that at 14 dpi, mice were sacrificed; blood samples obtained, and plasma samples were prepared and used for ELISA-based measurement for total OVA and Ad5-specific IgG with the indicated plasma dilutions. Bars with the indicated colors represent mean±SD for samples from different groups. Results are representative of two independent experiments. Statistical analysis was completed using One Way ANOVA followed by a Student-Newman-Keuls post-hoc test. A value of p<0.05 was deemed statistically significant.

FIG. 13 contains 2 panels, identified as panels A and B, depicting that co-injecting AdVCA0848 and AdGag results in significant inhibitory effects of Gag-specific T cell responses. Female 6-8 weeks old BALB/c mice (n=4) were i.m. co-injected in the tibialis anterior with viral particles of AdGag (5×10⁶ vps/mouse) along with 3 different doses (5×10⁷, 5×10⁸, or 5×10⁹ vps/mouse) of either AdNull or AdVCA0848, in the presence of an uninjected group of mice as control naive. At 14 dpi, mice were sacrificed and splenocytes (at 5×10⁵ cells/well) were ex vivo stimulated with the 15-mer HIV/Gag-derived immunogenic peptides AMQ (Panel A), or with UV-inactivated adenoviruses (Panel B) for the IFN-γ ELISPOT assays as described in materials and methods. Bars with the indicated colors represent mean±SD. Results are representative of two independent experiments. Statistical analysis was completed using One Way ANOVA followed by a Student-Newman-Keuls post-hoc test. A value of p<0.05 was deemed statistically significant. The (**) and (***) denote significance over naïve animals p<0.05 and p<0.001, respectively. The (a) denote significance over AdVCA0848 at the dose of 5×10⁹ vps/mouse (p<0.05).

FIG. 14 contains 3 panels, identified as panels A, B, and C, depicting that co-injecting AdVCA0848 and AdGag results in significant inhibitory effects of Gag-specific CD8+T cells. Female 6-8 weeks old BALB/c mice (n=4) were i.m. co-injected in the tibialis anterior with viral particles of AdGag (5×10⁶ vps/mouse) along with 3 different doses (5×10⁷, 5×10⁸, or 5×10⁹ vps/mouse) of either AdNull or AdVCA0848, in the presence of an uninjected group of mice as control naive. At 14 dpi, mice were sacrificed and splenocytes harvested and used at 1×10⁶ cells/well for tetramer staining using PE-labeled MHC class I tetramer folded with the AMQ peptide as described in materials and methods followed by FACS analysis for Tet* Gag-specific CD8⁺ T cells (Panel A). Multi-parameter staining was conducted to determine the overall frequency of IFN-γ (Panel B) and TNF-α (Panel C) producing CD8⁺ T cells followed by FACS analysis conducted on BD LSRII flow cytometer as described in methods and materials. Results are representative of two independent experiments. Bars with the indicated colors represent mean±SD. Statistical analysis was completed using One Way ANOVA followed by a Student-Newman-Keuls post-hoc test. A value of p<0.05 was deemed statistically significant. The (**) and (***) denote significance over naïve animals p<0.05 and p<0.001, respectively. The (a) denote significance over AdVCA0848 dose of 5×10⁹ vps/mouse (p<0.05).

FIG. 15 contains 4 panels, identified as panels A, B, C, and D, depicting that co-injecting AdVCA0848 resulted in significant inhibition of Gag and ToxB-specific B cell response. Female 6-8 weeks old BALB/c mice (n=4) were i.m. co-injected in the tibialis anterior with the indicated viral injections and as described in materials and methods of AdVCA0848 along with either AdGag or AdToxB in the presence of uninjected mice control naïves. At 14 dpi, mice were sacrificed and plasma samples collected. Total IgG levels of Gag-specific (plasma dilution 1:25) antibodies (Panel A) or Ad5-specific (plasma dilution 1:400) (Panel B) were measured to determine the effect of indicated does of AdVCA0848 on Gag-specific B cell response by ELISA. ELISA was also used to determine the effect of AdVCA0848 on ToxB-specific (Panel C) and Ad5-specific (Panel D) B cell response by measuring total IgG levels at the indicated plasma dilutions. Results are representative of two independent experiments. Bars with the indicated colors represent mean±SD. Statistical analysis was completed using One Way ANOVA followed by a Student-Newman-Keuls post-hoc test. A value of p<0.05 was deemed statistically significant. The (**) and (***) denote significance over naïve animals p<0.05 and p<0.001, respectively.

FIG. 16 shows co-administration of AdGag and AdVCA0848 does not inhibit the translation of Gag protein. Male 6-8 weeks old BALB/c WT mice were retro-orbitally i.v. injected with 1×10¹¹1×10¹¹ vps/mouse of AdGag alone (n=3), or co-injected with 1×10¹¹ vps/mouse AdVCA0848 (n=4), AdNull (n=3), or not injected (naïves) (n=3) as control.

FIG. 17 shows that AdVCA0848 produces significant amounts of c-di-GMP in mice which surpasses that produced by AdVCA0956. Male 6-8 weeks old BALB/c WT mice were retro-orbitally i.v. injected with 2×10¹¹ vps/mouse of AdVCA0956 (n=4), AdVCA0848 (n=4), AdNull (n=3), or not injected (naïves) (n=3) as control. At 24 hpi mice were sacrificed and liver samples were collected, and immediately snap frozen in liquid nitrogen. 20 mg of liver samples were used for c-di-GMP extraction as described in methods section. C-di-GMP production measurements were performed using liquid chromatography coupled with tandem mass spectrometry (LC-MS/MS). Bars represent mean±SD from different groups. Statistical analysis was completed using One Way ANOVA followed by a Student-Newman-Keuls post-hoc test. A value of p<0.05 was deemed statistically significant. “bd”, below detection.

FIG. 18 contains 6 panels, identified as panels A, B, C, D, E, and F, depicting that active VCA0848 stimulates strong induction of IFN-β and activates innate and adaptive immune cells. Male 6-10 weeks old C57BL/6 WT mice (n=3) were retro-orbitally i.v. injected with 1×1010 vps/mouse of AdVCA0848^{mut}, AdVCA848, or not injected (naive) as control. At 6 hpi mice were sacrificed and spleens and blood samples were obtained. Panel A shows an ELISA-based assay to determine the amount of IFN-β produced in plasma (diluted 1:2) from naive, mice injected with AdVCA0848^{mut}, or AdVCA0848. Splenocytes harvested and FACS analysis conducted as described in methods and materials. Effects of AdVCA0848^{mut} or AdVCA0848 (with representative results) on the activation of CD86⁺ CD11c⁺CD11b−DCs (Panel B), CD69⁺ NK1.1⁺ CD3⁻ NK cells (Panel C), CD69⁺ CD19⁺ CD3⁻ B cells (Panel D), CD69⁺ CD3⁺ CD8⁻ T cells (Panel E), and CD69⁺ CD3⁺ CD8⁺ T cells (Panel F). Bars with the indicated colors represent mean±SD. Statistical analysis was completed using One Way ANOVA followed by a Student-Newman-Keuls post-hoc test. A value of p<0.05 was deemed statistically significant.

FIG. 19 shows that AdVCA0848 enhances OVA-specific adaptive B cell responses when co-injected with OVA. Male 8-10 weeks old C57BL/6 mice (n=5) were injected with OVA alone, OVA+AdNull, OVA+AdVCA0848, or not injected (naïve) as described in materials and methods. At 14 dpi, mice were sacrificed; blood samples obtained, and plasma samples were prepared and used for ELISA-based measurement for total OVA and Ad5-specific IgG (plasma dilution 1:1000). Bars with the indicated colors represent mean±SD for samples from different groups. Results are representative of two independent experiments. Statistical analysis was completed using One Way ANOVA followed by a Student-Newman-Keuls post-hoc test. A value of p<0.05 was deemed statistically significant. The (**) and (***) denote significance over naïve animals p<0.05 and p<0.001, respectively.

FIG. 20 contains 3 panels, identified as panels A, B, and C, depicting that active VCA0848 results in significant inhibitory effects of Gag-specific T cell and B cell responses and significant enhancement of Ad5-specific T cell and B cell response by AdVCA0848 and AdGag co-administration. Female 6-8 weeks old BALB/c mice (n=3) were i.m. co-injected in the tibialis anterior with viral particles of AdGag (5×10⁶ vps/mouse) along with 5×10⁹ vps/mouse of either AdVCA0848^{mut} or AdVCA0848, in the presence of an uninjected group of mice as control naïve (n=2). At 14 dpi, mice were sacrificed and peripheral blood and spleens were collected. Panel A shows that splenocytes (at 1×10⁶ cells/well) were ex vivo stimulated with the 15-mer HIV/Gag-derived immunogenic peptides AMQ or with UV-inactivated adenoviruses for the IFN-γ ELISPOT assays as described in materials and methods. Total Gag-specific (Panel B), or Ad5-specific (Panel C) IgG levels at the indicated plasma dilutions were measured to determine the effect of indicated does of AdVCA0848 and AdVCA0848^{mut} on Gag-specific B cell response by ELISA. Bars with the indicated colors represent mean±SD. Statistical analysis was completed using One Way ANOVA followed by a Student-Newman-Keuls post-hoc test. A value of p<0.05 was deemed statistically significant.

FIG. 21 depicts the conserved protein domain for COG2199 (GGDEF domain, diguanylate cyclase (c-di-GMP synthetase) or its enzymatically inactive variants) provided from http://www.ncbi.nlm.nih.gov/Structure/cdd/cddsrv.cgi?ascbin=8&maxaln=10&seltype=2& uid=COG2199.

FIG. 22 depicts a sequence alignment of various DncV homologs from bacteria (from Figure S1 of Kranzusch P J et al. (2014) Cell 158(5):1011-21).

FIG. 23 lists the putative HYPR domains in Geobacter and Pelobacter and identifies the conserved residues. The bottom sequence (ccPleD/1-454) is a known GGDEF from Caulobacter crescentus for comparison.

Note that for every figure containing a histogram, the bars from left to right for each discreet measurement correspond to the figure boxes from top to bottom in the figure legend as indicated.

### DETAILED DESCRIPTION OF THE PRESENT INVENTION

The present invention is based, at least in part, on a novel approach to produce cyclic di-nucleotides (e.g., c-di-GMP, c-di-AMP, c-di-GAMP, or others) inside host cells as an adjuvant to exploit a host-pathogen interaction and initiate an innate immune response. Provided herein are compositions of matter comprising a vector (e.g., any gene therapy vector) having at least one cyclic di-nucleotide synthetase enzyme. As described herein, in some embodiments, c-di-GMP can be synthesized in vivo by transducing a diguanylate cyclase (DGC) gene (e.g., Vibrio cholerae DCGs, such as VCA0956 or VCA0848), into mammalian cells using a non-replicating adenovirus serotype 5 (Ad5) vector (e.g., AdVCA095 or AdVCA0848). Expression of the DGC led to the production of c-di-GMP in vitro and in vivo, and this was able to alter pro-inflammatory gene expression in murine tissues and increase the secretion of numerous cytokines and chemokines when administered into animals. Co-expression of the DGC modestly increased T-cell responses to a Clostridium difficile antigen expressed from an adenovirus vaccine. This adenovirus c-di-GMP delivery system offers a novel method to administer c-di-GMP as an adjuvant to stimulate innate immunity during vaccination. In some embodiments, AdVCA0848 is more potent than AdVCA0956 and produces elevated amounts of c-di-GMP when expressed in mammalian cells in vivo. As described herein, this novel platform improves induction of type I interferon R (IFN-0) and activation of innate and adaptive immune cells early after administration into mice as compared to control vectors. Co-administration of the extracellular antigen (e.g., protein ovalbumin (OVA)) and AdVCA0848 adjuvant significantly improved OVA-specific T cell responses as detected by IFN-γ and IL-2 ELISPOT, while also improving OVA-specific humoral B cell adaptive responses.

The data presented herein confirm that in vivo synthesis of cyclic di-nucleotides (e.g., c-di-GMP) stimulates strong innate immune responses that correlate with enhanced adaptive immune responses to concomitantly administered extracellular antigen, which can be utilized as an adjuvant to heighten effective immune responses for protein-based vaccine platforms against pathogenic infections and cancers. An extension of the compositions and methods decribed herein is to similarly express other cyclic di-nucleotide synthetase enzymes such as those containing a DAC domain (Hengge R. et. al. (2016) J Bacteriol. 198(1):15-26), DncV or other enzymes that synthesis bacterial cGAMP (Davies B. W. et. al. (2012) Cell. 149(2):358-70), Hypr-GGDEF enzymes that can make c-di-GMP, c-di-AMP, or cGAMP (Hallberg Z. F. et. al. (2016) Proc Nat Acad Sci 113(7):1790-5.), or cGAS (Sun L. et. al. (2013) Science. 339(6121):786-91; Gao P. (2013) Cell. 153(5):1094-107).

### I. Definitions

The articles “a” and “an” are used herein to refer to one or to more than one (i.e., to at least one) of the gra more than one element. mmatical object of the article. By way of example, “an element” means one element or more than one element.

As used herein, “adenoviruses” are DNA viruses with a 36-kb genome. There are 51 human adenovirus serotypes that have been distinguished on the basis of their resistance to neutralization by antisera to other known adenovirus serotypes. Adenoviruses as used herein encompass non-human or any adenovirus serotype developed as a gene transfer vector. Non-human adenovirus comprises an adenovirus selected from chimp, equine, bovine, mouse, chicken, pig, dog, or any mammalian or non-mammalian species. Although the majority of adenoviral vectors are derived from serotypes 2 and 5, other serotypes may also be used. The wild type adenovirus genome is divided into early (E1 to E4) and late (L1 to L5) genes, e.g., E1A, E1B, E2A, E2B, E3, E4, L1, L2, L3, L4, or L5. Adenovirus vectors can be prepared to be either replication competent or non-replicating. Replication defective adenoviral vectors may comprise at lease one deletion of any of the E1 to E4 or L1 to L5 genes. Replication deficient adenovirus based vectors are described in Hartman Z C et al. (2008) Virus Res. 132:1-14. In some embodiments, the replication defective adenovirus comprises deletions of the E1 and E3 genes. Foreign genes can be inserted into three areas of the adenovirus genome (E1, E3, or E4) as well as behind the major late promoter. The ability of the adenovirus genome to direct production of adenoviruses is dependent on sequences in E1.

Adenovirus vectors transduce large fragments of DNA into a wide range of cells in order to synthesize proteins in vivo, and gene expression can be modulated and even localized to specific cell types. Unlike other types of viral delivery systems, DNA delivered by adenovirus vectors does not integrate into the genome and thus circumvents the danger of insertional mutagenesis (Aldhamen Y A et al. (2011) Front. Immun. 2:1-12). Adenovirus vectors have been shown to induce innate immunity, which is partially due to inducing the STING DNA recognition pathway (Lam E et al. (2013) J. Virol. 88:974-981). Additionally, adenovirus vectors can be produced cost-efficiently in high abundance. Importantly, adenovirus vectors are currently being used in human clinical trials world-wide (Fukazawa T et al. (2010) Int. J. Mol. Med. 25:3-10).

The term “adjuvant” is used in its broadest sense as any substance or composition (e.g., AdVCA0848 or AdVCA0956) which enhances, increases, upwardly modulates or otherwise facilitates an immune response to an antigen be it added exogenously or already present such as a tumor associated antigen. The immune response may be measured by any convenient means such as antibody titre or level of cell-mediated response.

The term “body fluid” refers to fluids that are excreted or secreted from the body as well as fluids that are normally not (e.g., amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, peritoneal fluid, pus, saliva, sebum, semen, serum, sweat, synovial fluid, tears, urine, vaginal lubrication, vitreous humor, vomit). In a one embodiment, body fluids are restricted to blood-related fluids, including whole blood, serum, plasma, and the like.

The terms “cancer” or “tumor” or “hyperproliferative disorder” refer to the presence of cells possessing characteristics typical of cancer-causing cells, such as uncontrolled proliferation, immortality, metastatic potential, rapid growth and proliferation rate, and certain characteristic morphological features. Cancer is generally associated with uncontrolled cell growth, invasion of such cells to adjacent tissues, and the spread of such cells to other organs of the body by vascular and lymphatic menas. Cancer invasion occurs when cancer cells intrude on and cross the normal boundaries of adjacent tissue, which can be measured by assaying cancer cell migration, enzymatic destruction of basement membranes by cancer cells, and the like. In some embodiments, a particular stage of cancer is relevant and such stages can include the time period before and/or after angiogenesis, cellular invasion, and/or metastasis. Cancer cells are often in the form of a solid tumor, but such cells may exist alone within an animal, or may be a non-tumorigenic cancer cell, such as a leukemia cell. Cancers include, but are not limited to, B cell cancer, e.g., multiple myeloma, Waldenström's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, the cancer whose phenotype is determined by the method of the present invention is an epithelial cancer such as, but not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, brenner, or undifferentiated. In some embodiments, the present invention is used in the treatment, diagnosis, and/or prognosis of melanoma and its subtypes.

The term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

The term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “control” refers to any reference standard suitable to provide a comparison. In one embodiment, the control comprises obtaining a “control sample” from which expression product levels are detected and compared to the expression product levels from the test sample. Such a control sample may comprise any suitable sample, including but not limited to a sample from a control cancer patient or healthy patient (can be stored sample or previous sample measurement) with a known outcome; normal tissue or cells isolated from a subject, such as a healthy patient or the cancer patient, cultured primary cells/tissues isolated from a subject such as a normal subject or the cancer patient, adjacent normal cells/tissues obtained from the same organ or body location of the cancer patient, a tissue or cell sample isolated from a healthy subject, or a primary cells/tissues obtained from a depository. In another embodiment, the control may comprise a reference standard expression product level from any suitable source, including but not limited to housekeeping genes, an expression product level range from normal tissue (or other previously analyzed control sample), a previously determined expression product level range within a test sample from a group of patients, or a set of patients with a certain outcome (for example, survival for one, two, three, four years, etc.) or receiving a certain treatment (for example, standard of care cancer therapy). It will be understood by those of skill in the art that such control samples and reference standard expression product levels can be used in combination as controls in the methods of the present invention.

The term “cycli-di-nucleotides,” or c-di-nucleotides as used herein encompasses any cyclic di-nucleotides, including but not limited to, c-di-GMP, c-di-AMP, or cGAMP. C-di-nucleotides have been shown to bind to eukaryotic cytoplasmic receptors, such as STING, to stimulated a Type-I interferon response. All bacterial cyclic di-nucleotides including c-di-GMP, c-di-AMP, and cGAMP exists as cyclic rings with two 3′-5′ phosphodiester linkages.

The term “cyclic di-AMP” refers to a specific bacterial second messenger synthesized in bacteria that has important roles in cell-wall and metabolic homeostatis (Commichau F. M. et. al. (2015) Mol Microbiol. (2):189-204). C-di-AMP has also been shown to be an essential signaling molecule in Staphylococcus aureus (Corrigan R. M. (2013) Proc Natl Acad Sci 110(22):9084-9) and Listeria monocytogenes (Commichau F. M. (2015) Mol Microbiol. 97(2):189-204). C-di-AMP is secreted by invasive bacterial pathogens such as Listeria monocytogenes and Chlamydia trachomatis to upregulate inflammatory responses via STING (Barker J R et. al. (2013) MBio. 4(3):e00018-13; Woodward J J et. al. (2010) Science 328(5986):1703-5).

The term “cyclic di-GMP,” or “c-di-GMP” as used herein is a bacterial specific second messenger that controls a wide range of phenotypes including motility, biofilm formation, and virulence (Romling U et al. (2013) Microbiol. Mol. Biol. Rev. 77:1-52). C-di-GMP was first discovered in 1987 by Benziman et al. (Ross P et al. (1987) Nature 325:279-281), and since has been predicted to be utilized in >75% of all bacteria in representatives from every major bacterial phyla (Seshasayee A S N et al. (2010) Nucleic Acids Res. 38:5970-5981). Diguanylate cyclase enzymes (DGCs) which contain conserved GGDEF domains synthesize c-di-GMP from two GTP molecules. In contrast, c-di-GMP is hydrolyzed by c-di-GMP specific phosphodiesterase enzymes (PDEs) which contain conserved EAL or HD-GYP domains (Romling U et al. (2013) Microbiol. Mol. Biol. Rev. 77:1-52). Bacteria typically contain numerous DGCs and PDEs within their genomes; for example, the marine bacterium Vibrio cholerae encodes 70 predicted c-di-GMP turnover domains (Galperin M Y et al. (2001) FEMS Microbiol. Lett. 203:11-21).

Previous studies indicate that c-di-GMP is a potent stimulator of innate immunity in eukaryotic organisms. This occurs at least in part through the protein STING, which senses pathogen derived nucleic acids in the cytoplasm and subsequently activates a signaling cascade to stimulate a type-I interferon response (McWhirter S M et al. (2009) J. Exp. Med. 206:1899-1911; Sauer J D et al. (2011) Infect. Immun. 79:688-694). Studies show that the presence of c-di-GMP can trigger the production of IL-2, IL-4, IL-5, IL-6, IL-8, IL-12p40, IL-17, IP-10, TNF-α, KC, MIP-1α, MIP-1β, MIP-2, MCP-1, RANTES, IFN-β, IFN-γ, stimulate the NLRP3 inflammasome pathway, and promote the recruitment and activation of macrophages, NK cells, αβ conventional T cells, and enhance DC maturation (Sauer J D et al. (2011) Infect. Immun. 79:688-694; Ebensen T et al. (2007) Vaccine 25:1464-1469; Abdul-Sater A A et al. (2013) EMBO reports 14:900-906; Ebensen T et al. (2007) Clin. Vaccine Immunol. 14:952-958; Karaolis D K R et al. (2007) J. Immunol. 178:2171-2181; Karaolis D K R et al. (2007) Infect. Immun. 75:4942-4950; Yan H B et al. (2009) Biochem. Biophys. Res. Commun. 387:581-584; Gray P M et al. (2012) Cell Immunol. 278:113-119; Blaauboer S M et al. (2014) J. Immunol. 192:492-502). Furthermore, in vivo studies have shown that co-administration of purified c-di-GMP with an antigen confers increased protection of animals in several different murine challenge models, including those utilizing Staphylococcus aureus, Klebsiella pneumoniae, and Streptococcus pneumoniae (Karaolis D K R et al. (2007) J. Immunol. 178:2171-2181; Karaolis D K R et al. (2007) Infect. Immun. 75:4942-4950; Yan H B et al. (2009) Biochem. Biophys. Res. Commun. 387:581-584; Ogunniyi A D et al. (2008) Vaccine 26:4676-4685).

The term “cyclic GMP-AMP” (cGAMP) refers to a second messenger produced by both bacteria and eukaryotic cells (designated as cGMAP-ML). cGAMP has not been extensively studied in bacteria, but it has been shown to regulate virulence and chemotaxis in the bacterial pathogen Vibrio cholerae (Davies B. W. et. al. (2012) Cell. 149(2):358-70) and evidence suggests it could regulate exoelectrogenesis in Geobacter species (Nelson J. W. et. al. (2015) Proc Natl Acad Sci 112(17):5389-94) although this has not been fully demonstrated. All bacterial cyclic di-nucleotides including c-di-GMP, c-di-AMP, and cGAMP exists as cyclic rings with two 3′-5′ phosphodiester linkages. Recently, the eukaryotic protein cGAS, which is well known to activate Type I interferon pathways in response to cytoplasmic DNA, was shown to synthesize cGAMP with a mixed ring linkage of 2′-5′ and 3′-5′ (cGAMP-ML) (Sun L. et. al. (2013) Science. 339(6121):786-91; Gao P. (2013) Cell. 153(5):1094-107). cGAMP-ML then binds to STING to induce inflammation.

The term “cyclic di-nucleotide synthetase enzyme” as used herein refers to a class of enzymes which synthesizes cyclic-di nucleotides, including but not limited to, c-di-AMP, c-di-GMP, or cGAMP. Such cyclic di-nucleotide synthetase enzymes include but are not limited to diguanylate cyclase (DGC), Hypr-GGDEF, diadenylate cyclase (DAC), DncV, cGAS, and DisA (c-di-AMP synthesis). As noted in Burroughs A M et al. (2015) Nucleic Acids Res. 43(22):10633-54: “All synthetases that use NTPs as substrates to generate the above-mentioned cyclic and linear nucleotides belong to just four distinct superfamilies. The classical adenylyl and guanylyl cyclases (Mock M. et al. (1991) J. Bacteriol. 173:6265-6269) and GGDEF domains which generate c-di-GMP (Pei J. et. al. (2001) Proteins 42:210-216) belong to a large superfamily of enzymes that also includes most DNA polymerases, reverse transcriptases, viral RNA-dependent RNA polymerases and T7-like DNA-dependent RNA polymerases. Another distinct, large superfamily of nucleotidyltransferases, also including DNA polymerase ß (polß superfamily) (Aravind L. et al. (1999) Nucleic Acids Res. 27:1609-1618; Kuchta K. et al. (2009) Nucleic Acids Res. 37:7701-7714), contains several nucleotide-generating families; namely the CyaA-like bacterial adenylyl cyclases (Mock M. et al. (1991) J. Bacteriol 173:6265-6269; Aravind L. et al. (1999) Nucleic Acids Res. 27:1609-1618), the cyclic 2′-5′ GMP-AMP synthase (cGAS), bacterial 3′-5′ cGAMP synthetases typified by the V. cholerae DncV (formerly known as VC0179) (Davies. B. W. et al. (2012) Cell 149:358-370; Kato K. et al. (2015) Structure 23:843-850) and 2′-5′A synthetase (oligoadenylate synthetase: OAS). The characterized c-di-AMP synthetases belong to the DisA superfamily, members of which directly monitor DNA integrity via a fused DNA-binding domain (Bejerano-Sagie M. et al. (2006) Cell 125:679-69; Witte G. et al. (2008) Mol. Cell 30:167-178; Oppenheimer-Shaanan Y. et. al (2011) EMBO Rep. 12:594-601; Campos S. S. et al. (2014) J. Bacteriol. 196:568-578).”

Cyclic di-nucleotide synthetase enzyme genes may encompass those derived from any of the V cholerae strains, including but not limited to, O1 str. C6706 Contig_56 (Accession: NZ_AHGQ01000056.1 GI: 480994251); O1 str. C6706 Contig_20 (Accession: NZ_AHGQ01000020.1 GI: 480994215); O1 str. C6706 Contig_30 (Accession: NZ_AHGQ01000030.1 GI: 480994225); O1 str. C6706 Contig_42 (Accession: NZ_AHGQ01000042.1 GI: 480994237); O1 str. C6706 Contig_40 (Accession: NZ_AHGQ01000040.1 GI: 480994235); O1 str. C6706 Contig_37 (Accession: NZ_AHGQ01000037.1 GI: 480994232); O1 str. C6706 Contig_36 (Accession: NZ_AHGQ01000036.1 GI: 480994231); O1 str. C6706 Contig_62 (Accession: NZ_AHGQ01000062.1 GI: 480994257); O1 str. C6706 Contig_27 (Accession: NZ_AHGQ01000027.1 GI: 480994222); O1 biovar El Tor str. N16961 chromosome I (Accession: NC_002505.1 GI: 15640032); O1 biovar El Tor str. N16961 chromosome 2 (Accession: NC_002506.1 GI: 15600771); 2012EL-2176 chromosome 2 (NZ_CP007635.1 GI: 749293683); 2012EL-2176 chromosome 1 (Accession: CP007634.1 GI: 695931389); TSY216 chromosome 1 (Accession: CP007653.1 GI: 861210305); strain ATCC 25874 CFSAN20.contig.1 (Accession: LRIK01000002.1 GI: 977936890); strain ATCC 11629 CFSAN19.contig.4 (Accession: LOSM01000005.1 GI: 967485342); YB1A01 YB01_A01_contig_1 (Accession: LBCL01000001.1 GI: 940519882); YB2G05 YB02_G05_contig_7 (Accession: LBFZ01000007.1 GI: 940550115); InDRE 4262 chromosome I Chr1_contig7 (Accession: JZUB01000007.1 GI: 769091410); InDRE 4354 chromosome I Chr1_contig7 (Accession: JZUA01000007.1 GI: 769088978); YB8E08 YB08_E08_contig_18 (Accession: LBGN01000018.1 GI: 940599519); YB7A06 YB07_A06_contig_3 (Accession: LBGL01000003.1 GI: 940598755); YB7A09 YB07_A09_contig_12 (Accession: LBGM01000012.1 GI: 940597590); YB6A06 YB06_A06_contig_11 (Accession: LBGKO1000011.1 GI: 940592937); YB5A06 YB05_A06_contig_7 (Accession: LBGJ01000007.1 GI: 940588968); YB4G05 YB04_G05_contig_14 (Accession: LBGG01000014.1 GI: 940577186); YB4F05 YB04_F05_contig_14 (Accession: LBGF01000014.1 GI: 940572881); YB4B03 YB04_B03_contig_3 (Accession: LBGD01000003.1 GI: 940570625); YB4C07 YB04_C07_contig_32_consensus (Accession: LBGE01000031.1 GI: 940565209); YB3B05 YB03_B05_contig_2 (Accession: LBGB01000002.1 GI: 940562726); YB2G07 YB02_G07_contig_1 (Accession: LBGA01000001.1 GI: 940559910); YB1G06 YB01_G06_contig_1 (Accession: LBFVO1000001.1 GI: 940544222); YB2A05 YB02_A05_contig_14 (Accession: LBFW01000014.1 GI: 940540732); M1522 contig00012 (Accession: LQCA01000012.1 GI: 974047169); M988 contig00008 (Accession: LQBX01000008.1 GI: 974034339); O1 biovar El Tor strain FJ147 (Accession: CP009042.1 GI: 785752771); 2740-80 chromosome 2 (CP016325.1); O1 str. KW3 chromosome II (CP006948.1); TSY216 chromosome 2 (CP007654.1); O1 biovar El Tor strain FJ147 chromosome II (CP009041.1); 2012EL-2176 chromosome 2 (CP007635.1); MS6, chromosome 2 (AP014525.1); O1 str. 2010EL-1786 chromosome 2 (CP003070.1); MJ-1236 chromosome 2 (CP001486.1); 0395 chromosome II (CP001236.1); M66-2 chromosome II (CP001234.1); O395 chromosome 1 (CP000626.1); O1 biovar eltor str. N16961 chromosome II (AE003853.1); IEC224 chromosome II (CP003331.1); LMA3894-4 chromosome II (CP002556.1); 1154-74 (CP010811.1); or 10432-62 (CP010812.1). Cyclic di-nucleotide synthetase enzyme genes may also encompass those derived from any species, for example, but not limited to, Acinetobacter baumannii, Acinetobacter baylyi, Acinetobacter calcoaceticus, Acinetobacter haemolyticus, Acinetobacter junk Acinetobacter lwoffii, Acinetobacter nosocomialis, Acinetobacter pittii, Acinetobacter radioresistens, Actinobacillus lignieresii, Actinobacillus suis, Aeromonas ca viae, Aeromonas hydrophila, Aeromonas veronii subsp. sobria, Aggregatibacter actinomycetemcomitans, Arcobacter butzleri, Arcobacter nitrofigilis, Bacillus amyloliquefaciens, Bacillus anthracis, Bacillus bataviensis, Bacillus cellulosilyticus, Bacillus cereus, Bacillus clausii, Bacillus licheniformis, Bacillus megaterium, Bacillus pumilus, Bacillus subtilis, Bacillus thuringiensis, Bacteroides fragilis, Bordetella avium, Bordetella bronchiseptica, Bordetella pertusis, Bordetella petrii, Brucella abortus, Brucella melitensis, Brucella suis, Burkholderia cenocepacia, Burkholderia mallei, Burkholderia multivorans, Burkholderia pseudomallei, Burkholderia thailandensis, Campylobacter concisus, Campylobacter fetus subsp. fetus, Campylobacter fetus subsp. venerealis, Campylobacter gracilis, Campylobacter hominis, Campylobacter jejuni, Campylobacter rectus, Campylobacter showae, Campylobacter upsaliensis, Citrobacter freundii, Citrobacter koseri, Clostridium asparagiforme, Clostridium botulinum, Clostridium butyricum, Clostridium difficile, Clostridium perfringens, Clostridium saccharobutylicum, Clostridium tetani, Corynebacterium diphtheriae, Corynebacterium pseudotuberculosis, Enterobacter aerogenes, Enterobacter cloacae, Enterococcus faecalis, Enterococcus faecium, Erysipelothrix rhusiopathiae, Escherichia coli, Fusobacterium necrophorum, Fusobacterium nucleatum, Granulicatella adiacens, Granulicatella elegans, Haemophilus equigenitalis, Haemophilus influenzae, Haemophilus parainfluenzae, Haemophilus paragallinarum, Haemophilus parasuis, Haemophilus pleuropneumoniae, Haemophilus somnus, Helicobacter pylori, Klebsiella oxytoca, Klebsiella pneumoniae, Legionella oakridgensis, Legionella pneumophila, Leptospira biflexa, Leptospira illni, Leptospira interrogans, Listeria monocytogenes, Lysinibacillus fusiformis, Lysinibacillus sphaericus, Moraxella bovis, Morganella morganii, Mycobacterium abscesses, Mycobacterium africanum, Mycobacterium avium, Mycobacterium bovis, Mycobacterium leprae, Mycobacterium tuberculosis, Neisseria gonorrhoeae, Neisseria meningitidis, Pasteurella multocida, Plesiomonas shigelloides, Propionibacterium acnes, Proteus hanseri, Proteus mirabilis, Pseudomonas aeruginosa, Salmonella cholerasuis, Salmonella enterica subsp. enterica, Salmonella enteritidis, Salmonella paratyphi, Salmonella typhi, Serratia plymuthica, Shigella boydii, Shigella dysenteriae, Shigella flexneri, Staphylococcus arlettae, Staphylococcus aureus, Staphylococcus capitis, Staphylococcus caprae, Staphylococcus carnosus, Staphylococcus epidermidis, Staphylococcus equorum, Staphylococcus haemolyticus, Staphylococcus hominis, Staphylococcus lugdunensis, Staphylococcus pasteuri, Staphylococcus pettenkoferi, Staphylococcus pseudointermedius, Staphylococcus saprophyticus, Staphylococcus simiae, Staphylococcus simulans, Staphylococcus warneri, Stenotrophomonas maltophilia, Streptococcus agalactiae, Streptococcus dysgalactiae, Streptococcus dysgalactiae subsp. equisimilis, Streptococcus equi, Streptococcus pneumoniae, Streptococcus pyogenes, Streptococcus uberis, Streptococcus zooepidermicus, Taylorefta asinigenitalis, Taylorella equigenitalis, Treponema carateum, Treponema cuniculi, Treponema hyodisenteriae, Treponema pallidum, Treponema suis, Veillonella atypica, Veillonella dispar, Veillonella parvula, Veillonella ratti, Vibrio cholerae, Vibrio parahaemolyticus, Vibrio vulnificans, Yersinia enterocolitica, Yersinia pestis and Yersinia pseudotuberculosis.

The term “cGAS” refers a cytoplasmic eukaryotic receptor that responds to cytoplasmic DNA to produced cGAMP-ML (Sun L. et. al. (2013) Science. 339(6121):786-91; Gao P. (2013) Cell. 153(5):1094-107).

The term DAC refers to “diadenylate cyclase” enzymes encoded in bacteria that synthesis c-di-AMP. Bacteria encode a number of different DAC domain enzymes that may be targeted to the membrane of the cytoplasm (Commichau F. M. (2015) Mol Microbiol. 97(2):189-204). The first described DAC is DisA from Bacillus subtilis designated by COG1623 (Oppenheimer-Shaanan Y. et. al. (2011) EMBO Rep. 2011 June; 12(6):594-601).

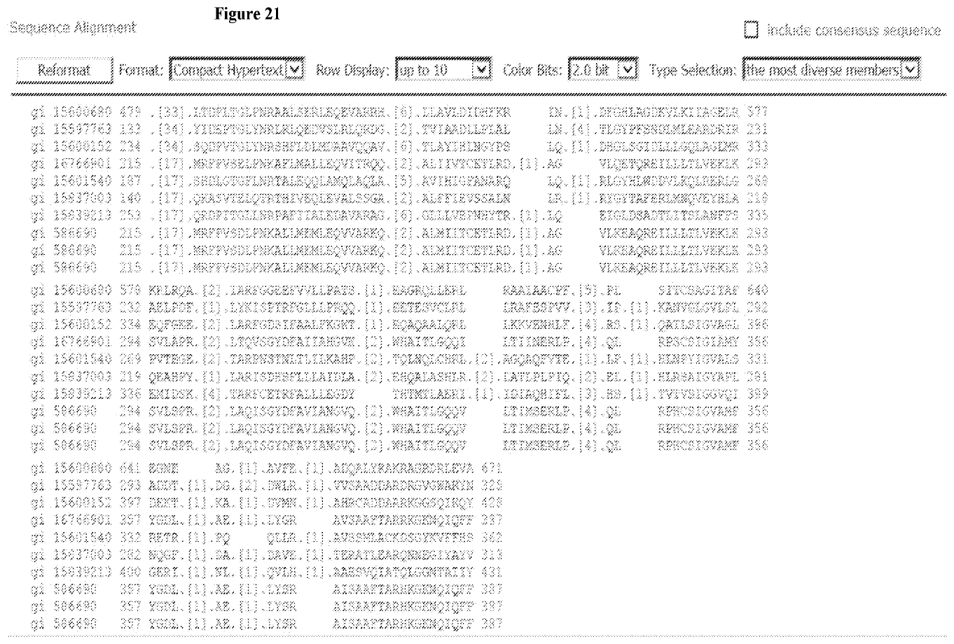

The term “diguanylate cyclase,” or “DGC”, unless otherwise specified, refers to known DGC RNA, DNA, and polypeptides, as well as its isoforms, and biologically active fragments thereof. DGC enzymes typically encode GGDEF domain that are described in the COG database as COG2199. V. cholerae encodes upwards of 40 unique DGCs, many of which have been shown to synthesize c-di-GMP in this bacterium (Beyhan, S et al. (2008) J Bacteriol 190: 7392-7405; Lim, B et al. (2006) Mol Microbiol 60: 331-348; Beyhan, S et al. (2007) Mol Microbiol 63: 995-1007; Massie, J P et al. (2012) Proc Natl Acad Sci USA 109(31):12746-51; Hunter, J L et al. (2014) BMC Microbiol 14: 22). These DGCs have highly divergent c-di-GMP synthesis activities (Shikuma, N J et al. (2012) PLoS Pathog 8: e1002719; Massie, J P et al. (2012) Proc Natl Acad Sci USA 109(31):12746-51). Approximately half of these DGCs are thought to be integral inner membrane proteins, while the other half are cytoplasmic. Each contains a unique N-terminal sensory domain that is predicted to be regulated by environmental or host derived cues (Galperin, MY (2004) Environ Microbiol 6: 552-567). Tens of thousands of DGCs have been identified across bacterial genomes (Hunter, J L et al. (2014) BMC Microbiol 14: 22). Thus, these genes offer a wide-range of unique enzymes possessing different properties that can be transduced by vectors to potentially modulate immune responses. DGC genes may encompass those derived from any of the V cholerae strains listed above, or any of the bacterial sources set forth above. Table 1, the Figures, and the Examples, below provide representative DGC sequences. For example, Table 1 provides DGC sequences encompassed within the scope of compositions-of-matter and methods of the present invention. However, any protein containing a protein domain belonging to the COG family COG2199 is considered a DGC (i.e., COG2199 which is the DGC (i.e., also called a GGDEF) domain that synthesizes c-di-GMP; see http://www.ncbi.nlm.nih.gov/Structure/cdd/cddsrv.cgi?ascbin=8&maxaln=10&seltype=2& uid=COG2199 at FIG. 21 and Galperin M Y et al. (2015) Nucleic Acids Res. 43(Database issue) D261-9; Ausmees N et al. (2001) Microbiol. Lett. 204(1):163-167; Paul R et al. (2004) Genes Dev. 18(6):715-727; Chan C et al. (2004) Proc. Natl. Acad. Sci. U.S.A. 101(49):17084-17089; Ryjenkov D A et al. (2005) J. Bacteriol. 187(5):1792-1798; Aldridge P et al. (1999) Mol. Microbiol. 1999 April; 32(2):379-391; Pei J et al. (2001) Proteins 2001 42(2):210-216; Tal R et al. (1998) J. Bacteriol. 180(17):4416-4425; Marcher-Bauer et al. (2015) Nucleic Acids Res. 43(Database issue):D222-6).

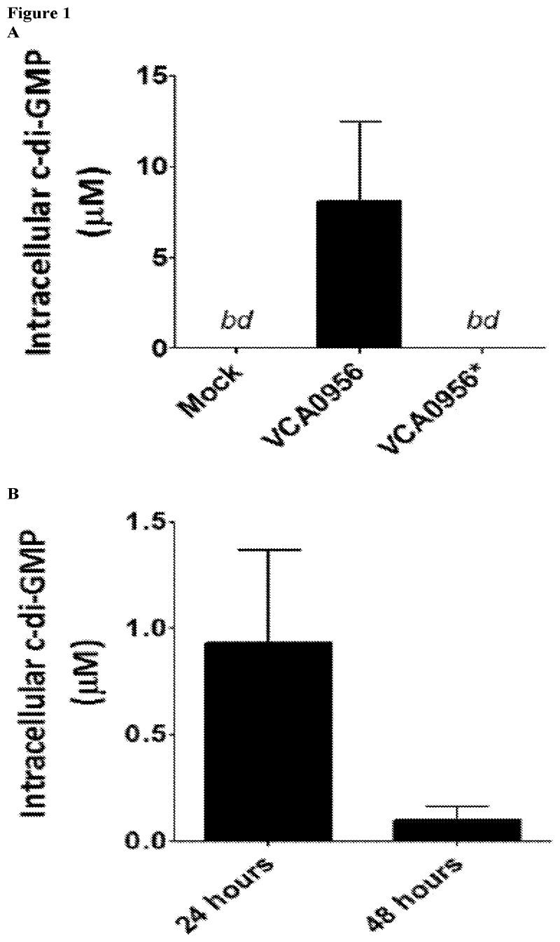

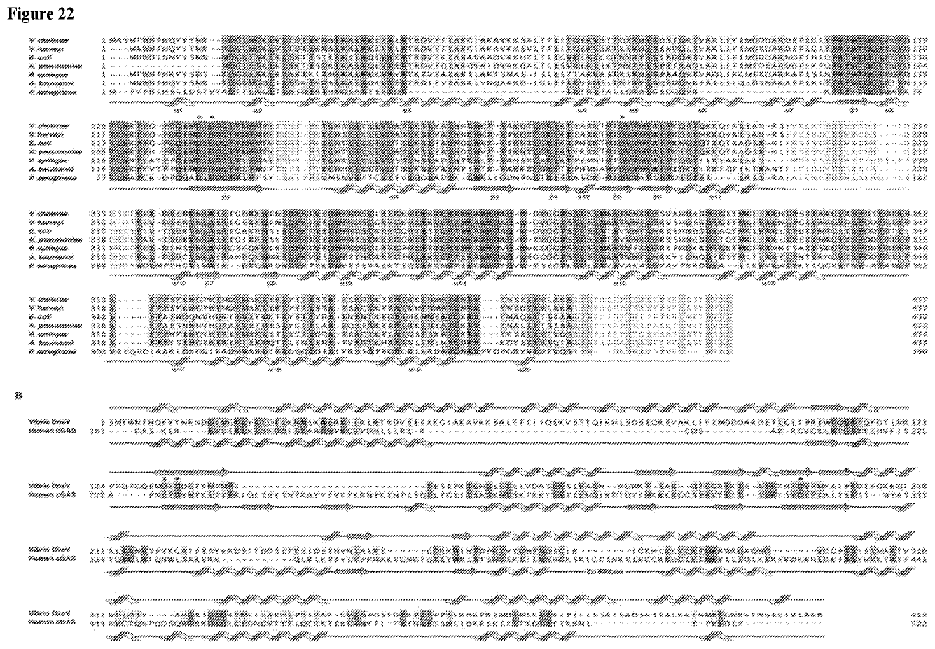

The term “DncV” refers to a bacterial enzyme encoded in V. cholerae that has been shown to synthesize cGAMP (Davies B. W. et. al. (2012) Cell. 149(2):358-70). As noted in Kranzusch P J et al. (2014) Cell 158(5):1011-21, in spite of the minimal sequence identity, the results in the paper showed that DncV is both a structural and functional homolog of mammalian cGAS, which demonstrates for the first time a direct connection between the biosynthetic machinery for generating dinucleotide signals in multiple kingdoms of life. The core of DncV adopts a template-independent nucleotidyl-transferase fold defined by B strands ß2-5, similar to the originally characterized CCA-adding enzyme (FIG. 1) (Xiong et al. (2004) Nature 430, pp. 640-645). In spite of minimal sequence identity (˜10%), the overall structure of DncV is remarkably similar to that of human cGAS (Kranzusch P J et al. (2014) Cell 158(5):1011-21). FIG. 22 from Kranzusch depicts a sequence alignment of various DncV homologs from bacteria.

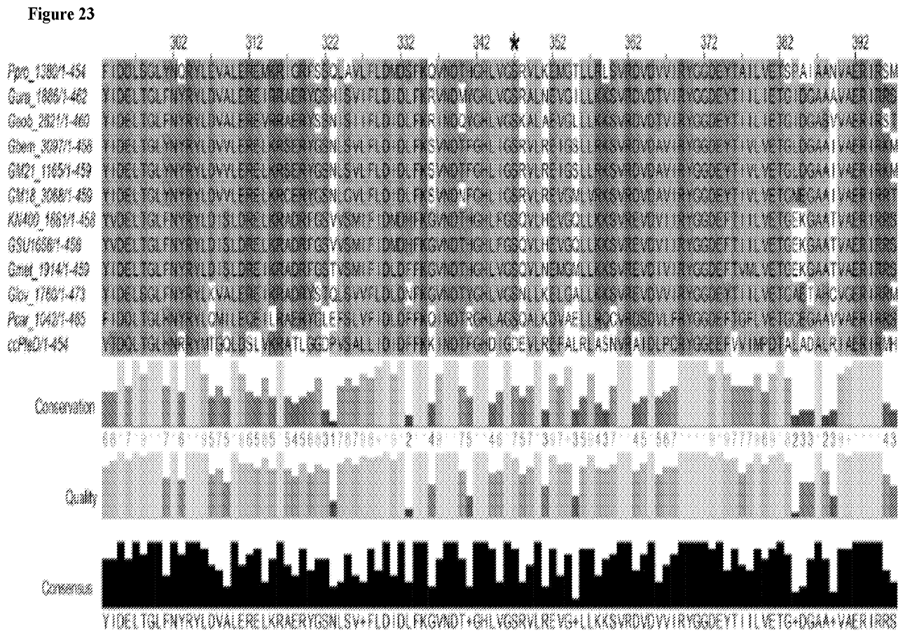

The term “Hypr-GGDEF” refers to a certain class of DGC enzymes that have a GGDEF domain that have been shown to synthesize cGAMP depending on the available nucleotide substrates (Hallberg Z. F. et. al. (2016) Proc Natl Acad Sci 113(7):1790-5.). As noted in Hallberg Z F et al (2016) Proc Natl Acad Sci USA. 113(7):1790-5, hybrid promiscuous (Hypr) GGDEF enzymes produce cyclic AMP-GMP (3′,3′-cGAMP) (see Fig. S9 (FIG. 23 herein) which lists the putative HYPR domains in Geobacter and Pelobacter and identifies the conserved residues. The bottom sequence (ccPleD/1-454) is a known GGDEF from Caulobacter crescentus for comparison).

DisA (c-di-AMP synthesis). NCBI lists the domain as pfam02457: DisA_N From the NCBI website: “DisA bacterial checkpoint controller nucleotide-binding: The DisA protein is a bacterial checkpoint protein that dimerizes into an octameric complex. The protein consists of three distinct domains. This domain is the first and is a globular, nucleotide-binding region; the next 146-289 residues constitute the DisA-linker family, pfam10635, that consists of an elongated bundle of three alpha helices (alpha-6, alpha-10, and alpha-11), one side of which carries an additional three helices (alpha7-9), which thus forms a spine like-linker between domains 1 and 3. The C-terminal residues, of domain 3, are represented by family HHH, pfam00633, the specific DNA-binding domain. The octameric complex thus has structurally linked nucleotide-binding and DNA-binding HhH domains and the nucleotide-binding domains are bound to a cyclic di-adenosine phosphate such that DisA is a specific di-adenylate cyclase. The di-adenylate cyclase activity is strongly suppressed by binding to branched DNA, but not to duplex or single-stranded DNA, suggesting a role for DisA as a monitor of the presence of stalled replication forks or recombination intermediates via DNA structure-modulated c-di-AMP synthesis.” pfam02457 is a member of the superfamily c110589 (see Marchler-Bauer A et al. (2015) Nucleic Acids Res. 43(Database issue):D222-6).

Examples of diseases or conditions wherein enhancement of a protective immune response is desired includes, but are not limited to viral, pathogenic, protozoal, bacterial, or fungal infections and cancer.

Viral infectious diseases include human papilloma virus (HPV), hepatitis A Virus (HAV), hepatitis B Virus (HBV), hepatitis C Virus (HCV), retroviruses such as human immunodeficiency virus (HIV-1 and HIV-2), herpes viruses such as Epstein Barr Virus (EBV), cytomegalovirus (CMV), HSV-1 and HSV-2, influenza virus, Hepatitis A and B, FIV, lentiviruses, pestiviruses, West Nile Virus, measles, smallpox, cowpox, ebola, coronavirus, retrovirus, herpesvirus, potato S virus, simian Virus 40 (SV40), Mouse Mammary Tumor Virus (MMTV) promoter, Moloney virus, ALV, Cytomegalovirus (CMV), Epstein Barr Virus (EBV), or Rous Sarcoma Virus (RSV). In addition, bacterial, fungal and other pathogenic diseases are included, such as Aspergillus, Brugia, Candida, Chikungunya, Chlamydia, Coccidia, Cryptococcus, Dengue, Dirofilaria, Gonococcus, Histoplasma, Leishmania, Mycobacterium, Mycoplasma, Paramecium, Pertussis, Plasmodium, Pneumococcus, Pneumocystis, P. vivax in Anopheles mosquito vectors, Rickettsia, Salmonella, Shigella, Staphylococcus, Streptococcus, Toxoplasma and Vibrio cholerae. Exemplary species include Neisseria gonorrhea, Mycobacterium tuberculosis, Candida albicans, Candida tropicalis, Trichomonas vaginalis, Haemophilus vaginalis, Group B Streptococcus sp., Microplasma hominis, Hemophilus ducreyi, Granuloma inguinale, Lymphopathia venereum, Treponema pallidum, Brucella abortus. Brucella melitensis, Brucella suis, Brucella canis, Campylobacter fetus, Campylobacter fetus intestinalis, Leptospira pomona, Listeria monocytogenes, Brucella ovis, Chlamydia psittaci, Trichomonas foetus, Toxoplasma gondii, Escherichia coli, Actinobacillus equuli, Salmonella abortus ovis, Salmonella abortus equi, Pseudomonas aeruginosa, Corynebacterium equi, Corynebacterium pyogenes, Actinobaccilus seminis, Mycoplasma bovigenitalium, Aspergillus fumigatus, Absidia ramosa, Trypanosoma equiperdum, Clostridium tetani, Clostridium botulinum; or, a fungus, such as, e.g., Paracoccidioides brasiliensis; or other pathogen, e.g., Plasmodium falciparum. Also included are National Institute of Allergy and Infectious Diseases (NIAID) priority pathogens. These include Category A compositions, such as variola major (smallpox), Bacillus anthracis (anthrax), Yersinia pestis (plague), Clostridium botulinum toxin (botulism), Francisella tularensis (tularaemia), filoviruses (Ebola hemorrhagic fever, Marburg hemorrhagic fever), arenaviruses (Lassa (Lassa fever), Junin (Argentine hemorrhagic fever) and related viruses); Category B compositions, such as Coxiella burnetti (Q fever), Brucella species (brucellosis), Burkholderia mallei (glanders), alphaviruses (Venezuelan encephalomyelitis, eastern & western equine encephalomyelitis), ricin toxin from Ricinus communis (castor beans), epsilon toxin of Clostridium perfringens; Staphylococcus enterotoxin B, Salmonella species, Shigella dysenteriae, Escherichia coli strain O157:H7, Vibrio cholerae, Cryptosporidium parvum; Category C compositions, such as nipah virus, hantaviruses, yellow fever in Aedes mosquitoes, and multidrug-resistant tuberculosis; helminths, such as Schistosoma and Taenia; and protozoa, such as Leishmania (e.g., L. mexicana) in sand flies, Plasmodium, Chagas disease in assassin bugs.

Other bacterial pathogens include, but are not limited to, bacterial pathogenic gram-positive cocci, which include but are not limited to: pneumococci; staphylococci; and streptococci. Pathogenic gram-negative cocci include: meningococci; and gonococci. Pathogenic enteric gram-negative bacilli include: enterobacteriaceae; Pseudomonas, acinetobacteria and Eikenella; melioidosis; Salmonella; shigellosis; hemophilus; chancroid; brucellosis; tularemia; Yersinia (pasteurella); Streptobacillus moniliformis and spirilum; Listeria monocytogenes; Erysipelothrix rhusiopathiae; diphtheria; cholera; anthrax; and donovanosis (Granuloma inguinale). Pathogenic anaerobic bacteria include; tetanus; botulism; other clostridia; tuberculosis; leprosy; and other mycobacteria. Pathogenic spirochetal diseases include: syphilis; treponematoses: yaws, pinta and endemic syphilis; and leptospirosis. Other infections caused by higher pathogen bacteria and pathogenic fungi include: actinomycosis; nocardiosis; cryptococcosis, blastomycosis, histoplasmosis and coccidioidomycosis; candidiasis, aspergillosis, and mucormycosis; sporotrichosis; paracoccidiodomycosis, petriellidiosis, torulopsosis, mycetoma and chromomycosis; and dermatophytosis. Rickettsial infections include rickettsial and rickettsioses. Examples of Mycoplasma and chlamydial infections include: Mycoplasma pneumoniae; lymphogranuloma venereum; psittacosis; and perinatal chlamydial infections. Pathogenic protozoans and helminths and infections eukaryotes thereby include: amebiasis; malaria; leishmaniasis; trypanosomiasis; toxoplasmosis; Pneumocystis carinii; giardiasis; trichinosis; filariasis; schistosomiasis; nematodes; trematodes or flukes; and cestode (tapeworm) infections. While not a disease or condition, enhancement of a protective immune response is also beneficial in a vaccine or as part of a vaccination regimen as is described herein.

The terms “enhance”, “promote” or “stimulate” in terms of an immune response includes an increase, facilitation, proliferation, for example a particular action, function or interaction associated with an immune response.

The term “homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

The term “host cell” is intended to refer to a cell into which any of the nucleotide sequence of the one or more cyclic di-nucleotide synthetase enzyme, or fragment thereof, such as a recombinant vector (e.g., gene therapy vector) of the present invention, has been introduced. The terms “host cell” and “recombinant host cell” are used interchangeably herein. It should be understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

As used herein, the term “immune cell” refers to cells that play a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes.

As used herein, the term “immune response” includes T cell mediated and/or B cell mediated immune responses. Exemplary immune responses include T cell responses, e.g., cytokine production and cellular cytotoxicity. In addition, the term immune response includes immune responses that are indirectly effected by T cell activation, e.g., antibody production (humoral responses) and activation of cytokine responsive cells, e.g., macrophages.

The term “immunotherapeutic composition” can include any molecule, peptide, antibody or other composition which can stimulate a host immune system to generate an immune response to a tumor or cancer in the subject.

As used herein, the term “inhibit” includes the decrease, limitation, or blockage, of, for example a particular action, function, or interaction. For example, a pathogenic infection or cancer is “inhibited” if at least one symptom of the pathogenic infection or cancer, such as hyperproliferative growth, is alleviated, terminated, slowed, or prevented. As used herein, cancer is also “inhibited” if recurrence or metastasis of the cancer is reduced, slowed, delayed, or prevented.

As used herein, the term “interaction,” when referring to an interaction between two molecules, refers to the physical contact (e.g., binding) of the molecules with one another. Generally, such an interaction results in an activity (which produces a biological effect) of one or both of said molecules. The activity may be a direct activity of one or both of the molecules. Alternatively, one or both molecules in the interaction may be prevented from binding their ligand, and thus be held inactive with respect to ligand binding activity (e.g., binding its ligand and triggering or inhibiting an immune response). To inhibit such an interaction results in the disruption of the activity of one or more molecules involved in the interaction. To enhance such an interaction is to prolong or increase the likelihood of said physical contact, and prolong or increase the likelihood of said activity.

A “kit” is any manufacture (e.g., a package or container) comprising at least one reagent (e.g, gene therapy vector of the present invention, an extracellular Ag) for use in stimulating or enhancing an immune response when adminitered. The kit may be promoted, distributed, or sold as a unit for performing the methods of the present invention.

The term “modulate” includes up-regulation and down-regulation, e.g., enhancing or inhibiting a response.

The term “sample” is typically whole blood, plasma, serum, saliva, urine, stool (e.g., feces), tears, and any other bodily fluid (e.g., as described above under the definition of “body fluids”), or a tissue sample such as a small intestine, colon sample, or surgical resection tissue. In certain instances, the method of the present invention further comprises obtaining the sample from the individual prior to detecting or determining the presence or level of at least one marker in the sample.

The term “synergistic effect” refers to the combined effect of two or more compositions of matter of the present invention that is greater than the sum of the separate effects of the compositions of matter alone.

The term “mammal” refers to any healthy animal, subject or human, or any animal, mammal or human afflicted with a condition of interest (e.g., pathogenic infection or cancer). The term “subject” is interchangeable with “patient.”

The term “purity” as used herein, refers to any of compositons or matter described herein which is substantially free of impurities or artifacts that may interfere in the efficacy of the composition when administered. Impurities or artifacts may include interfering antibody, polypeptide, peptide or fusion protein. In one embodiment, the language “purity of at least 75%, 80%, 85%, 90%, 95%, 98%, or 99%” includes preparations of vectors (e.g., gene therapy vectors), or pharmaceutical compositions, vaccines, adjuvants, combination vaccines (e.g., vector combined with an additional therapeutic agent), or the like, having less than about 30%, 20%, 15%, 10%, 5% (by dry weight) of impurities and/or artifacts.

The terms “treatment” “treat” and “treating” encompasses alleviation, cure or prevention of at least one symptom or other aspect of a infection, disorder, disease, illness or other condition (e.g., pathogenic infections or cancer), or reduction of severity of the condition, and the like. A composition of matter of the invention need not affect a complete cure, or eradicate every symptom or manifestation of a disease, to constitute a viable therapeutic composition. As is recognized in the pertinent field, drugs employed as therapeutic compositions may reduce the severity of a given disease state, but need not abolish every manifestation of the disease to be regarded as useful therapeutic compositions. Beneficial or desired clinical results include, but are not limited to, alleviation of symptoms, diminishment of extent of disease, stabilization (i.e., not worsening) of disease, delay or slowing of disease progression, amelioration or palliation of the disease state, remission (whether partial or total, whether detectable or undetectable) and prevention of relapse or recurrence of disease. Similarly, a prophylactically administered treatment need not be completely effective in preventing the onset of a condition in order to constitute a viable prophylactic composition. Simply reducing the impact of a disease (for example, by reducing the number or severity of its symptoms, or by increasing the effectiveness of another treatment, or by producing another beneficial effect), or reducing the likelihood that the disease will occur or worsen in a subject, is sufficient.

“Treatment” can also mean prolonging survival as compared to expected survival if not receiving treatment. In one embodiment, an indication that a therapeutically effective amount of a composition has been administered to the patient is a sustained improvement over baseline of an indicator that reflects the severity of the particular disorder.

By a “therapeutically effective amount” of a composition of the invention is meant an amount of the composition which confers a therapeutic effect on the treated subject, at a reasonable benefit/risk ratio applicable to any medical treatment. The therapeutic effect is sufficient to “treat” the patient as that term is used herein.

As used herein, a vaccine is a composition that provides protection against a pathogenic infection (e.g., protozoal, viral, or bacterial infection), cancer or other disorder or treatment for a pathogenic infection, cancer or other disorder. Protection against a pathogenic infection, cancer or other disorder will either completely prevent infection or the tumor or other disorder or will reduce the severity or duration of infection, tumor or other disorder if subsequently infected or afflicted with the disorder. Treatment will cause an amelioration in one or more symptoms or a decrease in severity or duration. For purposes herein, a vaccine results from infusion of injection (either concomitantly, sequentially or simultaneously) of an antigen and a composition of matter produced by the methods herein. As used herein, amelioration of the symptoms of a particular disorder by administration of a particular composition refers to any lessening, whether permanent or temporary, lasting or transient that can be attributed to or associated with administration of the compositions of matter described herein.

As used herein a “vaccination regimen” means a treatment regimen wherein a vaccine comprising an antigen and/or any of the gene therapy-vectors (alone or in combination) described herein, as an adjuvant, is administered to a subject in combination, simultaneously, in either separate or combined formulations, or sequentially at different times separated by minutes, hours or days, but in some way act together to provide the desired enhanced immune response to the vaccine in the subject as compared to the subject's immune response in the absence of a composition in accordance with the invention. In some embodiments of the methods described herein, the “antigen” is not delivered but is already present in the subject, such as those antigens which are associated with tumors. In some embodiments of the compositions described herein, the gene therapy vectors can have activity that is independent of their adjuvant properties. For example, and by no way limiting, STING activation has been shown to have a direct toxic effect on cancer cells.

As used herein, the term “vector”, used interchangeably with “construct”, refers to a nucleic acid capable of transporting another nucleic acid to which it has been linked. One type of vector is a “plasmid”, which refers to a circular double stranded DNA loop into which additional DNA segments may be ligated. Another type of vector is a viral vector (e.g., replication defective adenovirus, retroviruses, or lentivirus), wherein additional DNA segments may be ligated into the viral genome. Viral vectors may also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” or simply “expression vectors.” In general, expression vectors of utility in recombinant DNA techniques are often in the form of plasmids. In the present specification, “plasmid” and “vector” may be used interchangeably as the plasmid is the most commonly used form of vector. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. Also included are DNA-based vectors, which can be delivered “naked” or formulated with liposomes to help the uptake of naked DNA into cells.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA coding for a protein or polypeptide of the present invention (or any portion thereof) can be used to derive the protein or polypeptide amino acid sequence, using the genetic code to translate the DNA or RNA into an amino acid sequence. Likewise, for a protein or polypeptide amino acid sequence, corresponding nucleotide sequences that can encode the protein or polypeptide can be deduced from the genetic code (which, because of its redundancy, will produce multiple nucleic acid sequences for any given amino acid sequence). Thus, description and/or disclosure herein of a nucleotide sequence which encodes a protein or polypeptide should be considered to also include description and/or disclosure of the amino acid sequence encoded by the nucleotide sequence. Similarly, description and/or disclosure of a protein or polypeptide amino acid sequence herein should be considered to also include description and/or disclosure of all possible nucleotide sequences that can encode the amino acid sequence.

Finally, nucleic acid and amino acid sequence information for any cyclic di-nucleotide synthetase enzymes (e.g., any DGC, DAC, DncV, cGAS, Hypr-GGDEF, DisA) are well known in the art and readily available on publicly available databases, such as the National Center for Biotechnology Information (NCBI). For example, any protein containing a protein domain belonging to the COG family COG2199 is considered a DGC (i.e., COG2199 which is the DGC (i.e., also called a GGDEF) domain that synthesizes c-di-GMP; see http://www.ncbi.nlm.nih.gov/Structure/cdd/cddsrv.cgi?ascbin=8&maxaln=10&seltype=2&uid=COG2199 at FIG. 21 and Galperin M Y et al. (2015) Nucleic Acids Res. 43(Database issue) D261-9; Ausmees N et al. (2001) Microbiol. Lett. 204(1):163-167; Paul R et al. (2004) Genes Dev. 18(6):715-727; Chan C et al. (2004) Proc. Natl. Acad. Sci. U.S.A. 101(49):17084-17089; Ryjenkov D A et al. (2005) J. Bacteriol. 187(5):1792-1798; Aldridge P et al. (1999) Mol. Microbiol. 1999 April; 32(2):379-391; Pei J et al. (2001) Proteins 2001 42(2):210-216; Tal R et al. (1998) J. Bacteriol. 180(17):4416-4425; Marcher-Bauer et al. (2015) Nucleic Acids Res. 43(Database issue):D222-6). For example, exemplary cyclic di-nucleotide synthetase enzymes nucleic acid and amino acid sequences derived from publicly available sequence databases are provided below.

TABLE 1
DGC nucleotide and amino acid sequences

SEQ ID NO: 1 Vibrio cholerae O1 str. C6706 Contig_56 DNA Sequence
(GI:446210820 REGION 98731..100614)
tcacgcaaag tgatgcattt ccatggcggt gagtactgat atttggttgc gtcccgatgt
tttggattca tataaagcca gatcggctct tttgtagctt tggtcgggtg atgtgcaaac
atcggtaaca ccaccactta gggtcacttg ttgatggtgt aatgaagcga tatgcaggcg
tacgcggtta agtacttgtt cggcttcttc aatggaagtg taggggaaaa taatggcaaa
ctcttctccg ccaatccgtg cgataaaatc cgattcccgt aactgatctt ggatgccttt
cgcaacggtc cgtaacacta ggtccccttc gttgtgtccg aatttgtcgt taatgcgttt
aaagtggtcg atatcaatga tagcaaggca gctctgggct tgatcgggat aacggcgacg
cttagcgcac tctaaagaga tggtttgatc gaatttacgt cgattccaca aatcggttaa
cgcatctttt tcgctcagct cacgcaggcg attctccagc gccttgcgat gtgaaatatc
cacaaaagag gcaacgtaga attgaatgac attgtcttca tcgcggatgc tttgaatacg
gagaatttcg gtgatgcttt cgccatcttt gcgtttgttg atcacttcac cttcccatac
gccattgtct tgcagagctt tccacatctg catatagaat tcgactttgt gtaatccaga
agcaaaaatg gacggctgct tacctttgac atcttcaaaa gtgtaaccac ttaggcgggt
aaattcgttg tttactttga tgatgcgatt ctggcggtcg gtaatgacca ccgctgacat
gccatccatc gctgctcgag ccaatttact gtcaaggcta ttttttaaat ggttgatgtt
ccatgccgca aatccagccg caatgataga gagtagcgat aacactgtca ccgcttgact
catcagtgcc cagcgcgcat ttgcgtaggt cttatctatt tctgccttat tgatgcgcag
taccaatacc aaaggtttaa agtcaggtaa gacagaactg agatccactt tgatatagct
aaaccaggtt tgattggata gagcaaagcc ttgttggttg agttggattt tttgccaaag
ctctgggtgt tgggctgaaa agtggagtga agaggttgaa cgtgtaccgg atggcttgtg
ttcactgagc agtaattctc ctgccgaatt caaaatatcc ggtgaatcaa actgatcata
aataaaagag agacgttgat agagagactg tagcttcacc gtcacgacaa gaaaaccttg
ccgttggcct tgatgctcaa tacccgtcac aaaacgaaag gtcggcagca taccagaagg
cgtatctgct gacatcgcga cttgcgttgc ccaaacttga ggcgtcgtga gttgggcgta
ttgagccaca atttgctggc tgaacggatc tgtcgtttga gcagattcaa caaaggtgac
ttggtgccca tcgtaaatcg ctttaagttg ttcttttcct tgtctatcca gcaatctgaa
tgaagagaaa atcgcttgcg atcttaacgt cacatcccac aatgttttga gttgactgag
tgcttctttg cttggtgtgg tgacagccgt gaataaaagg tcatttttag ctaacagctg
ggtggcttgg tgtgtgcttt ccagcattcg taacaagtca tgctgactga actcaagctg
taagcgagtc tgtttttcaa cgctgctgac cgcttgagtc tcaagctggc tagcagcatg
tatgaaatac agtgtaggaa tgaaaccaag tacaaacgca acaatggcaa attgtatgaa
atatttacgg gctgaggtgt acat
SEQ ID NO: 2 Vibrio cholerae O1 str. C6706 Contig_56 amino acid Sequence
(WP_000288675.1)

| | |
|---|---|
| 1 | MYTSARKYFI QFAIVAFVLG FIPTLYFIHA ASQLETQAVS SVEKQTRLQL EFSQHDLLRM |
| 61 | LESTHQATQL LAKNDLLFTA VTTPSKEALS QLKTLWDVTL RSQAIFSSFR LLDRQGKEQL |
| 121 | KAIYDGHQVT FVESAQTTDP FSQQIVAQYA QLTTPQVWAT QVAMSADTPS GMLPTFRFVT |
| 181 | GIEHQGQRQG FLVVTVKLQS LYQRLSFIYD QFDSPDILNS AGELLLSEHK PSGTRSTSSL |
| 241 | HFSAQHPELW QKIQLNQQGF ALSNQTWFSY IKVDLSSVLP DFKPLVLVLR INKAEIDKTY |
| 301 | ANARWALMSQ AVTVLSLLSI IAAGFAAWNI NHLKNSLDSK LARAAMDGMS AVVITDRQNR |
| 361 | IIKVNNEFTR LSGYTFEDVK GKQPSIFASG LHKVEFYMQM WKALQDNGVW EGEVINKRKD |
| 421 | GESITEILRI QSIRDEDNVI QFYVASFVDI SHRKALENRL RELSEKDALT DLWNRRKFDQ |
| 481 | TISLECAKRR RYPDQAQSCL AIIDIDHFKR INDKFGHNEG DLVLRTVAKG IQDQLRESDF |
| 541 | IARIGGEEFA IIFPYTSIEE AEQVLNRVRL HIASLHHQQV TLSGGVTDVC TSPDQSYKRA |
| 601 | DLALYESKTS GRNQISVLTA MEMHHFA |

SEQ ID NO: 3 Vibrio cholerae O1 str. C6706 Contig_56 DNA Sequence
(GI:446272186 REGION 240951..242336)
ttatgaccag gtacgaaaga caacctggtt ctttccattc cgctttcctt cgtacattaa
gctatcggca tcgtgcaaac tgaatggccg agctgggtgt aggtaaaatg cacagcctag
gctgatggtg agtgacagag agtgttgggc attcactacc cacttttttt ctgcaactcg
ttggcaaatt cgctcagcta actgctgcga ctcttctgca tttttaccac gcgctacaat
agcaaactct tcaccaccaa tccttgcaaa gtaggtatcc gatgctaaag cttgtcgcac
acacccaacc acgaaacaga tggcattatc tcctgcgcca tgcccaaagc gatcgttaat
ggttttgaag tcatcaatat caaaaaccat caacgttaag ctgcctgatc gtgtttgttc
cgcttcaaga tgttcaaaaa acgaacggcg attagcaatg cccgtcaagc tatccgtttt
cgctaaatag gagagttttt gattggcttc ttcaagttgc tgtgttcgca atcgaacggt
acgccgtagc tgaagagtat aaataacgat actgagtaag agacctgaag cgagaatcgg
cattaagtaa cgtggataaa tcgtttcaat atgaacccat cgacttaaaa tacggttttt
ctcattgcta cttaattgtg caaacccctg ctctacttgc tctaataaat ccctattgcc
tttggcgacc gctggacgta attcctctga ataaagaaac ttcactggcg taaaatcttt
cgcgccattg gaaaccacta tatagaaatt ggcgacctga gtatcggcca caaaaccatc
taattctcgt cgctttgctg cagacatcat caattcattg ttggcgtact caatcaactt
aagttgagga tattctcgtt gcatgaactc ttgttcaaat ccccctttta ctacacctaa
tgagacgtta atggcccccg atagcagcgt atccaattta tcgcccaata acgtgcggtg
tacgtagagt tgtgtatcga ttgtcagtaa aggttctgca aaatcgagat acgctaatct
tgaagcagaa cggatcaaac cagcttgaac atcggatttg ccaagcttca ccgcttctag
ggaatcattc caatccatca gttggaattc aatatcgaca tgattcgctt caccaaaagc
caaccaaaaa tcaatcaata tgccagaagg ctgtccctgt tcatccaaat aagaataggg
tttccatgct tttgagttgg caatagtcaa ggtttggcgc tctacagcct cactcattga
tccgaataaa agcggccaag caatcatgag aagcagaaac agtttggtcg aaaagcgatg atccat
SEQ ID NO: 4 Vibrio cholerae O1 str. C6706 Contig_56 amino acid Sequence
(WP_000350041.1)

| | |
|---|---|
| 1 | MDHRFSTKLF LLLMIAWPLL FGSMSEAVER QTLTIANSKA WKPYSYLDEQ GQPSGILIDF |
| 61 | WLAFGEANHV DIEFQLMDWN DSLEAVKLGK SDVQAGLIRS ASRLAYLDFA EPLLTIDTQL |
| 121 | YVHRTLLGDK LDTLLSGAIN VSLGVVKGGF EQEFMQREYP QLKLIEYANN ELMMSAAKRR |
| 181 | ELDGFVADTQ VANFYIVVSN GAKDFTPVKF LYSEELRPAV AKGNRDLLEQ VEQGFAQLSS |
| 241 | NEKNRILSRW VHIETIYPRY LMPILASGLL LSIVIYTLQL RRTVRLRTQQ LEEANQKLSY |
| 301 | LAKTDSLTGI ANRRSFFEHL EAEQTRSGSL TLMVFDIDDF KTINDRFGHG AGDNAICFVV |
| 361 | GCVRQALASD TYFARIGGEE FAIVARGKNA EESQQLAERI CQRVAEKKWV VNAQHSLSLT |
| 421 | ISLGCAFYLH PARPFSLHDA DSLMYEGKRN GKNQVVFRTW S |

SEQ ID NO: 5 Vibrio cholerae O1 str. C6706 Contig_20 DNA Sequence
(GI:446493741 REGION 153278..154204)
atgatagaac ttaatagaat tgaagagctt tttgataacc aacagttctc cttgcacgaa
ctcgtgttga acgaactggg agtctatgtc ttcgtcaaaa atcgccgcgg cgagtatctc
tatgctaacc ctctgactct aaagttgttt gaagcggatg cacaatcgtt gtttggcaag
accgatcacg atttttttca tgatgatcaa ctcagtgata tcttggcggc cgatcaacag
gtgtttgaaa ctcgtctctc ggttatccat gaagaacgag ccatcgccaa atccaatggt
ttggttcgga tttatcgcgc agtcaaacac cctatcttgc accgagtgac aggcgaagtg
attgggctga ttggagtttc aaccgatatc accgatatcg tggaactgcg tgagcagcta
tatcagctcg ccaataccga ttctttaact cagctgtgta atcggcgtaa attgtgggcc
gattttcgcg ccgccttcgc tcgcgcaaaa cgtttaagac agccgttaag ttgcatctct
atcgatattg ataatttcaa actgatcaat gaccaatttg gtcacgataa aggtgatgaa
gtcctgtgtt ttctcgccaa actatttcag agcgtcatct ctgaccatca tttttgtggt
cgtgtgggag gtgaagagtt catcatcgtt ttggaaaata cgcacgtaga gacggctttt
catttggctg aacagatccg ccaacgtttt gcagagcatc cgttctttga acaaaacgag
cacatctacc tctgtgcggg ggtttccagc ttgcatcatg gtgatcatga cattgccgat
atttatcgac gctccgatca agcactgtat aaagccaagc gtaatggtcg taaccgttgc
tgtatctatc gccaatccac agaataa
SEQ ID NO: 6 Vibrio cholerae O1 str. C6706 Contig_20 amino acid Sequence
(WP_000571595.1)

| | |
|---|---|
| 1 | MIELNRIEEL FDNQQFSLHE LVLNELGVYV FVKNRRGEYL YANPLTLKLF EADAQSLFGK |
| 61 | TDHDFFHDDQ LSDILAADQQ VFETRLSVIH EERAIAKSNG LVRIYRAVKH PILHRVTGEV |
| 121 | IGLIGVSTDI TDIVELREQL YQLANTDSLT QLCNRRKLWA DFRAAFARAK RLRQPLSCIS |
| 181 | IDIDNFKLIN DQFGHDKGDE VLCFLAKLFQ SVISDHHFCG RVGGEEFIIV LENTHVETAF |
| 241 | HLAEQIRQRF AEHPFFEQNE HIYLCAGVSS LHHGDHDIAD IYRRSDQALY KAKRNGRNRC |
| 301 | CIYRQSTE |

SEQ ID NO: 7 Vibrio cholerae O1 str. C6706 Contig_20 DNA Sequence
(GI:446446879 REGION 171467..172840)
tcaaaagcga tagagtgggt tttgcctacg cttagcggta tacatacgtt catcggccag
tttgaacatt tcatcaggtg tggcaaacga ctggtcatac aaagcatatc cgatacttac
acgaacatgg ataagcttgt cgtcataaac gatgggcgtt tcagaaatcc tttttaaaat
attgtcactg actttaagca cgtcttgttc acgatgaatt cgtggaatta acacgagaaa
ctcatccccc ccaatccgcg ccaccagatc ggaaacccgc aggctcgatt taattctttc
cgcacaagcc accagcactt tatcgcctgc gctatgtcca tgggaatcgt tgatagattt
aaaacggtca atatcaatgt tcaacaaagc aaagttacct tcgctatgag agcgcttagc
attttcaaag tagtgttcaa tggtatagat aaaatagcgc cgattcggca agtgggttaa
agggtcatgt agcgcacgct cctccgcgac ttgataaagg cgcatgataa cgccaaagcc
tgccatcaat accaataaca ccgagtatcc caacaagcgc actgcatttc gggtatacca
agataactgc tgtagtaaat cttgcttttc agcgaccgca attcgccaac ttccgtaagg
gaaatagaca ttctcttgtg caaaagcgtg ctcaaatact cgaggctctc caaaaaacac
gtccccctca ctgccacggc tgtctaaacc acgaatcgca acctgaaaat gctccccaaa
gctgtaaata ctggttgctg aaagcaatga atcccaatcc atcaccacac tcagtacccc
ccaataacgc gtatccttcg gtgggtcgta gaatatcggt tctcgaatca ccagcgcgcg
cccaccttga acgagatcga caggtccaga gacgaacgtc tgtttgattt cacgtgcttt
ttttattgac tgccactgct gaggaacggt gcggtaatcc aaaccgagta gtgcattggt
ttgaggaagc ggatagctga aagcgaccac atcattaggg gcgataccta atgagcgtaa
gtgatcgcta ttcctgatca ccgccgctga aagcggctcc cattgataga tattgaggtc
gggatctagg gttaacaggg ttgttaaacc ttttacggta tagatatcac ccaaaatctc
agcttctaat tgaaaacgta cgatggaaag atcttcttta gcttgttgac gtaaaccctc
ttgtagatca cgtgtatggc taatatgaag ggattcaata accgcaatgc ccaaaaagag
taaggcgaga aaataaattg agacatactt atatttgtgc gaggttaacc cCat
SEQ ID NO: 8 Vibrio cholerae O1 str. C6706 Contig_20 amino acid Sequence
(WP_000524734.1)

| | |
|---|---|
| 1 | MGLTSHKYKY VSIYFLALLF LGIAVIESLH ISHTRDLQEG LRQQAKEDLS IVRFQLEAEI |
| 61 | LGDIYTVKGL TTLLTLDPDL NIYQWEPLSA AVIRNSDHLR SLGIAPNDVV AFSYPLPQTN |
| 121 | ALLGLDYRTV PQQWQSIKKA REIKQTFVSG PVDLVQGGRA LVIREPIFYD PPKDTRYWGV |
| 181 | LSVVMDWDSL LSATSIYSFG EHFQVAIRGL DSRGSEGDVF FGEPRVFEHA FAQENVYFPY |
| 241 | GSWRIAVAEK QDLLQQLSWY TRNAVRLLGY SVLLVLMAGF GVIMRLYQVA EERALHDPLT |
| 301 | HLPNRRYFIY TIEHYFENAK RSHSEGNFAL LNIDIDRFKS INDSHGHSAG DKVLVACAER |
| 361 | IKSSLRVSDL VARIGGDEFL VLIPRIHREQ DVLKVSDNIL KRISETPIVY DDKLIHVRVS |
| 421 | IGYALYDQSF ATPDEMFKLA DERMYTAKRR QNPLYRF |

SEQ ID NO: 9 Vibrio cholerae O1 str. C6706 Contig_20 DNA Sequence
(GI:446446879 REGION 171467..172840)
tcaaaagcga tagagtgggt tttgcctacg cttagcggta tacatacgtt catcggccag
tttgaacatt tcatcaggtg tggcaaacga ctggtcatac aaagcatatc cgatacttac
acgaacatgg ataagcttgt cgtcataaac gatgggcgtt tcagaaatcc tttttaaaat
attgtcactg actttaagca cgtcttgttc acgatgaatt cgtggaatta acacgagaaa
ctcatccccc ccaatccgcg ccaccagatc ggaaacccgc aggctcgatt taattctttc
cgcacaagcc accagcactt tatcgcctgc gctatgtcca tgggaatcgt tgatagattt
aaaacggtca atatcaatgt tcaacaaagc aaagttacct tcgctatgag agcgcttagc
attttcaaag tagtgttcaa tggtatagat aaaatagcgc cgattcggca agtgggttaa
agggtcatgt agcgcacgct cctccgcgac ttgataaagg cgcatgataa cgccaaagcc
tgccatcaat accaataaca ccgagtatcc caacaagcgc actgcatttc gggtatacca
agataactgc tgtagtaaat cttgcttttc agcgaccgca attcgccaac ttccgtaagg
gaaatagaca ttctcttgtg caaaagcgtg ctcaaatact cgaggctctc caaaaaacac
gtccccctca ctgccacggc tgtctaaacc acgaatcgca acctgaaaat gctccccaaa
gctgtaaata ctggttgctg aaagcaatga atcccaatcc atcaccacac tcagtacccc
ccaataacgc gtatccttcg gtgggtcgta gaatatcggt tctcgaatca ccagcgcgcg
cccaccttga acgagatcga caggtccaga gacgaacgtc tgtttgattt cacgtgcttt
ttttattgac tgccactgct gaggaacggt gcggtaatcc aaaccgagta gtgcattggt
ttgaggaagc ggatagctga aagcgaccac atcattaggg gcgataccta atgagcgtaa
gtgatcgcta ttcctgatca ccgccgctga aagcggctcc cattgataga tattgaggtc
gggatctagg gttaacaggg ttgttaaacc ttttacggta tagatatcac ccaaaatctc
agcttctaat tgaaaacgta cgatggaaag atcttcttta gcttgttgac gtaaaccctc
ttgtagatca cgtgtatggc taatatgaag ggattcaata accgcaatgc ccaaaaagag
taaggcgaga aaataaattg agacatactt atatttgtgc gaggttaacc ccat
SEQ ID NO: 10 Vibrio cholerae O1 str. C6706 Contig_20 amino acid Sequence
(WP_000524734.1)

| | |
|---|---|
| 1 | MGLTSHKYKY VSIYFLALLF LGIAVIESLH ISHTRDLQEG LRQQAKEDLS IVRFQLEAEI |
| 61 | LGDIYTVKGL TTLLTLDPDL NIYQWFPLSA AVIRNSDHLR SLGIAPNDVV AFSYPLPQTN |
| 121 | ALLGLDYRTV PQQWQSIKKA REIKQTFVSG PVDLVQGGRA LVIREPIFYD PPKDTRYWGV |
| 181 | LSVVMDWDSL LSATSIYSFG EHFQVAIRGL DSRGSEGDVF FGEPRVFEHA FAQENVYFPY |
| 241 | GSWRIAVAEK QDLLQQLSWY TRNAVRLLGY SVLLVLMAGF GVIMRLYQVA EERALHDPLT |
| 301 | HLPNRRYFIY TIEHYFENAK RSHSEGNFAL LNIDIDRFKS INDSHGHSAG DKVLVACAER |
| 361 | IKSSLRVSDL VARIGGDEFL VLIPRIHREQ DVLKVSDNIL KRISETPIVY DDKLIHVRVS |
| 421 | IGYALYDQSF ATPDEMFKLA DERMYTAKRR QNPLYRF |

SEQ ID NO: 11 Vibrio cholerae O1 str. C6706 Contig_20 DNA Sequence
(GI:446298852 REGION 177406..178581)
atggatagct ttgctggcaa ccaattaaaa gagatgacag agatgcgttttgctcgtaag
cagcatattg tcctgatcag ctctggtgtt gctaccgcta tttttcttgggtttgccctt
tactactatt ttaaccatca acccctgtca tccggtttat tgttattaagcggtattgtc
accttattga atatgatttc gctgaatcgt caccgcgaat tacacactcaagccgattta
attctgtcat taattctgct cacttatgcg ctggccttag tcagcaatgctcagcatgaa
ttatcgcatc tcttatggtt atatccgctc atcaccactt tagtcatgattaaccctttt
cggttaggct tggtttacag tgcagcgata tgcttagcga tgaccgcctctatccttttt
aatccggcac aaactggctc gtaccctatt gcacagacct attttttagtaagtctattt
acgctgacga ttatctgtaa taccgcttct ttctttttct caaaagcgatcaattatatt
cataccctat accaagaagg tattgaagag ttggcttatc ttgatccgttaacgggctta
gccaatcgtt ggagctttga aacttgggcc acagaaaagc tcaaagaacaacagagttcg
aataccatta ccgcgcttgt ttttctggat attgataatt tcaaacgcattaatgacagt
tacggccatg atgttggcga tcaggtgtta aaacattttg cacaccgtctacgcaataat
attcgtaata aagatcgagc caccaatcaa catgattatt ccattgctcgatttgctggt
gatgagtttg tgctcttgtt atatggtgtg cgaaatttgc gtgatctcgataatattctc
aaccgtatct gtaatctctt cgtcgaccgc tatcctgaga cggatatgctcaacaacctc
acggtgagta taggggcagc tatttatccc aaagatgcga tcactctgccggaactaacc
cgctgcgcag ataaagccat gtatgccgct aaacacggtg gaaaaaatcagtaccgctat
taccatgatg ccgctttccc tccggctgta gaaaccgtat taggcagtcagcccgttgag
gctcctaacg taactccact gaaaaaagcg cactaa
SEQ ID NO: 12 Vibrio cholerae O1 str. C6706 Contig_20 amino acid Sequence
(WP_000376707.1)

| | |
|---|---|
| 1 | MDSFAGNQLK EMTEMRFARK QHIVLISSGV ATAIFLGFAL YYYFNHQPLS SGLLLLSGIV |
| 61 | TLLNMISLNR HRELHTQADL ILSLILLTYA LALVSNAQHE LSHLLWLYPL ITTLVMINPF |
| 121 | RLGLVYSAAI CLAMTASILF NPAQTGSYPI AQTYFLVSLF TLTIICNTAS FFFSKAINYI |
| 181 | HTLYQEGIEE LAYLDPLTGL ANRWSFETWA TEKLKEQQSS NTITALVFLD IDNFKRINDS |
| 241 | YGHDVGDQVL KHFAHRLRNN IRNKDRATNQ HDYSIARFAG DEFVLLLYGV RNLRDLDNIL |
| 301 | NRICNLFVDR YPETDMLNNL TVSIGAAIYP KDAITLPELT RCADKAMYAA KHGGKNQYRY |
| 361 | YHDAAFPPAV ETVLGSQPVE APNVTPLKKA H |

SEQ ID NO: 13 Vibrio cholerae O1 str. C6706 Contig_30 DNA Sequence
(GI:446803291 REGION 173493..173939)
atgctagcgt tacctgcgga gtttgagcaa ttccattgga tggtcgatat ggttcagaat
gtcgatatgg gattgattgt gattaaccga gactacaacg tgcaagtgtg gaatgggttt
atgacccatc atagcggtaa gcaagctcat gatgttattg gtaaatctct gttcgagatt
tttccagaga tccctgtgga gtggtttaag ttaaaaacca aaccggtgta cgatctgggt
tgccgtagtt ttattacttg gcagcagcgc ccttatttgt tccattgccg taatgtgcgc
ccagtgactc agcaagccaa atttatgtat caaaacgtca cgcttaaccc aatgcgtaca
ccgacaggcg cgataaattc actcttctta tccattcaag atgcaacaag tgaagccctt
gtttctcaac aagcttcttc tcaataa
SEQ ID NO: 14 Vibrio cholerae O1 str. C6706 Contig_30 amino acid Sequence
(WP_000880547.1)

| | |
|---|---|
| 1 | MLALPAEFEQ FHWMVDMVQN VDMGLIVINR DYNVQVWNGF MTHHSGKQAH DVIGKSLFEI |
| 61 | FPEIPVEWFK LKTKPVYDLG CRSFITWQQR PYLFHCRNVR PVTQQAKFMY QNVTLNPMRT |
| 121 | PTGAINSLFL SIQDATSEAL VSQQASSQ |

SEQ ID NO: 15 Vibrio cholerae O1 str. C6706 Contig_42 DNA Sequence
(GI:446975354 REGION 107290..108807)
ttagacaaaa tttcgcacaa cgtatcgatc tcgtccgtgt tctttcgcat gataaagtgc
catatccgcc tgatggaaca aagagagata agactccatc tttggagaaa tagcatacac
accaccaatg ctcaccgtta gatattggca gagtgcatca accggatttg caatcgcgag
ctgctcgatt ttgcttctca tctgttgtgc atactgttct gcatcaaatg cacagtccga
agctaaaaca acacaaaact cttctccccc aaagcgcgcc acgattttct cgccatggaa
ctccaccgat tggagcacat cagcaacgga acataaggct tcatcgccag ccaaatgacc
aaagctgtca ttgaaacgtt tgaaaaaatc gatatcgaca agaaacagca ccagataggc
ttgcggacga tcgctcaaat aacttttaag ctgcttttct aaatggcgac gattggaaat
gcgggttagt ggatcatgct cagactgcca acgtaacact tgttgactat cctccaattg
tccgacgatt cggttgatcg tagtggcaaa ttctttcatc tccgatgaga taaaagtact
cgcatccggc atttttccgc ccgatgtttt aaattgttgc aacacttgac tggcggtcgt
gatcggtttg atcaaggcaa tcaccaccca taaattgact aagtacatca ccagtgaaaa
gaacagcaaa gcaagaattt cttcggttcg aatgaaggga ggatgcttaa tgtgatggtt
aattttaaac aacacactgg aattaccgct gtaatcgagt tgcttgatgt atgaaacatc
cacttcgtct tgcggtaagg gcgcatcatt tttacaggtt aagacttcaa tatcgacacc
agtggcttgc tcaaccacat tcgcaaactg ggcgcggact tttttaataa agattaagaa
acctttgtta caccctttcc catcactgtc acagacacga gccgtggcag ctaaataggg
ctcatcctcc accaccatat aacgaacgga agtcgagatt tcatccacac ttaaacgtgt
cgcctgctgt aaaatacgtg aaaaatccgg caataagtgc tcatagctag agctctgccc
cgttgctgcg tcatatttct tgccccaaac caaattgccc tcaggatcat agataaatac
gccatcgagg aattgtgaac tgaaagcgtg ctctccaata ttgctttgtg tgaactcaag
ggtgggtttt gcaatgaagt ctgccatttc atcccaagcg gcataatctg ccaaagaagc
ccccatcgcc ttacgttcta acgacaacaa ggtttcaacc cgctgcaact cggcctgttg
taactgcagc acttgcgcaa cttcacgatc atgtgaccag aaatatttaa aggtcagata
aaacattaaa aagcctaaca ccaccgctaa cgcattgagt gtcgttagcc agcgtaggct
aaagttattt aaattcat
SEQ ID NO: 16 Vibrio cholerae O1 str. C6706 Contig_42 amino acid Sequence
(WP_001052610.1)

| | |
|---|---|
| 1 | MNLNNFSLRW LTTLNALAVV LGFLMFYLTF KYFWSHDREV AQVLQLQQAE LQRVETLLSL |
| 61 | ERKAMGASLA DYAAWDEMAD FIAKPTLEFT QSNIGEHAFS SQFLDGVFIY DPEGNLVWGK |
| 121 | KYDAATGQSS SYEHLLPDFS RILQQATRLS VDEISTSVRY MVVEDEPYLA ATARVCDSDG |
| 181 | KGCNKGFLIF IKKVRAQFAN VVEQATGVDI EVLTCKNDAP LPQDEVDVSY IKQLDYSGNS |
| 241 | SVLFKINHHI KHPPFIRTEE ILALLFFSLV MYLVNLWVVI ALIKPITTAS QVLQQFKTSG |
| 301 | GKMPDASTFI SSEMKEFATT INRIVGQLED SQQVLRWQSE HDPLTRISNR RHLEKQLKSY |
| 361 | LSDRPQAYLV LFLVDIDFFK RFNDSFGHLA GDEALCSVAD VLQSVEFHGE KIVARFGGEE |
| 421 | FCVVLASDCA FDAEQYAQQM RSKIEQLAIA NPVDALCQYL TVSIGGVYAI SPKMESYLSL |
| 481 | FHQADMALYH AKEHGRDRYV VRNFV |

SEQ ID NO: 17 Vibrio cholerae O1 str. C6706 Contig_42 DNA Sequence
(GI:447036588 REGION 195345..197084)
ttagtggttt ggttgataaa ttgaggtctg attgcggcca ttcgctttgg cttggtataa
agcccgatcc gctagctcaa ccatttgctc aggtacatcc tcaggccgag gaataagcgt
cactatgcct aagctgacgg taatcctatc ggcaacctta gaatgatcat gtggaatcgc
taatccacga actttctcat ggattcgctc tgcgaccagt attgctccgg actgtggtgt
attgggcagc aaaataccaa actcttctcc cccgtagcgg gcaacacaat cagaatggcg
attggcgact tgagtaaagg caatcgctat ctgtttgagc gtctcatcgc ccatcaaatg
gccataagcg tcgttgtaat ctttgaaata atcgacatca cacagaatga tgcttaatgg
tttgccttca cgcacatgca aatgccagag ggtatgcagt tgttcatcaa aacgacgacg
attggcaaca tgagtcaagc tatctaaaaa gcttaggcgt tccagctctt ggttggcggc
ttctaattgt tcagcggcga gatagcgctc cgacacatct cgcgccatga tcagcacgcc
attggtgccc gaagccggat ctcgaaaagg cgatttcaca acatcaaacc agataaactc
accatctgag cgttcaattc tgtcgatgta gcgcagagac ttaccttggt gcaggacttg
gctatccgta tcggaaagac gcgcatagat gtgctcgggg atcacatctt gcagccgttt
accaaccaga tctgacactt ccgcgatccc gagagcttcc acaaacggct ggttacaggc
ttggtagacc atgttttcat tgaagatacc aatcgaatcg gggctagatt ctaagatgtt
ttgtaaaatc gtatcgcgct gtgccaatgc cacttcggtg tcacggcgtt tttccatctc
ttctcttaat tgacgctgca tgttgtacca gtcggtcaca tcatgactga tgccaagtag
cccaatattt tcaccttgcg gcgacatcaa tacccgttgg taggtttcta acagacagct
gcgcccatca ggcgtcacag tccagcaacg ctgactcgtg cgccctttca taatgccttt
aaaagtagcg ctgccctctt caatccggcc ttgccaaaac tgatcaaacg ctcggttggt
tgcgattaag tggccttcgg tacttttaat aaaaatcagc tcggagaggg aatcaagtgc
cgtgcgcgct atcgccagtg agtggcgctc ttgttgaatg tcatggctgg gacactcaaa
accaatcaca ttcactagcc ataatttctt cggccaacga cgtaagagcg aagctgagat
ctctagagtt tgggtcaaat tgcccggcac aggccaaagc agagggacgg aacgcttttg
ctgtgcactg ctggcgagcg ctcgataaaa agcttgctga ctctcttcac tctgctcggc
agaaaacaga tagtgacgtc ccaccaagcg gatccccagt aacaaatacg cggcaagatt
ggcacgtaaa acgcgatcct ctcctaccaa gagcatccct gacggtgcat ggtgaagtaa
ctgaatccac tgttgaggtt gaacatagcg ctgccatcct gaaaaaagcc ataacccacc
accaagcaca agcccggcag cgaacaagaa acgtacaaat tcagagagaa attcaggcat
SEQ ID NO: 18 Vibrio cholerae O1 str. C6706 Contig_42 amino acid Sequence
(WP_001113844.1)

| | |
|---|---|
| 1 | MPEFLSEFVR FLFAAGLVLG GGLWLFSGWQ RYVQPQQWIQ LLHHAPSGML LVGEDRVLRA |
| 61 | NLAAYLLLGI RLVGRHYLFS AEQSEESQQA FYRALASSAQ QKRSVPLLWP VPGNLTQTLE |
| 121 | ISASLLRRWP KKLWLVNVIG FECPSHDIQQ ERHSLAIART ALDSLSELIF IKSTEGHLIA |
| 181 | TNRAFDQFWQ GRIEEGSATF KGIMKGRTSQ RCWTVTPDGR SCLLETYQRV LMSPQGENIG |
| 241 | LLGISHDVTD WYNMQRQLRE EMEKRRDTEV ALAQRDTILQ NILESSPDSI GIFNENMVYQ |
| 301 | ACNQPFVEAL GIAEVSDLVG KRLQDVIPEH IYARLSDTDS QVLHQGKSLR YIDRIERSDG |
| 361 | EFIWFDVVKS PFRDPASGTN GVLIMARDVS ERYLAAEQLE AANQELERLS FLDSLTHVAN |
| 421 | RRRFDEQLHT LWHLHVREGK PLSIILCDVD YFKDYNDAYG HLMGDETLKQ IAIAFTQVAN |
| 481 | RHSDCVARYG GEEFGILLPN TPQSGAILVA ERIHEKVRGL AIPHDHSKVA DRITVSLGIV |
| 541 | TLIPRPEDVP EQMVELADRA LYQAKANGRN QTSIYQPNH |

SEQ ID NO: 19 Vibrio cholerae O1 str. C6706 Contig_40 DNA Sequence
(GI:446834936 REGION 93475..95058)
ttacataaag tcgaacatcc tacctgaatt gaaggcataa ttcgattcta ccttgctgca
ttgctgcgca atcgatacac gatttcgacc tttcgattta ctgagataga gctgatcatc
aacactctgt aaaatttccg gctcactgta ctcacagtta atgctcgccc caatactgat
ggttaaggtt aatggtgtct cggcattgag catcacaggt tctgcttcga ccactttacg
gatccgctct agataagtat aaagcgccgt ttcatcagta acggatgaca agatggcaaa
ctcatcaccg ccgaaacggg caaaaatatc cgattcaacc aactcttttt tgaccacatc
aaccacatgc gttaaagcgt aatcccccgc taaatgccca tagctgtcgt tgatttgctt
aaagcggtcg atatcaaatg aaatcaaggt aaaggattgt ttttcatcta acattttgca
caaatgctga ctaaagaagc ggcggttata gatgttggtc aaactgtcat gctccaccag
ataacgcagc tctgcggtac gctcctcaat catatctgtc agccgttgtt tctcttccag
ttgcattcgc atgatgtagc taagcagcag agaaataata acgccaccca accctaagcc
cagtagcacc cactcttcac tatggttaat cggctgatgc agttcaaact ccagcaccca
atcacggttt ggcaacacca atttgcgctc tattttgggt tcatcatccg ctcgccacat
cgggctttga taaagaaccg gactgtcttc cgaatcaaat ccggtgtcaa tcacgcgcat
atcgagatct tgttccatga cgctgatttg gaccagtttc tcgaaatagg tggataggcg
caccaccccg accatcacac caagtaagct gcgatcatct tctgaagaaa aaacagggtg
atagaccaac atgccatctt tgacgatcga cttatcaatc ccatcttgta gcaggcgcac
tttatccgaa acattcggcc gacgattaac gacaatatcc gccagtattc gtttgaaacg
ttcacgctcc gagtaaaagc ctaacagttt acgattgtca taattgagtg gataaatatc
cgataaaacg tatttcgctt ggtcatccgt accgaaaccg tatttgatct ctcccgtttt
tggcaccgtg tacaaagtga actcaggaaa acgttgctgc attcgcgcgg taaaagtttc
agcctgaggc ggctcaactt tcactaacca ttgtaaagca atcaggcttt gtgaaccttt
aagagtctct tctgcgaaag tgtgaaaacg cacccagtca tcgcttgtgc ttgagcggaa
aaagttggcg gcagagccga taaaatggat atcaccatcg acaaactgtt gcagtgccat
agtttgccta tccgcaaggt tttccagcag agtacgatta tggcgcagct gtaatgagta
tgcggtgtaa accacaaaca cagtcagaag cagagaaaac aacagtacca gcaagggcac
aatcacgcgc acatgtttga gcat
SEQ ID NO: 20 Vibrio cholerae O1 str. C6706 Contig_40 amino acid Sequence
(NP_000912192.1)

| | |
|---|---|
| 1 | MLKHVRVIVP LLVLLFSLLL TVFVVYTAYS LQLRHNRTLL ENLADRQTMA LQQFVDGDIH |
| 61 | FIGSAANFFR SSTSDDWVRF HTFAEETLKG SQSLIALQWL VKVEPPQAET FTARMQQRFP |
| 121 | EFTLYTVPKT GEIKYGFGTD DQAKYVLSDI YPLNYDNRKL LGFYSERERF KRILADIVVN |
| 181 | RRPNVSDKVR LLQDGIDKSI VKDGMLVYHP VFSSEDDRSL LGVMVGVVRL STYFEKLVQI |
| 241 | SVMEQDLDMR VIDTGFDSED SPVLYQSPMW RADDEPKIER KLVLPNRDWV LEFELHQPIN |
| 301 | HSEEWVLLGL GLGGVIISLL LSYIMRMQLE EKQRLTDMIE ERTAELRYLV EHDSLTNIYN |
| 361 | RRFFSQHLCK MLDEKQSFTL ISFDIDRFKQ INDSYGHLAG DYALTHVVDV VKKELVESDI |
| 421 | FARFGGDEFA ILSSVTDETA LYTYLERIRK VVEAEPVMLN AETPLTLTIS IGASINCEYS |
| 481 | EPEILQSVDD QLYLSKSKGR NRVSIAQQCS KVESNYAFNS GRMFDFM |

SEQ ID NO: 21 Vibrio cholerae O1 str. C6706 Contig_40 DNA Sequence
(GI:446533459 REGION 103406..104737)
tcagctcact aaactggtgt gatcgtgctt atcttggtgg gcgcaataca ccgtattgcc
ggattgatgt tttgcggtgt acatcgcctc atcagcaata cgcaataatt caggtacttg
ggtcgcttgc tctggatata aggcgacacc aatactcacc ccaatctcta agctctcttg
gttaagttga agcggctttt gtagtttttc tagcatctga taagccttat tgataacgcc
actgtgatcc tgcagcagat ctaggcatac cacaaattca tcccccccta agcgcccaca
aaaatccgat tctcgtatcg accctttgag ccgttgagcg atttcttgca agacaagatc
acctacttcg tgccctttgg tgtcattgat ttctttaaat ttatctaagt caaaaaacag
caaagccagc ttcatgtttg agcgcttcgc tttaattaac gcgtgactaa gctgctgttt
aaaggcacgg cggttcaaaa tacctgtcaa tgaatctctt tctgacaaga aacgtaattc
cgctttttga cgctctaatt tggcggtttt tcttgccact tccgcttgta actcatcttt
ggtaacggtt gtgctttgca gcgaagcctt catttgattg aaaaactgag ttaattgaac
aaactcttgt tcattatttt gagtggaaat tcggctggcg agatcccctt tcgccatttg
ttcaatccct tcttggagag ttttacatcc atgtcggaag cggcgtaaca ccaccagcgc
gataccacag acaaccgatg agaagagcag taagtgcgcc atggtggtta acaataaata
gcgttgatta ttaatgctct cttccatgac ttgacgctga aaataggcca actcctcatt
catgttttgc accaaaatat tgtatcgaga gtgaagtagc tcataagttc cgatgccatc
gaccaactta gtaatgcccg attcttcggc catgtagcgc tcttgttcta atagcccggc
taaactgtta ttcattcttt ggatgccggc taagtgttgc ccaaagaccg tttccatctc
gagctgccca gccaaaacct gctgcgcacg ataaacctgc tctaagctat gagcatcgtt
gtattgcaga aagacccaga gctggctacg caacatggca atgctgtttt ggatttccaa
aatcgtatcc agctcagcat tggtttgctg ctgccgctga tctaagttca gtaatgagaa
agcaataaaa ccaactaaca gcagtgatgc aataaacagt aacgtcattt tgcggtttaa
tgagttgatc aa
SEQ ID NO: 22 Vibrio cholerae O1 str. C6706 Contig_40 amino acid Sequence
(NP_000610805.1)

| | |
|---|---|
| 1 | MINSLNRKMT LLFIASLLLV GFIAFSLLNL DQRQQQTNAE LDTILEIQNS IAMLRSQLWV |
| 61 | FLQYNDAHSL EQVYRAQQVL AGQLEMETVF GQHLAGIQRM NNSLAGLLEQ ERYMAEESGI |
| 121 | TKLVDGIGTY ELLHSRYNIL VQNMNEELAY FQRQVMEESI NNQRYLLLTT MAHLLLFSSV |
| 181 | VCGIALVVLR RFRHGCKTLQ EGIEQMAKGD LASRISTQNN EQEFVQLTQF FNQMKASLQS |
| 241 | TTVTKDELQA EVARKTAKLE RQKAELRFLS ERDSLTGILN RRAFKQQLSH ALIKAKRSNM |
| 301 | KLALLFFDLD KFKEINDTKG HEVGDLVLQE IAQRLKGSIR ESDFCGRLGG DEFVVCLDLL |
| 361 | QDHSGVINKA YQMLEKLQKP LQLNQESLEI GVSIGVALYP EQATQVPELL RIADEAMYTA |
| 421 | KHQSGNTVYC AHQDKHDHTS LVS |

SEQ ID NO: 23 Vibrio cholerae O1 str. C6706 Contig_37 DNA Sequence
(GI:446848493 REGION 64235..66256)
atgctactta acgctttttc acgccgagtc ttcctttggc taggttggct attgatttcc
accagcagtt tagccgctac atctacgacg tataaggtcg ccaccgaagc ggatgacgtg
gtgactcgtg tgctttttga ttcgattgct caccacttca accttgaaat tgaatacgtc
aactacccca gttttaacga tattctggtg gcgatagaga ctggcaacgc cgattttgct
gccaacatta cttacactga tttgcgtgct caacgttttg atttttcaag accaaccaac
atcgagtaca cctatctcta cagttatggt ggcctacgtt tacccgagtt gcgcctcgtg
ggtatcccga aaggaaccac ctacgggacc ctactaaaag aacactatcc ctatatccag
caagttgagt atgaagggca tttagaagcg ctcactttgc tggaaagtgg ccgagtagac
ggagtggttg atgcgatcaa tcagctcaaa cctatgctac tgaaagggct tgatgtacaa
ctccttaacg accaattacc gattcagcct gtttctattg tgacgcctaa aggcaaacac
tcagcgctat tgggcaagat tgaaaaatac gcgcattcgg ctcacgtaca acgtttattg
cgtgaatcga tccaaaagta tcaattggac atccgtaagc aagctctgcg tcaatccgtg
gttgagagcg gactcaacgt gcagcgtgta ttgcgtgtta agctagagaa caacccgcaa
tatgcacttt atcagccaga cggttcggtt cgtgggatca gtgctgatgt tgtgtttcag
gcctgtgaga tgctactgct gaaatgcgaa ttggtcagta atggtcaaga aacatgggag
agcatgtttg atgatttaca ggataaaagc atcgatattt tggctcctat aacggtttct
cagcagcgta aaaacctcgc ttacttcagt gaaagctact accacccaca agcgattttg
gtcaaacgtg aacactataa agacgatgtg tatagcaatg tgtctgagtt ggtggctgaa
cgtattggcg tcatcaaaga cgattttttt gaagagctgt tacagcagat gctgccgaac
aagatcttgt tcagctacgc aagtcaggaa gagaaagttc aagccttact gaataaagag
gtggactaca tagtgctcaa tagagccaat tttaatctct tgcttcgcga gtcaacggag
atgttaccga ttgtagaaga caccatgatt ggcagtttct accaatatga cattgcgata
ggttttgcta aaaatccact tggtgcaact ctggcacctc ttttctctcg ggcaattaaa
atgctcaata ccgaacagat catacatacc tatgattatc agccaaattg gcgagccaca
ttacttgcgg aaaagaaata tcagcgcagt actcaatggc tttttgccat ggctttcatc
gttttgttta tggtggcgtt ttacctccat ggcatatcac ataccgataa ccttactaag
ttgcgcaatc gtcgcgcttt gtataaccga taccgccgcg ggttatcgcc tcgcctaagc
ttggtttatc ttgacgtgaa tacgtttaaa tcaatcaacg atcagtatgg acatgaagtg
ggtgacaaag tccttaagca gttggctcag cgcatcgaag cggtatggcg tgggcgcagc
tatcggattg gtggggatga atttatttta atcggtgaat gttctgctaa gcggcttgaa
catgtggttg cgcaatgtga acgttttatg tttgtggatg cagagcgcga tgtcagtttt
gaagtgagtg tggcgattgg tattgctaag aatcgtgagc ggaccgaatc actcaatgag
gtgatgcacc aagcggatat tgcgatgtat cgcgctaagg cggaatcgac gcaatcgcca
tttcaggctg ccagcaaggt aaaaggatta cacatcgttt aa
SEQ ID NO: 24 Vibrio cholerae O1 str. C6706 Contig_37 amino acid Sequence
(WP_000925749.1)

| | |
|---|---|
| 1 | MLLNAFSRRV FLWLGWLLIS TSSLAATSTT YKVATEADDV VTRVLFDSIA HHFNLEIEYV |
| 61 | NYPSFNDILV AIETGNADFA ANITYTDLRA QRFDFSRPTN IEYTYLYSYG GLRLPELRLV |
| 121 | GIPKGTTYGT LLKEHYPYIQ QVEYEGHLEA LTLLESGRVD GVVDAINQLK PMLLKGLDVQ |
| 181 | LLNDQLPIQP VSIVTPKGKH SALLGKIEKY AHSAHVQRLL RESIQKYQLD IRKQALRQSV |
| 241 | VESGLNVQRV LRVKLENNPQ YALYQPDGSV RGISADVVFQ ACEMLLLKCE LVSNGQETWE |
| 301 | SMFDDLQDKS IDILAPITVS QQRKNLAYFS ESYYHPQAIL VKREHYKDDV YSNVSELVAE |
| 361 | RIGVIKDDFF EELLQQMLPN KILFSYASQE EKVQALLNKE VDYIVLNRAN FNLLLRESTE |
| 421 | MLPIVEDTMI GSFYQYDIAI GFAKNPLGAT LAPLFSRAIK MLNTEQIIHT YDYQPNWRAT |
| 481 | LLAEKKYQRS TQWLFAMAFI VLFMVAFYLH GISHTDNLTK LRNRRALYNR YRRGLSPRLS |
| 541 | LVYLDVNTFK SINDQYGHEV GDKVLKQLAQ RIEAVWRGRS YRIGGDEFIL IGECSAKRLE |
| 601 | HVVAQCERFM FVDAERDVSF EVSVAIGIAK NRERTESLNE VMHQADIAMY RAKAESTQSP |
| 661 | FQAASKVKGL HIV |

SEQ ID NO: 25 Vibrio cholerae O1 str. C6706 Contig_36 DNA Sequence
(GI:446054248 REGION 42225. 43517)
ctatctgaac tgatcctgct tgagttcttt cgcactggga agaggcagga tctcttcccc
cattcgataa atatgatagc catgtttgcc tctgtatttg acccagtaca tggctttatc
ggcttgtagc agcagttttt ctaagtcaat gtgcagactg ttcatatgac taatcccgat
actgcaaccc acttgcgcac tctgctgacc caatccaatc ggctcagagg aggattcgat
caactgagcc gcaaaccgct cgatagattc ggcaacaaat tcatccagcg gaatgtaaat
agcaaactca tcaccaccga gccgtccgac cacaaaatca gaaaaatgtg tttgcgccaa
ggcataaaaa cgtttggcga tttcacgtaa tacctcatcg ccagccgcat gccccaaggt
atcattcacc tgcttaaaac catccaaatc aatcaatagc agcaccatag tggtgctagc
acgctgtttg cggagcacga acttctcaca acctaaacgg ttttttagtc ccgtcagcgt
gtcttgttcg gcaatggtgc gatagtagct ttcccaacgt tcaatctgtt gacgcagctc
gcgttcacgc agtaaagctt gatgggaagc gtcgataaat tcattgatgc ttttggccac
caaaccaatc tcgttgtggt gatcttctgc ggctaccgcc actttgcgat catgatctgg
ccgcacttcg gataacgcct gtgaaagatc cgtcaggggt ttaccgacca agcggcgaac
gatccagata agcgcaataa aagtcacgag aaactggatc aaaaccacgg ctatctgatc
aagaatctga ttaatggctt gctgacgaat cacctgatga tcctcatgaa tcatcagata
gccaatcaaa ttaccatcta cgggagaatc taatcggtag cggttcgcat cactccaata
attctgctct ttgtaggttg aggggatggt ggtgcgctca aagacaatgc catccacgct
ggctaactta accgcattga tctcttgatg aagcagcaac gcatccatca cctcggaggc
aatatcgtaa ttattcacat acagtgcaat ggccgccgag ttactcaagg agagcgcaag
cttctcttcc agctcttgtt tttgctgctc aacactctgt atgccgcgcg gaataatgat
ggccaaaatg atcagcaaat acccaagtgc acacagtgaa atcatcttca gcaagcgatt
aaccagtggc gaagttcgcg tttgatcagt cat
SEQ ID NO: 26 Vibrio cholerae O1 str. C6706 Contig_36 amino acid Sequence
(WP_000132103.1)

| | |
|---|---|
| 1 | MTDQTRTSPL VNRLLKMISL CALGYLLIIL AIIIPRGIQS VEQQKQELEE KLALSLSNSA |
| 61 | AIALYVNNYD IASEVMDALL LHQEINAVKL ASVDGIVFER TTIPSTYKEQ NYWSDANRYR |
| 121 | LDSPVDGNLI GYLMIHEDHQ VIRQQAINQI LDQIAVVLIQ FLVTFIALIW IVRRLVGKPL |
| 181 | TDLSQALSEV RPDHDRKVAV AAEDHHNEIG LVAKSINEFI DASHQALLRE RELRQQIERW |
| 241 | ESYYRTIAEQ DTLTGLKNRL GCEKFVLRKQ RASTTMVLLL IDLDGFKQVN DTLGHAAGDE |
| 301 | VLREIAKRFY ALAQTHFSDF VVGRLGGDEF AIYIPLDEFV AESIERFAAQ LIESSSEPIG |
| 361 | LGQQSAQVGC SIGISHMNSL HIDLEKLLLQ ADKAMYWVKY RGKHGYHIYR MGEEILPLPS |
| 421 | AKELKQDQFR |

SEQ ID NO: 27 Vibrio cholerae O1 str. C6706 Contig_62 DNA Sequence
(GI:480994257 REGION 1..1003)
agcgcatacg ctcaagtagg gcttgctcac gttgctccgc taagagtaag cgttcagaaa
gtgaagacat ctcgcgcagt aaaggcgcca ttttcagctt gagctgttct agctccgtct
gttctttgag cgcggtctgg ctacgagcca ctaaactgct cagctcgcca ttcatctctt
ggcggtgtgc catgtaactc tggctttgct caagattttg agtcgcgctt tttaggttat
tgccaatcga gagattcact tgctcgagaa aggcttcggc tgctttgcgc tcagcatggc
ttccatcgac gactaaacgc agtacttcaa gggtgagctc aagcagggta tgggtattga
cgccaagcag aagcttggtt cggatatcgg tcagttgatc acccgattca ccattgaaat
ccaactcagt aatcaagtgt tgtaaatcaa cggcaagtcg atgcagcagt tctcgatccg
cttgttgagt aagctcattg agcgccaaat tgggattggc acattgaatt ttgaccgcgc
gttcataaat ttccagcaaa cgcaaagctt gctgagtttt ttccagcggc tgtgcggcgc
taaaactcag cagatctcga agatcgcgtt tgatcttggc gggtaagccg gggacgcgca
gtagcgtttc accactgtgc tgtagctggc tatccagatg actcgtttgt ttgtccatgg
ccaatgactg ttgtttcaac atgcgttcca gtacggctaa tttcgggatc agcgtactga
tgtctttttg ttgttctaat gcaaaacaga gttcttctaa actttggttt agtcgagagc
tactgccgcg gcaagtcgta gccaaggaag tgaccattcg tttaagaact tgctgctctc
ggttaaattt gaacgaagta tccctttgtg tcaaacgtac ttgttctaac tgagatttca
gtttttgaag ctotgottgg atatcttgtt ctagaacgcc cat
SEQ ID NO: 28 Vibrio cholerae O1 str. C6706 Contig_62 amino acid Sequence
(NP_000538436.1)

| | |
|---|---|
| 1 | MGVLEQDIQA ELQKLKSQLE QVRLTQRDTS FKFNREQQVL KRMVTSLATT CRGSSSRLNQ |
| 61 | SLEELCFALE QQKDISTLIP KLAVLERMLK QQSLAMDKQT SHLDSQLQHS GETLLRVPGL |
| 121 | PAKIKRDLRD LLSFSAAQPL EKTQQALRLL EIYERAVKIQ CANPNLALNE LTQQADRELL |
| 181 | HRLAVDLQHL ITELDFNGES GDQLTDIRTK LLLGVNTHTL LELTLEVLRL VVDGSHAERK |
| 241 | AAEAFLEQVN LSIGNNLKSA TQNLEQSQSY MAHRQEMNGE LSSLVARSQT ALKEQTELEQ |
| 301 | LKLKMAPLLR EMSSLSERLL LAEQREQALL ERMRYSKDQM EALSDLAQDY RRRLEDQALR |
| 361 | AQLDPLTKVY NRSSFTERLE HEYRRWIRTQ HNLRVVLFDI DKFKSINDSF GYTAGDKALS |
| 421 | IIARTIKKEL RDSDTVARFS GEEFILLLPE RSDNESYQII HQIQLNVSKL PFKFRDKSLT |
| 481 | ITLSAASIRF MDSDTPETVL DRLNLTLSEA KHIGPSQLAW K |

SEQ ID NO: 29 Vibrio cholerae O1 str. C6706 Contig_27 DNA Sequence
(GI:480994257 REGION 1..563)
atagcaaaga tcagatggaa gccctgtctg atttggcaca agattatcgt cgccgccttg
aagatcaagc attgcgcgca caactcgatc ctctgaccaa agtgtacaac cgcagcagct
ttactgagcg acttgaacat gagtatcgcc gctggatccg tacgcaacac aatttgcggg
tagtgctgtt tgatattgat aaattcaaat cgatcaacga cagctttggc tacaccgcag
gcgataaggc cttaagtatc attgctcgca ccatcaaaaa agaattacga gacagtgaca
ccgtggctcg cttctctggt gaagagttca ttctgttact gcctgaacgc tccgataatg
agagttacca gattattcac cagatccagc tcaacgtgtc gaaactaccg ttcaagttcc
gcgataagag cctaaccatc acgctgtctg cggcgagtat ccgcttcatg gattcagata
cccccgaaac ggttcttgat cgtttaaatc tgacgctaag tgaagccaaa catatcggtc
caagtcagtt agtttggaaa taa
SEQ ID NO: 30 Vibrio cholerae O1 str. C6706 Contig_27 amino acid Sequence
(NP_001888804.1)

| | |
|---|---|
| 1 | MLKQQSLAMD KQTSHLDSQL QHSGETLLRV PGLPAKIKRD LRDLLSFSAA QPLEKTQQAL |
| 61 | RLLEIYERAV KIQCANPNLA LNELTQQADR ELLHRLAVDL QHLITELDFN GESGDQLTDI |
| 121 | RTKLLLGVNT HTLLELTLEV LRLVVDGSHA ERKAAEAFLE QVNLSIGNNL KSATQNLEQS |
| 181 | QSYMAHRQEM NGELSSLVAR SQTALKEQTE LEQLKMKMAP LLREMSSLSE RLLLAEQREQ |
| 241 | ALLERMRYSK DQMEALSDLA QDYRRRLEDQ ALRAQLDPLT KVYNRSSFTE RLEHEYRRWI |
| 301 | RTQHNLRVVL FDIDKFKSIN DSFGYTAGDK ALSIIARTIK KELRDSDTVA RFSGEEFILL |
| 361 | LPERSDNESY QIIHQIQLNV SKLPFKFRDK SLTITLSAAS IRFMDSDTPE TVLDRLNLTL |
| 421 | SEAKHIGPSQ LVWK |

SEQ ID NO: 31 Vibrio cholerae O1 biovar El Tor str. N16961 amino acid Sequence
(NP_233340.1 GI:15601709)

| | |
|---|---|
| 1 | MMTTEDFKKS TANLKKVVPL MMKHHVAATP VNYALWYTYV DQAIPQLNAE MDSVLKNFGL |
| 61 | CPPASGEHLY QQYIATKAET NINQLRANVE VLLGEISSSM SDTLSDTSSF ANVIDKSFKD |
| 121 | LERVEQDNLS IEEVMTVIRR LVSDSKDIRH STNFLNNQLN AATLEISRLK EQLAKVQKDA |
| 181 | LFDSLSGLYN RRAFDGDMFT LIHAGQQVSL IMLDIDHFKA LNDNYGHLFG DQIIRAIAKR |
| 241 | LQSLCRDGVT AYRYGGEEFA LIAPHKSLRI ARQFAESVRR SIEKLTVKDR RSGQSVGSIT |
| 301 | ASFGVVEKIE GDSLESLIGR ADGLLYEAKN LGRNRVMPL |

SEQ ID NO: 32 Vibrio cholerae O1 biovar El Tor str. N16961 DNA Sequence
(DQ776083.1 GI:109706432)

| | |
|---|---|
| 1 | atgatgacaa ctgaagattt caaaaaatcc acggctaact taaaaaaagt cgtaccttta |
| 61 | atgatgaaac atcatgtcgc ggccaccccc gtgaactatg ccttgtggta tacctacgtc |
| 121 | gaccaagcca ttccgcaact gaatgcggaa atggactctg tattgaaaaa ttttgggctt |
| 181 | tgcccacccg cttctggtga acatctttac caacaataca ttgcgaccaa agcagaaacc |
| 241 | aatattaatc agttacgtgc gaatgttgag gtacttcttg gtgaaattag cagttcaatg |
| 301 | agtgatacgc tcagtgacac cagttccttt gctaatgtga ttgataaaag ctttaaggat |
| 361 | ttagagcgcg tcgagcaaga caatctctcg attgaagaag taatgacggt gatccgccgc |
| 421 | ttggtgagtg actctaaaga tattcgacac tcaaccaatt tcctaaataa tcaactgaac |
| 481 | gcggcaacac tagaaatctc tcgtcttaaa gagcagctgg cgaaagttca gaaagatgct |
| 541 | ctgtttgaca gtttatctgg actctataac cgccgagctt ttgatggcga tatgttcacg |
| 601 | ctgatccatg caggtcaaca agtcagcctg atcatgctcg acatcgacca cttcaaagcc |
| 661 | cttaatgata actatggcca cctgtttggt gaccaaatta tccgtgcgat cgccaaacgt |
| 721 | cttcaaagcc tatgccgtga cggcgtgaca gcttatcgtt atggcggtga agagtttgca |
| 781 | ctgattgctc cgcacaaatc gctgcgtatt gcacgccagt ttgctgaatc ggtgcgacgt |
| 841 | tcaatagaaa agctcaccgt aaaagatcgg cgtagcggtc aatcggtcgg tagcattacc |
| 901 | gcttcgtttg gtgtagtaga aaagattgaa ggtgactctt tggagtctct tatcggtcga |
| 961 | gcggatggat tgctgtatga agcgaaaaat ctgggccgca atcgagtcat gccgctcttg |

SEQ ID NO: 33 Vibrio cholerae VCA0956 O1 biovar El Tor str. N16961
chromosome II DNA Sequence (gi|15600771:904820-905839, NC_002506.1)
GTGATGACAACTGAAGATTTCAAAAAATCCACGGCTAACTTAAAAAAAGTCGTACCTTTAATGATGAAAC
ATCATGTCGCGGCCACCCCCGTGAACTATGCCTTGTGGTATACCTACGTCGACCAAGCCATTCCGCAACT
GAATGCGGAAATGGACTCTGTATTGAAAAATTTTGGGCTTTGCCCACCCGCTTCTGGTGAACATCTTTAC
CAACAATACATTGCGACCAAAGCAGAAACCAATATTAATCAGTTACGTGCGAATGTTGAGGTACTTCTTG
GTGAAATTAGCAGTTCAATGAGTGATACGCTCAGTGACACCAGTTCCTTTGCTAATGTGATTGATAAAAG
CTTTAAGGATTTAGAGCGCGTCGAGCAAGACAATCTCTCGATTGAAGAAGTAATGACGGTGATCCGCCGC
TTGGTGAGTGACTCTAAAGATATTCGACACTCAACCAATTTCCTAAATAATCAACTGAACGCGGCAACAC
TAGAAATCTCTCGTCTTAAAGAGCAGCTGGCGAAAGTTCAGAAAGATGCTCTGTTTGACAGTTTATCTGG
ACTCTATAACCGCCGAGCTTTTGATGGCGATATGTTCACGCTGATCCATGCAGGTCAACAAGTCAGCCTG
ATCATGCTCGACATCGACCACTTCAAAGCCCTTAATGATAACTATGGCCACCTGTTTGGTGACCAAATTA
TCCGTGCGATCGCCAAACGTCTTCAAAGCCTATGCCGTGACGGCGTGACAGCTTATCGTTATGGCGGTGA
AGAGTTTGCACTGATTGCTCCGCACAAATCGCTGCGTATTGCACGCCAGTTTGCTGAATCGGTGCGACGT
TCAATAGAAAAGCTCACCGTAAAAGATCGGCGTAGCGGTCAATCGGTCGGTAGCATTACCGCTTCGTTTG
GTGTAGTAGAAAAGATTGAAGGTGACTCTTTGGAGTCTCTTATCGGTCGAGCGGATGGATTGCTGTATGA
AGCGAAAAATCTGGGCCGCAATCGAGTCATGCCGCTCTAA
SEQ ID NO: 34 Vibrio cholerae strain 2012EL-2176 chromosome 2 amino acid
Sequence (AIT31434.1)

| | |
|---|---|
| 1 | MDHRFSTKLF LLLMIAWPLL FGSMSEAVER QTLTIANSKA WKPYSYLDEQ GQPSGILIDF |
| 61 | WLAFGEANHV DIEFQLMDWN DSLEAVKLGK SDVQAGLIRS ASRLAYLDFA EPLLTIDTQL |
| 121 | YVHRTLLGDK LDTLLSGAIN VSLGVVKGGF EQEFMQREYP QLKLIEYANN ELMMSAAKRR |
| 181 | ELDGFVADTQ VANFYIVVSN GAKDFTPVKF LYSEELRPAV AKGNRDLLEQ VEQGFAQLSS |
| 241 | NEKNRILSRW VHIETIYPRY LMPILASGLL LSIVIYTLQL RRTVRLRTQQ LEEANQKLSY |
| 301 | LAKTDSLTDI ANRRSFFEHL EAEQTRSGSL TLMVFDIDDF KTINDRFGHG AGDNAICFVV |
| 361 | GCVRQALASD TYFARIGGEE FAIVARGKNA EESQQLAERI CQRVAEKKWV VNAQHSLSLT |
| 421 | ISLGCAFYLH PARPFSLHDA DSLMYEGKRN GKNQVVFRTW S |

SEQ ID NO: 35 Vibrio cholerae 2012EL-2176 chromosome 2 DNA Sequence
(GI:695934235 REGION 195154..196539)
atggatcatc gcttttcgac caaactgttt ctgcttctca tgattgcttg gccgctttta
ttcggatcaa tgagtgaggc tgtagagcgc caaaccttga ctattgccaa ctcaaaagca
tggaaaccct attcttattt ggatgaacag ggacagcctt ctggcatatt gattgatttt
tggttggctt ttggtgaagc gaatcatgtc gatattgaat tccaactgat ggattggaat
gattccctag aagcggtgaa gcttggcaaa tccgatgttc aagctggttt gatccgttct
gcttcaagat tagcgtatct cgattttgca gaacctttac tgacaatcga tacacaactc
tacgtacacc gcacgttatt gggcgataaa ttggatacgc tgctatcggg ggccattaac
gtctcattag gtgtagtaaa agggggattt gaacaagagt tcatgcaacg agaatatcct
caacttaagt tgattgagta cgccaacaat gaattgatga tgtctgcagc aaagcgacga
gaattagatg gttttgtggc cgatactcag gtcgccaatt tctatatagt ggtttccaat
ggcgcgaaag attttacgcc agtgaagttt ctttattcag aggaattacg tccagcggtc
gccaaaggca atagggattt attagagcaa gtagagcagg ggtttgcaca attaagtagc
aatgagaaaa accgtatttt aagtcgatgg gttcatattg aaacgattta tccacgttac
ttaatgccga ttctcgcttc aggtctctta ctcagtatcg ttatttatac tottcagcta
cggcgtaccg ttcgattgcg aacacagcaa cttgaagaag ccaatcaaaa actctcctat
ttagcgaaaa cggatagctt gacggacatt gctaatcgcc gttcgttttt tgaacatctt
gaagcggaac aaacacgatc aggcagctta acgttgatgg tttttgatat tgatgacttc
aaaaccatta acgatcgctt tgggcatggc gcaggagata atgccatctg tttcgtggtt
gggtgtgtgc gacaagcttt agcatcggat acctactttg caaggattgg tggtgaagag
tttgctattg tagcgcgtgg taaaaatgca gaagagtcgc agcagttagc tgagcgaatt
tgccaacgag ttgcagaaaa aaagtgggta gtgaatgccc aacactctct gtcactcacc
atcagcctag gctgtgcatt ttacctacac ccagctcggc cattcagttt gcacgatgcc
gatagcttaa tgtacgaagg aaagcggaat ggaaagaacc aggttgtctt tcgtacctgg tcataa
SEQ ID NO: 36 Vibrio cholerae strain 2012EL-2176 chromosome 2 amino acid
Sequence (AIT31434.1)

| | |
|---|---|
| 1 | MDHRFSTKLF LLLMIAWPLL FGSMSEAVER QTLTIANSKA WKPYSYLDEQ GQPSGILIDF |
| 61 | WLAFGEANHV DIEFQLMDWN DSLEAVKLGK SDVQAGLIRS ASRLAYLDFA EPLLTIDTQL |
| 121 | YVHRTLLGDK LDTLLSGAIN VSLGVVKGGF EQEFMQREYP QLKLIEYANN ELMMSAAKRR |
| 181 | ELDGFVADTQ VANFYIVVSN GAKDFTPVKF LYSEELRPAV AKGNRDLLEQ VEQGFAQLSS |
| 241 | NEKNRILSRW VHIETIYPRY LMPILASGLL LSIVIYTLQL RRTVRLRTQQ LEEANQKLSY |
| 301 | LAKTDSLTDI ANRRSFFEHL EAEQTRSGSL TLMVFDIDDF KTINDRFGHG AGDNAICFVV |
| 361 | GCVRQALASD TYFARIGGEE FAIVARGKNA EESQQLAERI CQRVAEKKWV VNAQHSLSLT |

SEQ ID NO: 37 Vibrio cholerae 2012EL-2176 chromosome 2 DNA Sequence
(G1:695934238 REGION 199457..200695)
ttagctagcg actttgacac aattgcgccc agcttgcttc gctttataaa gtgccccatc
cgctgctttg agtgcctcaa taggatggcg gtacagctca gaatcacaca cgccaatgct
gatggtaata gtgacaatgt cactgttact ttttcggctg cgtttttttg caccttcagc
atgacttttc gggcgctggt tggtgtcacg aatcaccaac tcgtaggact caatatcctg
ccgtaaggcc tcgatgaaag gcaaaacctc ctttgccaat tttcctttgt aaataatcga
gaactcctca ccaccatagc ggtaaactcg tgctttaccg ttgatttcac gtaatcgaga
ggcaaccagt cttaatacat cgtcccccgt atcatgcccg taagtatcgt taaacttctt
gaaatggtcg acatcgagca tagcgagggt aaattttcga cctatatgtt ttaaatcctg
atcaagcgct tgccgaccag gaatttgggt gagtgggtcg ttaaatgcca tctcatagcc
cgcggaaatg aggtaaacca gaataagcag cccagataag gtaaacatga tggtggaaat
ataaggcaca tgaaacagca caaacgcatt catgctcaat acaatcgaac tataaaccac
aacatcaaga atttgattgc gcgttaatac cgagatagca gcaatacctg cgagtgcgac
aagataggca acaaccacca agggtaagcg agaaatttgc ggtacaacga aaaatattcc
ctcggtgagg ctggaatggt ctgtttcacc tatgtgtagc tgggtcagcc aagcccaaaa
gatgaacagc aataaaatag ccaagtaact gagaaaggat ttgctgaata atccagcatt
cttgtaggcg taaggtaaaa aacaggccac aggcaaaagc aagctcagca taatgagttc
aagcatggtg gaattgacgg ttaaaggcgt ttgaagtcga atttggatca accagtaagc
cagtaacatc gtcatcgcta ccatggcgat tctgctttgt ttaaaaatgt gagcaacggt
tagcgcaatc aaaaagagaa tgtaggggag gttgaccgcc atgcctaagt tagactttat
caccaatacc acattgctca agcctagcca aatggctacc agcagcaata gaggaaaacc
gaaacggaac caaggtgaag taacaaagct agaagacat
SEQ ID NO: 38 Vibrio cholerae strain 2012EL-2176 chromosome 2 amino acid
Sequence (AIT31437.1)

| | |
|---|---|
| 1 | MSSSFVTSPW FRFGFPLLLL VAIWLGLSNV VLVIKSNLGM AVNLPYILFL IALTVAHIFK |
| 61 | QSRIAMVAMT MLLAYWLIQI RLQTPLTVNS TMLELIMLSL LLPVACFLPY AYKNAGLFSK |
| 121 | SFLSYLAILL LFIFWAWLTQ LHIGETDHSS LTEGIFFVVP QISRLPLVVV AYLVALAGIA |
| 181 | AISVLTRNQI LDVVVYSSIV LSMNAFVLFH VPYISTIMFT LSGLLILVYL ISAGYEMAFN |
| 241 | DPLTQIPGRQ ALDQDLKHIG RKFTLAMLDV DHFKKFNDTY GHDTGDDVLR LVASRLREIN |
| 301 | GKARVYRYGG EEFSIIYKGK LAKEVLPFIE ALRQDIESYE LVIRDTNQRP KSHAEGAKKR |
| 361 | SRKSNSDIVT ITISIGVCDS ELYRHPIEAL KAADGALYKA KQAGRNCVKV AS |
| 421 | ISLGCAFYLH PARPFSLHDA DSLMYEGKRN GKNQVVFRTW S |

SEQ ID NO: 39 Vibrio cholerae 2012EL-2176 chromosome 2 DNA Sequence
(GI:695934360 REGION 336934..338817)
atgtacacct cagcccgtaa atatttcata caatttgcca ttgttgcgtt tgtacttggt
ttcattccta cactgtattt catacatgct gctagccagc ttgagactca agcggtcagc
agcgttgaaa aacagactcg cttacagctt gagttcagtc agcatgactt gttacgaatg
ctggaaagca cacaccaagc cacccagctg ttagctaaaa atgacctttt attcacggct
gtcaccacac caagcaaaga agcactcagt caactcaaaa cattgtggga tgtgacgtta
agatcgcaag cgattttctc ttcattcaga ttgctggata gacaaggaaa agaacaactt
aaagcgattt acgatgggca ccaagtcacc tttgttgaat ctgctcaaac gacagatccg
ttcagccagc aaattgtggc tcaatacgcc caactcacga cgcctcaagt ttgggcaacg
caagtcgcga tgtcagcaga tacgccttct ggtatgctgc cgacctttcg ttttgtgacg
ggtattgagc atcaaggcca acggcaaggt tttcttgtcg tgacggtgaa gctacagtct
ctctatcaac gtctctcttt tatttatgat cagtttgatt caccggatat tttgaattcg
gcaggagaat tactgctcag tgaacacaag ccatccggta cacgttcaac ctcttcactc
cacttttcag cccaacaccc agagctttgg caaaaaatcc aactcaacca acaaggcttt
gctctatcca atcaaacctg gtttagctat atcaaagtgg atctcagttc tgtcttacct
gactttaaac ctttggtatt ggtactgcgc atcaataagg cagaaataga taagacctac
gcaaatgcgc gctgggcact gatgagtcaa gcggtgacag tgttatcgct actctctatc
attgcggctg gatttgcggc atggaacatc aaccatttaa aaaatagcct tgacagtaaa
ttggctcgag cagcgatgga tggcatgtca gcggtggtca ttaccgaccg ccagaatcgc
atcatcaaag taaacaacga atttacccgc ctaagtggtt acacttttga agatgtcaaa
ggtaagcagc cgtccatttt tgottctgga ttacacaaag tcgaattcta tatgcagatg
tggaaagctc tgcaagacaa tggcgtatgg gaaggtgaag tgatcaacaa acgcaaagat
ggcgaaagca tcaccgaaat tctccgtatt caaagcatcc gcgatgaaga caatgtcatt
caattctacg ttgcctcttt tgtggatatt tcacatcgca aggcgctgga gaatcgcctg
cgtgagctga gcgaaaaaga tgcgttaacc gatttgtgga atcgacgtaa attcgatcaa
accatctctt tagagtgcgc taagcgtcgc cgttatcccg atcaagccca gagctgcctt
gctatcattg atatcgacca ctttaaacgc attaacgaca aattcggaca caacgaaggg
gacctagtgt tacggaccgt tgcgaaaggc atccaagatc agttacggga atcggatttt
atcgcacgga ttggcggaga agagtttgcc attattttcc cctacacttc cattgaagaa
gccgaacaag tacttaaccg cgtacgcctg catatcgctt cattacacca tcaacaagtg
accctaagtg gtggtgttac cgatgtttgc acatcacccg accaaagcta caaaagagcc
gatctggctt tatatgaatc caaaacatcg ggacgcaacc aaatatcagt actcaccgcc
atggaaatgc atcactttgc gtga
SEQ ID NO: 40 Vibrio cholerae strain 2012EL-2176 chromosome 2 amino acid
Sequence (AIT31559.1)

| | |
|---|---|
| 1 | MYTSARKYFI QFAIVAFVLG FIPTLYFIHA ASQLETQAVS SVEKQTRLQL EFSQHDLLRM |
| 61 | LESTHQATQL LAKNDLLFTA VTTPSKEALS QLKTLWDVTL RSQAIFSSFR LLDRQGKEQL |
| 121 | KAIYDGHQVT FVESAQTTDP FSQQIVAQYA QLTTPQVWAT QVAMSADTPS GMLPTFRFVT |
| 181 | GIEHQGQRQG FLVVTVKLQS LYQRLSFIYD QFDSPDILNS AGELLLSEHK PSGTRSTSSL |
| 241 | HFSAQHPELW QKIQLNQQGF ALSNQTWFSY IKVDLSSVLP DFKPLVLVLR INKAEIDKTY |
| 301 | ANARWALMSQ AVTVLSLLSI IAAGFAAWNI NHLKNSLDSK LARAAMDGMS AVVITDRQNR |
| 361 | IIKVNNEFTR LSGYTFEDVK GKQPSIFASG LHKVEFYMQM WKALQDNGVW EGEVINKRKD |
| 421 | GESITEILRI QSIRDEDNVI QFYVASFVDI SHRKALENRL RELSEKDALT DLWNRRKFDQ |
| 481 | TISLECAKRR RYPDQAQSCL AIIDIDHFKR INDKFGHNEG DLVLRTVAKG IQDQLRESDF |
| 541 | IARIGGEEFA IIFPYTSIEE AEQVLNRVRL HIASLHHQQV TLSGGVTDVC TSPDQSYKRA |
| 601 | DLALYESKTS GRNQISVLTA MEMHHFA |

SEQ ID NO: 41 Vibrio cholerae 2012EL-2176 chromosome 2 DNA Sequence
(GI:695934436 REGION 430738..432621)
atggcaccga tcctttcaca ctcgatcccg atcccttcta gcatgcaggc aaattggcag
cagatgctca acctgctggc cgaagtgctg aaagtctcag ccaccctgat catgcgttta
cgccatcacg atcttgatgt gttttgtacc agtgtcggca gtgacaatcc ataccaagtc
ggcatgaccg aacgattagg cacaggcttg tattgtgaaa ctgtggtcaa tactcgccag
atattgttag tcagtaacgc cgacctcgac ccattgtgga aggataaccc agatctggaa
ttgggcatgc gcgcttactg tggcgtacca ttgcaatggc caaacggtga gctttttgga
tctttgtgtg tcaccgatcg tcaagctcgc cagtttctta gtaccgatca gcaattgata
aaaacctttg ctgaatcgat tgaagctcag cttaaaaccc tttaccaacg cgaaacgttg
ttgcaaatga accaagattt gcacttcaaa gttcgtcata aaatgcaaag catcgcctcg
ctgaaccaat ctctccatca agagatcgat aaacgccgtg ccgcagaaca gcagattgag
tatcagcgca gtcacgacct tgggactggc tttctgaatc gcacggcatt ggagcagcag
ctcgcgatgc agctggctca attggcggaa cacgaagagc tcgctgtgat tcatatcggt
tttgccaatg cccgccaatt acaggcgcgg ctgggttacc acctttggga tgatgtgcta
aagcagttac gtgagcgact tggtccggtg acggaggggg aattactgac cgctcgccct
aactcgacca atttgacgct gatcttaaaa gcccatccgc tcgacaccca attaaatcag
ctttgccatc gtttaattca cgctgggcaa gcgcaatttg tgacggaggg gctgcccgtt
cacctcaacc cttatattgg tgtggccctt agccgtgaaa cacgcgatcc gcagcagcta
ctgcgccatg ccgtcagcag catgttggcg tgtaaggact cgggatacaa agtgtttttt
cactctcccg cattagccga taaccatgca cggcaaaatc aattggaaaa ctatttactg
caagcggtgc gcaacaacga tctgctgctc tacttccaac ctaaagtcag catgaaaacc
cagcgctggg tcggtgctga ggcattgttg cgttggaagc atccggtgtt gggtgaattt
tccaatgaaa ccttgattca tatggcagag caaaatggtc ttatctttga agtggggcat
tttgttttgc accaagcttt aaaagccgcc agtgattggt tagcggtgtg cccaaccttt
tgtatcgcga tcaatgtctc ttccgtacag ctcaaaaaca gtggctttgt cgagcagatt
cgagatctgc tggcgctgta ttgcttccct gcgcatcagt tggaactgga aatcaccgaa
agtggcctga tcgtcgatga gccgaccgcg agtgatattc tcaaccgact acacacatta
ggcgtgacat tatcactcga tgattttggt acgggttacg cttcgtttca gtatctaaaa
aaattcccat ttgatggcat caagattgat aaaagtttta tggagcagat cgaacacagc
gaaagcgatc aagaaatcgt gcgttctatg ctgcatgtag cgaaaaaact gaacttaaac
gtggtggtgg aaggtattga gtcgacgcag caagagcagt tcattctgga acagggttgc
gatgtcggcc aaggcttttt atatggcaaa cctatgccca gtgaagtgtt taccctcaag
ctcgaaagcc acgctctggc gtaa
SEQ ID NO: 42 Vibrio cholerae strain 2012EL-2176 chromosome 2 amino acid
Sequence (AIT31635.1)

| | |
|---|---|
| 1 | MAPILSHSIP IPSSMQANWQ QMLNLLAEVL KVSATLIMRL RHHDLDVFCT SVGSDNPYQV |
| 61 | GMTERLGTGL YCETVVNTRQ ILLVSNADLD PLWKDNPDLE LGMRAYCGVP LQWPNGELFG |
| 121 | SLCVTDRQAR QFLSTDQQLI KTFAESIEAQ LKTLYQRETL LQMNQDLHFK VRHKMQSIAS |
| 181 | LNQSLHQEID KRRAAEQQIE YQRSHDLGTG FLNRTALEQQ LAMQLAQLAE HEELAVIHIG |
| 241 | FANARQLQAR LGYHLWDDVL KQLRERLGPV TEGELLTARP NSTNLTLILK AHPLDTQLNQ |
| 301 | LCHRLIHAGQ AQFVTEGLPV HLNPYIGVAL SRETRDPQQL LRHAVSSMLA CKDSGYKVFF |
| 361 | HSPALADNHA RQNQLENYLL QAVRNNDLLL YFQPKVSMKT QRWVGAEALL RWKHPVLGEF |
| 421 | SNETLIHMAE QNGLIFEVGH FVLHQALKAA SDWLAVCPTF CIAINVSSVQ LKNSGFVEQI |
| 481 | RDLLALYCFP AHQLELEITE SGLIVDEPTA SDILNRLHTL GVTLSLDDFG TGYASFQYLK |
| 541 | KFPFDGIKID KSFMEQIEHS ESDQEIVRSM LHVAKKLNLN VVVEGIESTQ QEQFILEQGC |
| 601 | DVGQGFLYGK PMPSEVFTLK LESHALA |

SEQ ID NO: 43 Vibrio cholerae 2012EL-2176 chromosome 2 DNA Sequence
(GI:695934490 REGION 491690..492670)
ttagaaaagt tcaacgtcat cagaaaatgg ccgttgcgcg ctggcaattt taccgttctc
acacagctgt tcatagcagt gcacctgatt ccgaccatgc tctttggcgt aatacaacgc
tttatcggca tggtcgagaa tggtaggtaa atagtcaccc ggcctgagtg agcaaaaacc
agcgctgaag ctcagttcac cgattctcgg gaagttatgg cgtcggatct gttgacggaa
gccatccaac tgttgcttga tttgtggctc attaccgctt gaaaaaataa tcacgaactc
ttcaccacca aagcgaaata gttgagaaga cggtccgaaa tagtgctgca tctgctgagc
gaacataagc agaatttcat caccaatcat gtgtccgaag tgatcattga tcgctttaaa
atggtcaata tccaacatcg cgatccagag tttgtgattc tcttctgtcg agggattgat
ggcaaaggtg tggcgcaatc ggtcttctaa cgttcgacga ttgagtaatc cggtcagctt
atcgcgttca ctctcatgca aaatcaccgt gtaattacgg taaattttcg caaatccgtt
gatcaacatg cgataaggtt caggatcttt attgaggatt aagcacagct ctgcggaaaa
gtgttcttct atcggaatcg ggcaaaagca ttgatattgg ccattcgctt gttgggaaaa
cgccatttcc gattgagagt gctggtaacc attgtcggca catacttggt cgtattgcca
ctggtactcc tttttacctg cagcattttt ggtaataatt aaacgtgcca ccataagggt
tgaacgtcca agatggtgaa ataaggtcgc cgtggagagc ggtaacaatt cagacaaggt
cgccaaaata ctgtaactga gtgccagcga atttttctgc tcagtaattt caataaccga
ctcaagcact ttgtcattca t
SEQ ID NO: 44 Vibrio cholerae strain 2012EL-2176 chromosome 2 amino acid
Sequence (AIT31689.1)

| | |
|---|---|
| 1 | MNDKVLESVI EITEQKNSLA LSYSILATLS ELLPLSTATL FHHLGRSTLM VARLIITKNA |
| 61 | AGKKEYQWQY DQVCADNGYQ HSQSEMAFSQ QANGQYQCFC PIPIEEHFSA ELCLILNKDP |
| 121 | EPYRMLINGF AKIYRNYTVI LHESERDKLT GLLNRRTLED RLRHTFAINP STEENHKLWI |
| 181 | AMLDIDHFKA INDHFGHMIG DEILLMFAQQ MQHYFGPSSQ LFRFGGEEFV IIFSSGNEPQ |
| 241 | IKQQLDGFRQ QIRRHNFPRI GELSFSAGFC SLRPGDYLPT ILDHADKALY YAKEHGRNQV |
| 301 | HCYEQLCENG KIASAQRPFS DDVELF |

SEQ ID NO: 45 Vibrio cholerae 2012EL-2176 chromosome 2 DNA Sequence
(GI:695934573 REGION 592066..592992)
atgatagaac ttaatagaat tgaagagctt tttgataacc aacagttctc cttgcacgaa
ctcgtgttga acgaactggg agtctatgtc ttcgtcaaaa atcgccgcgg cgagtatctc
tatgctaacc ctctgactct aaagttgttt gaagcggatg cacaatcgtt gtttggcaag
accgatcacg atttttttca tgatgatcaa ctcagtgata tcttggcggc cgatcaacag
gtgtttgaaa ctcgtctctc ggttatccat gaagaacgag ccatcgccaa atccaatggt
ttggttcgga tttatcgcgc agtcaaacac cctatcttgc accgagtgac aggcgaagtg
attgggctga ttggagtttc aaccgatatc accgatatcg tggaactgcg tgagcagcta
tatcagctcg ccaataccga ttctttaact cagctgtgta atcggcgtaa attgtgggcc
gattttcgcg ccgccttcgc tcgcgcaaaa cgtttaagac agccgttaag ttgcatctct
atcgatattg ataatttcaa actgatcaat gaccaatttg gtcacgataa aggtgatgaa
gtcctgtgtt ttctcgccaa actatttcag agcgtcatct ctgaccatca tttttgtggt
cgtgtgggag gtgaagagtt catcatcgtt ttggaaaata cgcacgtaga gacggctttt
catttggctg aacagatccg ccaacgtttt gcagagcatc cgttctttga acaaaacgag
cacatctacc tctgtgcggg ggtttccagc ttgcatcatg gtgatcatga cattgccgat
atttatcgac gctccgatca agcactgtat aaagccaagc gtaatggtcg taaccgttgc
tgtatctatc gccaatccac agaataa
SEQ ID NO: 46 Vibrio cholerae strain 2012EL-2176 chromosome 2 amino acid
Sequence (AIT31772.1)

| | |
|---|---|
| 1 | MIELNRIEEL FDNQQFSLHE LVLNELGVYV FVKNRRGEYL YANPLTLKLF EADAQSLFGK |
| 61 | TDHDFFHDDQ LSDILAADQQ VFETRLSVIH EERAIAKSNG LVRIYRAVKH PILHRVTGEV |
| 121 | IGLIGVSTDI TDIVELREQL YQLANTDSLT QLCNRRKLWA DFRAAFARAK RLRQPLSCIS |
| 181 | IDIDNFKLIN DQFGHDKGDE VLCFLAKLFQ SVISDHHFCG RVGGEEFIIV LENTHVETAF |
| 241 | HLAEQIRQRF AEHPFFEQNE HIYLCAGVSS LHHGDHDIAD IYRRSDQALY KAKRNGRNRC |
| 301 | CIYRQSTE |

SEQ ID NO: 47 Vibrio cholerae 2012EL-2176 chromosome 2 DNA Sequence
(GI:695934589 REGION 606596..607612)
atgacaactg aagatttcaa aaaatccacg gctaacttaa aaaaagtcgt acctttaatg
atgaaacatc atgtcgcggc cacccccgtg aactatgcct tgtggtatac ctacgtcgac
caagccattc cgcaactgaa tgcggaaatg gactctgtat tgaaaaattt tgggctttgc
ccacccgctt ctggtgaaca tctttaccaa caatacattg cgaccaaagc agaaaccaat
attaatcagt tacgtgcgaa tgttgaggta cttcttggtg aaattagcag ttcaatgagt
gatacgctca gtgacaccag ttcctttgct aatgtgattg ataaaagctt taaggattta
gagcgcgtcg agcaagacaa tctctcgatt gaagaagtaa tgacggtgat ccgccgcttg
gtgagtgact ctaaagatat tcgacactca accaatttcc taaataatca actgaacgcg
gcaacactag aaatctctcg tcttaaagag cagctggcga aagttcagaa agatgctctg
tttgacagtt tatctggact ctataaccgc cgagcttttg atggcgatat gttcacgctg
atccatgcag gtcaacaagt cagcctgatc atgctcgaca tcgaccactt caaagccctt
aatgataact atggccacct gtttggtgac caaattatcc gtgcgatcgc caaacgtctt
caaagcctat gccgtgacgg cgtgacagct tatcgttatg gcggtgaaga gtttgcactg
attgctccgc acaaatcgct gcgtattgca cgccagtttg ctgaatcggt gcgacgttca
atagaaaagc tcaccgtaaa agatcggcgt agcggtcaat cggtcggtag cattaccgct
tcgtttggtg tagtagaaaa gattgaaggt gactctttgg agtctcttat cggtcgagcg
gatggattgc tgtatgaagc gaaaaatctg ggccgcaatc gagtcatgcc gctctaa
SEQ ID NO: 48 Vibrio cholerae strain 2012EL-2176 chromosome 2 amino acid
Sequence (AIT31788.1)

| | |
|---|---|
| 1 | MTTEDFKKST ANLKKVVPLM MKHHVAATPV NYALWYTYVD QAIPQLNAEM DSVLKNFGLC |
| 61 | PPASGEHLYQ QYIATKAETN INQLRANVEV LLGEISSSMS DTLSDTSSFA NVIDKSFKDL |
| 121 | ERVEQDNLSI EEVMTVIRRL VSDSKDIRHS TNFLNNQLNA ATLEISRLKE QLAKVQKDAL |
| 181 | FDSLSGLYNR RAFDGDMFTL IHAGQQVSLI MLDIDHFKAL NDNYGHLFGD QIIRAIAKRL |
| 241 | QSLCRDGVTA YRYGGEEFAL IAPHKSLRIA RQFAESVRRS IEKLTVKDRR SCQSVCSITA |
| 301 | SFGVVEKIEG DSLESLIGRA DGLLYEAKNL GRNRVMPL |

SEQ ID NO: 49 Vibrio cholerae 2012EL-2176 chromosome 2 DNA Sequence
(GI:695934592 REGION 610255..611628)
tcaaaagcga tagagtgggt tttgcctacg cttagcggta tacatacgtt catcggccag
tttgaacatt tcatcaggtg tggcaaacga ctggtcatac aaagcatatc cgatacttac
acgaacatgg ataagcttgt cgtcataaac gatgggcgtt tcagaaatcc tttttaaaat
attgtcactg actttaagca cgtcttgttc acgatgaatt cgtggaatta acacgagaaa
ctcatccccc ccaatccgcg ccaccagatc ggaaacccgc aggctcgatt taattctttc
cgcacaagcc accagcactt tatcgcctgc gctatgtcca tgggaatcgt tgatagattt
aaaacggtca atatcaatgt tcaacaaagc aaagttacct tcgctatgag agcgcttagc
attttcaaag tagtgttcaa tggtatagat aaaatagcgc cgattcggca agtgggttaa
agggtcatgt agcgcacgct cctccgcgac ttgataaagg cgcatgataa cgccaaagcc
tgccatcaat accaataaca ccgagtatcc caacaagcgc actgcatttc gggtatacca
agataactgc tgtagtaaat cttgcttttc agcgaccgca attcgccaac ttccgtaagg
gaaatagaca ttctcttgtg caaaagcgtg ctcaaatact cgaggctctc caaaaaacac
gtccccctca ctgccacggc tgtctaaacc acgaatcgca acctgaaaat gctccccaaa
gctgtaaata ctggttgctg aaagcaatga atcccaatcc atcaccacac tcagtacccc
ccaataacgc gtatccttcg gtgggtcgta gaatatcggt tctcgaatca ccagcgcgcg
cccaccttga acgagatcga caggtccaga gacgaacgtc tgtttgattt cacgtgcttt
ttttattgac tgccactgct gaggaacggt gcggtaatcc aaaccgagta gtgcattggt
ttgaggaagc ggatagctga aagcgaccac atcattaggg gcgataccta atgagcgtaa
gtgatcgcta ttcctgatca ccgccgctga aagcggctcc cattgataga tattgaggtc
gggatctagg gttaacaggg ttgttaaacc ttttacggta tagatatcac ccaaaatctc
agcttctaat tgaaaacgta cgatggaaag atcttcttta gcttgttgac gtaaaccctc
ttgtagatca cgtgtatggc taatatgaag ggattcaata accgcaatgc ccaaaaagag
taaggcgaga aaataaattg agacatactt atatttgtgc gaggttaacc ccat
SEQ ID NO: 50 Vibrio cholerae strain 2012EL-2176 chromosome 2 amino acid
Sequence (AIT31791.1)

| | |
|---|---|
| 1 | MGLTSHKYKY VSIYFLALLF LGIAVIESLH ISHTRDLQEG LRQQAKEDLS IVRFQLEAEI |
| 61 | LGDIYTVKGL TTLLTLDPDL NIYQWEPLSA AVIRNSDHLR SLGIAPNDVV AFSYPLPQTN |
| 121 | ALLGLDYRTV PQQWQSIKKA REIKQTFVSG PVDLVQGGRA LVIREPIFYD PPKDTRYWGV |
| 181 | LSVVMDWDSL LSATSIYSFG EHFQVAIRGL DSRGSEGDVF FGEPRVFEHA FAQENVYFPY |
| 241 | GSWRIAVAEK QDLLQQLSWY TRNAVRLLGY SVLLVLMAGF GVIMRLYQVA EERALHDPLT |
| 301 | HLPNRRYFIY TIEHYFENAK RSHSEGNFAL LNIDIDRFKS INDSHGHSAG DKVLVACAER |
| 361 | IKSSLRVSDL VARIGGDEFL VLIPRIHREQ DVLKVSDNIL KRISETPIVY DDKLIHVRVS |
| 421 | IGYALYDQSF ATPDEMFKLA DERMYTAKRR QNPLYRF |

SEQ ID NO: 51 Vibrio cholerae 2012EL-2176 chromosome 2 DNA Sequence
(GI:695934597 REGION 616194 .617369)
atggatagct ttgctggcaa ccaattaaaa gagatgacag agatgcgttt tgctcgtaag
cagcatattg tcctgatcag ctctggtgtt gctaccgcta tttttcttgg gtttgccctt
tactactatt ttaaccatca acccctgtca tccggtttat tgttattaag cggtattgtc
accttattga atatgatttc gctgaatcgt caccgcgaat tacacactca agccgattta
attctgtcat taattctgct cacttatgcg ctggccttag tcagcaatgc tcagcatgaa
ttatcgcatc tcttatggtt atatccgctc atcaccactt tagtcatgat taaccctttt
cggttaggct tggtttacag tgcagcgata tgcttagcga tgaccgcctc tatccttttt
aatccggcac aaactggctc gtaccctatt gcacagacct attttttagt aagtctattt
acgctgacga ttatctgtaa taccgcttct ttctttttct caaaagcgat caattatatt
cataccctat accaagaagg tattgaagag ttggcttatc ttgatccgtt aacgggctta
gccaatcgtt ggagctttga aacttgggcc acagaaaagc tcaaagaaca acagagttcg
aataccatta ccgcgcttgt ttttctggat attgataatt tcaaacgcat taatgacagt
tacggccatg atgttggcga tcaggtgtta aaacattttg cacaccgtct acgcaataat
attcgtaata aagatcgagc caccaatcaa catgattatt ccattgctcg atttgctggt
gatgagtttg tgctcttgtt atatggtgtg cgaaatttgc gtgatctcga taatattctc
aaccgtatct gtaatctctt cgtcgaccgc tatcctgaga cggatatgct caacaacctc
acggtgagta taggggcagc tatttatccc aaagatgcga tcactctgcc ggaactaacc
cgctgcgcag ataaagccat gtatgccgct aaacacggtg gaaaaaatca gtaccgctat
taccatgatg ccgctttccc tccggctgta gaaaccgtat taggcagtca gcccgttgag
gctcctaacg taactccact gaaaaaagcg cactaa
SEQ ID NO: 52 Vibrio cholerae strain 2012EL-2176 chromosome 2 amino acid
Sequence (AIT31796.1)

| | |
|---|---|
| 1 | MDSFAGNQLK EMTEMRFARK QHIVLISSGV ATAIFLGFAL YYYFNHQPLS SGLLLLSGIV |
| 61 | TLLNMISLNR HRELHTQADL ILSLILLTYA LALVSNAQHE LSHLLWLYPL ITTLVMINPF |
| 121 | RLGLVYSAAI CLAMTASILF NPAQTGSYPI AQTYFLVSLF TLTIICNTAS FFFSKAINYI |
| 181 | HTLYQEGIEE LAYLDPLTGL ANRWSFETWA TEKLKEQQSS NTITALVFLD IDNFKRINDS |
| 241 | YGHDVGDQVL KHFAHRLRNN IRNKDRATNQ HDYSIARFAG DEFVLLLYGV RNLRDLDNIL |
| 301 | NRICNLFVDR YPETDMLNNL TVSIGAAIYP KDAITLPELT RCADKAMYAA KHGGKNQYRY |
| 361 | YHDAAFPPAV ETVLGSQPVE APNVTPLKKA H |

SEQ ID NO: 53 Vibrio cholerae 2012EL-2176 chromosome 2 DNA Sequence
(GI:695934700 REGION 737143..739053)
atgacgctat acaaacaact agtcgcaggg atgattgcgg tgtttattct gttgttgatt
tcggttttta ctatcgaatt caacaccact cgcaacagtc ttgaacaaca acaacgctct
gaagtcaaca acaccataaa tacggtgggt ttggctttag cgccttatct ggagaagaaa
gacaccattg cggtagagtc agtcatcaat gcgctgtttg atggcagtag ttactcgatc
gtacgtctga tttttctcga tgacggtacg gaaatcctgc gctcataccc tatccaaccc
aataatgtgc cggcttggtt tactcagtta aatctgtttg agcccatcca tgatcggcgt
gttgtaacca gtggttggat gcaattggcg gaagtggaaa tcgtcagcca toctggtgcg
gcttacgctc aactctggaa agcattaatt cgtttaagta tcgcgttttt ggcgatctta
gtgattggta tgtttgccgt cgccttcatt ttgaagcgct ctctaagacc actacaactc
atcgtcaaca aaatggagca ggttgctaac aaccaatttg gtgagcctct accgcgcccc
aacactcgag atctgattta tgtagtagat ggcatcaata agatgtctga acaggtcgag
aaagcgttta aagcccaagc caaagaggcg cagcaactgc gtgaacgtgc ttatcttgac
ccagtttctc atcttggcaa ccgagcatac tacatgagcc aattgagtgg ctggctctct
gaaagcggca tcggtggtgt agccattcta caagctgaat tcatcaaaga gctttatgaa
gagaagggct atgaagccgg tgatggcatg gtgcgcgaac tggcggatcg ccttaaaaac
tccatcacca tcaaggacat ctctatcgct cgtatctcca cttacgagtt cggtatcatc
atgcctaaca tggatgaaac tgagctcaaa atcgtggcag agagcatcat cacttgtgtg
gacgacatta accctgatcc tactggtatg gcgaaagcca atttatcgct tggcgtggta
agcaataagc gtcaatccag caccacaacg ctcttgtccc tgctggataa tgcgttagct
aaagcgaaat ccaatcctga gctgaactac ggctttatta gcagtgatac tgataaaatc
atcttgggca aacagcagtg gaaaactctg gtcgaagagg caatccataa cgactggttt
actttccgct accaagccgc caacagcagt tggggaaaaa cattccatcg cgaggtcttt
tctgcgtttg agaaagacgg cgtgcgttac acggcaaacc aattcttgtt tgcccttgaa
cagctcaatg ctagccatat cttcgatcag tacgtgattg aacgtgtgat tcaacagctt
gaaaaaggcg aactgaccga tccactcgcg atcaacatcg cacaaggcag tatctctcaa
ccgagcttta tccgttggat cagccaaacc ttaagcaagc atctttctgt ggccaactta
ctgcattttg agatcccaga aggctgtttc gtcaatgaac cgcattacac tgcgctattt
tgtaacgcag tacgcaatgc aggggcggac tttggggtag acaactacgg acgtaacttc
caatctctcg actacatcaa cgagttccgt cctaaatacg tcaaactgga ttatctattt
actcaccatt tggatgatga acgccagaaa tttaccctga cctcaatctc gcgcaccgcg
cataacttag ggatcaccac catcgcatca cgggttgaaa cacagactca gctcgatttt
ctttcagaac atttcatcga agtcttccaa ggcttcattg ttgataagta a
SEQ ID NO: 54 Vibrio cholerae strain 2012EL-2176 chromosome 2 amino acid
Sequence (AIT31899.1)

| | |
|---|---|
| 1 | MTLYKQLVAG MIAVFILLLI SVFTIEFNTT RNSLEQQQRS EVNNTINTVG LALAPYLEKK |
| 61 | DTIAVESVIN ALFDGSSYSI VRLIFLDDGT EILRSYPIQP NNVPAWFTQL NLFEPIHDRR |
| 121 | VVTSGWMQLA EVEIVSHPGA AYAQLWKALI RLSIAFLAIL VIGMFAVAFI LKRSLRPLQL |
| 181 | IVNKMEQVAN NQFGEPLPRP NTRDLIYVVD GINKMSEQVE KAFKAQAKEA QQLRERAYLD |
| 241 | PVSHLGNRAY YMSQLSGWLS ESGIGGVAIL QAEFIKELYE EKGYEAGDGM VRELADRLKN |
| 301 | SITIKDISIA RISTYEFGII MPNMDETELK IVAESIITCV DDINPDPTGM AKANLSLGVV |
| 361 | SNKRQSSTTT LLSLLDNALA KAKSNPELNY GFISSDTDKI ILGKQQWKTL VEEAIHNDWF |
| 421 | TFRYQAANSS WGKTFHREVF SAFEKDGVRY TANQFLFALE QLNASHIFDQ YVIERVIQQL |
| 481 | EKGELTDPLA INIAQGSISQ PSFIRWISQT LSKHLSVANL LHFEIPEGCF VNEPHYTALF |
| 541 | CNAVRNAGAD FGVDNYGRNF QSLDYINEFR PKYVKLDYLF THHLDDERQK FTLTSISRTA |
| 601 | HNLGITTIAS RVETQTQLDF LSEHFIEVFQ GFIVDK |

SEQ ID NO: 55 Vibrio cholerae 2012EL-2176 chromosome 2 DNA Sequence
(GI:695934774 REGION 830662..832242)
ctactcaaca cacacttggt tacggccatt ggctttggcg cgatacaaag ctttgtcagc
gcggtagaac gtacgttggg tattttcccc ctcgcgatgc aaggtgatac cgatactgac
cgtcagtccc cgttcgccaa gtacgtcttg ccatgggaaa tcaaaaatac gttggcgata
ggtttcggca tgcatttgtg ccatatcact ggtgacgttt tccaaaatca ccagaaattc
ctcgccaccg aaacgtacgc aggaggcacc acggaattta aagtaactcg ccagttcact
ggatacattg acaatcgctt tatcccctac caaatgactc aattcatcat tgatcgattt
aaagtggtca atatcaacga ctaagaaagc aaacggggtt tcgtgcagca gcagatcttt
cagcttcacg tccaaccaac ggcggttatg cagttttgtc agtggatcgg tgaacacatc
ttgctgtagt tgcaacaccg tattcttctg gctttcggtg gtttctttta gctcacgatt
ttctaattcc gacaaaatca gtttaagttg tagctcaaag cgcgataggc ggcgtagctg
aattgggcct aattcactga tggggatccg cttcatcaaa tcgctttcga tgcgaaatgc
tttcttttcg taaaccagtg cggttttgta cattccttcg agttcacaca cttcgctgaa
cgcttcatag aggcgttttt caaggaaagg ggaatgaatg ttttgtaagc gcttttcagt
gctacccagc agcatggtgg caaaatgcgc cttacctgct ttagagaggc aatgcgctaa
ctcgatgcgt agcatgcttg atagccaatc cgatggcgtc agcgatgacg aatactgtgc
attggcgagt gtcatcatcg ccttttgcac tttgccttgt tgcagataaa gcttggcttg
atagagcatg atctgcccag tcagcagttt atcgctgacc agaatgctca actcatcaca
ctcttttatc agatcattgg ccgctgcata acgaccaagg ctgatgtagc aagccagcat
atacagcttg taacgcaggc gcagtgagcg gctagaaatc gcatgatcta tgctgtcaat
tttttggtag tagcgtaacg cacggctgtg atcgccataa gcatcacata aattgcccat
tccgagcact gcaagtacgt agtcatcaat catgccatgc tcaacggcga tgttggatat
cgcaacgtat tcagacagtg ccgcgacata ttcaccatgg tcgagtaaac gctcactcaa
actgtgtttg accgagagca ttaattccag atccgtcggt aactctaata gggaaagagc
ggcgcgcagc tcttcaatac tggtttgcca ctgtttcatt tcgcggcggt attcggcgct
gatgatgtag ctttgtgcac gctcttgggc ggtggttgcc acgtgctgtc tgacatggtt
ccagaaaatg atcgcctctt caccagcgac agcggccgca tccagtcccg cttctttgat
cttattgagc agggtttcca t
SEQ ID NO: 56 Vibrio cholerae strain 2012EL-2176 chromosome 2 amino acid
Sequence (AIT31973.1)

| | |
|---|---|
| 1 | METLLNKIKE AGLDAAAVAG EEAIIFWNHV RQHVATTAQE RAQSYIISAE YRREMKQWQT |
| 61 | SIEELRAALS LLELPTDLEL MLSVKHSLSE RLLDHGEYVA ALSEYVAISN IAVEHGMIDD |
| 121 | YVLAVLGMGN LCDAYGDHSR ALRYYQKIDS IDHAISSRSL RLRYKLYMLA CYISLGRYAA |
| 181 | ANDLIKECDE LSILVSDKLL TGQIMLYQAK LYLQQGKVQK AMMTLANAQY SSSLTPSDWL |
| 241 | SSMLRIELAH CLSKAGKAHF ATMLLGSTEK RLQNIHSPFL EKRLYEAFSE VCELEGMYKT |
| 301 | ALVYEKKAFR IESDLMKRIP ISELGPIQLR RLSRFELQLK LILSELENRE LKETTESQKN |
| 361 | TVLQLQQDVF TDPLTKLHNR RWLDVKLKDL LLHETPFAFL VVDIDHFKSI NDELSHLVGD |
| 421 | KAIVNVSSEL ASYFKFRGAS CVRFGGEEFL VILENVTSDM AQMHAETYRQ RIFDFPWQDV |
| 481 | LGERGLTVSI GITLHREGEN TQRTFYRADK ALYRAKANGR NQVCVE |

SEQ ID NO: 57 Vibrio cholerae 2012EL-2176 chromosome 2 DNA Sequence
(GI:695934794 REGION 857071. 858171)
tcacgatgag gggctttttt gtaggaattt catttcatac atgtttttat ctgccagatg
gatcaactgg ctcaaattgg tgctgtcgag tggataagta ctgaccccga cgctggtgtt
gagcttggct cgtaaatcgc cacttaattc aaattcatgg tcgaaacact gtttgatcat
gcgctgcatc atcatctgct cggtcgaatt gatgctgctt aggatgatgg caaattcatc
tccccccatc cgaaacacac gataatcgaa tgaaggaatc gagttgttta agcgataagc
aacctgtttg agtaccgcat cgcccatttg atggccgtag gtatcattaa tttgtttaaa
accattcaga tcgagcaaaa agagagagaa tccaccgctg cggcggtggc gttctaattc
ggcgaacatg gctgtgcggt tttccagccc tgttaatggg tccgttaagg ccaagactct
atggtgcgtg gcctctttat gcaaaataaa actcaccagt cccacacagc taaacgtcaa
caaaattaac gcaaactgga tgcgactgag gtaattcagt ttctcttttt gctctacata
caaaggactt tgcattccaa atgtgcggtt tatgaactga ataaaaatct ccagctcttg
ttgggcggca acaataaaag tttgtaagct ttctggattt ttggccgcaa gcagtagcgg
ttcaagttgt ttaaagcgcg caaacgcggc ttggaagaat tcgcgagtgc tgggcatgcc
tataatgccg tcggcttctg ggctattgag gatcagatca aaacggctcc aagtcagctc
atatttcacc atcacatcgc gctggttgct ctccgactcc aataggtagg gggagagtgc
cagcatctca gtaaactctt tattgagctg gaataagaac cagatcgctt ggttagtatg
cgaagagtaa gacttagata aatcgcgagt actgttgatc aaatacaaat tggccaaaat
cagaatcgcc gacatgaaga tcagcagtgt tttggcatgt aagatcagcg ggtggagcgt
tttctgagtt tgtgtattca t
SEQ ID NO: 58 Vibrio cholerae strain 2012EL-2176 chromosome 2 amino acid
Sequence (AIT31993.1)

| | |
|---|---|
| 1 | MNTQTQKTLH PLILHAKTLL IFMSAILILA NLYLINSTRD LSKSYSSHTN QAIWFLFQLN |
| 61 | KEFTEMLALS PYLLESESNQ RDVMVKYELT WSRFDLILNS PEADGIIGMP STREFFQAAF |
| 121 | ARFKQLEPLL LAAKNPESLQ TFIVAAQQEL EIFIQFINRT FGMQSPLYVE QKEKLNYLSR |
| 181 | IQFALILLTF SCVGLVSFIL HKEATHHRVL ALTDPLTGLE NRTAMFAELE RHRRSGGFSL |
| 241 | FLLDLNGFKQ INDTYGHQMG DAVLKQVAYR LNNSIPSFDY RVFRMGGDEF AIILSSINST |
| 301 | EQMNMQRMIK QCFDHEFELS GDLRAKLNTS VGVSTYPLDS TNLSQLIHLA DKNMYEMKFL |
| 361 | QKSPSS |

SEQ ID NO: 59 Vibrio cholerae 2012EL-2176 chromosome 2 DNA Sequence
(GI:695934800 REGION 864637..866460)
ttaggctaca ttcgtttctt ttctccagcg ttcaatcatc acactcggta aatcaggtcg
actgaagtaa tacccttgaa tttgctcaca gcccatttga tagagtttat ccagtgcttg
ttggttctct accccctcag cgacgagatc gagtttaagc tggttagcaa gctgaataat
caaccacacg atactctcag aggtttggtt ggtaagtagg ttacgcacaa atgcagcatc
aatcttgatg caatcaatcg gataactgtg aatgtagtta aggctcgaat aacctgtccc
aaaatcatcc aaggcaattt taaaacccaa ttcacgcaat atggtgagaa tactgcatac
ttctgcggcc ttagagagta aaaccgtttc tgtcagctca atagtgaact cgtcggcttg
aaaaccatag gctttaatgg tttttaatag atgctcaagg taacgattgg aatgcgtcag
ctcatcggcg gagcagttga tgcttaagcg aattttttgg tcaatacctt gttctaattc
ttgtttcgcg atgcaggcca attcgagaat acgttcgcca aattcgacaa tcaggccaga
ttgctctgct gcttcaatga attccaatgg cgttaccaca ccgagcgtac tgctattcca
acgcgttaag atctcaaaat agtcccaatt tctttgatgt tttttcacga tcggttgcac
gaccacatac agctcagttt gatggatagg cttactcaat tcactacgca gagcttcgat
gatttgtgta cgccgatagt attgattgct gagtaagttg tcgtagaaac gaatgcgtgt
gttatggttc cgtttacact cttttaaagc gagacttgca ttgaacagta attgatcggc
attgagcttt tcaccactgt atttggtaat accaatactg acactgattt tgagtcgacg
atcttgatcg atataatctt gcgccagctt gttgagtatg gtttggcaga tcttcatcgg
ctcacgatct gtggttaaaa aagcaaattc atcagcggcg attcgaaagg cgtatccttc
ttcggggacg gcttgtttta tcgcatccgc gacaaatttc agcacaagat ctcccaaata
gtgcccatgc agatcgttta tcgaacgaaa ttcatcaata tcaagaaagg ccagagtgaa
atgatgtcta tcttcttgaa cgagagccgt cagtttctcg gctaaatcat tacgattcat
taaacccgtt aagttgtcgt gagatatttc atgacgtaat tgattgatta ggctctgaga
gcgtacctcc atctgtttac attccagatc atgagcgatc atctgagcca aaatctggtg
aactaacacg agattagcaa agtcgtctaa ctgacgcgta aaagtcgaga tcaaaacgcc
gtagttttcg ccatttgaaa aataaatcgg gatacccaga tacgcctcaa tatggttctc
aactaaataa gcatcgttag gaaaaagttc cgcgactttg cttgcaaata ggcaataagg
ttgtctttgt aatccgactt gctcacaagg tgtgccttgt agttcgtaat acagctctaa
actgctgggt tcgacactgg cacaacttaa gttatgagct ttgtagcgca ttttatctag
ctcaatgacc attgagctgt ggctattgaa ggtgcggtgg agaaactgag tgatttgtga
gagcaactcc aaccccccca gctgactgaa gtgatgtatg gaatctaggc tcagtttttc
tgttatcagt tgagtcttgg tcat
SEQ ID NO: 60 Vibrio cholerae strain 2012EL-2176 chromosome 2 amino acid
Sequence (AIT31999.1)

| | |
|---|---|
| 1 | MTKTQLITEK LSLDSIHHFS QLGGLELLSQ ITQFLHRTFN SHSSMVIELD KMRYKAHNLS |
| 61 | CASVEPSSLE LYYELQGTPC EQVGLQRQPY CLFASKVAEL FPNDAYLVEN HIEAYLGIPI |
| 121 | YFSNGENYGV LISTFTRQLD DFANLVLVHQ ILAQMIAHDL ECKQMEVRSQ SLINQLRHEI |
| 181 | SHDNLTGLMN RNDLAEKLTA LVQEDRHHFT LAFLDIDEFR SINDLHGHYL GDLVLKFVAD |
| 241 | AIKQAVPEEG YAFRIAADEF AFLTTDREPM KICQTILNKL AQDYIDQDRR LKISVSIGIT |
| 301 | KYSGEKLNAD QLLFNASLAL KECKRNHNTR IRFYDNLLSN QYYRRTQIIE ALRSELSKPI |
| 361 | HQTELYVVVQ PIVKKHQRNW DYFEILTRWN SSTLGVVTPL EFIEAAEQSG LIVEFGERIL |
| 421 | ELACIAKQEL EQGIDQKIRL SINCSADELT HSNRYLEHLL KTIKAYGFQA DEFTIELTET |
| 481 | VLLSKAAEVC SILTILRELG FKIALDDFGT GYSSLNYIHS YPIDCIKIDA AFVRNLLTNQ |
| 541 | TSESIVWLII QLANQLKLDL VAEGVENQQA LDKLYQMGCE QIQGYYFSRP DLPSVMIERW |
| 601 | RKETNVA |

SEQ ID NO: 61 Vibrio cholerae 2012EL-2176 chromosome 2 DNA Sequence
(GI:695934874 REGION 956091..958088)
gtggcaggtc acaccttact ctcttccaac acgtttacgc cgctagaagc gtatcctgaa
gccttttggg catgggctgc gcagtttgat acttccgatg gtttgatccc ttttgccatc
aatacctgtc gctggaacta tttgccagtg atgggcggtg agtcgtttat ttttatgctg
gataatcatc ctcagcatcg gacttatctg atcattcaag cggcatgcgt cgataaagta
cacctgagca ctcaatccgg tgagttggat tttttacagt taattgcagc gaaatggcaa
tgcttacgag cggaaattga agcatcgaaa gagtttaaaa atcgtgattt acgtgaggcg
cagtacctta gtgaaattcg tcagcgagag cagtttattg acaacatgaa gctggtgcat
caagtcgcgc tcgagttgtc caaccccgcc aatcttgatg agctacaccg cgcatcggtc
gaggctatgc gacatcgtct cgggtttgat cgatccgcgc tcttgttgct tgatatgaaa
aagcgttgct tcagcggtac ttatggtacc gatgagcacg gtaatacgat tgatgaacag
cacacccagt atgatctgca ccaattagag cctcaatatc tcgaagcttt atccaatgaa
gagtgcactt tgatggtggt ggaagatgtg cctttgtaca ccgtcggaca ggtagtggga
caaggctgga atgccatgct gattttgcgt gatggtaatg acaccatagg ctggattgcc
atcgacaact atatcaatcg gcagccgatt accgagtatc aaaagcagat gcttgagtcg
tttggctcat tgctcgcgca aatttatatt cgtaaaaagc aggaacaaaa cgtacgtatg
ctgcatgcca gcatggtcga actgtctcgc tgtatgacag tcagtgaagt gtgtaaatcg
gcagtcacct ttgcgatcaa ccgaatgggg attgatcgca tggcggtgtt tttgacggat
gaagcttgct cttatattca ggggacgtgg gggacggata ttcaaggcaa tattgtcgat
gaatcctatt tccgtggttc aacgcatgaa aatgacattg tcgaccttgc caaagtgtac
ccaaacgaag tggtgtttaa agagagtgtt cccatctatc acgactgtaa aattgtcggt
tatggttgga cggcgatgac catgctcacc gacaaaggca ccccgattgc ctttattgcg
gcggataatt tgatccgacg ttcccccttg acttcacaac tgcgtgaagt gattcgtatg
tttgcttcaa acctcaccga agtcttgatg cgagccaaag cccaagaagc gatctcggta
ctcaatgaaa cgctggagct tgaggtgcgt aatcgcactc gtgatttgca aaaggccaac
gaaaaactcg atttaatggc gaaattagat ccgctgactc gtttagggaa tcgccgtatg
cttgagcacc aactggagca aacttgcgaa cagaccatca aagaggtggt caattatggc
gtgatcttgc ttgatattga ccatttcggg cttttcaaca actgctatgg tcatcttgaa
ggcgatattg ctctgatgcg gattggtaat atcctcagtc gacatgcgca atctgagcat
gaactgttct gtcgtattgg tggggaagag tttctgcttt tagtcgccaa tcgaagcgcc
gaggagattc acttactggc tgaaaatatt cgtaaaagta ttgaagcaga atgcattgaa
cactgcgaaa atcccagtgg tgagctactg accgtatcga ttggttatgc tgcttctcgt
tataaaccgc gagagattca atttgatcag ctctatgcag aagcggataa agccttgtac
agagcgaaaa gccaaggacg gaatcaggtt attggcgtta ttgttgaaaa tatcgactgc
atacaggcag aaatgtag
SEQ ID NO: 62 Vibrio cholerae strain 2012EL-2176 chromosome 2 amino acid
Sequence (AIT32073.1)

| | |
|---|---|
| 1 | MAGHTLLSSN TFTPLEAYPE AFWAWAAQFD TSDGLIPFAI NTCRWNYLPV MGGESFIFML |
| 61 | DNHPQHRTYL IIQAACVDKV HLSTQSGELD FLQLIAAKWQ CLRAEIEASK EFKNRDLREA |
| 121 | QYLSEIRQRE QFIDNMKLVH QVALELSNPA NLDELHRASV EAMRHRLGFD RSALLLLDMK |
| 181 | KRCFSGTYGT DEHGNTIDEQ HTQYDLHQLE PQYLEALSNE ECTLMVVEDV PLYTVGQVVG |
| 241 | QGWNAMLILR DGNDTIGWIA IDNYINRQPI TEYQKQMLES FGSLLAQIYI RKKQEQNVRM |
| 301 | LHASMVELSR CMTVSEVCKS AVTFAINRMG IDRMAVFLTD EACSYIQGTW GTDIQGNIVD |
| 361 | ESYFRGSTHE NDIVDLAKVY PNEVVFKESV PIYHDCKIVG YGWTAMTMLT DKGTPIAFIA |
| 421 | ADNLIRRSPL TSQLREVIRM FASNLTEVLM RAKAQEAISV LNETLELEVR NRTRDLQKAN |
| 481 | EKLDLMAKLD PLTRLGNRRM LEHQLEQTCE QTIKEVVNYG VILLDIDHFG LFNNCYGHLE |
| 541 | GDIALMRIGN ILSRHAQSEH ELFCRIGGEE FLLLVANRSA EEIHLLAENI RKSIEAECIE |
| 601 | HCENPSGELL TVSIGYAASR YKPREIQFDQ LYAEADKALY RAKSQGRNQV IGVIVENIDC |
| 661 | IQAEM |

SEQ ID NO: 63 Vibrio cholerae 2012EL-2176 chromosome 2 DNA Sequence
(GI:695934896 REGION 980640..981086)
atgctagcgt tacctgcgga gtttgagcaa ttccattgga tggtcgatat ggttcagaat
gtcgatatgg gattgattgt gattaaccga gactacaacg tgcaagtgtg gaatgggttt
atgacccatc atagcggtaa gcaagctcat gatgttattg gtaaatctct gttcgagatt
tttccagaga tccctgtgga gtggtttaag ttaaaaacca aaccggtgta cgatctgggt
tgccgtagtt ttattacttg gcagcagcgc ccttatttgt tccattgccg taatgtgcgc
ccagtgactc agcaagccaa atttatgtat caaaacgtca cgcttaaccc aatgcgtaca
ccgacaggcg cgataaattc actcttctta tccattcaag atgcaacaag tgaagccctt
gtttctcaac aagcttcttc tcaataa
SEQ ID NO: 64 Vibrio cholerae strain 2012EL-2176 chromosome 2 amino acid
Sequence (AIT32095.1)

| | |
|---|---|
| 1 | MLALPAEFEQ FHWMVDMVQN VDMGLIVINR DYNVQVWNGF MTHHSGKQAH DVIGKSLFEI |
| 61 | FPEIPVEWFK LKTKPVYDLG CRSFITWQQR PYLFHCRNVR PVTQQAKFMY QNVTLNPMRT |
| 121 | PTGAINSLFL SIQDATSEAL VSQQASSQ |

SEQ ID NO: 65 Vibrio cholerae 2012EL-2176 chromosome 2 DNA Sequence
(GI:695934918 REGION 1008191..1009270)
tcagcgatga ccatgagttg aacccaatag cgcatgacaa tggtcaccat tgagttcaat
gacatgctct tcatcgaagc tgacgcggtt tttccccatt tttttcgaat gatagagagc
ttggtctgcg cgtttgaacc actgctccgg atcatcggtg cgaagtgctt cggctaaacc
gacactgacg gtgactttgg catggtatgg gtagtgcgtt tgttgaatcc gacaaccaat
atgactcatc acgagtgtag cgtcggttaa cgacgtattt tcaaacagca gtaaaaattc
atcgccccct aatcgaaaca acagatctaa ctcacggcag tgagtattca ttatttcaac
aacttgggta atgactttat ctcctgtgtc gtgtccataa aggtcattaa cagatttgaa
gtgatcgata tcgatcacgg cgatcaccgc cgattcattg gcgagctggc ggtggcgaag
acattttttc aaaaaaccat ccagttgatg acgattcaat gtgcccgtta atgcatgacg
agtggaaaga taaaaaagct cagtgtgcag cttacggata gcatctacca ccacatacat
gatggcggca caagcgctga tcgcaaggct aaagcgcaag gtgacttcgg cggtttgatg
gggaattaaa acccatatgc tggctggaat gataatggtg atggtcaata agttatcttt
ctgggggagt agaaaagcaa tcgcaatgag cacgggaaat agccagtagc tggcgagggt
gccgaaaatg tgaatagcca tcaccacgat gactaccacc aatgccagtg gaagcctaaa
accccatggt gttttctttt gataatagat agccgtaatt tcaatgagga gcgtgcattg
gaatacgatg atcaacccgc caagaagaac gtagtcaatc agcaagtttt taacggcgag
tggaaagaaa accaaactag aaataaaacc aataaaaagc gacacccgac gttgatagta
agtgttcagt aactctgaac cggtaaaagc aggagagtga gtcgattttg tcatcgtcat
SEQ ID NO: 66 Vibrio cholerae strain 2012EL-2176 chromosome 2 amino acid
Sequence (AIT32117.1)

| | |
|---|---|
| 1 | MTMTKSTHSP AFTGSELLNT YYQRRVSLFI GFISSLVFFP LAVKNLLIDY VLLGGLIIVF |
| 61 | QCTLLIEITA IYYQKKTPWG FRLPLALVVV IVVMAIHIFG TLASYWLFPV LIAIAFLLPQ |
| 121 | KDNLLTITII IPASIWVLIP HQTAEVTLRF SLAISACAAI MYVVVDAIRK LHTELFYLST |
| 181 | RHALTGTLNR HQLDGFLKKC LRHRQLANES AVIAVIDIDH FKSVNDLYGH DTGDKVITQV |
| 241 | VEIMNTHCRE LDLLFRLGGD EFLLLFENTS LTDATLVMSH IGCRIQQTHY PYHAKVTVSV |
| 301 | GLAEALRTDD PEQWFKRADQ ALYHSKKMGK NRVSFDEEHV IELNGDHCHA LLGSTHGHR |

SEQ ID NO: 67 Vibrio cholerae 2012EL-2176 chromosome 2 DNA Sequence
(GI:695934235)
atggatcatc gcttttcgac caaactgttt ctgcttctca tgattgcttg gccgctttta
ttcggatcaa tgagtgaggc tgtagagcgc caaaccttga ctattgccaa ctcaaaagca
tggaaaccct attcttattt ggatgaacag ggacagcctt ctggcatatt gattgatttt
tggttggctt ttggtgaagc gaatcatgtc gatattgaat tccaactgat ggattggaat
gattccctag aagcggtgaa gcttggcaaa tccgatgttc aagctggttt gatccgttct
gcttcaagat tagcgtatct cgattttgca gaacctttac tgacaatcga tacacaactc
tacgtacacc gcacgttatt gggcgataaa ttggatacgc tgctatcggg ggccattaac
gtctcattag gtgtagtaaa agggggattt gaacaagagt tcatgcaacg agaatatcct
caacttaagt tgattgagta cgccaacaat gaattgatga tgtctgcagc aaagcgacga
gaattagatg gttttgtggc cgatactcag gtcgccaatt tctatatagt ggtttccaat
ggcgcgaaag attttacgcc agtgaagttt ctttattcag aggaattacg tccagcggtc
gccaaaggca atagggattt attagagcaa gtagagcagg ggtttgcaca attaagtagc
aatgagaaaa accgtatttt aagtcgatgg gttcatattg aaacgattta tccacgttac
ttaatgccga ttctcgcttc aggtctctta ctcagtatcg ttatttatac tcttcagcta
cggcgtaccg ttcgattgcg aacacagcaa cttgaagaag ccaatcaaaa actctcctat
ttagcgaaaa cggatagctt gacggacatt gctaatcgcc gttcgttttt tgaacatctt
gaagcggaac aaacacgatc aggcagctta acgttgatgg tttttgatat tgatgacttc
aaaaccatta acgatcgctt tgggcatggc gcaggagata atgccatctg tttcgtggtt
gggtgtgtgc gacaagcttt agcatcggat acctactttg caaggattgg tggtgaagag
tttgctattg tagcgcgtgg taaaaatgca gaagagtcgc agcagttagc tgagcgaatt
tgccaacgag ttgcagaaaa aaagtgggta gtgaatgccc aacactctct gtcactcacc
atcagcctag gctgtgcatt ttacctacac ccagctcggc cattcagttt gcacgatgcc
gatagcttaa tgtacgaagg aaagcggaat ggaaagaacc aggttgtctt tcgtacctgg tcataa
SEQ ID NO: 68 Vibrio cholerae VCA0848 01 biovar El Tor str. N16961
chromosome II DNA Sequence (gi|15600771:c790898-789918; NC_002506.1)
ATGAATGACAAAGTGCTTGAGTCGGTTATTGAAATTACTGAGCAGAAAAATTCGCTGGCACTCAGTTACA
GTATTTTGGCGACCTTGTCTGAATTGTTACCGCTCTCCACGGCGACCTTATTTCACCATCTTGGACGTTC
AACCCTTATGGTGGCACGTTTAATTATTACCAAAAATGCTGCAGGTAAAAAGGAGTACCAGTGGCAATAC
GACCAAGTATGTGCCGACAATGGTTACCAGCACTCTCAATCGGAAATGGCGTTTTCCCAACAAGCGAATG
GCCAATATCAATGCTTTTGCCCGATTCCGATAGAAGAACACTTTTCCGCAGAGCTGTGCTTAATCCTCAA
TAAAGATCCTGAACCTTATCGCATGTTGATCAACGGATTTGCGAAAATTTACCGTAATTACACGGTGATT
TTGCATGAGAGTGAACGCGATAAGCTGACCGGATTACTCAATCGTCGAACGTTAGAAGACCGATTGCGCC
ACACCTTTGCCATCAATCCCTCGACAGAAGAGAATCACAAACTCTGGATCGCGATGTTGGATATTGACCA
TTTTAAAGCGATCAATGATCACTTCGGACACATGATTGGTGATGAAATTCTGCTTATGTTCGCTCAGCAG
ATGCAGCACTATTTCGGACCGTCTTCTCAACTATTTCGCTTTGGTGGTGAAGAGTTCGTGATTATTTTTT
CAAGCGGTAATGAGCCACAAATCAAGCAACAGTTGGATGGCTTCCGTCAACAGATCCGACGCCATAACTT
CCCGAGAATCGGTGAACTGAGCTTCAGCGCTGGTTTTTGCTCACTCAGGCCGGGTGACTATTTACCTACC
ATTCTCGACCATGCCGATAAAGCGTTGTATTACGCCAAAGAGCATGGTCGGAATCAGGTGCACTGCTATG
AACAGCTGTGTGAGAACGGTAAAATTGCCAGCGCGCAACGGCCATTTTCTGATGACGTTGAACTTTTCTA
A
SEQ ID NO: 69 Vibrio cholerae strain O1 biovar El Tor str. N16961 amino acid
Sequence (NP_233234.1)

| | |
|---|---|
| 1 | MNDKVLESVI EITEQKNSLA LSYSILATLS ELLPLSTATL FHHLGRSTLM VARLIITKNA |
| 61 | AGKKEYQWQY DQVCADNGYQ HSQSEMAFSQ QANGQYQCFC PIPIEEHFSA ELCLILNKDP |
| 121 | EPYRMLINGF AKIYRNYTVI LHESERDKLT GLLNRRTLED RLRHTFAINP STEENHKLWI |
| 181 | AMLDIDHFKA INDHFGHMIG DEILLMFAQQ MQHYFGPSSQ LFRFGGEEFV IIFSSGNEPQ |
| 241 | IKQQLDGFRQ QIRRHNFPRI GELSFSAGFC SLRPGDYLPT ILDHADKALY YAKEHGRNQV |
| 301 | HCYEQLCENG KIASAQRPFS DDVELF |

SEQ ID NO: 70 Vibrio cholerae strain O1 biovar El Tor str. N16961 Vc DncV DNA
Sequence NC_002505.1; gi|15640032:180419-181729)
GTGAGAATGACTTGGAACTTTCACCAGTACTACACAAACCGAAATGATGGCTTGATGGGCAAGCTAGTTC
TTACAGACGAGGAGAAGAACAATCTAAAGGCATTGCGTAAGATCATCCGCTTAAGAACACGAGATGTATT
TGAAGAAGCTAAGGGTATTGCCAAGGCTGTGAAAAAAAGTGCTCTTACGTTTGAAATTATTCAGGAAAAG
GTGTCAACGACCCAAATTAAGCACCTTTCTGACAGCGAACAACGAGAAGTGGCTAAGCTTATTTACGAGA
TGGATGATGATGCTCGTGATGAGTTTTTGGGATTGACACCTCGCTTTTGGACTCAGGGAAGCTTTCAGTA
TGACACGCTGAATCGCCCGTTTCAGCCTGGTCAAGAAATGGATATTGATGATGGAACCTATATGCCAATG
CCTATTTTTGAGTCAGAGCCTAAGATTGGTCATTCTTTACTAATTCTTCTTGTTGACGCGTCACTTAAGT
CACTTGTAGCTGAAAATCATGGCTGGAAATTTGAAGCTAAGCAGACTTGTGGGAGGATTAAGATTGAGGC
AGAGAAAACACATATTGATGTACCAATGTATGCAATCCCTAAAGATGAGTTCCAGAAAAAGCAAATAGCT
TTAGAAGCAAATAGATCATTTGTTAAAGGTGCCATTTTTGAATCATATGTTGCAGATTCAATTACTGACG
ATAGTGAAACTTATGAATTAGATTCAGAAAACGTAAACCTTGCTCTTCGTGAAGGTGATCGGAAGTGGAT
CAATAGCGACCCCAAAATAGTTGAAGATTGGTTCAACGATAGTTGTATACGTATTGGTAAACATCTTCGT
AAGGTTTGTCGCTTTATGAAAGCGTGGAGAGATGCGCAGTGGGATGTTGGAGGTCCGTCATCGATTAGTC
TTATGGCTGCAACGGTAAATATTCTTGATAGCGTTGCTCATGATGCTAGTGATCTCGGAGAAACAATGAA
GATAATTGCTAAGCATTTACCTAGTGAGTTTGCTAGGGGAGTAGAGAGCCCTGACAGTACCGATGAAAAG
CCACTCTTCCCACCCTCTTATAAGCATGGCCCTCGGGAGATGGACATTATGAGCAAACTAGAGCGTTTGC
CAGAGATTCTGTCATCTGCTGAGTCAGCTGACTCTAAGTCAGAGGCCTTGAAAAAGATTAATATGGCGTT
TGGGAATCGTGTTACTAATAGCGAGCTTATTGTTTTGGCAAAGGCTTTACCGGCTTTCGCTCAAGAACCT
AGTTCAGCCTCGAAACCTGAAAAAATCAGCAGCACAATGGTAAGTGGCTGA
SEQ ID NO: 71 Homo sapiens Mab-21 domain containing 1 (MB21D1), Human cGAS,
transcript variant X1, mRNA (XM_017010232.1)

| | |
|---|---|
| 1 | gcgacttccc agcctggggt tccccttcgg gtcgcagact cttgtgtgcc cgccagtagt |
| 61 | gcttggtttc caacagctgc tgctggctct tcctcttgcg gccttttcct gaaacggatt |
| 121 | cttctttcgg ggaacagaaa gcgccagcca tgcagccttg gcacggaaag gccatgcaga |
| 181 | gagcttccga ggccggagcc actgccccca aggcttccgc acggaatgcc aggggcgccc |
| 241 | cgatggatcc caccgagtct ccggctgccc ccgaggccgc cctgcctaag gcgggaaagt |
| 301 | tcggccccgc caggaagtcg ggatcccggc agaaaaagag cgccccggac acccaggaga |
| 361 | ggccgcccgt ccgcgcaact ggggcccgcg ccaaaaaggc ccctcagcgc gcccaggaca |
| 421 | cgcagccgtc tgacgccacc agcgcccctg gggcagaggg gctggagcct cctgcggctc |
| 481 | gggagccggc tctttccagg gctggttctt gccgccagag gggcgcgcgc tgctccacga |
| 541 | agccaagacc tccgccoggg ccctgggacg tgoccagocc cggcctgccg gtctoggccc |
| 601 | ccattctcgt acggagggat gcggcgcctg gggcctcgaa gctccgggcg gttttggaga |
| 661 | agttgaagct cagccgcgat gatatctcca cggcggcggg gatggtgaaa ggggttgtgg |
| 721 | accacctgct gctcagactg aagtgcgact ccgcgttcag aggcgtcggg ctgctgaaca |
| 781 | ccgggagcta ctatgagcac gtgaagattt ctgcacctaa tgaatttgat gtcatgttta |
| 841 | aactggaagt ccccagaatt caactagaag aatattccaa cactcgtgca tattactttg |
| 901 | tgaaatttaa aagaaatccg aaagaaaatc ctctgagtca gtttttagaa ggtgaaatat |
| 961 | tatcagcttc taagatgctg tcaaagttta ggaaaatcat taaggaagaa attaacgaca |
| 1021 | ttaaagatac agatgtcatc atgaagagga aaagaggagg gagccctgct gtaacacttc |
| 1081 | ttattagtga aaaaatatct gtggatataa ccctggcttt ggaatcaaaa agtagctggc |
| 1141 | ctgctagcac ccaagaaggc ctgcgcattc aaaactggct ttcagcaaaa gttaggaagc |
| 1201 | aactacgact aaagccattt taccttgtac ccaagcatgc aaaggaagga aatggtttcc |
| 1261 | aagaagaaac atggcggcta tccttctctc acatcgaaaa ggaaattttg aacaatcatg |
| 1321 | gaaaatctaa aacgtgctgt gaaaacaaag aagagaaatg ttgcaggaaa gattgtttaa |
| 1381 | aactaatgaa atacctttta gaacagctga aagaaaggtt taaagacaaa aaacatctgg |
| 1441 | ataaattctc ttcttatcat gtgaaaactg ccttctttca catggagtct cgctctgtcg |
| 1501 | cccaggctgg agtccagtgg catgatcttg gctcactgca agctctgctt cctgggttca |
| 1561 | tgccattctc ctgcctcagc cttccgagta gctgggacta caggtgcccg ccaccacatc |
| 1621 | cggctaattt tttgtatttt tagtaaagat ggggtttcac catgttagcc aggatggtct |
| 1681 | cgatctcctt accttgtgat ccgcccgcct tggcctccca aagtgctggg attacaggtg |
| 1741 | tgagccacca cgcctggctg aaatacataa tcttaaaaga aaacataaga tactttattt |
| 1801 | taatatacgt gactaaatgt aaaacctaac ttattttctg ttatctattt atttttactt |
| 1861 | tcagtaacac tttttttatt ttaggtagca ttcagcctag aggcaactgc tgtttgttaa |
| 1921 | atatttcctg ttcatatatt ttgcacattt tcttatgggt tagttttctt ctcattgttt |
| 1981 | tgggaagttc ttaatatatt tggggtattt atctttcatt cgttgtctgt gtaacaaata |
| 2041 | acttctgcca tatgggttgt ctgcacattt tttggtgtct tttagtaaac aaggtttttt |
| 2101 | tgttttgtat tgttttgttt attgtaaaga tttttaaatt ttaatggagt tgatttcttt |
| 2161 | tctcattcaa gcttttgaga ataaattgga gttgaatttt t |

SEQ ID NO: 72 Homo sapiens Mab-21 domain containing 1 (MB21D1), Human
cyclic GMP-AMP synthase isoform X1 (cGAS) amino acid sequence (XP_016865721.1)
MQPWHGKAMQRASEAGATAPKASARNARGAPMDPTESPAAPEAA
LPKAGKFGPARKSGSRQKKSAPDTQERPPVRATGARAKKAPQRAQDTQPSDATSAPGA
EGLEPPAAREPALSRAGSCRQRGARCSTKPRPPPGPWDVPSPGLPVSAPILVRRDAAP
GASKLRAVLEKLKLSRDDISTAAGMVKGVVDHLLLRLKCDSAFRGVGLLNTGSYYEHV
KISAPNEFDVMFKLEVPRIQLEEYSNTRAYYFVKFKRNPKENPLSQFLEGEILSASKM
LSKFRKIIKEEINDIKDTDVIMKRKRGGSPAVTLLISEKISVDITLALESKSSWPAST
QEGLRIQNWLSAKVRKQLRLKPFYLVPKHAKEGNGFQEETWRLSFSHIEKEILNNHGK
SKTCCENKEEKCCRKDCLKLMKYLLEQLKERFKDKKHLDKFSSYHVKTAFFHMESRSV
AQAGVQWHDLGSLQALLPGFMPFSCLSLPSSWDYRCPPPHPANFLYF
SEQ ID NO: 73 Peptoclostridium difficile 630, complete genome-DisA
DNA sequence (NCBI Reference Sequence: NC 009089.1, gi|126697566:46917-47987)
ATGGAGAATTTTCTAGATAATAAAAATATGCTATATGCATTAAAAATGATATCTCCTGGAACTCCACTTA
GATTAGGTCTAAACAATGTACTAAGAGCTAAGACTGGTGGATTAATTGTAATTGCAACAAACGAAGATGT
AATGAAAATAGTAGATGGAGGATTTGCTATAAATGCAGAATATTCACCATCATATCTATATGAATTAGCT
AAAATGGATGGAGCTATAGTTTTAAGTGGTGATGTAAAGAAAATATTATTTGCTAATGCACAACTTATAC
CTGACTATTTTATAGAAACATCAGAGACAGGAACAAGACATAGAACAGCAGAAAGAGTAGCAAAACAAAC
TGGTGCTATAGTCATAGGAATTTCACAAAGAAGAAATGTTATAACAGTTTATAGAGGAAATGAGAAGTAT
GTAGTCGAAGATATATCTAAGATATTTACTAAGGCAAATCAGGCTATACAAACTCTGGAAAAATATAAGA
CAGTATTGGACCAAGCTGTAACAAATTTAAATGCCTTAGAGTTTAATGATTTGGTAACTATTTATGATGT
TGCATTAGTCATGCAAAAGATGGAAATGGTAATGAGAGTTACAAGTATAATTGAAAAATATGTGATAGAA
TTGGGTGATGAAGGAACTTTAGTAAGTATGCAATTAGAAGAATTAATGGGTACAACCAGAATAGACCAGA
AATTAATATTCAAAGATTATAATAAAGAAAACACAGAAATAAAAGAACTTATGAAAAAGGTCAAAAATTT
AAATTCAGAAGAACTAATAGAATTGGTTAATATGGCAAAACTATTAGGGTATAGTGGTTTTTCAGAAAGT
ATGGATATGCCTATAAAAACAAGAGGTTATAGGATTCTTAGCAAAATACATAGACTACCAACAGCAATAA
TAGAAAACTTAGTAAATTATTTTGAAAACTTTCAACAAATTTTAGATGCATCTATTGAAGAATTAGATGA
GGTTGAAGGAATAGGTGAAATAAGAGCAACATATATAAAAAATGGACTCATAAAAATGAAACAATTAGTC
TTATTAGATAGACACATATGA
SEQ ID NO: 74 DNA integrity scanning protein DisA [Bacillus subtilis] DNA
sequence (GenBank: KIX80328.1)
atggaaaaag agaaaaaagg ggcgaaacac gagttagacc tgtcatctat attgcagttt
gttgctccgg gtacaccgct cagagcgggg atggaaaacg tcttgagagc aaatacaggc
ggtctgattg ttgttggata taatgataaa gtaaaagaag tggtggacgg cggctttcac
ataaacacgg ctttttctcc ggcgcattta tatgagctgg ctaaaatgga tggagcgatc
attttaagtg attctggtca aaagatccta tacgcgaata ctcagctgat gccggatgcc
acaatttctt catcagaaac aggaatgcgg cacagaactg ccgaaagagt agctaagcaa
actggctgtc ttgtaatcgc catttctgaa agaagaaatg tcataacgtt atatcaggaa
aacatgaagt atacactaaa agacatagga tttattttaa ccaaggcgaa ccaagccatt
caaacacttg aaaaatataa gacaatcctc gataaaacga ttaatgcact gaacgcgtta
gagtttgagg aacttgttac cttcagtgat gtcttgtctg tcatgcatcg ttatgaaatg
gtactgagaa tcaaaaacga aattaatatg tatatcaaag agctggggac agaagggcat
ctgatcaaac tgcaagtcat tgaattgatt acggatatgg aagaagaggc cgctttattt
attaaggact atgtaaaaga aaagattaaa gatccgtttg ttctcttgaa ggagctgcag
gatatgtcca gttatgatct gctggatgat tccattgtgt ataagcttct cggttaccct
gcttctacta atcttgatga ttatgtattg ccgagaggat acaggctgtt aaataagata
ccgcgtcttc cgatgccgat tgttgaaaat gttgtagaag catttggagt cctgccaagg
attattgagg cgagtgcaga agaattagat gaagtagagg gaatcggtga agtacgagcc
caaaaaatca aaaaaggatt aaaacgcctg caagagaagc attatttaga cagacaactg tga
SEQ ID NO: 75 DNA integrity scanning protein DisA [Bacillus subtilis] amino acid
sequence (UniProtKB: sp|P37573|DISA_BACSU)

| | |
|---|---|
| 1 | MEKEKKGAKH ELDLSSILQF VAPGTPLRAG MENVLRANTG GLIVVGYNDK VKEVVDGGFH |
| 61 | INTAFSPAHL YELAKMDGAI ILSDSGQKIL YANTQLMPDA TISSSETGMR HRTAERVAKQ |
| 121 | TGCLVIAISE RRNVITLYQE NMKYTLKDIG FILTKANQAI QTLEKYKTIL DKTINALNAL |
| 181 | EFEELVTFSD VLSVMHRYEM VLRIKNEINM YIKELGTEGH LIKLQVIELI TDMEEEAALF |
| 241 | IKDYVKEKIK DPFVLLKELQ DMSSYDLLDD SIVYKLLGYP ASTNLDDYVL PRGYRLLNKI |
| 301 | PRLPMPIVEN VVEAFGVLPR IIEASAEELD EVEGIGEVRA QKIKKGLKRL QEKHYLDRQL |

SEQ ID NO: 76 response regulator receiver modulated diguanylate cyclase
[Pelobacter propionicus DSM 2379] amino acid sequence (GenBank: ABK98996.1)

| | |
|---|---|
| 1 | MRRILVVEDD RFFRDLFYDL LVGQGYDVDR ASSGEEGLDR LSTYAFDLVV TDLVMPGVDG |
| 61 | MDILARAREN DPSADVIMVT GNANLESAIF ALKHGARDYF VKPINPDEFL HSVAQCLEQR |
| 121 | RILDENEELK SMLNLYQISQ AIAGCLDMER LQHLIFDAFT REIGTSRGMC LFATETGLEL |
| 181 | CEVKGVETAV AERCIASVLE RLSEDHPDEC NSLRISFQGG GDDSGIEAAI LIPLRGKGSQ |
| 241 | RGVVVAFNEP GLGLPELGAR KKNILFLLEQ SLLALENASS YSLAKDMLFI DDLSGLYNQR |
| 301 | YLEVALEREM KRIGRFSSQL AVLFLDMDSF KQVNDTHGHL VGSRVLKEMG TLLRLSVRDV |
| 361 | DVVIRYGGDE YTAILVETSP AIAANVAERI RSMVASHVFL ADEGYDIRLT CSIGYSCCPE |
| 421 | DALTKEELLE MADQAMYTGK GRGKNCVVRF TKTS |

SEQ ID NO: 77 response regulator receiver modulated diguanylate cyclase
[Geobacter uraniireducens Rf4] amino acid sequence (GenBank: ABQ26076.1)

| | |
|---|---|
| 1 | MERILVVEDD SFFREVFADL LIEDGFHVDV AASGEQALVM VQNREYQLVV TDLVMPDITG |
| 61 | LDILSKVKQL DPTIDVIMVT GHANMETAIF ALKNGARDYL VKPINHDEFK HAVALCFEQR |
| 121 | RLLDENQELK GLINLYHVSQ TIANCLDLER IHTLLVDSLA KEFAVSRGLG YFLDGADNLE |
| 181 | LKALKGVSEA SAGRLGELIL SRYNVQGEDS RSFVLLHDFM QPDADFGLGT DGDMKEAMLF |
| 241 | FVRSRTVLQG IVILFSEPGT SFPADIQFKN INFLLDQSSL ALENAVRYNN AKNLLYIDEL |
| 301 | TGLFNYRYLD VALEREIRRA ERYGSHISVI FLDIDLFKRV NDMYGHLVGS RALNEVGILL |
| 361 | KKSVRDVDTV IRYGGDEYTI ILIETGIDGA AAVAERIRRS IEAHGFMAAD GLNLKLTASL |
| 421 | GYACYPEDAK TKTELLELAD QAMYRGKADG KNRVFYVSAK NN |

SEQ ID NO: 78 response receiver-modulated diguanylate cyclase [Geobacter
daltonii FRC-32] amino acid sequence (GenBank: ACM20971.1)

| | |
|---|---|
| 1 | MERILVVEDD SFFREVFADL LRDDGFAVDV ACSGEKALEM LRSSEYALVV TDLVMPDITG |
| 61 | LDLLSKVKQF DPSIDVILVT GHANTETAVF ALKNGARDYL VKPINSEEFK HAVALCFEQR |
| 121 | RLLDENQELK GLLNLFQISQ TIANSLDFDR IHTILVDSLA KEFGLSRLTG YFQNDDGTLE |
| 181 | LKEIKGFDEE TASSLGELIF DIFDVREEDN RSFVLLNDLE QRSRFFAEHS VTEAMLFFVR |
| 241 | AKTALLGIII VFNESQSVFP AHLDFKNINF LLDQASLALE NASRYNNAKN LLYIDELTGL |
| 301 | FNYRYLDVAL EREVRRAERY SSNISIIFLD IDLFKRINDQ YGHLVGSKAL AEVGLLLKKS |
| 361 | VRDVDTVIRY GGDEYTIILI ETGIDGASVV AERIRSTIEG HVFIQSEGLD IKLTASLGCA |
| 421 | SYPEDACTKL ELLELADQAM YRSKACGKNM VFHISAYKKQ |

Included in Table 1 are variations of 1, 2, 3, 4, 5, 6, 7, 8, 9, 10, 11, 12, 13, 14, 15, 16, 17, 18, 19, 20, or more nucleotides or amino acids on the 5′ end, on the 3′ end, or on both the 5′ and 3′ ends, of the domain sequences as long as the sequence variations maintain the recited function and/or homology
Included in Table 1 are nucleic acid or polypeptide molecules comprising, consisting essentially of, or consisting of:
1) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with a nucleic acid or amino acid sequence of SEQ ID NO: 1-78, or a biologically active fragment thereof;
2) a nucleic acid or amino acid sequence having at least 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3500, 4000, 4500, 5000, 5500, 6000, 6500, 7000, 7500, 8000, 8500, 9000, 9500, 10000, or more nucleotides or amino acids, or any range in between, inclusive such as between 110 and 300 nucleotides or amino acids;
3) a biologically active fragment of a nucleic acid or amino acid sequence of SEQ ID NO: 1-78 having at least 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2625, or more nucleotides or amino acids, or any range in between, inclusive such as between 110 and 300 nucleotides or amino acids;
4) a biologically active fragment of a nucleic acid or amino acid sequence of SEQ ID NO: 1-78 having 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2625, or fewer nucleotides or amino acids, or any range in between, inclusive such as between 110 and 300 nucleotides or amino acids;

### II. Compositions of Matter—Cyclic Di-Nucleotide Synthetase Enzyme Containing Vectors, Pharmaceutical Compositions, Vaccine, Adjuvants

Novel cyclic di-nucleotide synthetase enzyme containing compositions are provided herein. Such compositions (e.g., vectors, pharmaceutical compositions, adjuvants, vaccines)) are useful for the prevention and treatment of diseases, conditions, or disorders, for which an upregulation of an immune response would be beneficial. For example, the compositions may be used in the prevention or treatment of pathogenic infections, such as viral, protozoal, fungal, or bacterial infections, or cancers. Such compositions comprise any cyclic di-nucleotide synthetase enzyme (e.g., one or more DGCs, DACs, Hypr-GGDEFs, DncV, DisA, cGAS, or any sequences that encode GGDEF domains belonging to the COG2199 protein domain family) listed herein, the Figures, and the Examples, or any subset thereof. In some embodiments, the compositions are provided alone or in combined with antigens (e.g., epitopes, tumor-associated antigens, or pathogen associated antigens) to enhance, stimulate, and/or increase an immune response.

In one embodiment, the DGC comprise any sequences that encode GGDEF domains belonging to the COG2199 protein domain family, or fragment thereof. As used herein, the term “nucleic acid molecule” is intended to include DNA molecules (i.e., cDNA or genomic DNA) and RNA molecules (i.e., mRNA) and analogs of the DNA or RNA generated using nucleotide analogs. The nucleic acid molecule can be single-stranded or double-stranded, but preferably is double-stranded DNA. An “isolated” nucleic acid molecule is one which is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecules corresponding to the one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof, can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived (i.e., bacterial strain, V. cholera strain). Moreover, an “isolated” nucleic acid molecule, such as a cDNA molecule, can be substantially free of other cellular material, or culture medium when produced by recombinant techniques, or chemical precursors or other chemicals when chemically synthesized.

A cyclic di-nucleotide synthetase enzyme nucleic acid molecule of the present invention, e.g., a nucleic acid molecule comprising the nucleotide sequence of one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof, or a nucleotide sequence which is at least about 50%, preferably at least about 60%, more preferably at least about 70%, yet more preferably at least about 80%, still more preferably at least about 90%, and most preferably at least about 95% or more (e.g., about 98%) homologous to the nucleotide sequence of one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or a portion thereof (i.e., 100, 200, 300, 400, 450, 500, or more nucleotides), can be isolated using standard molecular biology techniques and the sequence information provided herein. For example, a human cDNA can be isolated from a human cell line (from Stratagene, La Jolla, CA, or Clontech, Palo Alto, CA) using all or portion of the nucleic acid molecule, or fragment thereof, as a hybridization probe and standard hybridization techniques (i.e., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, NY, 1989). Moreover, a nucleic acid molecule encompassing all or a portion of the nucleotide sequence of one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof, or a nucleotide sequence which is at least about 50%, preferably at least about 60%, more preferably at least about 70%, yet more preferably at least about 80%, still more preferably at least about 90%, and most preferably at least about 95% or more homologous to the nucleotide sequence, or fragment thereof, can be isolated by the polymerase chain reaction using oligonucleotide primers designed based upon the sequence of the one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Example, or a biologically active fragment thereof, or the homologous nucleotide sequence. For example, mRNA can be isolated from cells of interest and cDNA can be prepared using reverse transcriptase (i.e., Moloney MLV reverse transcriptase, available from Gibco/BRL, Bethesda, MD; or AMV reverse transcriptase, available from Seikagaku America, Inc., St. Petersburg, FL). Synthetic oligonucleotide primers for PCR amplification can be designed according to well-known methods in the art. A nucleic acid of the present invention can be amplified using cDNA or, alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to the nucleotide sequence of one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, can be prepared by standard synthetic techniques, i.e., using an automated DNA synthesizer.

Probes based on the nucleotide sequences of one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof, can be used to detect transcripts or genomic sequences encoding the same or homologous sequences. In some embodiments, the probe further comprises a label group attached thereto, i.e., the label group can be a radioisotope, a fluorescent compound, an enzyme, or an enzyme co-factor. Such probes can be used as a part of a diagnostic test kit for identifying cells or tissue which express one or more cyclic di-nucleotide synthetase enzyme (e.g., DGCs, DACs, Hypr-GGDEFs, DncVDisA, cGAS, any sequences that encode GGDEF domains belonging to the COG2199 protein domain family) listed herein, the Figures, and the Examples, or any subset thereof, such as by measuring a level of nucleic acid in a sample of cells from a subject, i.e., detecting mRNA levels of one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof.

Nucleic acid molecules corresponding to one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof, from different species are also contemplated. In one embodiment, the nucleic acid molecule(s) of the present invention encodes a cyclic di-nucleotide synthetase enzyme or portion thereof which includes a nucleic acid sequence sufficiently similar to the nucleic acid sequence of one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof, such that the enzyme or portion thereof has enzymatic activity as described herein. Such homologous nucleic acids and encoded polypeptides can be readily produced by the ordinarily skilled artisan based on the sequence information provided herein, the Figures, and the Examples.

As used herein, the language “sufficiently homologous” refers to nucleic acids or portions thereof which have nucleic acid sequences which include a minimum number of identical or equivalent (e.g., a cognate pair of nucleotides for maintaining nucleic acid secondary structure) to a nucleic acid sequence of the cyclic di-nucleotide synthetase enzyme, or fragment thereof, such that the nucleic acid thereof modulates (e.g., enhances) one or more of the following biological activities: a) increase c-di-GMP, c-di-AMP, cGAMP, and/or any cyclic di-nucleotide; b) enhance innate immue response; c) stimulate adaptive immune response; and d) increase humoral immune response.

Portions of nucleic acid molecules of the one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof, are preferably biologically active portions of the protein. As used herein, the term “biologically active portion” of one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof, is intended to include a portion, e.g., a domain/motif, that has one or more of the biological activities of the full-length protein.

The invention further encompasses nucleic acid molecules that differ from the nucleotide sequence of the one or more cyclic di-nucleotide synthetase enzyme (e.g., DGCs, DACs, Hypr-GGDEFs, DncV, DisA, cGAS any sequences that encode GGDEF domains belonging to the COG2199 protein domain family) listed herein, the Figures, and the Examples, or any subset thereof, or fragment thereof due to degeneracy of the genetic code and thus encode the same protein as that encoded by the nucleotide sequence, or fragment thereof. In another embodiment, an isolated nucleic acid molecule of the present invention has a nucleotide sequence having a nucleic acid sequence of one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof, or fragment thereof, or having a nucleic acid sequence which is at least about 70%, 75%, 80%, 85%, 90%, 91%, 92%, 93%, 94%, 95%, 96%, 97%, 98%, 99% or more homologous to the amino acid sequence of the one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof, or fragment thereof. In another embodiment, a nucleic acid encoding a polypeptide consists of nucleic acid sequence encoding a portion of a full-length fragment of interest that is at least 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3500, 4000, 4500, 5000, 5500, 6000, 6500, 7000, 7500, 8000, 8500, 9000, 9500, 10000, or more nucleotides, or any range in between, inclusive such as between 110 and 300 nucleotides; or more nucleotides, or any range in between, inclusive such as between 110 and 300 nucleotides; or 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2625, or fewer nucleotides, or any range in between, inclusive such as between 110 and 300 nucleotides.

It will be appreciated by those skilled in the art that DNA sequence polymorphisms that lead to changes in the amino acid sequences of the one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof, may exist within a population. Such genetic polymorphisms may exist among individuals within a population due to natural allelic variation. As used herein, the terms “gene” and “recombinant gene” refer to nucleic acid molecules comprising an open reading frame encoding one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof, preferably bacterial, e.g., V. cholerae DGC. Such natural allelic variations can typically result in 1-5% variance in the nucleotide sequence of the one or more cyclic di-nucleotide synthetase enzyme (e.g., DGCs, DACs, Hypr-GGDEFs, DncV, DisA, cGAS any sequences that encode GGDEF domains belonging to the COG2199 protein domain family) listed herein, the Figures, and the Examples, or any subset thereof. Any and all such nucleotide variations and resulting amino acid polymorphisms in the one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof, that are the result of natural allelic variation and that do not alter the functional activity of the one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof, are intended to be within the scope of the present invention. Moreover, nucleic acid molecules encoding one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof, from other species.

In addition to naturally-occurring allelic variants of the one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof, sequence that may exist in the population, the skilled artisan will further appreciate that changes can be introduced by mutation into the nucleotide sequence, or fragment thereof, thereby leading to changes in the amino acid sequence of the encoded one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof, without altering the functional ability of the one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof. For example, nucleotide substitutions leading to substitutions at “non-essential” nucleotide positions can be made in the sequence, or fragment thereof. A “non-essential” amino acid position is a position that can be altered from the wild-type sequence of the one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof, without substantially altering the activity of the one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof, whereas an “essential” amino acid residue is required for the activity of the one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof. Other positions, however, (e.g., those that are not conserved or only semi-conserved between mouse and human) may not be essential for activity and thus are likely to be amenable to alteration without altering the activity of the one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof.

The term “sequence identity or homology” refers to the sequence similarity between two polypeptide molecules or between two nucleic acid molecules. When a position in both of the two compared sequences is occupied by the same base or amino acid monomer subunit, e.g., if a position in each of two DNA molecules is occupied by adenine, then the molecules are homologous or sequence identical at that position. The percent of homology or sequence identity between two sequences is a function of the number of matching or homologous identical positions shared by the two sequences divided by the number of positions compared×100. For example, if 6 of 10, of the positions in two sequences are the same then the two sequences are 60% homologous or have 60% sequence identity. By way of example, the DNA sequences ATTGCC and TATGGC share 50% homology or sequence identity. Generally, a comparison is made when two sequences are aligned to give maximum homology. Unless otherwise specified “loop out regions”, e.g., those arising from, from deletions or insertions in one of the sequences are counted as mismatches.

The comparison of sequences and determination of percent homology between two sequences can be accomplished using a mathematical algorithm. Preferably, the alignment can be performed using the Clustal Method. Multiple alignment parameters include GAP Penalty=10, Gap Length Penalty=10. For DNA alignments, the pairwise alignment parameters can be Htuple=2, Gap penalty=5, Window=4, and Diagonal saved=4. For protein alignments, the pairwise alignment parameters can be Ktuple=1, Gap penalty=3, Window=5, and Diagonals Saved=5.

In some embodiment, the percent identity between two amino acid sequences is determined using the Needleman and Wunsch (J. Mol. Biol. (48):444-453 (1970)) algorithm which has been incorporated into the GAP program in the GCG software package (available online), using either a Blossom 62 matrix or a PAM250 matrix, and a gap weight of 16, 14, 12, 10, 8, 6, or 4 and a length weight of 1, 2, 3, 4, 5, or 6. In yet another embodiment, the percent identity between two nucleotide sequences is determined using the GAP program in the GCG software package (available online), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0) (available online), using a PAM120 weight residue table, a gap length penalty of 12 and a gap penalty of 4.

An isolated nucleic acid molecule encoding a protein homologous to one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof, or fragment thereof, can be created by introducing one or more nucleotide substitutions, additions or deletions into the nucleotide sequence, or fragment thereof, or a homologous nucleotide sequence such that one or more amino acid substitutions, additions or deletions are introduced into the encoded protein. Mutations can be introduced by standard techniques, such as site-directed mutagenesis and PCR-mediated mutagenesis.

The levels of one or more cyclic di-nucleotide synthetase enzyme (e.g., DGCs, DACs, Hypr-GGDEFs, DncV, DisA, cGAS any sequences that encode GGDEF domains belonging to the COG2199 protein domain family) listed herein, the Figures, and the Examples, or any subset thereof, levels may be assessed by any of a wide variety of well-known methods for detecting expression of a transcribed molecule or protein. Non-limiting examples of such methods include immunological methods for detection of proteins, protein purification methods, protein function or activity assays, nucleic acid hybridization methods, nucleic acid reverse transcription methods, and nucleic acid amplification methods.

In some embodiments, the levels of one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof, levels are ascertained by measuring gene transcript (e.g., mRNA), by a measure of the quantity of translated protein, or by a measure of gene product activity. Expression levels can be monitored in a variety of ways, including by detecting cyclic di-nucleotide synthetase enzyme levels or activity, any of which can be measured using standard techniques. Detection can involve quantification of the level of gene expression (e.g., genomic DNA, cDNA, transcribed RNA, cyclic di-nucleotide synthetase enzyme activity), or, alternatively, can be a qualitative assessment of the level of gene expression, in particular in comparison with a control level. The type of level being detected will be clear from the context.

In a particular embodiment, the RNA expression level can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The isolated RNA can be used in hybridization or amplification assays that include, but are not limited to, Southern or Northern analyses, polymerase chain reaction analyses and probe arrays. One diagnostic method for the detection of RNA levels involves contacting the isolated RNA with a nucleic acid molecule (probe) that can hybridize to the RNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to an RNA or genomic DNA encoding one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof. Other suitable probes for use in the diagnostic assays of the present invention are described herein. Hybridization of an RNA with the probe indicates that one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof, is being expressed.

In one format, the RNA is immobilized on a solid surface and contacted with a probe, for example by running the isolated RNA on an agarose gel and transferring the RNA from the gel to a membrane, such as nitrocellulose. In an alternative format, the probe(s) are immobilized on a solid surface and the RNA is contacted with the probe(s), for example, in a gene chip array, e.g., an Affymetrix™ gene chip array. A skilled artisan can readily adapt known RNA detection methods for use in detecting the level of the one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof, RNA expression levels.

An alternative method for determining RNA expression level in a sample involves the process of nucleic acid amplification, e.g., by RT-PCR (the experimental embodiment set forth in Mullis, 1987, U.S. Pat. No. 4,683,202), ligase chain reaction (Barany, 1991, Proc. Natl. Acad. Sci. USA, 88:189-193), self-sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well-known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, RNA does not need to be isolated from the cells prior to detection. In such methods, a cell or tissue sample is prepared/processed using known histological methods. The sample is then immobilized on a support, typically a glass slide, and then contacted with a probe that can hybridize to the one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof.

As an alternative to making determinations based on the absolute expression level, determinations may be based on the normalized expression level of one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof. Expression levels are normalized by correcting the absolute expression level by comparing its expression to the expression of a non-cyclic di-nucleotide synthetase enzyme gene, e.g., a housekeeping gene that is constitutively expressed. Suitable genes for normalization include housekeeping genes such as the actin gene, or epithelial cell-specific genes. This normalization allows the comparison of the expression level in one sample, e.g., a subject sample, to another sample, e.g., a normal sample, or between samples from different sources.

The level or activity of a protein corresponding to one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof, can also be detected and/or quantified by detecting or quantifying the activity, such as effects on associate polypeptides like transcription factors or nuclear receptors. The associated polypeptide can be detected and quantified by any of a number of means well known to those of skill in the art. These may include analytic biochemical methods such as electrophoresis, capillary electrophoresis, high performance liquid chromatography (HPLC), thin layer chromatography (TLC), hyperdiffusion chromatography, liquid chromatrography tandem mass spectrometry (LC-MS/MS) and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, Western blotting, and the like. A skilled artisan can readily adapt known protein/antibody detection methods for use in determining whether cells express the cyclic di-nucleotide synthetase enzyme of interest.

a. Cyclic Di-Nucleotide Synthetase Enzyme Gene Containing Vectors

In some embodiments, vectors and/or host cells are further provided. One aspect of the present invention pertains to the use of recombinant vectors (e.g., gene therapy vectors), containing a nucleic acid encoding a cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof, or a portion or ortholog thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of recombinant vectors (e.g., viral vectors, replication defective adenoviruses, any human or non-human adenovirus, AAV, DNA-based vector, retroviruses, or lentiviruses), which serve equivalent functions. In one embodiment, vectors comprising a cyclic di-nucleotide synthetase enzyme nucleic acid molecule are used.

The recombinant vectors (e.g., gene thereapy vectors) of the present invention comprise any of the nucleic acid encoding a cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof, or a portion or ortholog thereof, in a form suitable for expression of the nucleic acid in a host cell, which means that the recombinant vectors include one or more regulatory sequences, selected on the basis of the host cells to be used for expression, which is operatively linked to the nucleic acid sequence to be expressed. Within a recombinant vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, CA (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the recombinant vector (e.g., gene therapy vector) can depend on such factors as the choice of the host cell to be transformed, the level of expression of protein desired, etc. The recombinant vectors (e.g., gene therapy vectors) of the present invention can be introduced into host cells to thereby produce proteins or peptides, including fusion proteins or peptides, encoded by nucleic acids as described herein.

The recombinant vectors (e.g., gene therapy vectors) of the present invention comprising any of the nucleic acid encoding a cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof, or a portion or ortholog thereof, can be designed for expression of the desired cyclic di-nucleotide synthetase enzyme in prokaryotic or eukaryotic cells. For example, a cyclic di-nucleotide synthetase enzyme can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, CA (1990). Alternatively, the recombinant vector can be transcribed and translated in vitro, for example using T7 promoter regulatory sequences and T7 polymerase. Examples of suitable inducible non-fusion E. coli vectors include pTrc (Amann et al., (1988) Gene 69:301-315) and pET 11d (Studier et al., Gene Expression Technology: Methods in Enzymology 185, Academic Press, San Diego, California (1990) 60-89). Examples of suitable yeast vectors include pYepSec1 (Baldari, et al., (1987) EMBO J. 6:229-234), pMFa (Kurjan and Herskowitz, (1982) Cell 30:933-943), pJRY88 (Schultz et al., (1987) Gene 54:113-123), and pYES2 (Invitrogen Corporation, San Diego, CA). Examples of suitable baculovirus vectors useful for insect cell hosts include the pAc series (Smith et al. (1983) Mol. Cell Biol. 3:2156-2165) and the pVL series (Lucklow and Summers (1989) Virology 170:31-39). Examples of suitable mammalian vectors include CMV-containing vectors, such as pCDM8 (Seed, B. (1987) Nature 329:840), and pMT2PC (Kaufman et al. (1987) EMBO J. 6:187-195).

In another embodiment, the recombinant vector (e.g., gene theray vector) comprising any of the nucleic acid encoding a cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof, or a portion or ortholog thereof, is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters such as in melanoma cancer cells are well-known in the art (see, for example, Pleshkan et al. (2011) Acta Nat. 3:13-21).

The present invention further provides a recombinant vector (e.g., gene therapy vector) comprising any of the nucleic acid encoding a cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof, or a portion or ortholog thereof, cloned into the recombinant vector (e.g., gene therapy vector) in an antisense orientation. That is, the DNA molecule is operatively linked to a regulatory sequence in a manner which allows for expression (by transcription of the DNA molecule) of an RNA molecule which is antisense to a cyclic di-nucleotide synthetase enzyme mRNA described herein. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense vector can be in the form of a recombinant plasmid, phagemid or attenuated virus in which antisense nucleic acids are produced under the control of a high efficiency regulatory region, the activity of which can be determined by the cell type into which the vector is introduced.

Another aspect of the present invention pertains to host cells into which a recombinant vector comprising any of the nucleic acid encoding a cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof, or a portion or ortholog thereof has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, cyclic di-nucleotide synthetase enzyme protein can be expressed in bacterial cells such as E. coli, insect cells, yeast or mammalian cells (such as Fao hepatoma cells, primary hepatocytes, Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, NY, 1989), and other laboratory manuals.

A cell culture includes host cells, media and other byproducts. Suitable media for cell culture are well known in the art. A cyclic di-nucleotide synthetase enzyme polypeptide or fragment thereof, may be secreted and isolated from a mixture of cells and medium containing the polypeptide. Alternatively, a cyclic di-nucleotide synthetase enzyme polypeptide or fragment thereof, may be retained cytoplasmically and the cells harvested, lysed and the protein or protein complex isolated. A cyclic di-nucleotide synthetase enzyme polypeptide or fragment thereof, may be isolated from cell culture medium, host cells, or both using techniques known in the art for purifying proteins, including ion-exchange chromatography, gel filtration chromatography, ultrafiltration, electrophoresis, and inmmunoaffinity purification with antibodies specific for particular epitopes of a cyclic di-nucleotide synthetase enzyme or a fragment thereof. In other embodiments, heterologous tags can be used for purification purposes (e.g., epitope tags and FC fusion tags), according to standards methods known in the art.

Thus, a nucleotide sequence encoding all or a selected portion of a cyclic di-nucleotide synthetase enzyme polypeptide may be used to produce a recombinant form of the protein via microbial or eukaryotic cellular processes. Ligating the sequence into a polynucleotide construct, such as an recombinant vector (e.g., gene therapy vector), and transforming or transfecting into hosts, either eukaryotic (yeast, avian, insect or mammalian) or prokaryotic (bacterial cells), are standard procedures. Similar procedures, or modifications thereof, may be employed to prepare recombinant cyclic di-nucleotide synthetase enzyme polypeptides, or fragments thereof, by microbial means or tissue-culture technology in accord with the subject invention.

A host cell of the present invention, such as a prokaryotic or eukaryotic host cell in culture, can be used to produce (i.e., express) cyclic di-nucleotide synthetase enzyme protein. Accordingly, the invention further provides methods for producing cyclic di-nucleotide synthetase enzyme protein using the host cells of the present invention. In one embodiment, the method comprises culturing the host cell of invention (into which a recombinant vector encoding a cyclic di-nucleotide synthetase enzyme has been introduced) in a suitable medium until cyclic di-nucleotide synthetase enzyme protein is produced. In another embodiment, the method further comprises isolating the cyclic di-nucleotide synthetase enzyme portein from the medium or the host cell.

The host cells of the present invention can also be used to produce nonhuman transgenic animals. The nonhuman transgenic animals can be used in screening assays designed to identify compositions or compounds, e.g., drugs, pharmaceuticals, etc., which are capable of modulation (e.g., upregulating) an immune response. For example, in one embodiment, a host cell of the present invention is a fertilized oocyte or an embryonic stem cell into which cyclic di-nucleotide synthetase enzyme encoding sequences, or fragments thereof, have been introduced. Such host cells can then be used to create non-human transgenic animals in which exogenous cyclic di-nucleotide synthetase enzyme sequences have been introduced into their genome or homologous recombinant animals in which endogenous cyclic di-nucleotide synthetase enzyme sequences have been altered. Such animals are useful for studying the function and/or activity of cyclic di-nucleotide synthetase enzyme, or fragments thereof, and for identifying and/or evaluating modulators of cyclic di-nucleotide synthetase enzyme activity. As used herein, a “transgenic animal” is a nonhuman animal, preferably a mammal, more preferably a rodent such as a rat or mouse, in which one or more of the cells of the animal includes a transgene. Other examples of transgenic animals include nonhuman primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a nonhuman animal, preferably a mammal, more preferably a mouse, in which an endogenous cyclic di-nucleotide synthetase enzyme gene has been altered by homologous recombination between the endogenous gene and an exogenous DNA molecule introduced into a cell of the animal, e.g., an embryonic cell of the animal, prior to development of the animal.

A transgenic animal of the present invention can be created by introducing nucleic acids encoding a cyclic di-nucleotide synthetase enzyme, or a fragment thereof, into the male pronuclei of a fertilized oocyte, e.g., by microinjection, retroviral infection, and allowing the oocyte to develop in a pseudopregnant female foster animal. Human cyclic di-nucleotide synthetase enzyme cDNA sequence can be introduced as a transgene into the genome of a nonhuman animal. Alternatively, a nonhuman homologue of the human cyclic di-nucleotide synthetase enzyme gene can be used as a transgene. Intronic sequences and polyadenylation signals can also be included in the transgene to increase the efficiency of expression of the transgene. A tissue-specific regulatory sequence(s) can be operably linked to the cyclic di-nucleotide synthetase enzyme transgene to direct expression of cyclic di-nucleotide synthetase enzyme protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the cyclic di-nucleotide synthetase enzyme transgene in its genome and/or expression of cyclic di-nucleotide synthetase enzyme mRNA in tissues or cells of the animals. A transgenic founder animal can then be used to breed additional animals carrying the transgene. Moreover, transgenic animals carrying a transgene encoding a cyclic di-nucleotide synthetase enzyme can further be bred to other transgenic animals carrying other transgenes.

To create a homologous recombinant animal, a vector is prepared which contains at least a portion of cyclic di-nucleotide synthetase enzyme gene into which a deletion, addition or substitution has been introduced to thereby alter, e.g., functionally disrupt, the cyclic di-nucleotide synthetase enzyme gene. The cyclic di-nucleotide synthetase enzyme gene can be a bacterial gene. The cyclic di-nucleotide synthetase enzyme gene can be a human gene, but more preferably, is a non-human homologue of a human cyclic di-nucleotide synthetase enzyme gene. For example, a mouse cyclic di-nucleotide synthetase enzyme gene can be used to construct a homologous recombination vector suitable for altering an endogenous cyclic di-nucleotide synthetase enzyme gene, respectively, in the mouse genome. In another embodiment, the vector is designed such that, upon homologous recombination, the endogenous cyclic di-nucleotide synthetase enzyme gene is functionally disrupted (i.e., no longer encodes a functional protein; also referred to as a “knock out” vector). Alternatively, the vector can be designed such that, upon homologous recombination, the endogenous DGC gene is mutated or otherwise altered but still encodes functional protein (e.g., the upstream regulatory region can be altered to thereby alter the expression of the endogenous cyclic di-nucleotide synthetase enzyme protein). In the homologous recombination vector, the altered portion of the cyclic di-nucleotide synthetase enzyme gene is flanked at its 5′ and 3′ ends by additional nucleic acid of the cyclic di-nucleotide synthetase enzyme gene to allow for homologous recombination to occur between the exogenous cyclic di-nucleotide synthetase enzyme gene carried by the vector and an endogenous cyclic di-nucleotide synthetase enzyme gene in an embryonic stem cell. The additional flanking cyclic di-nucleotide synthetase enzyme gene nucleic acid is of sufficient length for successful homologous recombination with the endogenous gene. Typically, several kilobases of flanking DNA (both at the 5′ and 3′ ends) are included in the vector (see e.g., Thomas, K. R. and Capecchi, M. R. (1987) Cell 51:503 for a description of homologous recombination vectors). The vector is introduced into an embryonic stem cell line (e.g., by electroporation) and cells in which the introduced cyclic di-nucleotide synthetase enzyme gene has homologously recombined with the endogenous cyclic di-nucleotide synthetase enzyme gene are selected (see e.g., Li, E. et al. (1992) Cell 69:915). The selected cells are then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic nonhuman animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the nonhuman transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyst and then transferred to pseudopregnant female foster animal. The offspring borne of this female foster animal will be a clone of the animal from which the cell, e.g., the somatic cell, is isolated.

Nucleic acid molecules of the present invention can also be engineered as fusion constructs using recombinant DNA techniques. A “chimeric cyclic di-nucleotide synthetase enzyme” or “fusion cyclic di-nucleotide synthetase enzyme” comprises a cyclic di-nucleotide synthetase enzyme polypeptide described herein operatively linked to a non-cyclic di-nucleotide synthetase enzyme nucleic acid sequence. Within the fusion construct, the term “operatively linked” is intended to indicate that the cyclic di-nucleotide synthetase enzyme nucleic acid sequence and the non-cyclic di-nucleotide synthetase enzyme nucleic acid sequence are fused in a rame to each other. The cyclic di-nucleotide synthetase enzyme polypeptide can be fused to the 5′ end, the 3′ end, or in between the 5′ and 3′ ends of the cyclic di-nucleotide synthetase enzyme nucleic acid sequence. The fusion protein can function as a nucleic acid (e.g., a MS2 loop structure) or encode a protein for translation, such as using an internal ribosome entry sequence (IRES). For example, in one embodiment the fusion protein is a cyclic di-nucleotide synthetase enzyme—GST and/or cyclic di-nucleotide synthetase enzyme—Fc fusion protein. Such fusion proteins can facilitate the purification, expression, and/or bioavailability of recombinant cyclic di-nucleotide synthetase enzyme constructs. In certain host cells (e.g., mammalian host cells), expression and/or secretion of the cyclic di-nucleotide synthetase enzyme fusion construct can be increased through use of a heterologous signal sequence.

Preferably, a cyclic di-nucleotide synthetase enzyme chimeric or fusion constructs (e.g., gene therapy vectors comprising cyclic di-nucleotide synthetase enzyme) of the present invention is produced by standard recombinant DNA techniques. For example, DNA fragments coding for the different sequences are ligated together in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A cyclic di-nucleotide synthetase enzyme-encoding nucleic acid can be cloned into such an expression vector such that the fusion moiety is linked in-frame to the cyclic di-nucleotide synthetase enzyme protein.

Systematic substitution of one or more amino acids of a polypeptide amino acid sequence with a D-amino acid of the same type (e.g., D-lysine in place of L-lysine) can be used to generate more stable peptides. In addition, constrained peptides comprising a polypeptide amino acid sequence of interest or a substantially identical sequence variation can be generated by methods known in the art (Rizo and Gierasch (1992) Annu. Rev. Biochem. 61:387, incorporated herein by reference); for example, by adding internal cysteine residues capable of forming intramolecular disulfide bridges which cyclize the peptide.

The amino acid sequences disclosed herein will enable those of skill in the art to produce polypeptides corresponding peptide sequences and sequence variants thereof. Such polypeptides can be produced in prokaryotic or eukaryotic host cells by expression of polynucleotides encoding the peptide sequence, frequently as part of a larger polypeptide. Alternatively, such peptides can be synthesized by chemical methods. Methods for expression of heterologous proteins in recombinant hosts, chemical synthesis of polypeptides, and in vitro translation are well known in the art and are described further in Maniatis et al. Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken I. M. (1981) CRC Crit. Rev. Biochem. 11: 255; Kaiser et al. (1989) Science 243:187; Merrifield, B. (1986) Science 232:342; Kent, S. B. H. (1988) Annu. Rev. Biochem. 57:957; and Offord, R. E. (1980) Semisynthetic Proteins, Wiley Publishing, which are incorporated herein by reference).

Peptides can be produced, typically by direct chemical synthesis. Peptides can be produced as modified peptides, with nonpeptide moieties attached by covalent linkage to the N-terminus and/or C-terminus. In certain embodiments, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal-modifications such as amidation, as well as other terminal modifications, including cyclization, can be incorporated into various embodiments of the present invention. Certain amino-terminal and/or carboxy-terminal modifications and/or peptide extensions to the core sequence can provide advantageous physical, chemical, biochemical, and pharmacological properties, such as: enhanced stability, increased potency and/or efficacy, resistance to serum proteases, desirable pharmacokinetic properties, and others. Peptides disclosed herein can be used therapeutically to treat disease.

b. Pharmaceutical Compositions, Adjuvants, Vaccines

In another aspect, the present invention provides pharmaceutically acceptable compositions, adjuvants, and vaccines which comprise a therapeutically-effective amount of a recombinant vector (e.g., gene therapy vector comprising any of the nucleotide sequence of the one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof, or fragment thereof) which increases cyclic di-nucleotide synthetase enzyme, c-di-GMP, c-di-AMP, c-di-GAMP, any cyclic di-nucleotide, or immune response levels and/or activity, formulated together with one or more pharmaceutically acceptable carriers (additives) and/or diluents. In some embodiments, the pharmaceutical compositions, adjuvants, and vaccines comprises a first gene therapy vector (e.g., gene therapy vector containing any of the nucleotide sequence of the one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof, or fragment thereof) in combination with a extracellular antigen, epitope, or peptide (naked or provided in an gene therapy vector). In some embodiments, the pharmaceutical compositions, adjuvants, and vaccines can be combined with any immune modulating, anti-viral, anti-bacterial, anti-cancer, chemotherapeutic, or immunotherapeutic compositions.

Immunotherapeutic compositions, include, but are not limited to, ipilimumab (Yervoy®), trastuzumab (Herceptin®), rituximab (Rituxan®), bevacizumab (Avastin®), pertuzumab (Omnitarg®), tositumomab (Bexxar®), edrecolomab (Panorex®), and G250. Compounds of the present invention can also be combined with, or used in combination with, anti-TNF-α antibodies. Large molecule active compositions may be administered in the form of anti-cancer vaccines. For example, compositions that secrete, or cause the secretion of, cytokines such as IL-2, G-CSF, and GM-CSF can be used in the methods, pharmaceutical compositions, and kits provided herein. See, e.g., Emens, L. A., et al., Curr. Opinion Mol. Ther. 3(1):77-84 (2001).

Second active compositions that are small molecules can also be used to in combination with the compositions of the present invention. Examples of small molecule second active compositions include, but are not limited to, anti-cancer compositions, antibiotics, antivirals, immunosuppressive compositions, and steroids.

In some embodiments, well known “combination chemotherapy” regimens can be used. In one embodiment, the combination chemotherapy comprises a combination of two or more of cyclophosphamide, hydroxydaunorubicin (also known as doxorubicin or adriamycin), oncovorin (vincristine), and prednisone. In another embodiment, the combination chemotherapy comprises a combination of cyclophsophamide, oncovorin, prednisone, and one or more chemotherapeutics selected from the group consisting of anthracycline, hydroxydaunorubicin, epirubicin, and motixantrone.

Examples of other anti-cancer compositions include, but are not limited to: acivicin; aclarubicin; acodazole hydrochloride; acronine; adozelesin; aldesleukin; altretamine; ambomycin; ametantrone acetate; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib (COX-2 inhibitor); chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cyclosporin A; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (e.g., Gleevec®), imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim; Erbitux, human chorionic gonadotrophin; monophosphoryl lipid A+myobacterium cell wall sk; mopidamol; mustard anticancer composition; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; nilutamide; nisamycin; nitric oxide modulators; nitroxide antioxidant; nitrullyn; oblimersen (Genasense®); O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer. Specific second active compositions include, but are not limited to, chlorambucil, fludarabine, dexamethasone (Decadron®), hydrocortisone, methylprednisolone, cilostamide, doxorubicin (Doxil®), forskolin, rituximab, cyclosporin A, cisplatin, vincristine, PDE7 inhibitors such as BRL-50481 and IR-202, dual PDE4/7 inhibitors such as IR-284, cilostazol, meribendan, milrinone, vesnarionone, enoximone and pimobendan, Syk inhibitors such as fostamatinib disodium (R406/R788), R343, R-112 and Excellair® (ZaBeCor Pharmaceuticals, Bala Cynwyd, Pa.).

Antiviral, antifungal, and/or antibacterial compositions, include but not limited, cidofovir and interleukin-2, Cytarabine (also known as ARA-C), isoniazid, rifampicin, pyrazinamide, ethambutol, streptomycin, kanamycin, amikacin, capreomycin, ofloxacin, levofioxacin, moxifioxacin, cycloserine, para-aminosaicylic acid, ethioamide, prothionamide, thioacetazone, clofazimine, amoxicilin with clavulanate, imipenem, linezolid, clarithromycin, thioridazine, bicyclic nitroimidazoles (e.g., (S)-6,7-dihydro-2-nitro-6-[[4-(trifluoromethoxy)phenyl]methoxy]-5H-imidazo[2,1-b][1,3]oxazine (PA-824) and TBA-354, available from TB Alliance), bedaquiline (TMC-207), delamanid (OPC67683), oxazolidinone, 2-[(2S)-2-methyl-1,4-dioxa-8-azaspiro[4.5]decan-8-yl]-8-nitro-6-trifluoromethyl-4H-1,3-benzothiazin-4-one (BTZ043), imidazopyridines (e.g., Q201, available from Quro Science Inc.), anti-interleukin 4 neutralizing antibodies, high-dose intravenous immunoglobulin, 16a-bromoepiandosterone (HE2000), RUTI® vaccine, DNA vaccine with HSP65, Ag85, MPT-64, and MPT-83, dzherelo (plant extracts from the Ukraine), cytokines (such as Interleukin 2, Interleukin 7, Interleukin 15, Interleukin 27, Interleukin 12, Interferon γ, corticosteroids, thalidomide, etanercept, steroids, prednisone, (NNRTIs), such as efavirenz (Sustiva), etravirine (Intelence) and nevirapine (Viramune); Nucleoside reverse transcriptase inhibitors (NRTIs), such as Abacavir (Ziagen), and the combination drugs emtricitabine and tenofovir (Truvada), and lamivudine and zidovudine (Combivir); Protease inhibitors (Pis), such as atazanavir (Reyataz), darunavir (Prezista), fosamprenavir (Lexiva) and ritonavir (Norvir); Entry or fusion inhibitors, such enfuvirtide (Fuzeon) and maraviroc (Selzentry); and Integrase inhibitors, such as Raltegravir (Isentress).

As described in detail below, the pharmaceutical compositions, adjuvants, and vaccines of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “therapeutically-effective amount” as used herein means that amount of a composition of matter of the present invention that modulates immune response levels and/or activity, which is effective for producing some desired therapeutic effect, e.g., pathogenic infection or cancer treatment, at a reasonable benefit/risk ratio.

The phrase “pharmaceutically acceptable” is employed herein to refer to those pharmaceutical compositions, adjuvants, vaccines, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering compositions, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Formulations useful in the methods of the present invention include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form will generally be that amount of the compound which produces a therapeutic effect. Generally, out of one hundred percent, this amount will range from about 1% to about 99% of active ingredient, preferably from about 5% to about 70%, most preferably from about 10% to about 30%.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an composition as an active ingredient. A compound may also be administered as a bolus, electuary or paste.

In solid dosage forms for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating compositions, such as agar-agar, calcium carbonate, potato or tapioca starch, alginic acid, certain silicates, and sodium carbonate; (5) solution retarding compositions, such as paraffin; (6) absorption accelerators, such as quaternary ammonium compounds; (7) wetting compositions, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring compositions. In the case of capsules, tablets and pills, the pharmaceutical compositions may also comprise buffering compositions. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing composition. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing compositions in the form of sterile solid compositions, which can be dissolved in sterile water, or some other sterile injectable medium immediately before use. These compositions may also optionally contain opacifying compositions and may be of a composition that they release the active ingredient(s) only, or preferentially, in a certain portion of the gastrointestinal tract, optionally, in a delayed manner. Examples of embedding compositions, which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing compositions and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting compositions, emulsifying and suspending compositions, sweetening, flavoring, coloring, perfuming and preservative compositions.

Suspensions, in addition to the active composition may contain suspending compositions as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more therapeutic compositions with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active composition.

Formulations which are suitable for vaginal administration also include pessaries, tampons, creams, gels, pastes, foams or spray formulations containing such carriers as are known in the art to be appropriate.

Dosage forms for the topical or transdermal administration of an composition that modulates (e.g., increases) immune response levels and/or activity include powders, sprays, ointments, pastes, creams, lotions, gels, solutions, patches and inhalants. The active component may be mixed under sterile conditions with a pharmaceutically-acceptable carrier, and with any preservatives, buffers, or propellants which may be required.

The ointments, pastes, creams and gels may contain, in addition to a therapeutic composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an composition that modulates (e.g., increases) immune response levels and/or activity, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The composition that modulates (e.g., increases) immune response levels and/or activity, can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the composition to shear, which can result in degradation of the compound.

Ordinarily, an aqueous aerosol is made by formulating an aqueous solution or suspension of the composition together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a therapeutic composition to the body. Such dosage forms can be made by dissolving or dispersing the composition in the proper medium. Absorption enhancers can also be used to increase the flux of the peptidomimetic across the skin. The rate of such flux can be controlled by either providing a rate controlling membrane or dispersing the peptidomimetic in a polymer matrix or gel.

Ophthalmic formulations, eye ointments, powders, solutions and the like, are also contemplated as being within the scope of this invention.

Pharmaceutical compositions of this invention suitable for parenteral administration comprise one or more therapeutic compositions in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening compositions.

Examples of suitable aqueous and nonaqueous carriers which may be employed in the pharmaceutical compositions of the present invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting compositions, emulsifying compositions and dispersing compositions. Prevention of the action of microorganisms may be ensured by the inclusion of various antibacterial and antifungal compositions, for example, paraben, chlorobutanol, phenol sorbic acid, and the like. It may also be desirable to include isotonic compositions, such as sugars, sodium chloride, and the like into the compositions. In addition, prolonged absorption of the injectable pharmaceutical form may be brought about by the inclusion of compositions which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of an composition that modulates (e.g., increases) immune response levels and/or activity, in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

When the compositions of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

Actual dosage levels of the active ingredients in the pharmaceutical compositions of this invention may be determined by the methods of the present invention so as to obtain an amount of the active ingredient, which is effective to achieve the desired therapeutic response for a particular subject, composition, and mode of administration, without being toxic to the subject.

The cyclic di-nucleotide synthetase enzyme containing vectors can be used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054 3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., adenoviral viral vectors, the pharmaceutical preparation can include one or more cells which produce the gene delivery system.

### III. Uses and Methods of the Present Invention

The compositions of matter of the present invention comprising a vector (e.g., any gene therapy vector compring the nucleotide sequence of one or more cyclic di-nucleotide synthetase enzyme (e.g., DGCs, DACs, Hypr-GGDEFs, DncV, DisA, cGAS any sequences that encode GGDEF domains belonging to the COG2199 protein domain family) listed herein, the Figures, and the Examples, or any subset thereof or a portion thereof) can be used in one or more of the following methods: a) method of inducing or enhancing an immune response in a mammal and b) methods of treatment (e.g., therapeutic and prophylactic) in a mammal (e.g., human) having a condition that would benefit from upregulation of an immune response.

In one aspect, the present invention provides a method for preventing in a subject a pathogenic infection, by administering to the subject the compositions of matter of the present invention which modulates cyclic di-nucleotide synthetase enzyme expression or at least one activity of the cyclic di-nucleotide synthetase enzyme. Administration of such compositions can occur prior to the manifestation of symptoms characteristic of the pathogenic infection, such that an infection is prevented or, alternatively, delayed in its progression.

Another aspect of the present invention pertains to methods of modulating the expression or activity of one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof, or fragments thereof, for therapeutic purposes. Accordingly, the activity and/or expression of the cyclic di-nucleotide synthetase enzyme can be modulated in order to modulate the immune response.

The present invention also contemplates a method for enhancing an immune response comprising the administration to a subject the compositions of the present invention as part of a vaccination regimen. The present invention is particularly useful in pharmaceutical vaccines and genetic vaccines in humans.

Adjuvants promote the immune response in a number of ways such as to modify the activities of immune cells that are involved with generating and maintaining the immune response. Additionally, adjuvants modify the presentation of antigen to the immune system. The compositions of the invention (e.g., the recombinant vectors (e.g., gene therapy vectors) containing at least one nucleic acid encoding a cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof, or a portion or ortholog thereof) may be used as adjuvants in a vaccination regimen.

Another aspect of the invention pertains to therapeutic methods of modulating an immune response, e.g., enhancing or increasing an immune response by transducing DGC using an adenovirus to increase c-di-GMP levels.

Modulatory methods of the present invention involve contacting a cell, such as an immune cell with any of the compositions of matter (e.g., any gene therapy vector comprising the nucleotide sequence of one or more cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof or a portion thereof). Exemplary compositions useful in such methods are described above. Such compositions can be administered in vitro or ex vivo (e.g., by contacting the cell with the composition) or, alternatively, in vivo (e.g., by administering the compositions to a subject). As such, the present invention provides methods useful for treating an individual afflicted with a condition that would benefit from an increased immune response, such as a pathogenic infection or a cancer.

Compositions that upregulate immune responses can be in the form of enhancing an existing immune response or eliciting an initial immune response. Thus, enhancing an immune response using the subject compositions and methods is useful for treating cancer, but can also be useful for treating an infectious disease (e.g., bacteria, viruses, or parasites), a parasitic infection, and an immunosuppressive disease.

Exemplary infectious disorders include viral skin diseases, such as Herpes or shingles, in which case such a composition can be delivered topically to the skin. In addition, systemic viral diseases, such as influenza, the common cold, and encephalitis might be alleviated by systemic administration of such compositions.

Immune responses can also be enhanced in an infected patient through an ex vivo approach, for instance, by removing immune cells from the patient, contacting immune cells in vitro with an composition described herein and reintroducing the in vitro stimulated immune cells into the patient.

In certain instances, it may be desirable to further administer other compositions that upregulate immune responses. Such additional compositions and therapies are described further below.

Compositions that upregulate an immune response can be used prophylactically in vaccines against various polypeptides (e.g., polypeptides derived from pathogens). Immunity against a pathogen (e.g., a virus) can be induced by vaccinating with a viral protein or antigen along with a recombinant vector (e.g., gene therapy vector contining cyclic di-nucleotide synthetase enzyme) as an appropriate adjuvant for upregulatingan immune response.

In another embodiment, upregulation or enhancement of an immune response function, as described herein, is useful in the induction of tumor immunity.

In another embodiment, the immune response can be stimulated by the methods described herein, such that preexisting tolerance, clonal deletion, and/or exhaustion (e.g., T cell exhaustion) is overcome. For example, immune responses against antigens to which a subject cannot mount a significant immune response, such as a pathogen specific or tumor specific antigens can be induced by administering appropriate compositions described herein that upregulate the immune response. In one embodiment, an extracellular antigen, such as a pathogen-specific or tumor-specific antigen, can be coadministered. In another embodiment, the subject compositions can be used as adjuvants to boost responses to foreign antigens in the process of active immunization.

In still another embodiment, compositions described herein useful for upregulating immune responses can further be linked, or operatively attached, to toxins using techniques that are known in the art, e.g., crosslinking or via recombinant DNA techniques. Such compositions can result in cellular destruction of desired cells. In one embodiment, a toxin can be conjugated to an antibody, such as a bispecific antibody. Such antibodies are useful for targeting a specific cell population, e.g., using a marker found only on a certain type of cell. The preparation of immunotoxins is, in general, well known in the art (see, e.g., U.S. Pat. No. 4,340,535, and EP 44167). Numerous types of disulfide-bond containing linkers are known which can successfully be employed to conjugate the toxin moiety with a polypeptide. In one embodiment, linkers that contain a disulfide bond that is sterically “hindered” are preferred, due to their greater stability in vivo, thus preventing release of the toxin moiety prior to binding at the site of action. A wide variety of toxins are known that may be conjugated to polypeptides or antibodies of the invention. Examples include: numerous useful plant-, fungus- or even bacteria-derived toxins, which, by way of example, include various A chain toxins, particularly ricin A chain, ribosome inactivating proteins such as saporin or gelonin, α-sarcin, aspergillin, restrictocin, ribonucleases, such as placental ribonuclease, angiogenic, diphtheria toxin, and Pseudomonas exotoxin, etc. A preferred toxin moiety for use in connection with the invention is toxin A chain which has been treated to modify or remove carbohydrate residues, deglycosylated A chain. (U.S. Pat. No. 5,776,427). Infusion of one or a combination of such cytotoxic compositions, (e.g., ricin fusions) into a patient may result in the death of immune cells.

In another embodiment, certain combinations work synergistically in the treatment of conditions that would benefit from the modulation of immune responses. Second active compositions can be large molecules (e.g., proteins) or small molecules (e.g., synthetic inorganic, organometallic, or organic molecules). For example, anti-virals or anti-cancer compositions can be further combined with the compositions of the present invention to enhance or stimulate an immune response.

In one embodiment, anti-cancer immunotherapy is administered in combination to subjects described herein. The term “immunotherapy” refers to any therapy that acts by targeting immune response modulation (e.g., induction, enhancement, suppression, or reduction of an immune response). In certain embodiments, immunotherapy is administered that ativates T cells that recognize neoantigens (e.g., mutants that change the normal protein coding sequence and can be processed by the antigen presentation system, bind to MHC and recognized as foreign by T cells).

The term “immune response” includes T cell-mediated and/or B cell-mediated immune responses. Exemplary immune responses include T cell responses, e.g., cytokine production and cellular cytotoxicity. In addition, the term “immune response” includes immune responses that are indirectly effected by T cell activation, e.g., antibody production (humoral responses) and activation of cytokine responsive cells, e.g., macrophages. The term “inhibit” includes the decrease, limitation, or blockage, of, for example a particular action, function, or interaction. In some embodiments, cancer is “inhibited” if at least one symptom of the cancer is alleviated, terminated, slowed, or prevented. As used herein, cancer is also “inhibited” if recurrence or metastasis of the cancer is reduced, slowed, delayed, or prevented. The term “promote” has the opposite meaning.

The term “immunotherapeutic composition” can include any molecule, peptide, antibody or other composition which can modulate a host immune system in response to an antigen, such as expressed by a tumor or cancer in the subject. Immunotherapeutic strategies include administration of vaccines, antibodies, cytokines, chemokines, as well as small molecular inhibitors, anti-sense oligonucleotides, and gene therapy, as described further below (see, for example, Mocellin et al. (2002) Cancer Immunol. Immunother. 51:583-595; Dy et al. (2002) J. Clin. Oncol. 20: 2881-2894).

Immunotherapies that are designed to elicit or amplify an immune response are referred to as “activation immunotherapies.” Immunotherapies that are designed to reduce or suppress an immune response are referred to as “suppression immunotherapies.” Any composition believed to have an immune system effect on the genetically modified transplanted cancer cells can be assayed to determine whether the composition is an immunotherapy and the effect that a given genetic modification has on the modulation of immune response. In some embodiments, the immunotherapy is cancer cell-specific.

Immunotherapy can involve passive immunity for short-term protection of a host, achieved by the administration of pre-formed antibody directed against a cancer antigen or disease antigen (e.g., administration of a monoclonal antibody, optionally linked to a chemotherapeutic composition or toxin, to a tumor antigen). Immunotherapy can also focus on using the cytotoxic lymphocyte-recognized epitopes of cancer cell lines.

In one embodiment, immunotherapy comprises adoptive cell-based immunotherapies. Well known adoptive cell-based immunotherapeutic modalities, including, without limitation, Irradiated autologous or allogeneic tumor cells, tumor lysates or apoptotic tumor cells, antigen-presenting cell-based immunotherapy, dendritic cell-based immunotherapy, adoptive T cell transfer, adoptive CAR T cell therapy, autologous immune enhancement therapy (AIET), cancer vaccines, and/or antigen presenting cells. Such cell-based immunotherapies can be further modified to express one or more gene products to further modulate immune responses, such as expressing cytokines like GM-CSF, and/or to express tumor-associated antigen (TAA) antigens, such as Mage-1, gp-100, patient-specific neoantigen vaccines, and the like.

In another embodiment, immunotherapy comprises non-cell-based immunotherapies. In one embodiment, compositions comprising antigens with or without vaccine-enhancing adjuvants are used. Such compositions exist in many well known forms, such as peptide compositions, oncolytic viruses, recombinant antigen comprising fusion proteins, and the like. In still another embodiment, immunomodulatory interleukins, such as IL-2, IL-6, IL-7, IL-12, IL-17, IL-23, and the like, as well as modulators thereof (e.g., blocking antibodies or more potent or longer lasting forms) are used. In yet another embodiment, immunomodulatory cytokines, such as interferons, G-CSF, imiquimod, TNFalpha, and the like, as well as modulators thereof (e.g., blocking antibodies or more potent or longer lasting forms) are used. In another embodiment, immunomodulatory chemokines, such as CCL3, CCL26, and CXCL7, and the like, as well as modulators thereof (e.g., blocking antibodies or more potent or longer lasting forms) are used. In another embodiment, immunomodulatory molecules targeting immunosuppression, such as STAT3 signaling modulators, NFkappaB signaling modulators, and immune checkpoint modulators, are used. The terms “immune checkpoint” and “anti-immune checkpoint therapy” are described above.

In still another embodiment, immunomodulatory drugs, such as immunocytostatic drugs, glucocorticoids, cytostatics, immunophilins and modulators thereof (e.g., rapamycin, a calcineurin inhibitor, tacrolimus, ciclosporin (cyclosporin), pimecrolimus, abetimus, gusperimus, ridaforolimus, everolimus, temsirolimus, zotarolimus, etc.), hydrocortisone (cortisol), cortisone acetate, prednisone, prednisolone, methylprednisolone, dexamethasone, betamethasone, triamcinolone, beclometasone, fludrocortisone acetate, deoxycorticosterone acetate (doca) aldosterone, a non-glucocorticoid steroid, a pyrimidine synthesis inhibitor, leflunomide, teriflunomide, a folic acid analog, methotrexate, anti-thymocyte globulin, anti-lymphocyte globulin, thalidomide, lenalidomide, pentoxifylline, bupropion, curcumin, catechin, an opioid, an IMPDH inhibitor, mycophenolic acid, myriocin, fingolimod, an NF-xB inhibitor, raloxifene, drotrecogin alfa, denosumab, an NF-xB signaling cascade inhibitor, disulfiram, olmesartan, dithiocarbamate, a proteasome inhibitor, bortezomib, MG132, Prol, NPI-0052, curcumin, genistein, resveratrol, parthenolide, thalidomide, lenalidomide, flavopiridol, non-steroidal anti-inflammatory drugs (NSAIDs), arsenic trioxide, dehydroxymethylepoxyquinomycin (DHMEQ), I3C (indole-3-carbinol)/DIM(di-indolmethane) (13C/DIM), Bay 11-7082, luteolin, cell permeable peptide SN-50, IKBa.-super repressor overexpression, NFKB decoy oligodeoxynucleotide (ODN), or a derivative or analog of any thereo, are used. In yet another embodiment, immunomodulatory antibodies or protein are used. For example, antibodies that bind to CD40, Toll-like receptor (TLR), OX40, GITR, CD27, or to 4-1BB, T-cell bispecific antibodies, an anti-IL-2 receptor antibody, an anti-CD3 antibody, OKT3 (muromonab), otelixizumab, teplizumab, visilizumab, an anti-CD4 antibody, clenoliximab, keliximab, zanolimumab, an anti-CD11 a antibody, efalizumab, an anti-CD18 antibody, erlizumab, rovelizumab, an anti-CD20 antibody, afutuzumab, ocrelizumab, ofatumumab, pascolizumab, rituximab, an anti-CD23 antibody, lumiliximab, an anti-CD40 antibody, teneliximab, toralizumab, an anti-CD40L antibody, ruplizumab, an anti-CD62L antibody, aselizumab, an anti-CD80 antibody, galiximab, an anti-CD147 antibody, gavilimomab, a B-Lymphocyte stimulator (BLyS) inhibiting antibody, belimumab, an CTLA4-Ig fusion protein, abatacept, belatacept, an anti-CTLA4 antibody, ipilimumab, tremelimumab, an anti-eotaxin 1 antibody, bertilimumab, an anti-a4-integrin antibody, natalizumab, an anti-IL-6R antibody, tocilizumab, an anti-LFA-1 antibody, odulimomab, an anti-CD25 antibody, basiliximab, daclizumab, inolimomab, an anti-CD5 antibody, zolimomab, an anti-CD2 antibody, siplizumab, nerelimomab, faralimomab, atlizumab, atorolimumab, cedelizumab, dorlimomab aritox, dorlixizumab, fontolizumab, gantenerumab, gomiliximab, lebrilizumab, maslimomab, morolimumab, pexelizumab, reslizumab, rovelizumab, talizumab, telimomab aritox, vapaliximab, vepalimomab, aflibercept, alefacept, rilonacept, an IL-1 receptor antagonist, anakinra, an anti-IL-5 antibody, mepolizumab, an IgE inhibitor, omalizumab, talizumab, an IL12 inhibitor, an IL23 inhibitor, ustekinumab, and the like.

Nutritional supplements that enhance immune responses, such as vitamin A, vitamin E, vitamin C, and the like, are well known in the art (see, for example, U.S. Pat. Nos. 4,981,844 and 5,230,902 and PCT Publ. No. WO 2004/004483) can be used in the methods described herein.

Similarly, compositions and therapies other than immunotherapy or in combination thereof can be used with in combination with the compositions of the present invention to stimulate an immune response to thereby treat a condition that would benefit therefrom. For example, chemotherapy, radiation, epigenetic modifiers (e.g., histone deacetylase (HDAC) modifiers, methylation modifiers, phosphorylation modifiers, and the like), targeted therapy, and the like are well known in the art.

In one embodiment, chemotherapy is used. Chemotherapy includes the administration of a chemotherapeutic composition. Such a chemotherapeutic composition may be, but is not limited to, those selected from among the following groups of compounds: platinum compounds, cytotoxic antibiotics, antimetabolities, anti-mitotic compositions, alkylating compositions, arsenic compounds, DNA topoisomerase inhibitors, taxanes, nucleoside analogues, plant alkaloids, and toxins; and synthetic derivatives thereof. Exemplary compounds include, but are not limited to, alkylating compositions: cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic compositions: halichondrin, colchicine, and rhizoxin. Compositions comprising one or more chemotherapeutic compositions (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiments, PARP (e.g., PARP-1 and/or PARP-2) inhibitors are used and such inhibitors are well known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6(5H)-phenanthridinone (Trevigen); benzamide (U.S. Pat. Re. 36,397); and NU1025 (Bowman et al.). The mechanism of action is generally related to the ability of PARP inhibitors to bind PARP and decrease its activity. PARP catalyzes the conversion of .beta.-nicotinamide adenine dinucleotide (NAD+) into nicotinamide and poly-ADP-ribose (PAR). Both poly (ADP-ribose) and PARP have been linked to regulation of transcription, cell proliferation, genomic stability, and carcinogenesis (Bouchard V. J. et. al. Experimental Hematology, Volume 31, Number 6, June 2003, pp. 446-454(9); Herceg Z.; Wang Z.-Q. Mutation Research/Fundamental and Molecular Mechanisms of Mutagenesis, Volume 477, Number 1, 2 Jun. 2001, pp. 97-110(14)). Poly(ADP-ribose) polymerase 1 (PARP1) is a key molecule in the repair of DNA single-strand breaks (SSBs) (de Murcia J. et al. 1997. Proc Natl Acad Sci USA 94:7303-7307; Schreiber V, Dantzer F, Ame J C, de Murcia G (2006) Nat Rev Mol Cell Biol 7:517-528; Wang Z Q, et al. (1997) Genes Dev 11:2347-2358). Knockout of SSB repair by inhibition of PARP1 function induces DNA double-strand breaks (DSBs) that can trigger synthetic lethality in cancer cells with defective homology-directed DSB repair (Bryant H E, et al. (2005) Nature 434:913-917; Farmer H, et al. (2005) Nature 434:917-921). The foregoing examples of chemotherapeutic compositions are illustrative, and are not intended to be limiting. Additional examples of chemotherapeutic and other anti-cancer compositions are described in US Pat. Publs. 2013/0239239 and 2009/0053224.

In still another embodiment, the term “targeted therapy” refers to administration of compositions that selectively interact with a chosen biomolecule to thereby treat cancer. For example, bevacizumab (Avastin®) is a humanized monoclonal antibody that targets vascular endothelial growth factor (see, for example, U.S. Pat. Publ. 2013/0121999, WO 2013/083499, and Presta et al. (1997) Cancer Res. 57:4593-4599) to inhibit angiogenesis accompanying tumor growth. In some cases, targeted therapy can be a form of immunotherapy depending on whether the target regulates immunomodulatory function.

The term “untargeted therapy” referes to administration of compositions that do not selectively interact with a chosen biomolecule yet treat cancer. Representative examples of untargeted therapies include, without limitation, chemotherapy, gene therapy, and radiation therapy.

Regarding irradiation, a sublethal dose of irradiation is generally within the range of 1 to 7.5 Gy whole body irradiation, a lethal dose is generally within the range of 7.5 to 9.5 Gy whole body irradiation, and a supralethal dose is within the range of 9.5 to 16.5 Gy whole body irradiation.

Depending on the purpose and application, the dose of irradiation may be administered as a single dose or as a fractionated dose. Similarly, administering one or more doses of irradiation can be accomplished essentially exclusively to the body part or to a portion thereof, so as to induce myeloreduction or myeloablation essentially exclusively in the body part or the portion thereof. As is widely recognized in the art, a subject can tolerate as sublethal conditioning ultra-high levels of selective irradiation to a body part such as a limb, which levels constituting lethal or supralethal conditioning when used for whole body irradiation (see, for example, Breitz (2002) Cancer Biother Radiopharm. 17:119; Limit (1997) J. Nucl. Med. 38:1374; and Dritschilo and Sherman (1981) Environ. Health Perspect. 39:59). Such selective irradiation of the body part, or portion thereof, can be advantageously used to target particular blood compartments, such as specific lymph nodes, in treating hematopoietic cancers.

The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In another embodiment, hormone therapy is used. Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

### IV. Administration of Compositions of Matter—Cyclic Di-Nucleotide Synthetase Enzyme Containing Vectors, Pharmaceutical Compositions, Vaccine, Adjuvants

The compositions of the invention (e.g., the recombinant vectors (e.g., any gene therapy vectors), containing at least one nucleic acid encoding a cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof, or a portion or ortholog thereof, and pharmaceutical compositions, vaccines, and adjuvants comprising same) are administered to subjects in a biologically compatible form suitable for pharmaceutical administration in vivo, to either enhance immune cell mediated immune responses. By “biologically compatible form suitable for administration in vivo” is meant a form of the compositions described herein to be administered in which any toxic effects are outweighed by the therapeutic effects of the compositions. The term “subject” is intended to include living organisms in which an immune response can be elicited, e.g., mammals. Examples of subjects include humans, dogs, cats, mice, rats, and transgenic species thereof. Administration of a compositions as described herein can be in any pharmacological form including a therapeutically active amount of an composition alone or in combination with a pharmaceutically acceptable carrier.

Administration of a therapeutically active amount of the therapeutic composition of the present invention is defined as an amount effective, at dosages and for periods of time necessary, to achieve the desired result. For example, a therapeutically active amount of a vaccine may vary according to factors such as the disease state, age, sex, and weight of the individual, and the ability of peptide to elicit a desired response in the individual. Dosage regimens can be adjusted to provide the optimum therapeutic response. For example, several divided doses can be administered daily or the dose can be proportionally reduced as indicated by the exigencies of the therapeutic situation.

The compositions of the present invention described herein can be administered in a convenient manner such as by injection (subcutaneous, intravenous, etc.), oral administration, inhalation, transdermal application, or rectal administration. Depending on the route of administration, the active compound can be coated in a material to protect the compound from the action of enzymes, acids and other natural conditions which may inactivate the compound. For example, for administration of compositions, by other than parenteral administration, it may be desirable to coat the composition with, or co-administer the composition with, a material to prevent its inactivation.

An composition can be administered to an individual in an appropriate carrier, diluent or adjuvant, co-administered with enzyme inhibitors or in an appropriate carrier such as liposomes. Pharmaceutically acceptable diluents include saline and aqueous buffer solutions. Adjuvant is used in its broadest sense and includes any immune stimulating compound such as interferon. Additional adjuvants may to combine with the compositions of the present invention include resorcinols, non-ionic surfactants such as polyoxyethylene oleyl ether and n-hexadecyl polyethylene ether. Enzyme inhibitors include pancreatic trypsin inhibitor, diisopropylfluorophosphate (DEEP) and trasylol. Liposomes include water-in-oil-in-water emulsions as well as conventional liposomes (Sterna et al. (1984) J. Neuroimmunol. 7:27).

The composition may also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions of compositions suitable for injectable use include sterile aqueous solutions (where water soluble) or dispersions and sterile powders for the extemporaneous preparation of sterile injectable solutions or dispersion. In all cases the composition will preferably be sterile and must be fluid to the extent that easy syringeability exists. It will preferably be stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal compositions, for example, parabens, chlorobutanol, phenol, ascorbic acid, thimerosal, and the like. In many cases, it is preferable to include isotonic compositions, for example, sugars, polyalcohols such as manitol, sorbitol, sodium chloride in the composition. Prolonged absorption of the injectable compositions can be brought about by including in the composition an composition which delays absorption, for example, aluminum monostearate and gelatin.

Sterile injectable solutions can be prepared by incorporating a composition of the present invention (e.g., vector (e.g., any gene therapy vector comprising at least one cyclic di-nucleotide synthetase enzyme, such as AdVCA0956 or AdVCA0848)) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the composition plus any additional desired ingredient from a previously sterile-filtered solution thereof.

When the composition is suitably protected, as described above, the protein can be orally administered, for example, with an inert diluent or an assimilable edible carrier. As used herein “pharmaceutically acceptable carrier” includes any and all solvents, dispersion media, coatings, antibacterial and antifungal compositions, isotonic and absorption delaying compositions, and the like. The use of such media and compositions for pharmaceutically active substances is well known in the art. Except insofar as any conventional media or composition is incompatible with the active compound, use thereof in the therapeutic compositions is contemplated. Supplementary active compounds can also be incorporated into the compositions.

It is especially advantageous to formulate parenteral compositions in dosage unit form for ease of administration and uniformity of dosage. “Dosage unit form”, as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the present invention are dictated by, and directly dependent on, (a) the unique characteristics of the active compound and the particular therapeutic effect to be achieved, and (b) the limitations inherent in the art of compounding such an active compound for the treatment of sensitivity in individuals.

In one embodiment, a composition of the present invention is a vector (e.g., any gene therapy vector comprising at least one cyclic di-nucleotide synthetase enzyme, such as AdVCA0956 or AdVCA0848). As defined herein, a therapeutically effective amount of the adenovirus (i.e., an effective dosage) ranges from about 1×10⁴ to 1×10¹² infectious particles/kg. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a vector (e.g., any gene therapy vector comprising at least one cyclic di-nucleotide synthetase enzyme, such as AdVCA0956 or AdVCA0848) can include a single treatment or, preferably, can include a series of treatments. In some embodiments, a subject is treated with a vector (e.g., any gene therapy vector comprising at least one cyclic di-nucleotide synthetase enzyme, such as AdVCA0956 or AdVCA0848) in the range of between about 1×10⁴ to 1×10¹² infectious particles/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of vector (e.g., any gene therapy vector comprising at least one cyclic di-nucleotide synthetase enzyme, such as AdVCA0956 or AdVCA0848) used for treatment may increase or decrease over the course of a particular treatment. Changes in dosage may result from the results of diagnostic assays. In addition, a vector (e.g., any gene therapy vector comprising at least one cyclic di-nucleotide synthetase enzyme, such as AdVCA0956 or AdVCA0848) of the present invention can also be administered in combination therapy with, e.g., chemotherapeutic compositions, hormones, antiangiogens, radiolabelled, compounds, or with surgery, cryotherapy, and/or radiotherapy. A vector (e.g., any gene therapy vector comprising at least one cyclic di-nucleotide synthetase enzyme, such as AdVCA0956 or AdVCA0848) of the present invention can also be administered in conjunction with other forms of conventional therapy, either consecutively with, pre- or post-conventional therapy. For example, the vector (e.g., any gene therapy vector comprising at least one cyclic di-nucleotide synthetase enzyme, such as AdVCA0956 or AdVCA0848) can be administered with a therapeutically effective dose of chemotherapeutic composition. In another embodiment, the vector (e.g., any gene therapy vector comprising at least one cyclic di-nucleotide synthetase enzyme, such as AdVCA0956 or AdVCA0848) can be administered in conjunction with chemotherapy to enhance the activity and efficacy of the chemotherapeutic composition. The Physicians' Desk Reference (PDR) discloses dosages of chemotherapeutic compositions that have been used in the treatment of various cancers. The dosing regimen and dosages of these aforementioned chemotherapeutic drugs that are therapeutically effective will depend on the particular immune disorder being treated, the extent of the disease and other factors familiar to the physician of skill in the art and can be determined by the physician.

In addition, the compositions of the present invention described herein can be administered using nanoparticle-based composition and delivery methods well known to the skilled artisan. For example, nanoparticle-based delivery for improved nucleic acid therapeutics are well known in the art (Expert Opinion on Biological Therapy 7:1811-1822).

### V. Kits

The present invention also encompasses kits for treating disorders that would benefit from upregulated immunot responses, such as pathogenic infections and cancers, using the compositions of the invention (e.g., the recombinant vectors (e.g., adeonovirial vectors), containing a nucleic acid encoding a cyclic di-nucleotide synthetase enzyme (e.g., DGCs, DACs, Hypr-GGDEFs, DncV, DisA, cGAS, any sequences that encode GGDEF domains belonging to the COG2199 protein domain family) listed herein, the Figures, and the Examples, or any subset thereof, or a portion or ortholog thereof, and pharmaceutical compositions, vaccines, and adjuvants comprising same). For example, the kit can comprise the recombinant vectors (e.g., any gene therapy vector comprising at least one cyclic di-nucleotide synthetase enzyme, such as AdVCA0956 or AdVCA0848, extracellular antigen, or Ad containing Ag) in hydrophilized, dried, or liquid form that is packaged in a suitable container. The kit can further comprise instructions for using such compositions to treat pathogenic infections and/or cancers in a patient in need thereof. The kit may also contain other components, such as administration tools like packaged in a separate container.

This invention is further illustrated by the following examples which should not be construed as limiting. The contents of all references, patents and published patent applications cited throughout this application, as well as the Figures, are incorporated herein by reference.

### EXEMPLIFICATION

This invention is further illustrated by the following examples, which should not be construed as limiting.

### Example 1: Materials and Methods for Examples 2-5

All of the DNA manipulation and plasmid construction was performed as previously described (Sambrook J et al. (2001) Molecular Cloning—A Laboratory Manual, 3rd ed. Cold Spring Harbor Laboratory Press, Cold Spring Harbor, NY). The VCA0956 gene was amplified from Vibrio cholerae El tor strain C6706 using the DNA polymerase Phusion (New England Biolabs) and the oligonucleotides 5′-ATAGGTACCCCACCGTGATGACAACTGAAGATTTCA-3′ and 5′-ATACTCGAGTTAGAGCGGCATGACTCGAT-3′ (IDT). This product was then inserted into the plasmid pShuttle-CMV (Seregin S S et al. (2010) Hum. Gene Ther. 22:1083-1094) by digesting with Kpn1 and XhoI (Fermentas), and then ligated with a T4 DNA ligase (Invitrogen). Escherichia coli strain DH10B (Invitrogen) was used for harboring plasmid DNA, and sequence fidelity was confirmed by sequencing (Genewiz). The active site mutant allele was generated using the QuickChange Lightning site-directed mutagenesis kit (Agilent) with the primer 5′-TGACAGCTTATCGTTATGCCGCTGAAGAGTTTGCACTGAT-3′.

A first-generation, human Ad type 5 (Ad5) replication deficient vector (deleted for the E1 and E3 genes) was used in this study (Seregin S S et al. (2009) Gene Ther. 16:1245-1259). Recombination, viral propagation of the Ad5 vectors, and subsequent virus characterization was performed as previously described (Seregin S S et al. (2009) Gene Ther. 16:1245-1259; Seregin S S et al. (2010) Blood 116:1669-1677). Viral particle number was determined by optical density measurement at 260 nm and validated as previously described (Amalfitano A et al. (1998) J. Virol. 72:926-933). Construction of the Ad5-Null and Ad5-TA is described elsewhere (Morgan J et al. (2002) Construction of First-Generation Adenoviral Vectors, p. 389-414, Gene Therapy Protocols, vol. 69. Springer New York; Seregin S S et al. (2012) Vaccine 30:1492-1501). All virus constructs were confirmed to be replication-competent adenovirus (RCA) negative using RCA PCR and direct sequencing methods (Seregin S S et al. (2009) Gene Ther. 16:1245-1259) and the bacterial endotoxin content was found to be <0.15 EU per mL (Seregin S S et al. (2009) Gene Ther. 16:1245-1259). All procedures with recombinant adenovirus constructs were performed under BSL-2 conditions.

All transfections of plasmid DNA into HeLa cells was performed with the TransIT-HeLaMONSTER transfection kit (Mirus) in 6-well plates with 2.5 μg plasmid DNA. For HeLa cell infections with adenovirus vectors, cells were infected with 2.0*10⁹ viral particles (M.O.I. of 500). Cell cultures were checked for confluence and morphology before and after transfection and infection using microscopy. After 24 hours of growth at 37° C. in 5% CO₂, the cells were dissociated using 300 μL 0.25% trypsin, and then cells were resuspended in 4 mL PBS and then pelleted by centrifugation at 1600 RPM at 4° C. Afterwards the cells were resuspended in 100 μL extraction buffer (40% acetonitrile, 40% methanol, and 0.1 N formic acid). The cell lysate was incubated at −20° C. for 30 minutes, and then centrifuged at max speed for 10 minutes. The extraction buffer was removed from the pelleted debris and stored at −80° C. until analysis.

Immediately prior to analysis, the extraction buffer was evaporated using a vacuum manifold, and the samples were rehydrated in 100 μL water. C-di-GMP was quantified using an Acquity Ultra Performance liquid chromatography system (Waters) coupled with a Quattro Premier XE mass spectrometer (Waters) as previously described (Massie J P et al. (2012) Proc. Natl. Acad. Sci. U.S.A 109:12746-12751). The concentration of c-di-GMP was determined by generating an 8-point standard curve (1:2 dilutions) of chemically synthesized c-di-GMP (Biolog) ranging from 1.9 to 250 nM. The intracellular concentration was estimated by dividing the total molar amount of c-di-GMP extracted by the estimated total intracellular volume of HeLa cells extracted using cell counts and size measurements determined using a Countess Automated cell counter (Life Technologies). The transfection efficiency was determined to be 18.2%, which was obtained by transfecting HeLa cells with plasmid containing GFP under CMV promoter control and measuring the percent of GFP positive cells using flow cytometry. The infection efficiency of HeLa cells was determined to be 82.2%, which was determined by infecting HeLa cells with Ad5-gfp (Seregin S S et al. (2010) Blood 116:1669-1677) and quantifying the percent of GFP positive cells using flow cytometry.

Adult BALB/c WT male mice (6-8 weeks old) were used for all animal experiments (Jackson Laboratory). For c-di-GMP quantification and innate studies, mice were anesthetized using isofluorane, and 2×10¹¹ adenovirus viral particles (vp) per mouse (200 μL total volume, suspended in 1× sterile PBS) were administered intravenously (IV) via retro-orbital injection. After administration, mice were monitored every 6 hours by lab personnel for mortality and other health parameters in accordance with Michigan State University EHS and IACUC. After 24 hours the mice were sacrificed, and the spleen and the left lobe of the liver were isolated from each animal. Each tissue was placed in 500 μL PBS, and then the tissue suspension was homogenized using an Omni Tissue Homogenizer (Omni International). 300 μL of homogenate was added to an equal volume of equilibrated Phenol Solution (Sigma). The homogenate-phenol solution was vortexed and centrifuged at 15,000 rpm for 10 minutes. The aqueous phase was removed and added to 500 μL chloroform. The mixture was vortexed and then centrifuged at 15,000 rpm for 10 minutes. The aqueous phase was then removed and stored at −80° C. until analysis.

Quantitative PCR was used to determine adenovirus abundance from DNA extracted from liver tissue as previously described (Seregin S S et al. (2009) Mol. Ther. 17:685-696). Ad5 genome copy numbers were quantified using an ABI 7900HT Fast Real-Time PCR system and the SYBR Green PCR Mastermix (Applied Biosystems) in a 15 μL reaction using a primer set for the Ad5 Hexon gene that has been previously described (Appledorn D M et al. (2008) Gene Ther. 15:885-901). All PCRs were subjected to the following procedure: 95.0° C. for 10 minutes, followed by 40 cycles of 95.0° C. for 15 seconds and 60.0° C. for 1 minute. Standard curves to determine the number of viral genomes per liver cell were run in duplicate and consisted of 6 half-log dilutions using DNA extracted from purified Ad5 virus (Seregin S S et al. (2009) Gene Ther. 16:1245-1259). As an internal control, liver DNA was quantified using primers spanning the GAPDH gene (Seregin S S et al. (2009) Mol. Ther. 17:685-696) and standard curves were generated from total genomic DNA. Melting curve analysis was performed to confirm the quality and specificity of the PCR (data not shown).

To determine relative abundance of specific liver-derived RNA transcript, reverse transcription was performed on RNA derived from the liver tissue using SuperScript III (Invitrogen) and random hexamers (Applied Biosystems) as per the manufacturer's instruction. RT reactions were diluted to a total volume of 60 μL, and 2 μL from each sample was used as template for subsequent PCR. Quantitative PCR was subsequently performed as described above using an ABI 7900HT Fast Real-Time PCR system and SYBR Green PCR Mastermix (Applied Biosystems) using primer sets that have been previously described (Seregin S S et al. (2009) Gene Ther. 16:1245-1259). The comparative Ct method was used to determine relative gene expression using GAPDH to standardize expression levels across all samples. Relative expression changes were calculated by comparing experimental levels of liver transcript to levels of liver transcript derived from mock-treated animals.

IFN-β was quantified using the Verikine Mouse IFN Beta ELISA kit (PBL Assay Science) as per manufacturer's instruction. Cytokine and chemokine concentrations were quantified from plasma samples using a Bio-Plex multiplex bead array system (Bio-Rad). At 6 and 24 hours, blood samples were taken from mice using heparinized capillary tubes and EDTA-coated microvettes (Sarstedt). The samples were centrifuged at 3,400 rpm for 10 minutes to isolate plasma. Samples were assayed for 12 independent cytokines and chemokines (IL-1α, IL-4, IL-6, IL12-p40, IFN-γ, G-CSF, Eotaxin, KC, MCP-1, MIP-1α, MIP-1β, and RANTES) as per the manufacturer's instructions (Bio-Rad) via Luminex 100 technology (Luminex).

For adaptive immunity studies, mice were administered adenovirus ranging from 1×10⁶ to 5×10⁹ vp per mouse suspended in 25 μL PBS via IM injection into the tibialis anterior of the right hindlimb. To measure antigen specific recall responses, mice were sacrificed and the spleen was harvested after 14 days. Splenocytes were isolated and ex vivo stimulated with immunogenic peptides from C. difficile TA library as previously described (Seregin S S et al. (2012) Vaccine 30:1492-1501). ELISpot analysis was performed as previously described (Seregin S S et al. (2012) Vaccine 30:1492-1501) using 96-well multiscreen high-protein binding Immobilon-P membrane plates (Millipore) and the Ready-Set Go IFN-γ mouse ELISpot kit (eBioscience). Spots were photographed and counted using an automated ELISpot reader system (Cellular Technology). To determine TA-specific IgG titers, ELISA based tittering was used on plasma samples taken from the mice 14 d.p.i as previously described (Seregin S S et al. (2012) Vaccine 30:1492-1501).

All animal procedures were reviewed and approved by the Michigan State University EHS and IACUC. Care for the mice was provided in accordance with PHS and AAALAC standards. Plasma and tissue samples were collected and handled in accordance with the Michigan State University Institutional Animal Care and Use Committee.

### Example 2: Generating an Adenovirus Harboring a V. cholerae DGC

Cdi-GMP is an exciting new adjuvant that stimulates the innate immune system (Chen W X et al. (2010) Vaccine 28:3080-3085). These studies most frequently used chemically synthesized c-di-GMP. Because c-di-GMP is synthesized from GTP and GTP is abundant in the cytoplasm of eukaryotic organisms, it was postulated that a DGC expressed under the control of a strong eukaryotic promoter/enhancer element would lead to c-di-GMP synthesis within the eukaryotic cell and subsequent enhancement of downstream innate immune responses. This approach would offer a novel, alternative method to administer c-di-GMP as a vaccine adjuvant as opposed to direct delivery of the synthesized molecule. To identify a DGC that would produce c-di-GMP in the cytoplasm of a eukaryotic cell, DGCs from V. cholerae was examined, as V. cholerae is a well-studied model system for c-di-GMP signaling and many V. cholerae DGCs have been shown to synthesize c-di-GMP in high concentrations (Massie J P et al. (2012) Proc. Natl. Acad. Sci. U.S.A 109:12746-12751). The DGC VCA0956 was selected due to the fact that it had no predicted N-terminal regulatory or trans-membrane domains. Furthermore, VCA0956 has a canonical GGDEF domain and active site motif, and ectopic expression of VCA0956 has been shown to increase biofilm formation in both V. cholerae and Vibrio vulnificus (Massie J P et al. (2012) Proc. Natl. Acad. Sci. U.S.A 109:12746-12751; Nakhamchik A et al. (2008) Appl. Environ. Microbiol. 74:4199-4209), repress motility in V. cholerae (Hunter J L et al. (2014) BMC Microbiol. 14:22), and increase intracellular c-d-GMP in V. cholerae and Shewanella oneidensis (Koestler B J et al. (2013) Appl. Environ. Microbiol. 79:5233-5241; Tamayo R et al. (2008) Infect. Immun. 76:1617-1627; Thormann K M et al. (2006) J. Bacteriol. 188:2681-2691).

To determine if VCA0956 is able to synthesize c-di-GMP in a eukaryotic cytoplasm, a plasmid containing VCA0956 under the control of the constitutive CMV promoter/enhancer in the plasmid pShuttleCMV was constructed. A second vector containing the same VCA0956 allele with a mutation in the active site of the GGDEF domain (GGEEF->AAEEF) was also constructed. These plasmids were transfected into HeLa cells, and c-di-GMP levels were measured in cell lysates after 24 hours using liquid chromatography coupled with tandem mass spectrometry (LC-MS/MS). It was found that eukaryotic cells transfected with the VCA0956 allele produced detectable levels of c-di-GMP (FIG. 1A). In contrast, no detectable c-di-GMP was observed in both cells transfected with the active site mutant allele or a mock treatment controls (FIG. 1A). The estimated intracellular c-di-GMP concentrations of HeLa cells grown in 6-well dishes expressing VCA0956 are greater than the K_{d} range of the c-di-GMP binding protein STING (2.5-4.9 μM) (Burdette D L et al. (2011) Nature 478:515-518; Yin Q et al. (2012) Mol. Cell 46:735-745). Cell cultures were checked by microscopy and no discernible morphological differences was observed between expression of VCA0956 and the controls. Furthermore, trypan blue staining indicated that treatment with VCA0956 did not appear to impact overall cell viability. Additionally, HeLa cells grown in t75 flasks transfected with the VCA0956 plasmid and measured 48 hours later had less intracellular c-di-GMP, suggesting that c-di-GMP synthesis is transient (FIG. 1B). It was speculated that c-di-GMP could be degraded in eukaryotic cells by nonspecific phosphodiesterase enzymes. Less c-di-GMP production in these experiments was observed which may be a function of decreased transfection efficiency in the flasks. Nevertheless, these results indicate that VCA0956 is capable of transiently synthesizing c-di-GMP in the cytoplasm of eukaryotic cells grown in vitro.

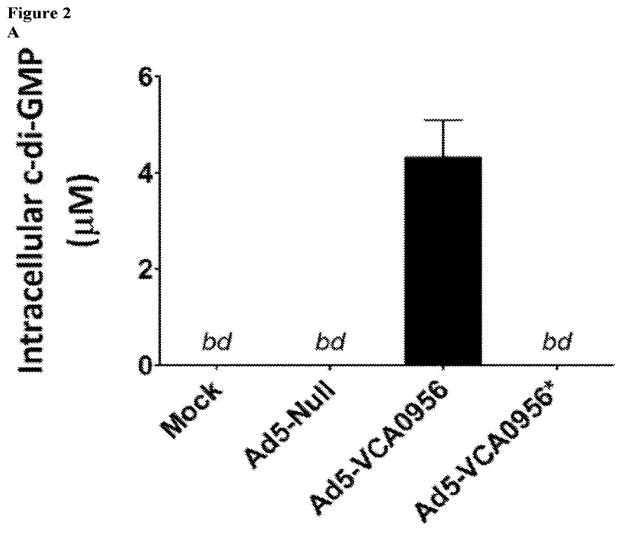

The pShuttleCMV-VCA0956 plasmid and its mutant allele counterpart were then used to construct and purify to high concentration the respective recombinant Ad5-based vectors. To confirm that the VCA0956 Ad5 construct, herein referred to as Ad5-VCA0956, was able to produce c-di-GMP in a eukaryotic cytoplasm, HeLa cells (500 multiplicity of infection, M.O.I.) were infected with the Ad5-VCA0956 and Ad5-VCA0956 mutant allele (Ad5-VCA0956*) adenovirus vectors and measured c-di-GMP using LC-MS/MS after 24 hours. The Ad5-Null vector, an adenovirus construct carrying no transgene, was also included as a negative control. It was found that cells infected with the Ad5-VCA0956 produced high concentrations of c-di-GMP comparable to transfection of the pShuttleCMV-VCA0956 plasmid, whereas cells infected with the Ad5-VCA0956* or the Ad5-Null produced no detectable c-di-GMP (FIG. 2). Importantly, similar to VCA0956 plasmid transfections, infection with Ad5-VCA0956 had no noticeable impact on cell morphology or viability. These results demonstrate that an adenovirus vector can be used to deliver VCA0956 into HeLa cells to synthesize c-di-GMP.

### Example 3: Synthesis of c-di-GMP in Vivo

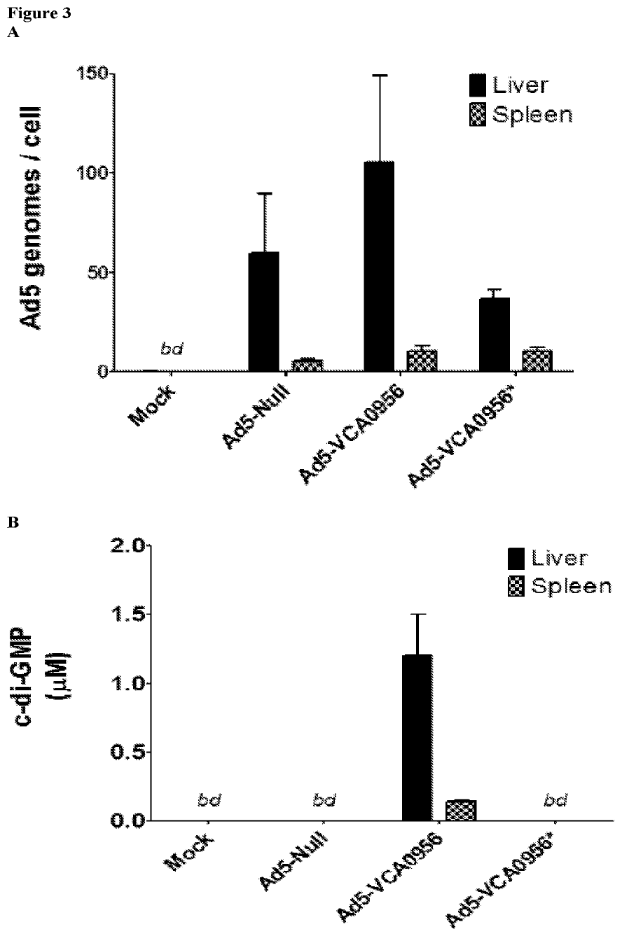

As the Ad5-VCA0956 vector is capable of producing c-di-GMP in HeLa cells in vitro, it was next determined if this vector produces c-di-GMP in vivo in a murine model system. BALB/c mice (n=3) were IV injected with the Ad5-Null, Ad5-VCA0956, or the Ad5-VCA0956* vectors and quantitative PCR was utilized to measure adenovirus genomes in the spleen and liver of injected mice at 24 hours post injection (h.p.i.). Using quantitative RT-PCR comparable Ad5 genome counts were observed for each treatment in both the liver and spleen (FIG. 3A). Consistent with previous reports that the predominant tropism of adenovirus is in the liver (Appledorn D M et al. (2008) Gene Ther. 15:885-901; Everett R S et al. (2003) Hum. Gene Ther. 14:1715-1726; Nakamura T et al. (2003) J. Virol. 77:2512-2521) there were significantly more Ad5 genomes in the liver cells than in the spleen cells. C-di-GMP in both the liver and spleen using LC-MS/MS was then measured, and found that the Ad5-VCA0956 vector produced detectable c-di-GMP in both tissues, whereas the Ad5-Null and Ad5-VCA0956* vectors produced no detectable c-di-GMP (FIG. 3B). The concentration of c-di-GMP was consistent with the abundance of Ad5-VCA0956 genomes per cell, as the amount of c-di-GMP was significantly higher in the liver tissue than the spleen. These data indicate that the Ad5-VCA0956 vector is capable of initiating c-di-GMP synthesis in a mouse.

### Example 4: c-Di-GMP Synthesized In Vivo Stimulates Innate Immunity in a Mouse Model

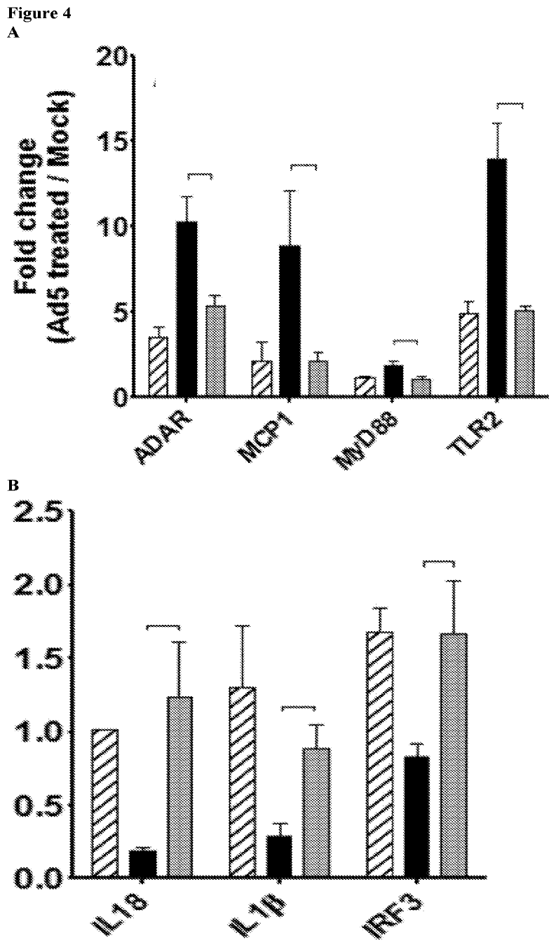

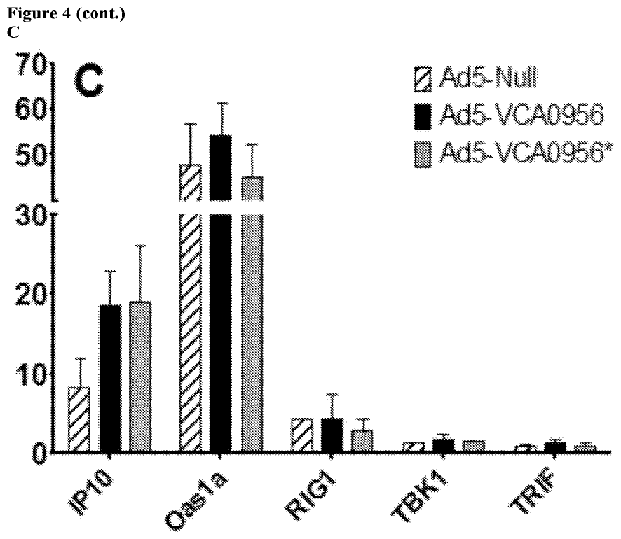

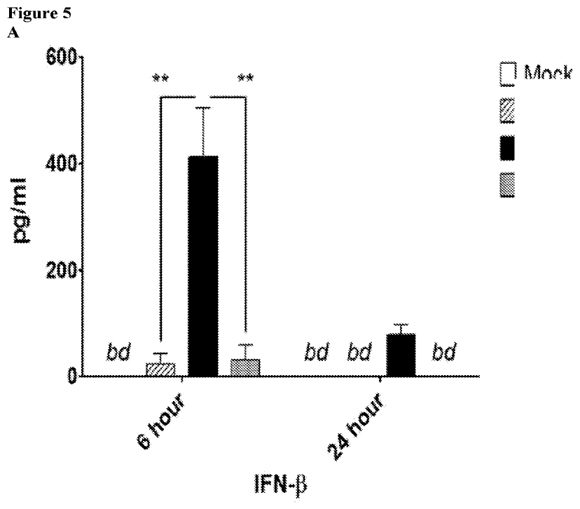

It has been previously shown that adenovirus vectors stimulate several pro-inflammatory innate immune response genes (Hartman Z C et al. (2008) Virus Res. 132:1-14; Seregin S S et al. (2009) Gene Ther. 16:1245-1259; Seregin S S et al. (2009) Mol. Ther. 17:685-696). To examine if the Ad5-VCA0956 alters the profile of innate immune gene expression compared to the Ad5 vector alone, Balb/c mice (n=3) were IV injected with Ad5-Null, Ad5-VCA0956, and Ad5-VCA0956* and qRT-PCR was utilized to quantify the expression levels of several liver gene transcripts at 24 hours post infection (h.p.i.). Infection with Ad5-VCA0956 had no observable effect on the health of the mice. It was found that the Ad5-Null treatment was able to stimulate 6 of the 12 markers examined (>2-fold; ADAR, MCP-1, TLR2, IP10, Oas1a, RIG1) (FIG. 4). These results are consistent with previous studies demonstrating that the adenovirus vector alone is capable of altering gene expression in the liver (Seregin S S et al. (2010) Hum. Gene Ther. 22:1083-1094; Seregin S S et al. (2009) Gene Ther. 16:1245-1259). The expression of four genes was significantly (p<0.05) higher in the Ad5-VCA0956 treatment compared to the Ad5-VCA0956* treatment (FIG. 4A); these include the IFN-responsive gene ADAR, the monocyte and basophil chemotractant protein-1 MCP-1, the toll-like receptor (TLR) signaling pathway gene MyD88, and the pattern recognition receptor TLR2. It is worth noting that c-di-GMP sensing in the cytoplasm is thought to be independent of TLRs (Karaolis D K R et al. (2007) J. Immunol. 178:2171-2181). Additionally, the expression of three genes was significantly (p<0.05) repressed in the Ad5-VCA0956 treatment compared to the Ad5-VCA0956* treatment (FIG. 4B): the pro-inflammatory interleukin genes IL18 and IL1β, and the interferon transcription factor IRF3. Interestingly, IRF3 has been shown to interact with STING to initiate a c-di-GMP-mediated host type I interferon response (McWhirter S M et al. (2009) J. Exp. Med. 206:1899-1911; Tanaka Y et al. (2012) Sci. Signal. 5:ra20; de Almeida L A et al. (2011) PLoS ONE 6:e23135).

In the cytoplasm, c-di-GMP interacts with STING to initiate a type-I interferon response and activates IRF3, NF-κβ, and the p38/JNK/ERK MAP kinase signaling pathways, resulting in increased production of numerous cytokines and chemokines (McWhirter S M et al. (2009) J. Exp. Med. 206:1899-1911). To determine if Ad5-VCA0956 is capable of inducing type-I interferons, the concentration of IFN-β in the plasma of mice I.V. treated with Ad5-Null, Ad5-VCA0956, or Ad5-VCA0956* at 6 h.p.i. and 24 h.p.i. were measured. It was found that at 6 h.p.i., IFN-β concentrations were significantly higher in mice treated with Ad5-VCA0956 compared to the other controls (FIG. 5). At 24 h.p.i., IFN-β concentrations were undetectable in the control mice, whereas mice treated with Ad5-VCA0956 demonstrated IFN-β concentrations that were detectable, although lower than those at the 6 h.p.i. timepoint. These data indicate that Ad5-VCA0956 is capable of inducing a type-I interferon response in mice.

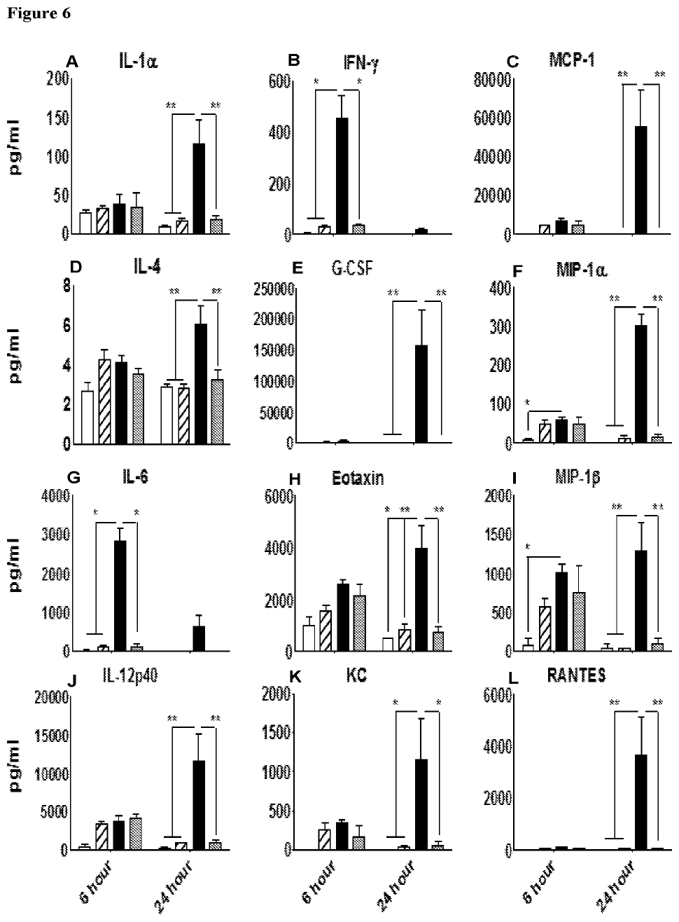

In addition to IFN-0, it was further determined if other cytokines and chemokines were induced by Ad5-VCA0956. To this end, the abundance of cytokines and chemokines in the plasma of mice treated with Ad5-VCA0956 using a multiplexed assay system at 6 and 24 h.p.i. were directly quantified. Consistent with prior studies showing that the adenovirus vector stimulates the secretion of pro-inflammatory cytokines and chemokines (27, 28), it was observed that 9 cytokines and chemokines were modestly induced in the Ad5-Null treated mice compared to the naïve mice (IFN-γ, MCP-1, G-CSF, MIP-1α, IL-6, MIP-1β, IL-12p40, KC, RANTES; >3-fold), and these differences were greatest at the 6-hour time point (FIG. 6). It was found that 12 cytokines and chemokines, shown in FIG. 6 were significantly increased in the plasma of the Ad5-VCA0956 treated mice compared to the control Ad5-VCA0956* treated mice at one or both of the two timepoints. Furthermore, for the majority of cytokines and chemokines examined, the largest differences observed were at the 24 hour time point, indicating that the effect of Ad5-VCA0956 is both more potent and longer lasting than that of the adenovirus vector alone. The induction of most of these cytokines and chemokines are consistent with other studies examining the immunostimulatory effects of c-di-GMP (Ebensen T et al. (2007) Vaccine 25:1464-1469; Ebensen T et al. (2007) Clin. Vaccine Immunol. 14:952-958; Karaolis D K R et al. (2007) J. Immunol. 178:2171-2181; Karaolis D K R et al. (2007) Infect. Immun. 75:4942-4950; Yan H B et al. (2009) Biochem. Biophys. Res. Commun. 387:581-584; Gray P M et al. (2012) Cell Immunol. 278:113-119). Interestingly, increases in IL-1α, G-CSF, and Eotaxin levels in the Ad5-VCA0956 injected mice were observed, which have not been previously reported to be induced by c-di-GMP. These data together indicate that the Ad5-VCA0956 vector is capable of inducing a robust innate response beyond that of the adenovirus vector alone in a murine model system.

### Example 5: Ad5-VCA0956 Lowers the Effective Dose for a T-Cell Response to a Clostridium difficile Antigen

The function of an adjuvant is to enhance the efficacy of a paired antigen by increasing the longevity, potency, or reducing the effective dose. Previous data showed that Ad5-VCA0956 strongly upregulates inflammatory responses. To test if the Ad5-VCA0956 construct functions as a vaccine adjuvant, it was determined if Ad5-VCA0956 could enhance the adaptive response to a C. difficile antigen. C. difficile, a Gram-positive spore-forming anaerobic bacteria, is the leading causative composition of nosocomial infections leading to diarrheal disease in the developed world. C. difficile associated diarrhea (CDAD) represents nearly 1% of all hospital stays in the United States and can lead to septicemia, renal failure, and toxic megacolon (Lucado J et al. (2012. Clostridium difficile Infections (CDI) in Hospital Stays, 2009. Agency for Healthcare Research and Quality). Incidents and mortality of C. difficile infections are rising in the U.S., and the economic burden on the health care system is reported to be in the billions of dollars (Lucado J et al. (2012. Clostridium difficile Infections (CDI) in Hospital Stays, 2009. Agency for Healthcare Research and Quality; Morris A M et al. (2002) Arch. Surg. 137:1096-1100; Redelings M D et al. (2007) Increase in Clostridium difficile-related mortality rates, United States, 1999-2004. Emerg Infect Dis; Kyne L et al. (2002) Clin. Infect. Dis. 34:346-353; Dubberke E R et al. (2009) Epidemiol. 30:57-66). Furthermore, to date there are no approved effective vaccine treatments available for CDAD treatment or prevention (Aslam S et al. (2005) Lancet Inf Dis. 5:549-557).

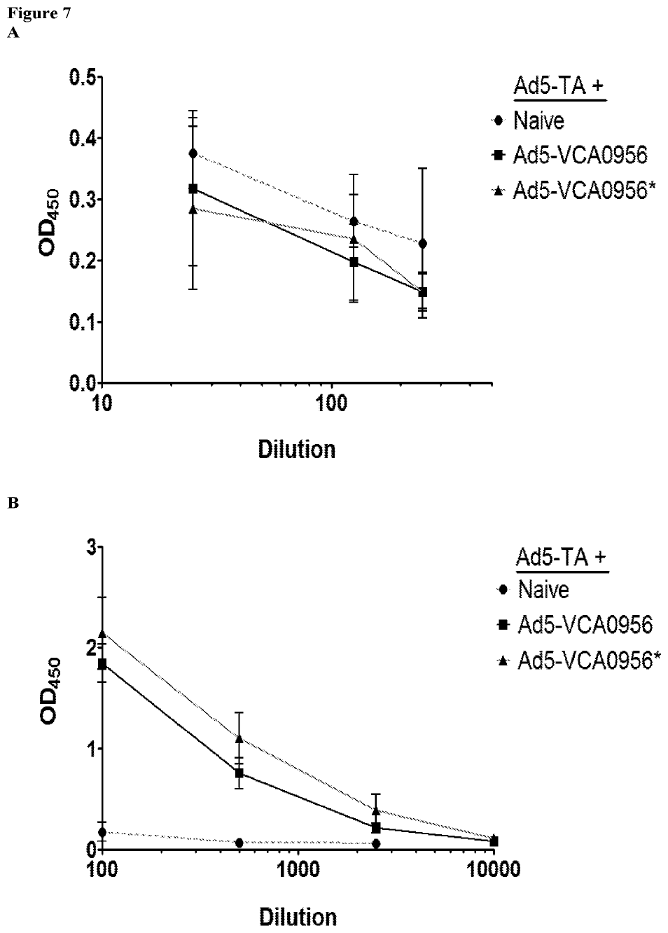

An adenovirus vector that expresses the immunogenic portion of the C. difficile toxin A (Ad5-TA) was previously developed and demonstrated to protect mice from a toxin challenge by generating a humoral and T-cell response specific to toxin A in a murine model system (Seregin S S et al. (2012) Vaccine 30:1492-1501). It was hypothesized that supplementing this vaccine with the Ad5-VCA0956 adjuvant would enhance this humoral and T-cell response due to the strong innate immune stimulatory activity of VCA0956. Therefore mice were vaccinated by IM injection with varying concentrations of the Ad5-TA vector in combination with the Ad5-VCA0956 vector in equal ratio ranging from 1×10⁶ to 5×10⁹ viral particles (vp). After two weeks, TA-specific IgG titers in the plasma of the vaccinated mice were measured. At the 1×10⁷ dose, no significant changes in TA-specific IgG in the plasma of any of the treated mice were observed compared to the mock treatment, indicating that this dose of Ad5-TA and Ad5-VCA0956 is not sufficient to produce a robust IgG response in mice (FIG. 7A). In contrast, the 5×10⁹ dose resulted in significantly increased TA-specific IgG in both the Ad5-VCA0956 and Ad5-VCA0956*, however the TA-specific IgG titers in the Ad5-VCA0956* treated animals was modestly higher (2-way ANOVA, p<0.05) than those treated with Ad5-VCA0956 (FIG. 7B), suggesting that higher doses of c-di-GMP has a negative impact on humoral immunity.

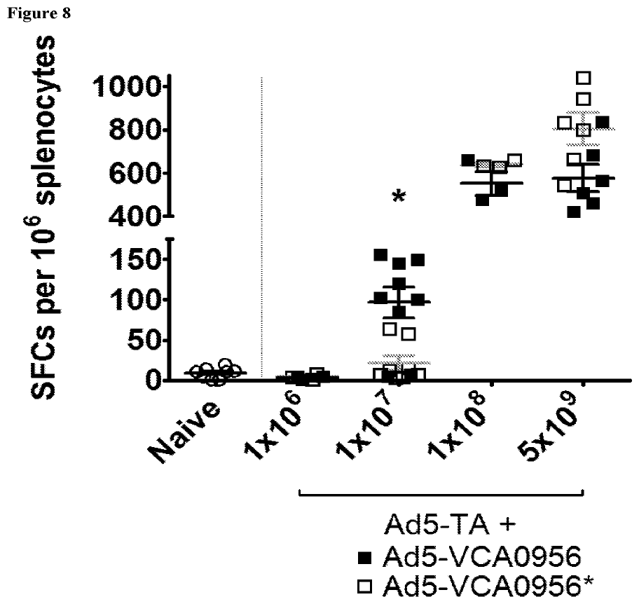

TA specific T-cell responses in the spleens of the naïve and vaccinated animals were also assessed using an IFN-γ ELISpot assay, utilizing the 15-mer peptide (VNGSRYYFDTDTAIA) that has been previously shown to elicit the secretion of IFN-γ in splenocytes of mice immunized with the Ad5-TA vector (Seregin S S et al. (2012) Vaccine 30:1492-1501). It was found that co-injection of equal amounts of the Ad5-TA and the mutant DGC allele vector Ad5-VCA0956*produced no induction of IFN-γ secreting T-cells over that of naïve splenocytes at viral doses of 1×10⁶ and 1×10⁷, but did generate significant IFN-γ producing T-cells at 1×10⁸ and 5×10⁹ (FIG. 8, white squares). The number of spot-forming cells (SFCs) in the mice treated with Ad5-TA and Ad5-VCA0956* at the 5×10⁹ dose was consistent with SFCs of mice vaccinated with Ad5-TA alone (Seregin S S et al. (2012) Vaccine 30:1492-1501). These data indicate that antigen-specific T-cells responses in splenocytes plateaus at high levels of Ad5-TA independent of the addition of c-di-GMP. Although co-injection of 1×10⁶ Ad5-TA with Ad5-VCA0956 did not produce increased IFN-γ levels, we observed significantly increased (p<0.05) IFN-γ producing T-cells at a dose of 1×10⁷, as compared to cells derived from the DGC mutant treated control (FIG. 8, black squares). However, the number of IFN-γ splenocytes did not reach those of the mice injected with higher concentrations of Ad5-TA and Ad5-VCA0956, suggesting only a modest improvement compared to the negative controls. IFN-γ producing T-cells at injections of 1×10⁸ and 5×10⁹ Ad5-TA and Ad5-VCA0956 were similar to the DGC mutant control. No c-di-GMP was detected in the liver of mice infected with Ad5-VCA0956 at the 5×10⁹ dose after 14 days, suggesting that even at high doses intramuscular administration of Ad5-VCA0956 does not lead to long-lasting c-di-GMP production at distal sites (data not shown). Thus, it was concluded that although it does not increase a humoral response, c-di-GMP synthesized by Ad5-VCA0956 modestly lowers the effective dose to generate a T-cell response to Ad5-TA in a murine model system.

### Discussion

With a current demand for novel vaccines that target difficult-to-treat diseases, it is crucial to have adjuvants to pair with these vaccines to optimize efficacy. Currently, there are a limited number of adjuvants available for clinical use, and there is a need for new adjuvants which can enhance the efficacy of vaccines to improve immunological protection (Coffman R L et al. (2010) Immunity 33:492-503; Reed S G et al. (2009) Trends Immunol. 30:23-32). Numerous studies have implicated c-di-GMP as a promising novel adjuvant. Indeed, this second messenger molecule has been shown to stimulate a robust type I interferon response and increase the secretion of numerous cytokines and chemokines to initiate a balanced Th1/Th2 response, as well as stimulate the inflammasome pathway and immune cell activation/recruitment (Sauer J D et al. (2011) Infect. Immun. 79:688-694; Ebensen T et al. (2007) Vaccine 25:1464-1469; Abdul-Sater A A et al. (2013) EMBO reports 14:900-906; Ebensen T et al. (2007) Clin. Vaccine Immunol. 14:952-958; Karaolis D K R et al. (2007) J. Immunol. 178:2171-2181; Karaolis D K R et al. (2007) Infect. Immun. 75:4942-4950; Yan H B et al. (2009) Biochem. Biophys. Res. Commun. 387:581-584; Gray P M et al. (2012) Cell Immunol. 278:113-119; Blaauboer S M et al. (2014) J. Immunol. 192:492-502). Described herein is a novel approach in that it utilizes an adenovirus vector to deliver c-di-GMP producing enzyme DNA into cells, thereby synthesizing the adjuvant in vivo. Adenovirus vectors are promising in that they are cost-efficient to produce and can efficiently deliver specific antigens or adjuvants into cells for in vivo production.

It was demonstrated that an adenovirus vector carrying a bacterial DGC is capable of synthesizing c-di-GMP in both human and mouse model systems. Similar to previous studies, it was demonstrated that c-di-GMP synthesized by Ad5-VCA0956 is able to induce a type-I interferon response (FIG. 5). Furthermore, synthesis of c-di-GMP by Ad5-VCA0956 increases the secretion of numerous cytokines and chemokines (Ebensen T et al. (2007) Vaccine 25:1464-1469; Ebensen T et al. (2007) Clin. Vaccine Immunol. 14:952-958; Karaolis D K R et al. (2007) J. Immunol. 178:2171-2181; Karaolis D K R et al. (2007) Infect. Immun. 75:4942-4950; Yan H B et al. (2009) Biochem. Biophys. Res. Commun. 387:581-584; Gray P M et al. (2012) Cell Immunol. 278:113-119; Blaauboer S M et al. (2014) J. Immunol. 192:492-502). Importantly, it was demonstrated that Ad5-VCA0956 induces an innate response beyond that of the adenovirus vector alone, which is capable of stimulating the STING system (Lam E et al. (2013) J. Virol. 88:974-981). These cytokines and chemokines induced by Ad5-VCA0956 include signals characteristic of both Th1 (e.g. IFN-γ, IL-12) and Th2 (e.g. IL-4, IL-6) type responses. Additionally, c-di-GMP production from Ad5-VCA0956 enhances activation of the innate immune system by activating TLR signaling (e.g. TLR2, MyD88). It appears however that c-di-GMP synthesized in vivo negatively regulates the expression of inflammasome-dependent pathways in hepatocytes (FIG. 4, IL-1β, IL-18). The significance of this finding is unclear, especially as it has been reported that c-di-GMP activates the NLRP3 inflammasome pathway (Abdul-Sater A A et al. (2013) EMBO reports 14:900-906). Importantly, no signs of poor cell physiology or health were observed in cell cultures and animal models. Furthermore, the data described herein indicated that the c-di-GMP synthesized by the Ad5-VCA0956 vector is transient, and thus should enhance antigen recognition and response while minimizing any potentially unwanted long term effects associated with administration, such as autoimmune activation (53). The mechanism by which c-di-GMP is being eliminated from cell cultures is unknown. It is speculated that native eukaryotic phosphodiesterases are able to hydrolyze the second messenger.

As shown herein, c-di-GMP synthesized in vivo modestly reduces the effective antigen dose of Ad5-TA to produce a T-cell response to a vaccine antigen which targets the toxin of the human pathogen C. difficile. Reducing the dose required to initiate an adaptive immune response is of particular significance as high viral particle doses can lead to global toxicities, endothelial cell activation, and liver damage (Seregin S S et al. (2009) Mol. Ther. 17:685-696; Everett R S et al. (2003) Hum. Gene Ther. 14:1715-1726; Wolins N et al. (2003) Br. J. Haematol. 123:903-905; Appledorn D M et al. (2008) i. 15:1606-1617; Schiedner G et al. (2000) Hum. Gene Ther. 11:2105-2116). The data herein suggest that increased c-di-GMP did not enhance the humoral response, however, and modestly decreased antibody production against the C. difficile toxin was observed. Whether these observations are specific to toxin A from C. difficile or more generally applicable to other antigens is under investigation.

While it was demonstrated that Ad5-VCA0956 is capable of in vivo c-di-GMP synthesis and has the potential to act as a vaccine adjuvant, further optimization is required to enhance this response. V. cholerae contains 40 predicted DGC alleles within its genome, and it has been shown that ectopic expression of these different DGCs results in different intracellular c-di-GMP concentrations (Massie J P et al. (2012) Proc. Natl. Acad. Sci. U.S.A. 109:12746-12751). Hence intracellular expression of other DGCs could produce different amounts of c-di-GMP in eukaryotic cells to optimize the intracellular concentration of c-di-GMP for different applications. Alternatively, other types of second messengers could be used to stimulate innate immunity. One example would be to express a diadenylate cyclase to synthesize the related bacterial second messenger c-di-AMP in vivo. C-di-AMP has similarly been shown to induce a robust innate immune response through STING mediated recognition (Barker J R et al. (2013) STING-Dependent Recognition of Cyclic di-AMP Mediates Type I Interferon Responses during Chlamydia trachomatis Infection. MBio 4; Woodward J J et al. (2010) Science 328:1703-1705). Another example is the dinucleotide cyclic guanosine monophosphate-adenosine monophosphate (cGAMP), a host second messenger produced in response to foreign DNA to activate a STING-dependent type-1 interferon response (Sun L et al. (2012) Science 339:786-791; Wu J et al. (2013) Science 339:826-830; Gao D et al. (2013) Science 341:903-906; Li X-D et al. (2013) Science 341:1390-1394). As these second messengers stimulate a STING-mediated innate immune response, they are good alternative candidates for Ad-5 mediated in vivo synthesis. Different promoters could be used in lieu of the CMV promoter to produce localized or temporally controlled c-di-GMP production in the body. Finally, the kinetics of adjuvant production by DGCs and antigen expression could be key factors in stimulating increased adaptive responses.

Other research studies suggest that STING-dependent inflammation inhibits the development of cell-mediated immunity. Archer et. al. recently showed that production of c-di-AMP by the intracellular bacterial pathogen Listeria monocytogenes inhibits cell-mediated immunity while inducing inflammatory cytokines in a STING dependent manner (Archer K A et al. (2014) PLoS Pathog 10:e1003861). No significant inhibition of either antibody production or IFN-γ producing memory T-cells was observed. Whether, these differences are due to the delivery route (L. monocytogenes versus Ad5 transduction), the levels of the signal, or other factors remains to be determined but addressing this question has significant implications for using c-di-GMP or c-di-AMP as a vaccine adjuvant.

C-di-GMP has been shown to enhance protection against other pathogens including S. aureus, K pneumoniae, and S. pneumoniae (Karaolis D K R et al. (2007) J. Immunol. 178:2171-2181; Karaolis D K R et al. (2007) Infect. Immun. 75:4942-4950; Yan H B et al. (2009) Biochem. Biophys. Res. Commun. 387:581-584; Ogunniyi A D et al. (2008) Vaccine 26:4676-4685), indicating that c-di-GMP has broad antigen-adjuvant synergy. Although the results of this study imply that that c-di-GMP produced from adenovirus vectors may not enhance vaccines that rely on antibody production, such as those targeting bacterial toxins, the Ad5-VCA0956 stimulated c-di-GMP innate immune response could enhance protection of vaccines that drive cell-mediated immunity such as those targeting viral infections or cancers. Consistent with this idea, c-di-GMP has been shown to exhibit anti-cancer properties in a number of studies (Miyabe H et al. (2014) J. Control. Release 184:20-27; Chandra D et al. (2014) Cancer Immunology Research. 2(9):901-10; Karaolis D K R et al. (2005) Biochem. Biophys. Res. Commun. 329:40-45), which is thought to be mediated through stimulation of a Type I interferon response as observed here. Miyabe et. al. showed that enhancing c-di-GMP entry into cancer cells using liposomes increased its efficacy (Miyabe H et al. (2014) J. Control. Release 184:20-27); adenovirus delivery of DGCs to tumors could function similarly by driving synthesis of c-di-GMP in cancer cells. One advantage of using adenovirus for this purpose over general administration is that modified adenovirus vectors have been constructed to target specific tissue types (Reetz J et al. (2014) Viruses 6:1540-1563), and c-di-GMP could be directly delivered to tumor cells or other tissue.

### Example 6: Materials and Methods for Examples 7-13

### 1. Vector Construction

Adenovirus-based vectors used in this study were all replication-deficient. AdNull and AdGag were constructed as previously described (Aldhamen, Y A et al. (2011) J Immunol 186: 722-732; Seregin, S S et al. (2010) Blood 116: 1669-1677). AdVCA0848 was constructed similarly to AdVCA0956 as previously described in Examples 1-5. Briefly, the V. cholerae gene VCA0848 gene (GeneBank sequence: CP007635.1) was sub-cloned into pShuttle-CMV as previously described (Appledorn, D M et al. (2010) PLoS One 5: e9579). Primers used for AdVCA0848 construction were: forward: 5′-ATAGGTACCCCACCATGAATGACAAAGTGCT-3′ and reverse: 5′-ATACTCGAGTTAGAAAAGTTCAACGTCATCAGAA-3′. The mutant version of AdVCA0848, AdVCA0848^{mut}, carrying the following amino acid changes: GGEEF>AAEEF in the GGDEF domain of VCA0848 allele was mutated using the QuikChange Lightning site-directed mutagenesis kit (Agilent) with the primer 5′-GTCTTCTCAACTATTTCGCTTTGCTGCTGAAGAGTTCGTGATTATTTTTT-3′. AdToxB was constructed as previously described (Seregin, S S et al. (2012) Vaccine 30: 1492-1501). Briefly, a synthetic gene was designed based on the Clostridium difficile toxin B sequence data from previous studies (Barroso, L A et al. (1990) Nucleic Acids Res 18: 4004; Kink, J A et al. (1998) Infect Immun 66: 2018-2025) and ordered from GENEART (Regensburg, Germany). The synthetic gene representing the C-terminal portion of Toxin B, including 617 amino acids (residues 1750-2366), was sub-cloned into pShuttle-CMV as previously described (Appledorn, D M et al. (2010) PLoS One 5: e9579). Primers used for AdToxB construction: forward: 5′-GCTACTACGAGGACGGCCTG-3′ and reverse: 5′-CTCATCGATGATCAGCTTGCC-3′. The C-terminal region of the new synthetic gene did not contain the enzymatic domain, and recombination and viral propagation were carried out as described above in Examples 1-5 (Appledorn, D M et al. (2010) PLoS One 5: e9579; Aldhamen, Y A et al. (2012) J Immunol 189: 1349-1359). Constructs were confirmed to be replication-competent adenovirus (RCA) negative using RCA PCR and direct sequencing methods as previously described (Seregin, S S et al. (2010) Blood 116: 1669-1677; Seregin, S S et al. (2009) Mol Ther 17: 685-696). All procedures with recombinant adenovirus constructs were performed under BSL-2 conditions.

### 2. Animal Procedures

The Michigan State University Institutional Animal Care and Use Committee (IACUC) approved the animal procedures conducted in this study. Care was provided to mice in this study in accordance with PHS and AAALAC standards. Mice were purchased from Taconic Biosciences, (Germantown, NY).

To determine the amount of c-di-GMP produced by the AdVCA0848 vector, male 6-8 weeks old Balb/c mice, were intravenously (i.v.) injected (retro-orbitally) with AdNull (n=3), AdVCA0956 (n=4), or AdVCA0848 (n=4) in 200 μl of a phosphate-buffered saline solution (PBS, pH 7.4) containing 2×10¹¹ viral particles (vps)/mouse; or not injected (naïves) (n=3) as previously described (30). The same viral dose was also used for additional experiments in which mice were injected with AdVCA0848, AdVCA0848^{mut}, or not injected (naïves). At 24 hours post-injection (hpi), mice were sacrificed and liver samples were collected, immediately snap frozen, and used later for c-di-GMP quantification as described below.

For innate immunity studies, 6-10 weeks old male C57BL/6 mice (n=4) were i.v. injected (retro-orbitally) with AdNull or AdVCA0848 in 100 μl of a phosphate-buffered saline solution (PBS, pH 7.4) containing 1×10¹⁰ vps/mouse or not injected (Naïve). The same viral dose was also used for additional experiments in which mice were injected with AdVCA0848, AdVCA0848^{mut}, or not injected (naïves). At 6 hpi, mice were sacrificed. Blood samples were collected and used for ELISA analysis and splenocytes were harvested, counted and used for immune cell surface staining. Liver samples were immediately stored at −80° C. for c-di-GMP quantification.

To determine the effect of AdVCA0848 on adaptive immune responses against OVA, male 8-10 weeks old C57BL/6 mice (n=4) were co-injected with AdVCA0848 or AdNull in 30 μl of a phosphate-buffered saline solution (PBS, pH 7.4) containing 1×10¹⁰ vps/mouse via i.m. injection and 100 g/mouse OVA via intraperitoneal (i.p.) injection, with an additional group of mice which were not injected (naïves). At 6 days post-injection (dpi), retro-orbital bleeding was used to collect blood samples for ELISA analysis. At 14 dpi, mice were sacrificed, peripheral blood samples collected and spleen was harvested in 2% FBS RPMI media.

To determine the effect of AdVCA0848 on the adaptive immune response against the HIV-1-derived Gag antigen, we initially conducted a dose-dependent study to determine the optimum AdVCA0848 dose that would significantly modulate adaptive immunity specific to the co-injected 5×10⁶ vps/mouse dose of AdGag. 6-8 weeks old male BALB/c mice (n=4) were intramuscularly (i.m.) co-injected in the tibialis anterior with viral particles in a phosphate-buffered saline solution in 30 μl (PBS, pH 7.4) containing a dose of 5×10⁶ vps of AdGag along with 3 different doses of 5×10⁷, 5×10⁸, or 5×10⁹ vps/mouse of either AdNull or AdVCA0848. An additional group of mice were not injected (naive). Additional experiments were conducted in which mice were co-injected with AdGag at 5×10⁶ vps/mouse and 5×10⁹ vps/mouse of AdVCA0848 or AdVCA0848^{mut}, or not injected (naïves). At 14 dpi, mice were sacrificed, peripheral blood samples collected and spleen was harvested in 2% FBS media. To determine the effect of AdVCA0848 on the adaptive immune response against C. difficile-derived Toxin B antigen, female 6-8 weeks old C57BL/6 mice (n=4) were i.m. co-immunized in the tibialis anterior with viral particles of AdToxB (5×10⁸ vps/mouse) along with 5×10⁸ vps/mouse of either AdGFP or AdVCA0848. At 21 dpi, mice were terminally sacrificed, and blood samples were collected for B cell analysis with ELISA. To verify the expression of Gag protein in the injected mice, 6-8 weeks old male BALB/c mice were i.v. injected with 1×10¹¹ vps/mouse of AdGag only (n=3), or co-injected of 1×10¹¹ vps/mouse of AdGag along with 1×10¹¹ vps/mouse of either AdNull or AdVCA0848. At nearly 24 hpi, mice were humanely sacrificed and liver samples were obtained and frozen at −80° C. until analysis by western blot for Gag protein levels.

3. Quantification of in Vivo c-di-GMP Synthesis

Liver samples were harvested from mice injected with 2×10⁹ vps/mouse AdVCA0848, or 2×10¹¹ vps/mouse of AdVCA0848, AdVCA0848^{mut}, AdVCA0956, AdNull, or not injected (naïves) as described in the animal procedures. 20 mg from each liver sample was placed in 500 μL PBS and homogenized using an Omni Tissue Homogenizer (Omni International). 300 μL of homogenate was added to an equal volume of equilibrated Phenol Solution (Sigma-Aldrich, St. Louis, MO). The homogenate-phenol solution was then vortexed and centrifuged at 15,000 rpm for 10 minutes. The aqueous phase was removed and added to 500 μL chloroform. The mixture was vortexed and then centrifuged at 15,000 rpm for 10 minutes. The aqueous phase was removed and stored at −80° C. until analysis. Quantification of c-di-GMP was conducted by liquid chromatography coupled with tandem mass spectrometry (LC-MS/MS) at Michigan State University spectrometry & metabolomics core facility as previously described (Massie, J P et al. (2012) Proc Natl Acad Sci USA 109: 12746-12751).

### 4. Western Blot for Gag Protein

Liver samples from mice injected with AdGag alone, or co-injected with AdGag and AdNull or AdVCA0848 as described above were harvested, and later were homogenized in ice cold lysis buffer containing 1% Triton and complete protease Inhibitor. Supernatant was collected and analyzed for protein concentration (BCA protein kit; Sigma-Aldrich, St. Louis, MO). Total protein of 15 μg was heated at 100° C. for 5 min with Laemmli sample buffer (Sigma Aldrich, St. Louis, MO), and samples were loaded on 1 mm-thick 10% gel Mini-Protean TGX Precast Gels (BIO-RAD, Hercules, CA, USA). Transfer was completed overnight at 4° C. using a 0.2 um Nitrocellulose membrane (Millipore, Billerica, MA). The membrane was blocked for 1 h in Odyssey® Blocking Buffer (Licor Biosciences—U.S., Lincoln, NE), then incubated for 1 hour at room temperature with primary monoclonal mouse anti Gag (1:10,000) antibody (183-H12-5C) obtained from the NIH-AIDS research and reference reagent program (gift from Dr. Y-H Zheng, Michigan State University), and mouse anti-ß-actin (1:3000) (#8224; Abcam, Cambridge, MA) diluted in Odyssey Blocking Buffer (#927-40000, Licor, Lincoln, NE). The blot was washed with TBS-T three times, and then incubated with labeled anti-mouse secondary antibody (#926-32210; Licor, Lincoln, NE) diluted in blocking buffer (1:10,000) for 1 hour at room temperature. The blotted membrane was washed and developed on the Licor Odyssey (Licor, Lincoln, NE).

### 5. ELISA

Effects of AdVCA0848 on IFN-β induction was determined by quantifying IFN-0 using the VeriKine™ mouse IFN-β ELISA kit (PBL Assay Science, Piscataway, NJ) according to the manufacturer's instructions. To determine the effect of AdVCA0848 on B cell adaptive immune responses specific to antigens delivered by the co-administered AdGag or AdToxB, or the extracellular antigen OVA with the use of AdNull or AdVCA0848^{mut} as a negative control, ELISA-based titering experiments were conducted as previously described (Appledom, D M et al. (2011) Clin Vaccine Immunol 18: 150-160). Briefly, 5×10⁸ vps/well of inactivated Ad5 particles, 0.2 mg/well of Gag protein, 50 μg/well of OVA, or 100 ng/well of ToxB (each diluted in PBS) was used to coat wells of a 96-well plate overnight at 4° C. Plates were washed with PBS-Tween 20 (0.05%) solution, and blocking buffer (3% BSA in PBS) was added to each well and incubated for 1-3 h at room temperature. For measuring total IgG Abs, plasma from injected mice was serially diluted in PBS buffer. Following dilution, plasma was added to the wells and incubated at room temperature for 1 h. Wells were washed using PBS-Tween 20 (0.05%), and HRP-conjugated rabbit anti-mouse Ab (Bio-Rad, Hercules, CA) was added at a 1:5000 dilution in PBS-Tween 20. Tetramethylbenzidine (Sigma-Aldrich, St. Louis, MO) substrate was added to each well, and the reaction was stopped with 2 N sulfuric acid. Optical density (O.D.) was then obtained by reading the plates at 450 nm in a microplate spectrophotometer.

### 6. ELISPOT

Splenocytes were harvested from individual mice and red blood cells were lysed using ACK lysis buffer (Invitrogen, Grand Island, NY). Ninety-six-well Multi-Screen high protein binding Immobilon-P membrane plates (Millipore, Billerica, MA) were wetted with 70% ethanol, coated with mouse anti-IFN-γ or IL-2 capture Abs, incubated overnight, and blocked prior to the addition of 5×10⁵ (AdGag studies) or 1×10⁶ (OVA studies) splenocytes/well. Additional studies were conducted using AdVCA0848^{mut} as a control (AdGag studies) with the use of 1×10⁶ splenocytes/well. Ex vivo stimulation included incubation of splenocytes in 100 μl media alone (unstimulated) or media containing 4 μg/ml Gag-specific AMQMLKETI (AMQ) peptide (GenScript, Piscataway, NJ) for the AdVCA0848 and AdGag studies, or 10 μg/ml OVA or SIINFEKL (MHC class I-restricted OVA-derived peptide (Ahlen, G et al. (2012) PLoS One 7: e46959)) for AdVCA0848 and OVA studies, overnight in a 370 C, 5% CO₂ incubator. Staining of plates was completed per the manufacturer's protocol. Spots were counted and photographed by an automated ELISPOT reader system (Cellular Technology, Cleveland, OH). Ready-SET-Go! IFN-γ and IL-2 mouse ELISPOT kits were purchased from eBioscience (San Diego, CA).

### 7. Flow Cytometry Analysis

To investigate innate immune responses following AdVCA0848 vaccination, mice were injected with 1×10¹⁰ vps/mouse of AdVCA0848 vector and activation of innate immune cells was evaluated 6 hours following i.v. injection. Splenocytes were stained with various combinations of the following antibodies: PE-CD69 (clone: H1.2F3), allophycocyanin-Cy7-CD3 (clone: 145-2C11), PerCP-Cy5.5-CD19 (clone: 1D3), Pacific Blue-CD8α (clone: 53-6.7), and PE-Cy7-NK1.1 (clone: PK136) (4 μg/ml). To assess the effect of AdVCA0848 on dendritic cells (DCs), splenocytes were stained with combinations of the following antibodies: PE-Cy7-CD11c (clone: HL3), allophycocyanin (APC)-Cy7-CD11b (clone: M1/70), Alexa Fluor 700-CD8a (clone: 53-6.7), FITC-CD40 (clone: HM40-3), PerCP-Cy5.5-CD80 (clone: 16-10A1), and V450-CD86 (clone: GL1) (4 μg/ml). All antibodies were obtained from BD Biosciences. To determine the intracellular cytokine levels 14 dpi of AdVCA0848 and AdGag co-injections, intracellular staining was performed as previously described (Aldhamen, Y A et al. (2012) J Immunol 189: 1349-1359). Briefly, splenocytes (2.5×10⁶/well) were stimulated with Gag-specific AMQ peptide for 6 hours with Brefeldin A (BFA) (Sigma-Aldrich, St. Louis, MO) for 30 minutes and stored at 4° C. overnight. Cells were washed twice with FACS buffer and surface stained with APC-CD3, Alexa Fluor 700-CD8a, and CD16/32 Fc-block Abs, fixed with 2% formaldehyde (Polysciences, Warrington, PA), permeabilized with 0.2% saponin (Sigma-Aldrich, St. Louis, MO), and stained for intracellular cytokines with PE-Cy7-TNF-α, and Alexa Fluor 488-IFN-γ (4 μg/ml) (all obtained from BD Biosciences, San Diego, CA). We included a violet fluorescent reactive dye (ViViD; Invitrogen) as a viability marker to exclude dead cells from the analysis. Tetramer staining of splenocytes at 1×10⁶ cell/well was performed using PE-labeled MHC class I tetramer folded with the AMQ peptide (generated at the NIH Tetramer Core Facility (Atlanta, GA)) for 30 minutes at room temperature, and for memory T cell staining, a mixture of the following antibodies (at 2 μg/ml) were used: APC-CD3, Alexa Fluor 700-CD8a, PerCP-Cy5.5-CD127, FITC-CD62L, and CD16/32 Fc-block Abs. All antibodies were purchased from BD Biosciences (San Diego, CA). After washing with FACS buffer, data for stained cells were collected with the use of BD LSR II instrument and analyzed using FlowJo software (Tree Star, San Carlos, CA). Gating strategy was based on negative control results (naïves) that were applied consistently across all samples examined. Representative examples from this gating approach are presented here for activation of innate immunity cells and for the frequency of cytokine-producing CD8⁺ T cells.

### 8. Statistical Analysis

Statistically significant differences in innate immune responses were determined using a one-way ANOVA with a Student-Newman-Keuls post hoc test (p value of <0.05 was deemed statistically significant). The ELISPOT and ELISA studies were all analyzed using one-way ANOVA with a Student-Newman-Keuls post hoc test (p value of <0.05 was deemed statistically significant). For flow cytometry, a one-way ANOVA with a Student-Newman-Keuls post hoc test was used (p value of <0.05 was deemed statistically significant). Statistical analyses were performed using GraphPad Prism (GraphPad Software).

### Example 7: AdVCA0848 Produces Significant Amounts of c-di-GMP In Vivo in Mice

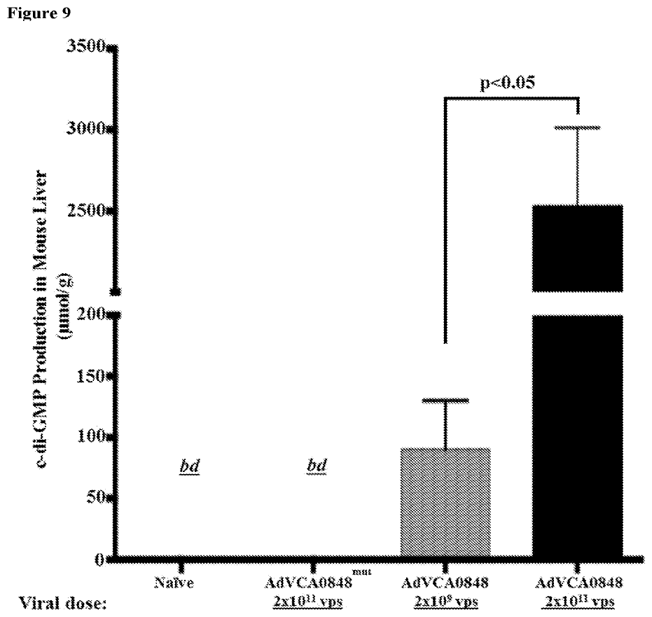

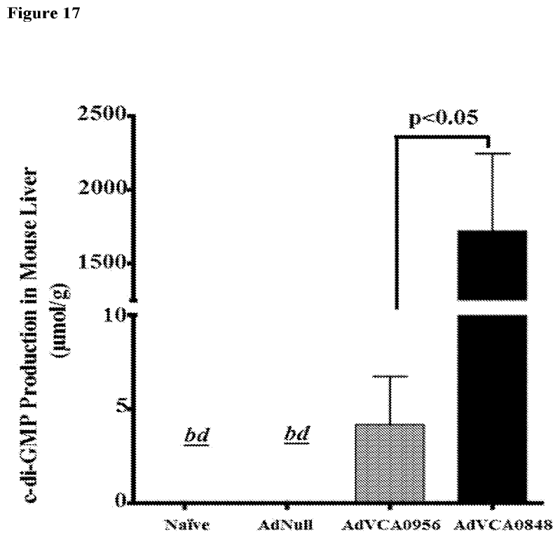

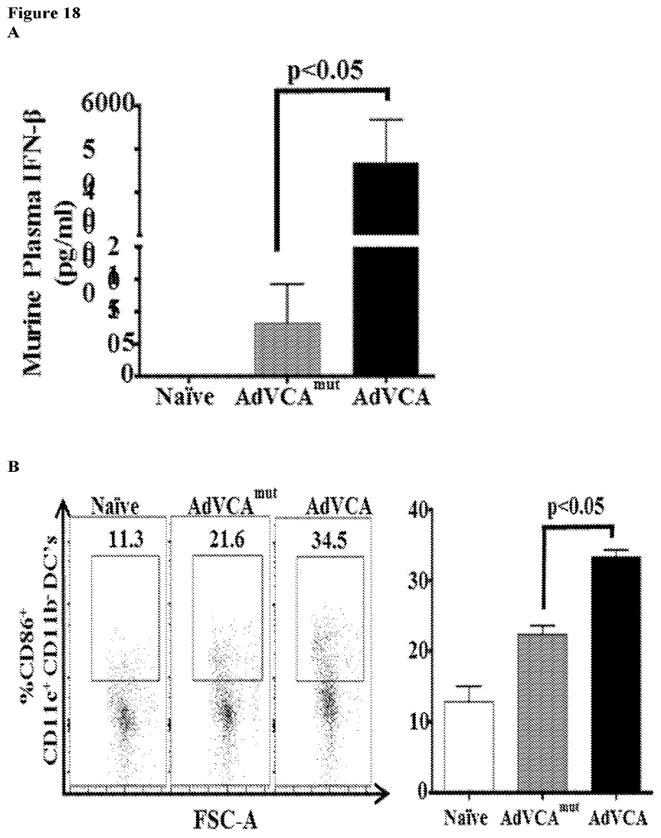

Examples 1-5 above demonstrated the feasibility of in vitro and in vivo production of c-di-GMP in mammalian cells by using Ad5 vectors to transduce DGCs. Prior unpublished studies by the inventors suggested that use of an alternative DGC, VCA0848, which has greater enzymatic activities, might generate a significantly elevated amount of c-di-GMP in vivo. An Ad5 vector with a CMV enhancer/promoter element to drive VCA0848 expression in mammalian cells was constructed. The use of the AdVCA0848 platform resulted in a significant in vivo c-di-GMP production measured in the liver of injected mice. Injecting with increasing viral loads of 2×10⁹ vps/mouse and 2×10¹¹ vps/mouse of AdVCA0848 resulted in approximately 130 μmol/g and 3000 μmol/g c-di-GMP in the liver, respectively. This confirms that the in vivo c-di-GMP production is entirely due to the enzymatic activity of the delivered VCA0848 as AdVCA0848^{mut} vectors and naïve mice failed to produce detectable levels of c-di-GMP (FIG. 9). Additionally, when compared to an earlier DGC-expressing platform that was constructed using the exact same adenovirus vector backbone, the AdVCA0848 platform produces significantly higher levels of c-di-GMP in the mouse liver (˜400-fold increase) than that produced by an equal viral dose of the AdVCA0956 platform per gram of mouse liver (p<0.05). As expected, similar to AdVCA0848^{mut} control, the AdNull vectors, which lack the DGC gene, did not produce detectable levels of c-di-GMP (FIG. 17). These results confirm the feasibility of transducing the bacterial DGC VCA0848 using Ad5 to synthesize in vivo larger amounts of c-di-GMP in vivo.

### Example 8: AdVCA0848 Activates Innate Immune Responses

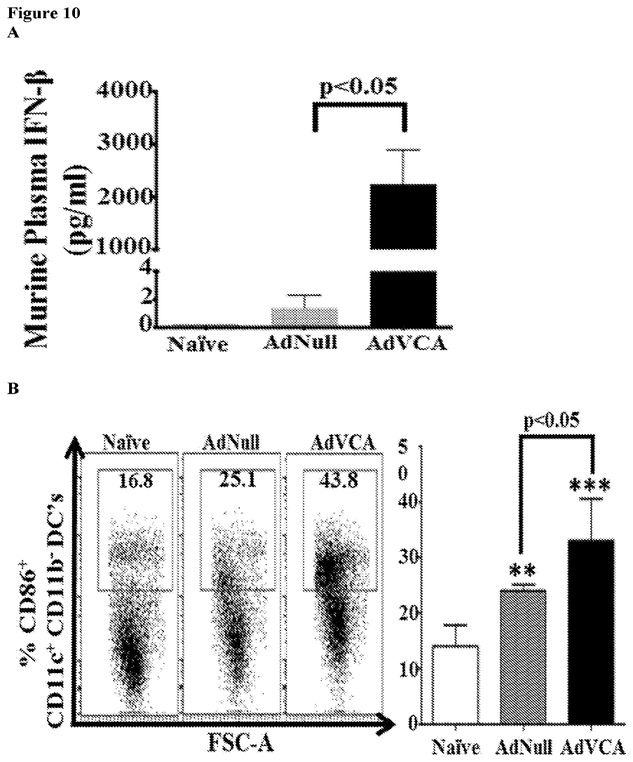

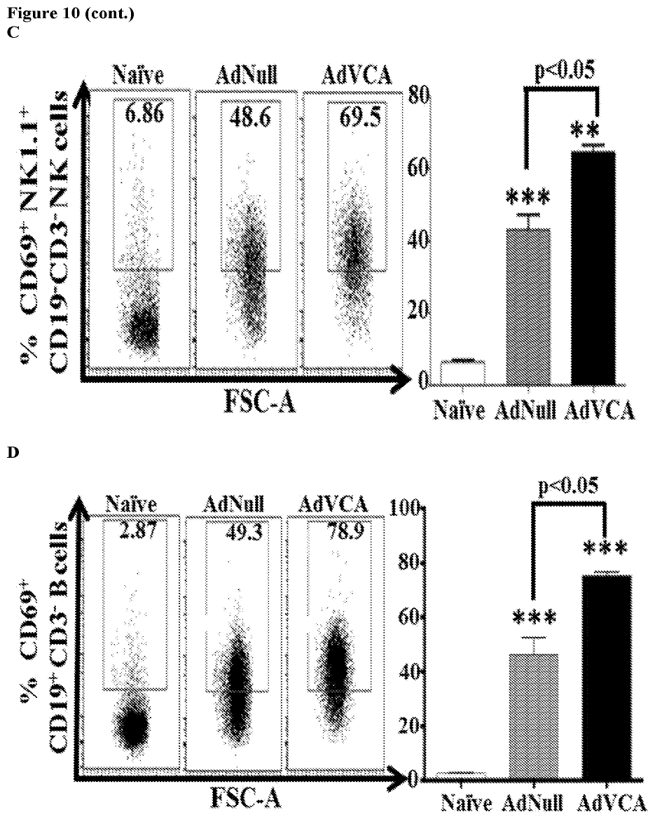

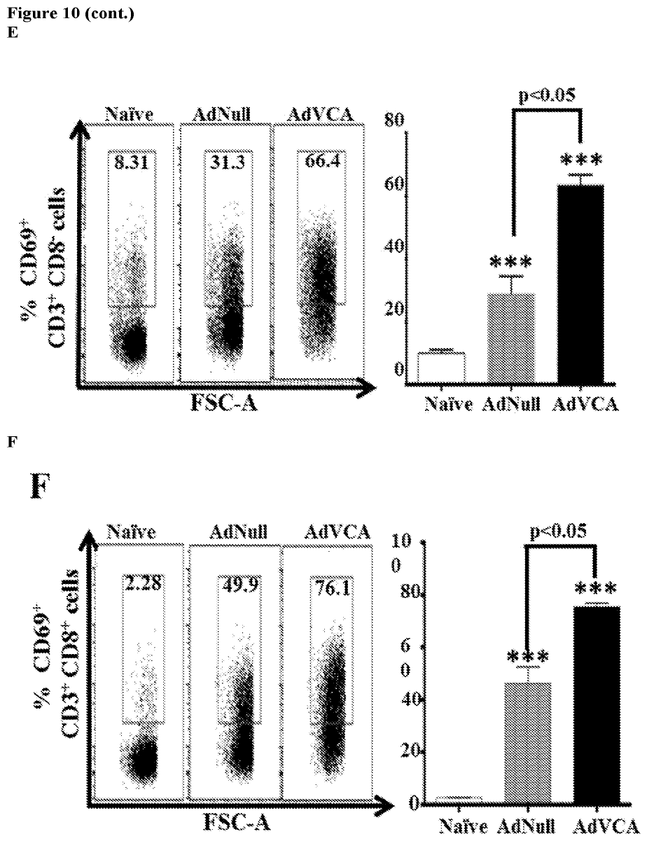

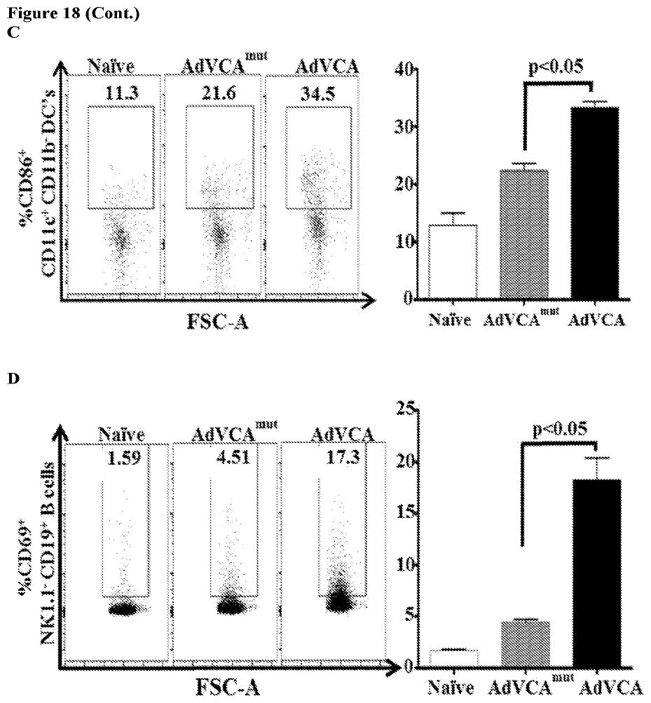

It was thought that activation of beneficial innate immune responses by adjuvants is the underlying mechanism that is critical for achieving effective and long-lived, antigen-specific, adaptive immune responses. Intravenous administration of AdVCA0848 dramatically induced plasma levels of IFN-β (p<0.05) nearly 1000-fold compared to the level produced by the AdNull control (FIG. 10A). Importantly, administration of AdVCA0848^{mut} control produced similar levels of IFN-β, as compared to AdNull, suggesting the increased IFN-β levels following AdVCA0848 is due to the enzymatic activity of the transduced VCA08484 (FIG. 18A). Also, administration of AdVCA0848 significantly induced DC maturation and NK activation as compared to an identical cell population derived from AdNull controls (p<0.05) (FIGS. 10B & 10C). Furthermore, administration of AdVCA0848 resulted in increased numbers of CD69-expressing B cells, CD3⁺CD8⁻ and CD3⁺CD8⁺ T cells, as compared to the use of the AdNull vector in this experiment (p<0.05) (FIGS. 10D-10F). Utilization of AdVCA0848^{mut} control suggested that the activation of immune cells is largely due to the enzymatic activity of the transduced VCA0848 (FIGS. 18B-18F). Our results also confirmed previous findings that the Ad5 vector itself results in increased activation of NK cells, macrophages, CD3⁺CD8⁻ T cells, CD3⁺CD8⁺ T cells, and B cells as indicated by the significant expression of the activation marker CD69 (Aldhamen, Y A et al. (2012) J Immunol 189: 1349-1359). Together, these data suggest a significant induction of innate immune responses by AdVCA0848 in the mouse model, surpassing that caused by the adenovirus itself.

### Example 9: AdVCA0848 Enhances Induction of Antigen-Specific Adaptive T Cell Immune Responses

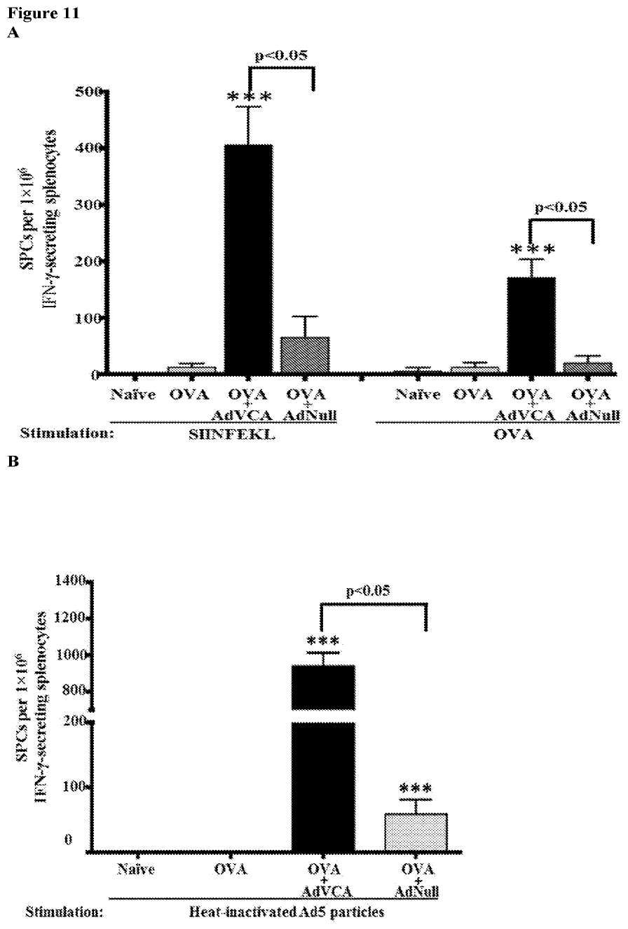

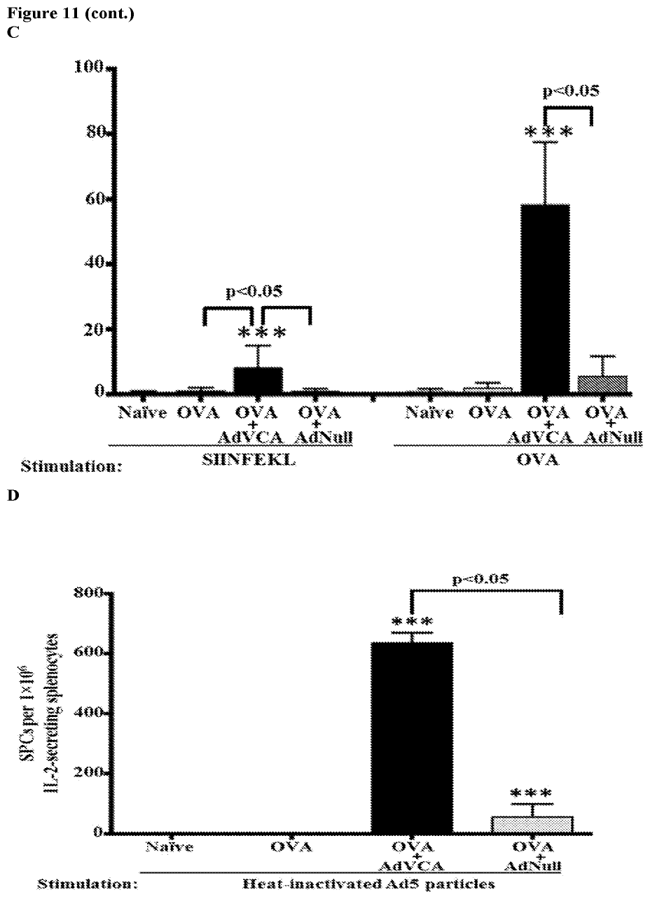

Direct administration of the ovalbumin (OVA) protein is a model antigen frequently used to study antigen-specific adaptive immune responses (Basto, A P et al. (2015) Mol Immunol 64: 36-45; Garulli, B et al. (2008) Clin Vaccine Immunol 15: 1497-1504). C57BL/6 mice were vaccinated with 100 g/mL OVA alone, or simultaneously with AdNull or AdVCA0848; and a fourth untreated group served as a naïve control. At 14 dpi, IFN-γ ELISPOT results from the experimental and control animals indicated that OVA-specific T cell responses from mice co-administered with AdVCA0848 and OVA were significantly higher (upon ex vivo stimulation with the entire OVA protein or the OVA-derived MHC class I-restricted peptide SIINFEKL) as compared to splenocytes derived from mice receiving only OVA, or OVA concomitant with the AdNull control vector (p<0.05) (FIG. 11A). The simultaneous use of AdVCA0848 with OVA vaccination also increased the number of SIINFEKL and the intact OVA protein-specific IL-2-secreting T cells present in the splenocytes of OVA-treated mice as compared to mice injected with OVA alone, or concomitant with AdNull control (p<0.05) (FIG. 11C). The noticeable variability of T cell responses resulted from the ex vivo stimulation with whole OVA protein and the MHC class I-restricted SIINFEKL peptide likely suggest a CD8⁺ T cell-driven response indicated by higher SIINFEKL-specific IFN-γ producing T cells and smaller SIINFEKL-specific IL-2 producing T cells. Interestingly, splenocytes harvested from mice co-injected with AdVCA0848 and OVA also had dramatically increased numbers of Ad5 capsid-specific IFN-γ-secreting T cells and IL-2 secreting T cells, as compared to mice injected with OVA alone, or concomitant with AdNull control (p<0.05) (FIGS. 11B and 11D). These results indicate that AdVCA0848 provides enhancement of OVA-specific adaptive T cell immune responses when co-injected with the extracellular antigen OVA.

### Example 10: AdVCA0848 Enhances Induction of Antigen-Specific Adaptive B Cell Immune Responses

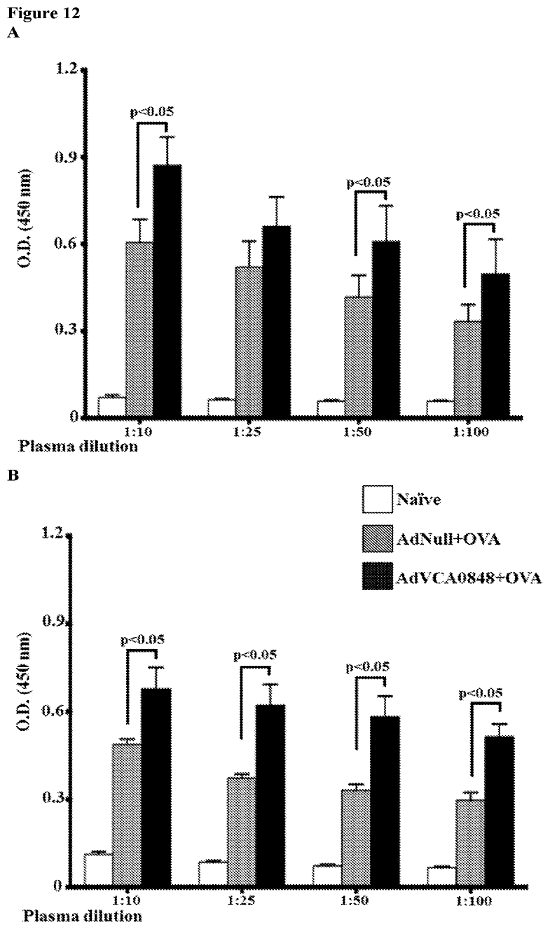

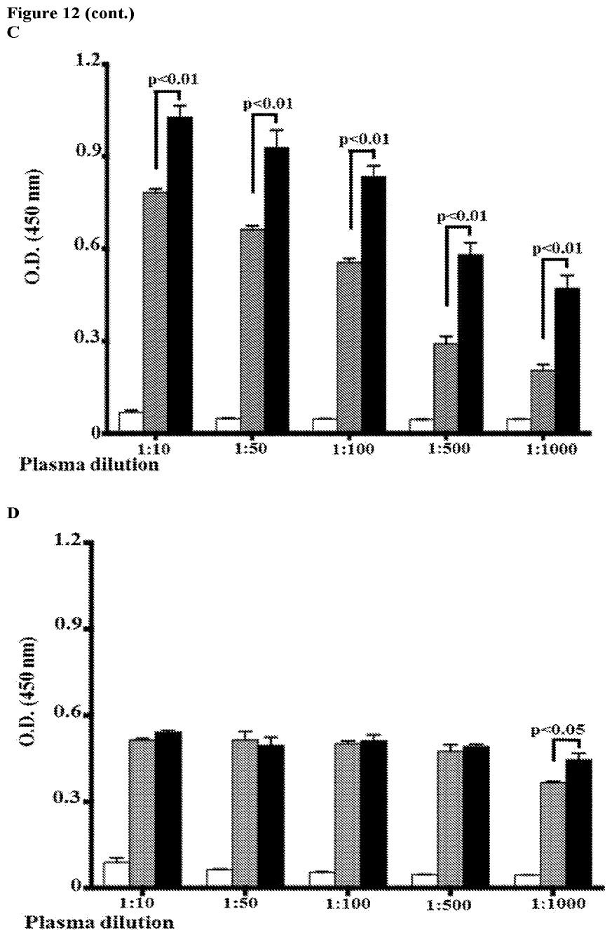

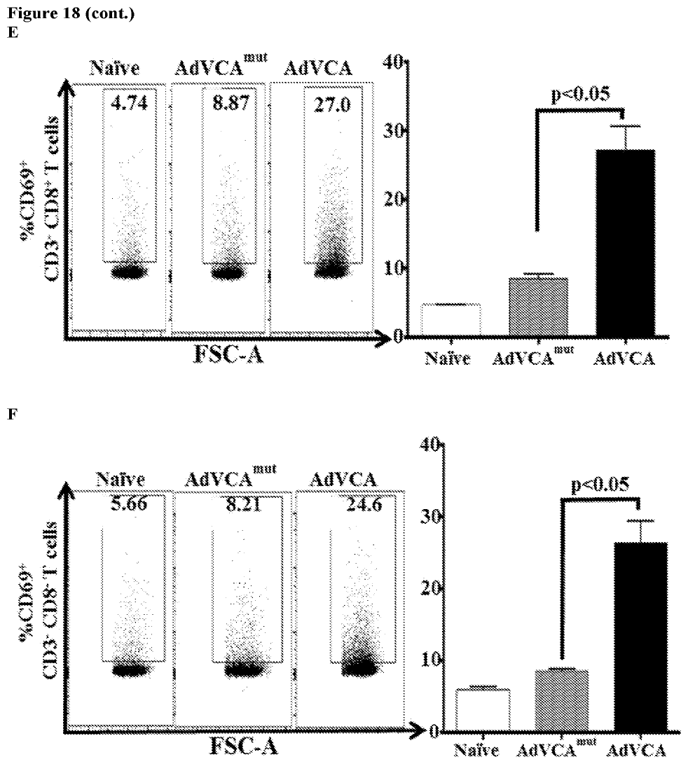

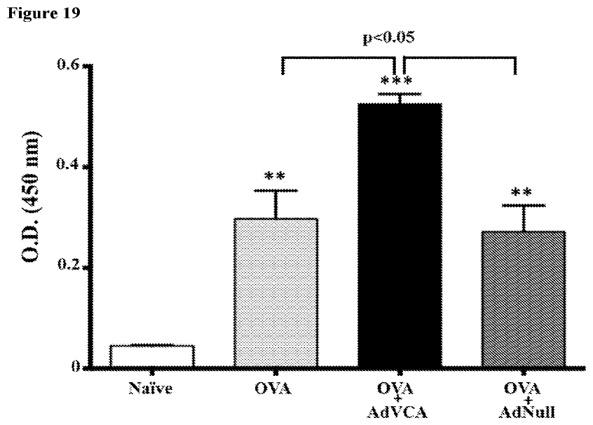

Co-administering AdVCA0848 and OVA also resulted in enhancement of OVA-specific (FIG. 12A) and Ad5-specific (FIG. 12B) B cell responses 6 dpi. At 14 dpi, OVA-specific B cell response was enhanced compared to mice co-injected with the AdNull control vector (FIG. 12C) or when injected with OVA alone (p<0.05) (FIG. 19). Ad5-specific IgG antibody B cell responses were also detected in those mice that received either of the Ad5 vectors. While the presence of AdVCA0848 significantly increased the Ad5-specific B cell response compared to that exerted by the AdNull control (p<0.05) when measured at 6 dpi, this effect was observed to be minimal when measured at 14 dpi (FIG. 12D). Despite the transient enhancement of humoral response against the delivering vector, these results demonstrate the beneficial effects of AdVCA0848 on the OVA-specific adaptive B cell response from a single administration of OVA.

### Example 11: Sustained High-Level Production of c-Di-GMP can Inhibit T Cell Responses to Antigens Expressed from Viral Vectors

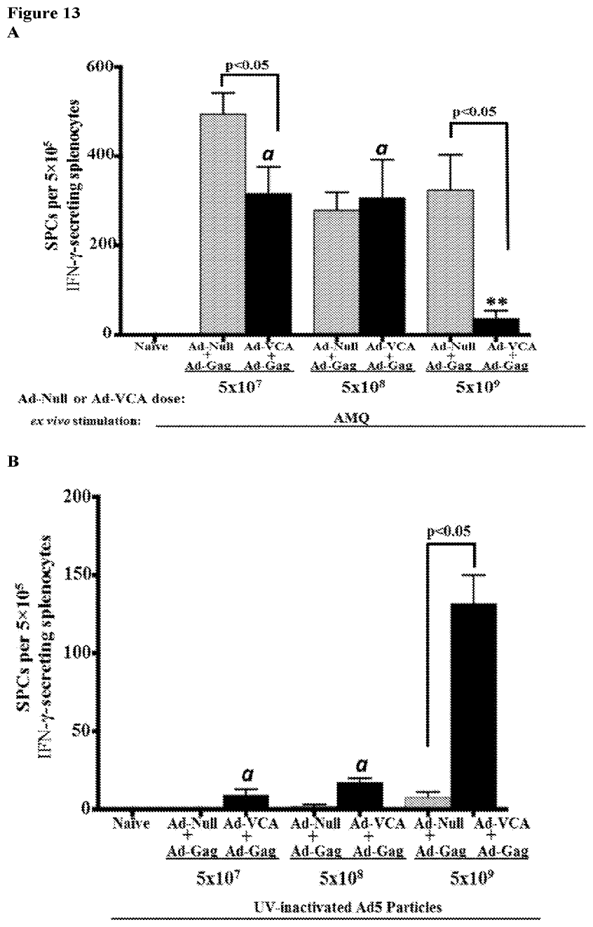

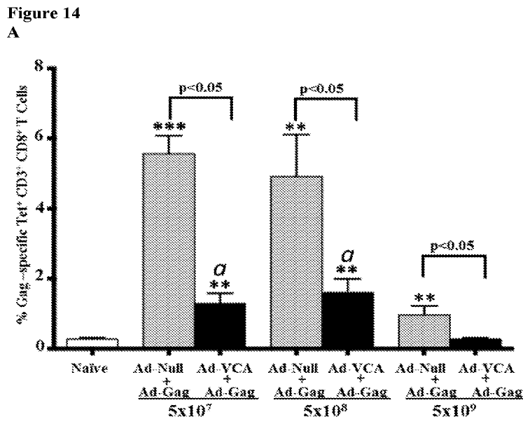

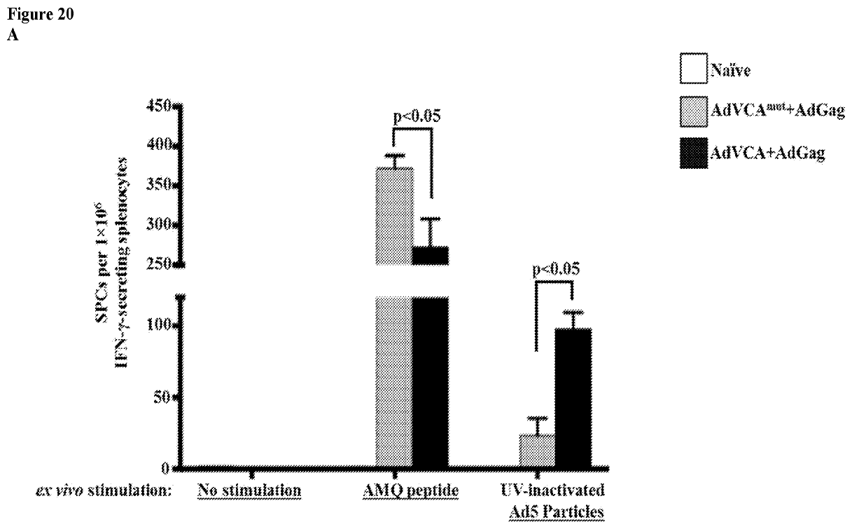

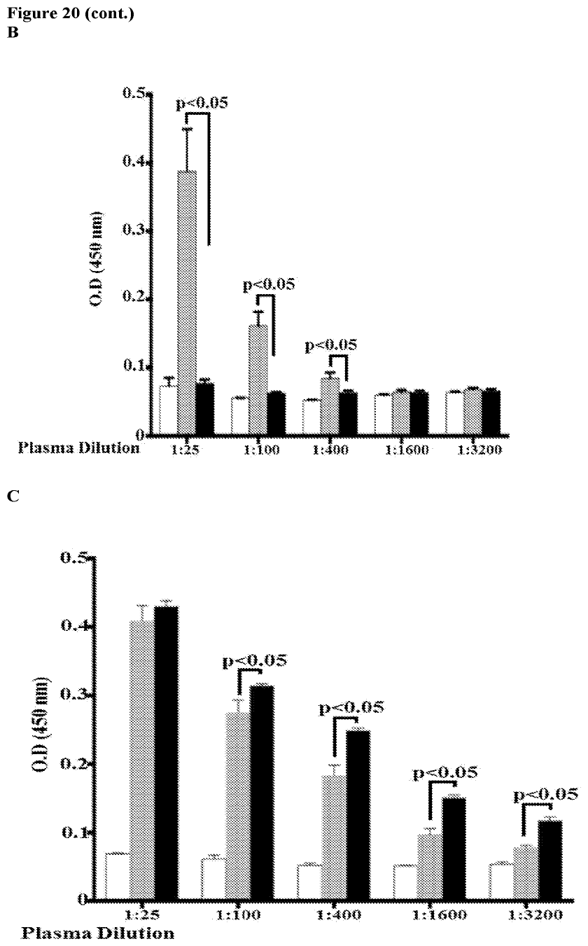

The previous results indicated a modest, although significant, enhancement of adaptive immune responses specific against antigens expressed from Ad5-based vaccines co-injected with AdVCA0956, a vector expressing a less active DGC (Examples 1-5). Therefore, it was assessed whether the enhanced ability of AdVCA0848 to produce c-di-GMP in vivo would also improve adaptive immune responses specific for adenovirus-expressed antigens. An adenovirus-based vector was previously used to express the Gag protein, an HIV-1-derived antigen, and demonstrated the platform's ability to induce Gag-specific humoral and cellular immune responses (Aldhamen, Y A et al. (2011) J Immunol 186: 722-732; Appledorn, D M et al. (2010) PLoS One 5: e9579; Appledorn, D M et al. (2011) Clin Vaccine Immunol 18: 150-160; Gabitzsch, E S et al. (2009) Immunol Lett 122: 44-51). Based on the previous work, the AdGag vaccine was administered at the dose of 5×10⁶ vps/mouse along with escalating doses (5×10⁷, 5×10⁸, or 5×10⁹ vps/mouse) of AdVCA0848 or the AdNull control. After 14 days, Gag-specific memory T cell immune responses were evaluated by IFN-γ ELISPOT assay. The results demonstrated that concurrent administration of AdVCA0848 along with the AdGag vaccine inhibited T cell responses to the Gag antigen, which were especially significant at the highest AdVCA0848 dose of 5×10⁹ vps/mouse compared to that seen from the concurrent administration of AdNull control along with AdGag vaccine (p<0.05) (FIG. 13A). Similar to the previous observations (Schuldt, N J et al. (2011) PLoS One 6: e24147), as the viral load of AdNull co-injected with AdGag increased, the Gag-specific T cell response measured by IFN-γ ELISPOT decreased in a dose-dependent manner (p<0.05). In contrast, ELISPOT assays demonstrated a dramatic enhancement of Ad5-specific IFN-γ-producing T cells at 5×10⁹ vps/mouse of AdVCA0848 compared to the AdNull control group (p<0.05), while the first two doses of 5×10⁷ and 5×10⁸ vps/mouse showed minimal Ad5-specific T cell response (FIG. 13B). It was confirmed that the inhibitory effects on IFN-γ-secreting T cells was lost in a VCA0848 mutant that cannot synthesize c-di-GMP (FIG. 20A).

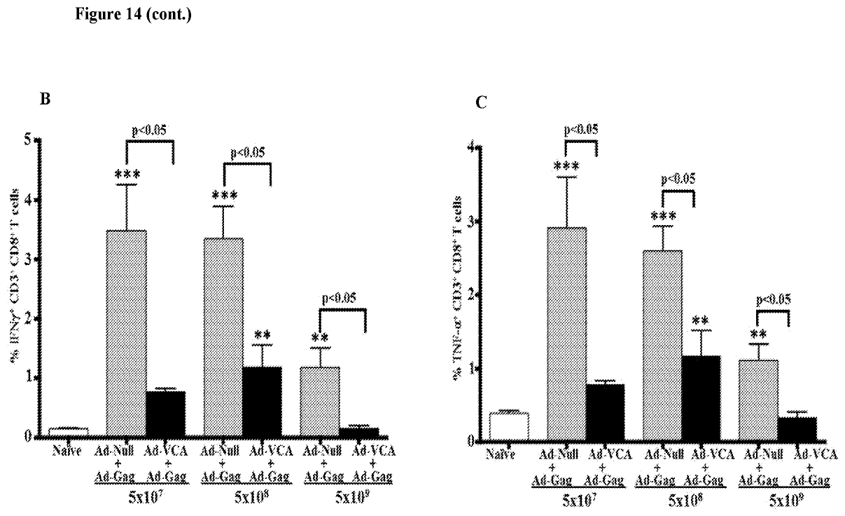

A multi-parameter tetramer-binding assay showed a significantly decreased number of Gag-specific Tet⁺CD8⁺ T cells present in mice co-injected with three different doses of AdVCA0848 along with AdGag as compared to mice co-injected with AdGag and the AdNull control vector (p<0.05) (FIG. 14A), confirming the negative impact of AdVCA0848 on the induction of Gag-specific CD8⁺ T cells. Intracellular staining (ICS) and FACS analysis was also performed to evaluate the impact of AdVCA0848 on the numbers of Gag-specific CD8⁺ T cells upon ex vivo stimulation with the Gag-specific peptide, AMQ. The number of IFN-γ and TNF-α-producing CD8⁺ T cells specific for this potent Gag peptide were significantly inhibited in mice co-injected with AdVCA0848 as compared to equal viral loads of AdNull (p<0.05) with the highest dose of AdVCA0848 of 5×10⁹ vps/mouse showing the strongest inhibitory effects (FIGS. 14B & 14C). The effect of AdVCA0848 on Gag-specific IFN-γ, TNF-α and IL-2-producing CD4⁺ T cells was also looked at and no significant effect was observed (data not shown). Together, these data strongly suggested that despite a strong induction of innate immunity, and improved induction of adaptive immune responses to extracellular proteins such as the OVA protein and the Ad5 capsid, expressing high levels of c-di-GMP using VCA0848 from an Ad5 vector significantly inhibited induction of antigen specific CD8⁺ T cell responses to antigens expressed intracellularly by another Ad5 vector.

### Example 12: Sustained High-Level Production of c-Di-GMP can Also Inhibit B Cell Responses to Antigens Expressed from Viral Vectors

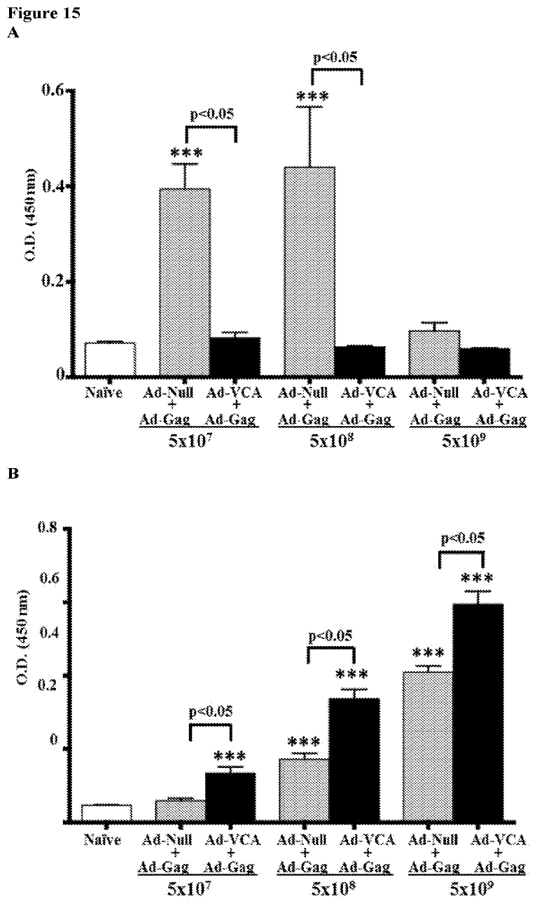

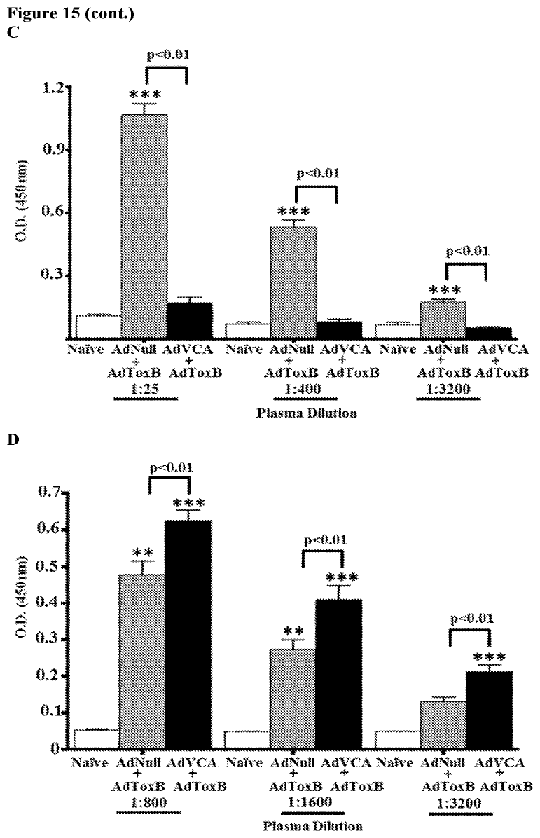

Humoral B cell responses following AdVCA0848 co-administration with AdGag were evaluated. Similar to its effect on T cell responses, the presence of AdVCA0848 resulted in significant inhibition of HIV-1/Gag-specific B cell responses as compared to those mice administered with equal amounts of the AdNull control vector (p<0.05) (FIG. 15A). The inhibition of Gag-specific B cell responses by AdVCA0848 was very potent at the doses of 5×10⁷ and 5×10⁸ vps/mouse (compared to AdNull, p<0.05). AdNull exhibited inhibition similar to AdVCA0848 at the highest dose of 5×10⁹ vps/mouse (FIG. 15A). Alternatively, increasing doses of both the AdNull and AdVCA0848 increased B cell responses against the Ad5 vector in a dose-dependent manner (FIG. 15B). The inhibitory effects on Gag-specific B cell responses were lost using the AdVCA0848^{mut} that cannot synthesize c-di-GMP (FIG. 20B). The ability of AdVCA0848 to enhance Ad5-specific B cell response compared to that shown by AdVCA0848^{mut} was confirmed (FIG. 20C).

To confirm this interesting observation using a different antigen expressed by an Ad5-based vaccine, we co-administered AdVCA0848 along with an Ad5 vector expressing the truncated form of the C. difficile-derived Toxin B protein (AdToxB). The presence of AdVCA0848 with AdToxB also resulted in significantly reduced ToxB-specific B cell responses as compared to control vaccinations (p<0.001) (FIG. 15C). Importantly, significantly (p<0.01) increased Ad5-specific IgG titers in mice vaccinated with AdVCA0848 and AdToxB was again obsereved, as compared to controls (FIG. 15D). These results further confirm the inhibitory effects of the strong c-di-GMP producer, AdVCA0848, on another antigen intracellularly expressed from an adenovirus vector (AdToxB).

### Example 13: Co-Administration of AdGag and AdVCA0848 Doesn't Inhibit Gag Expression

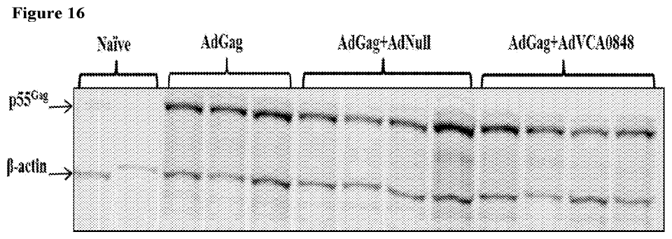

One possible explanation for the inhibition of response to Ad-expressed antigens is that the presence of the AdVCA0848 vector inhibits in trans the in vivo expression of the Ad expressed antigens. However, mice co-injected with AdVCA0848 and AdGag demonstrated the presence of the HIV-1 derived Gag protein whether delivered by the AdGag platform alone, or when co-injected with the AdNull control, or with AdVCA0848, (FIG. 16). These results suggest that inhibitory effects exerted by AdVCA0848 on B cell and T cell adaptive immune responses against Gag are not due to lack of Gag expression and translation in vivo.

### Discussion

Understanding the molecular mechanisms underlying how a putative adjuvant acts to enhance the efficacy of a specific vaccine will help to guide the formulation of newer generation vaccines that efficiently generate specific long-term immunity against difficult antigens derived from pathogens or cancer cells (Rueckert, C et al. (2012) PLoS Pathog 8: e1003001). The use of pure c-di-GMP has been demonstrated to be an immune-modulatory molecule with potential therapeutic and prophylactic properties (Karaolis, D K. et al. (2007) J Immunol 178: 2171-2181). While the presence of nucleic acids can be sensed by AIM2, and signals the activation of caspase-1 (Hornung, V et al. (2009) Nature 458: 514-518; Fernandes-Alnemri, T et al. (2009) Nature 458: 509-513), the presence of cytosolic c-di-GMP can be sensed by other sensors including the STING and helicase DDX41 pathways, and subsequently lead to the release of IFN-β, primarily from CD11b⁺ DCs (Huang, L et al. (2013) J Immunol 191: 3509-3513). Additionally, c-di-GMP has been shown to stimulate the MYPS/STING-dependent induction of TNF-α and IL-22, not type I IFN, when used as a nasal mucosal adjuvant, suggesting c-di-GMP may have different effects on different innate immunity pathways (Blaauboer, S M et al. (2014) J Immunol 192: 492-502; Blaauboer, S M et al. (2015) eLife 4).

In this study, the ability of a potent, bacterial derived DGC to be delivered by an Ad5 vector (AdVCA0848) that produced more than 400-fold more c-di-GMP than the Ad5 DGC vector described above (Examples 1-5) was demonstrated, resulting in a robust induction of several innate immune responses, including IFN-β induction. By using a mutant version of VCA0848 delivered by AdVCA0848^{mut}, the data herein suggests that these significant levels of c-di-GMP are products of the enzymatic activity of the transduced VCA0848. These strong innate immune responses allowed the induction of enhanced adaptive immune responses to an extracellular antigen, i.e. OVA, co-administered with the AdVCA0848, but also suppressed adaptive immune responses to virally expressed antigens. The recent characterization of mammalian endogenous cyclic GMP-AMP (2′3′-cGAMP) synthetase (cGAS) (Wu, J et al. (2013) Science 339: 826-830; Ablasser, A et al. (2013) Nature 503: 530-534; Zhang, X et al. (2013) Mol Cell 51: 226-235) provided the rationale for testing cGAMP as a vaccine adjuvant, and initial studies demonstrated its usefulness in stimulating innate immune responses and improving antigen-specific adaptive immune responses (Li, X D et al. (2013) Science 341: 1390-1394; Gao, D et al. (2013) Science 341: 903-906; Skrnjug, I et al. (2014) PLoS One 9: e110150). When compared to the bacterial c-di-GMP, cGAMP had higher binding affinity to STING. However, it has also been shown that c-di-GMP results in higher IFN-β induction than that induced by 2′3′-cGAMP or its isomers, suggesting that higher binding affinity to STING does not correlate with IFN-β induction. These results may be attributable to possible differences in biological stability between c-di-GMP and the mammalian cGAMP (Zhang, X et al. (2013) Mol Cell 51: 226-235).

The adenovirus-based platforms utilized in the present studies described herein are also expected to activate multiple innate immune responses. The vector is known to activate innate immune responses via interactions with extracellular and intracellular TLRs, and can simultaneously trigger early pro-inflammatory responses such as the induction of IP-10 (Tibbles, L A. et al. (2002) J Virol 76: 1559-1568) and the activation of the P13K signaling cascade (Verdino, P et al. (2010) Science 329: 1210-1214). It has been also demonstrated that upon penetrating host cells and escaping the endosomal compartment, adenoviral vectors have the ability to ignite the MAPK and NFκB signaling pathways through TLR-dependent (TLR2, 3, 4, and 9) and non-TLR dependent mechanisms (Appledorn, D M et al. (2008) J Immunol 181: 2134-2144; Zhu, J et al. (2007) J Virol 81: 3170-3180; Appledorn, D M et al. (2009) J Innate Immun 1: 376-388) leading to the induction of several chemokines and cytokines, fostering its utility as a vaccine platform in and of itself. Additionally, the adenoviral dsDNA genome can be sensed by cytoplasmic sensors such as DAI (leading to type I IFN induction) (Ishii, K J et al. (2008) Nature 451: 725-729) and AIM-2 resulting in activating the inflammasome and the induction of caspase-1-dependent IL-1$ (Hornung, V et al. (2009) Nature 458: 514-518). Recent data also suggest that STING is central and acts as a major PRR after vaccination with Ad5-based platforms including Ad5 vectors (Quinn, K M et al. (2015) J Clin Invest 125: 1129-1146). With these facts in mind, it is clear that these results confirm that the additional production of c-di-GMP from an already immunogenic platform such as Ad is significant enough to further promote the induction of pro-inflammatory immune responses beyond that provided by the Ad vector platform itself. Whether expression of DGCs from other vaccine platforms will yield similar results awaits future studies beyond the scope of this manuscript.

The broad impact of the AdVCA0848 platform on innate immune responses clearly demonstrates its promising potential for use as a vaccine adjuvant to enhance adaptive immune responses. For example, relative to enhancing adaptive immune responses to extracellular antigens, plasmacytoid dendritic cell precursors (pDC) are thought to be the major source of IFN-β (Soumelis, V et al. (2006) Eur J Immunol 36: 2286-2292). In agreement with previous reports that demonstrated the stimulatory effects of c-di-GMP on murine and human DCs (Elahi, S et al. (2014) PLoS One 9: e109778; Karaolis, D K. et al. (2007) J Immunol 178: 2171-2181), AdVCA0848 improved the induction of CD11c⁻CD11b⁻ CD86⁺ DCs. Ultimately, pDCs can differentiate into typical DCs capable of stimulating naïve T cells in an antigen-specific manner (Renneson, J et al. (2005) Clinical and experimental immunology 139: 468-475). IFN-β has also been shown to enhance DC maturation, the efficiency of DC's to activate the cross-priming of CD8⁺ T cells, and increase induction of CD4⁺ Th I differentiation (Huber, J P et al. (2011) Immunology 132: 466-474). In addition to increasing the number of CD86⁺CD11c⁺CD11b⁻ DCs and activating CD69⁺NK1.1⁺ NK cells that are involved in regulating innate immune responses, AdVCA0848 activated cells directly involved in adaptive immune responses such as B cells and CD4⁺ and CD8⁺ T cells.

AdVCA0848 also enhanced induction of OVA-specific B cell and T cell adaptive responses. These results parallel recent studies evaluating the beneficial effects of direct administration of c-di-GMP as an adjuvant during vaccination with OVA (Blaauboer, S M et al. (2014) J Immunol 192: 492-502; Wu, J et al. (2013) Science 339: 826-830), and 4-Hydroxy-3-nitrophenylacetyl-Chicken Gamma Globulin, NP-CGG, in which c-di-GMP was shown to have the capacity to enhance germinal center (GC) development (Gray, P M et al. (2012) Cell Immunol 278: 113-119). Additionally, the presence of c-di-GMP in an adjuvant formulation containing chitosan (CSN) improved adaptive immune responses to H5N1 antigens (Svindland, S C et al. (2013) Influenza Other Respir Viruses 7: 1181-1193), and (along with a conventional aluminum salt-based adjuvant) improved adaptive immune responses specific to the hepatitis B surface antigen (HBsAg) (Gray, P M et al. (2012) Cell Immunol 278: 113-119). Recently, it was demonstrated that nasal administration of c-di-GMP significantly increases the MYPS-mediated uptake of OVA antigen via endocytosis and pinocytosis in vivo. This generates mucosal adjuvant activities that are mediated by type II and type III interferon but not type I interferon suggesting variable c-di-GMP pleiotropic effects on innate immune responses against extracellular antigens. The in vivo production of c-di-GMP by i.m. administration of our AdVCA0848 platform potentially enhanced the OVA uptake and processing by DCs, and subsequently resulted in improved OVA-specific adaptive immune responses (Blaauboer, S M et al. (2015) eLife 4). As a proof of principle, our results suggest that adenovirus-based platforms expressing DGCs may also be used to promote improved immunity against other disease specific antigens, such as those found in current cholera, diphtheria, and tetanus vaccines, as each are examples of protein-based vaccines. In addition, as our approach also enhances activation of antigen-presenting cells (APCs) and induction of antigen CD8⁺ cytotoxic T lymphocytes (CTLs), future studies using tumor antigen specific peptides may also enhance the induction of anti-tumor cellular immune responses (Miyabe, H et al. (2014) J Control Release 184: 20-27; Chandra, D et al. (2014) Cancer Immunol Res 2: 901-910; Karaolis, D K et al. (2005) Biochem Biophys Res Commun 329: 40-45; Joshi, V B et al. (2014) Expert review of vaccines 13: 9-15).

The results described herein also revealed the potential for inhibitory effects on adaptive immune responses to antigens expressed intracellularly, simultaneous with provision of high levels of c-di-GMP. Although, the dose of 5×10⁸ vps/mouse of AdVCA0848 did not show significant inhibition of IFN-γ-secreting splenocytes compared to that shown by the AdNull control, this dose caused significant inhibition of Gag-specific IFN-γ and TNF-α-secreting CD8⁺ T cells, suggesting that CD8⁺ T cells may be the specific targets for these inhibitory effects. Furthermore, increasing the AdVCA0848 dose to 5×10⁹ vps/mouse further inhibited Gag-specific T cell responses. Of note, the use of higher doses of the AdNull control vector also resulted in decreased induction of Gag-specific CD8⁺ T cell responses. Despite this, the provision of elevated c-di-GMP levels resulted in additional inhibitory effects on Gag-specific adaptive immune responses.

Examples 1-5 show that increasing the dose of AdVCA0956 to 5×10⁹ vps/mouse did not improve B cell responses specific for an antigen delivered by an Ad5 vector in mice (Examples 1-5). Specifically, AdVCA0956 moderately suppressed B cell responses against the C. difficile-derived Toxin A antigen expressed from the co-injected Ad5 vector at the dose of 5×10⁹ vps/mouse. The results herein suggest that those trends were likely real. Even stronger inhibitory effects were noted after administration of the more potent AdVCA0848 on B cell and T cell adaptive immune responses against the intracellularly expressed Gag and ToxB antigens. These results suggest that in mice the magnitude of inhibitory effects on adaptive immune responses to intracellularly expressed antigens is likely to increase with excessive amounts of c-di-GMP production.

There is also the possibility that the transduced DGC, and ultimately the synthesized c-di-GMP, interferes with the expression of these antigens when using the CMV expression cassette (used in constructing the vectors). This possibility was explored in vitro herein, and found enhanced GFP expression in HEK293 cells co-infected with AdVCA0848 and an Ad5 vector expressing GFP (AdGFP) from the same CMV enhancer/promoter elements used in these studies (data not shown). These data also suggest that co-administration of the AdGag vaccine along with the strong c-di-GMP producing AdVCA0848 did not prevent Gag translation. It remains unclear how the significant induction of c-di-GMP and subsequently high levels of type I IFN can inhibit the T cell and B cell responses of an intracellularly expressed antigen (Quinn, K M et al. (2015) J Clin Invest 125: 1129-1146), and the impact of strong type I IFN induction on the availability of intracellular antigen-loaded APCs requires further investigation. It is noted that the production of another bacterial second messenger, c-di-AMP, by the intracellular pathogen Listeria monocytogenes was shown to induce IFN-β in a STING-dependent manner leading to the inhibition of T cell-mediated immunity, similar to our results with excessive production of c-di-GMP (Archer, K A et al. (2014) PLoS Pathog 10: e1003861).

In summary, demonstrated herein is the feasibility of in vivo synthesis of extremely large amounts of c-di-GMP via an Ad5-based platform expressing a highly potent DGC. While high amounts of c-di-GMP production can inhibit adaptive immune responses to antigens expressed simultaneously with significant increasing c-di-GMP levels, this unique platform appears to preferentially improve antigen specific B cell and T cell adaptive immune responses specific for co-administered extracellular antigens. This approach can be utilized to develop and improve protein-based prophylactic and therapeutic vaccines targeting infectious diseases and cancers.

### INCORPORATION BY REFERENCE

The contents of all references, patent applications, patents, and published patent applications, as well as the Figures and the Sequence Listing, cited throughout this application are hereby incorporated by reference.

### EQUIVALENTS

Those skilled in the art will recognize, or be able to ascertain using no more than routine experimentation, many equivalents to the specific embodiments of the present invention described herein. Such equivalents are intended to be encompassed by the following claims.

1. A vector comprising at least one cyclic di-nucleotide synthetase enzyme gene.

2. (canceled)

3. The vector of claim 1, wherein the vector is selected from the group consisting of adenovirus, adeno-associated virus (AAV), a replication defective adenoviral vector, retrovirus, and lentivirus.

4. The vector of claim 1, which is a DNA-based vector.

5-6. (canceled)

7. The vector of claim 1, wherein the at least one cyclic di-nucleotide synthetase enzyme gene is derived from a bacterial, fungal, protozoal, viral, or pathogenic strain.

8-9. (canceled)

10. The vector of claim 1, wherein the at least one cyclic di-nucleotide synthetase enzyme gene is selected from the group consisting of diadenylate cyclase (DAC), DncV, Hypr-GGDEF, DisA, cGAS, and diguanylate cyclase (DGC).

11. (canceled)

12. The vector of claim 10, wherein the DGC comprises a sequence which is at least 50% identical to the sequences set forth in Table 1.

13. The vector of claim 10, wherein the DGC gene is VCA0956 gene, and said VCA0956 gene comprises a nucleotide sequence which is at least 80% identical to SEQ ID NO: 33; or the DGC gene is VCA0848 gene, and said VCA0848 gene comprises a nucleotide sequence which is at least 80% identical to SEQ ID NO: 68.

14-16. (canceled)

17. The vector of claim 3, which comprises an adenovirus selected from non-human, human adenovirus serotype, or any adenovirus serotype developed as a gene transfer vector.

18. (canceled)

19. The vector of claim 3, wherein the adenovirus is human adenovirus serotype 5, said adenovirus has at least one mutation or deletion in at least one adenoviral gene, wherein said adenoviral gene is selected from the group consisting of E1A, E1B, E2A, E2B, E3, E4, L1, L2, L3, L4, and L5, or combinations thereof.

20-23. (canceled)

24. A combination comprising the vector of claim 1 further comprising at least one therapeutic agent, wherein the agent is another vaccine, an immunomodulatory drug, a checkpoint inhibitor, or a small molecule inhibitor.

25-28. (canceled)

29. A pharmaceutical composition comprising the vector of claim 1, and a pharmaceutically acceptable composition selected from the group consisting of excipients, diluents, and carriers.

30. (canceled)

31. An adjuvant comprising the vector of claim 1.

32. A vaccine comprising the vector of claim 1.

33. The vaccine of claim 32 further comprising an antigen, wherein the antigen is a viral-associated antigen, pathogenic-associated antigen, protozoal-associated antigen, bacterial-associated antigen, fungal antigen, or tumor-associated antigen.

34-37. (canceled)

38. The vaccine of claim 32, wherein the antigen is selected from the group consisting of Ovalbumin (OVA)-specific, HIV-1-derived Gag Ag, Clostridium difficile-derived toxin B, and Clostridium difficile-derived toxin A.

39. A method of inducing or enhancing an immune response in a mammal, comprising: administering to the mammal a pharmaceutically effective amount of the vaccine of claim 32 such that the immune response is enhanced or stimulated.

40. A method of treating a mammal having a condition that would benefit from upregulation of an immune response comprising administering to the subject a therapeutically effective amount of the vaccine of claim 32 such that the condition that would benefit from upregulation of an immune response is treated.

41. The method of claim 40, further comprising administering one or more additional compositions or therapies that upregulates an immune response or treats the condition, wherein the one or more additional compositions or therapies is selected from the group consisting of anti-viral therapy, immunotherapy, chemotherapy, radiation, and surgery.

42. (canceled)

43. The method of claim 40, wherein the condition that would benefit from upregulation of an immune response is selected from the group consisting of cancer, a viral infection, a bacterial infection, fungal infection, and a protozoan infection.

44. (canceled)

45. The method of claim 40, wherein the vaccine increases or stimulates cyclic di-GMP (c-di-GMP), cyclic di-AMP (c-di-AMP), cyclic GMP-AMP (cGAMP), any cyclic di-nucleotide, or combinations thereof, levels in said mammal.

46-51. (canceled)

52. The method of claim 40, wherein the mammal is a human.

53-66. (canceled)


SEQUENCE LISTING (170 sequences)

SEQ ID NO: 1 - DNA, 1884 bp, Vibrio cholerae
  source 1..1884: mol_type = genomic DNA
tcacgcaaagtgatgcatttccatggcggtgagtactgatatttggttgcgtcccgatgttttggattcatataaagccagatcggctcttttgtagctttggtcgggtgatgtgcaaacatcggtaacaccaccacttagggtcacttgttgatggtgtaatgaagcgatatgcaggcgtacgcggttaagtacttgttcggcttcttcaatggaagtgtaggggaaaataatggcaaactcttctccgccaatccgtgcgataaaatccgattcccgtaactgatcttggatgcctttcgcaacggtccgtaacactaggtccccttcgttgtgtccgaatttgtcgttaatgcgtttaaagtggtcgatatcaatgatagcaaggcagctctgggcttgatcgggataacggcgacgcttagcgcactctaaagagatggtttgatcgaatttacgtcgattccacaaatcggttaacgcatctttttcgctcagctcacgcaggcgattctccagcgccttgcgatgtgaaatatccacaaaagaggcaacgtagaattgaatgacattgtcttcatcgcggatgctttgaatacggagaatttcggtgatgctttcgccatctttgcgtttgttgatcacttcaccttcccatacgccattgtcttgcagagctttccacatctgcatatagaattcgactttgtgtaatccagaagcaaaaatggacggctgcttacctttgacatcttcaaaagtgtaaccacttaggcgggtaaattcgttgtttactttgatgatgcgattctggcggtcggtaatgaccaccgctgacatgccatccatcgctgctcgagccaatttactgtcaaggctattttttaaatggttgatgttccatgccgcaaatccagccgcaatgatagagagtagcgataacactgtcaccgcttgactcatcagtgcccagcgcgcatttgcgtaggtcttatctatttctgccttattgatgcgcagtaccaataccaaaggtttaaagtcaggtaagacagaactgagatccactttgatatagctaaaccaggtttgattggatagagcaaagccttgttggttgagttggattttttgccaaagctctgggtgttgggctgaaaagtggagtgaagaggttgaacgtgtaccggatggcttgtgttcactgagcagtaattctcctgccgaattcaaaatatccggtgaatcaaactgatcataaataaaagagagacgttgatagagagactgtagcttcaccgtcacgacaagaaaaccttgccgttggccttgatgctcaatacccgtcacaaaacgaaaggtcggcagcataccagaaggcgtatctgctgacatcgcgacttgcgttgcccaaacttgaggcgtcgtgagttgggcgtattgagccacaatttgctggctgaacggatctgtcgtttgagcagattcaacaaaggtgacttggtgcccatcgtaaatcgctttaagttgttcttttccttgtctatccagcaatctgaatgaagagaaaatcgcttgcgatcttaacgtcacatcccacaatgttttgagttgactgagtgcttctttgcttggtgtggtgacagccgtgaataaaaggtcatttttagctaacagctgggtggcttggtgtgtgctttccagcattcgtaacaagtcatgctgactgaactcaagctgtaagcgagtctgtttttcaacgctgctgaccgcttgagtctcaagctggctagcagcatgtatgaaatacagtgtaggaatgaaaccaagtacaaacgcaacaatggcaaattgtatgaaatatttacgggctgaggtgtacat

SEQ ID NO: 2 - protein, 627 aa, Vibrio cholerae
MYTSARKYFIQFAIVAFVLGFIPTLYFIHAASQLETQAVSSVEKQTRLQLEFSQHDLLRMLESTHQATQLLAKNDLLFTAVTTPSKEALSQLKTLWDVTLRSQAIFSSFRLLDRQGKEQLKAIYDGHQVTFVESAQTTDPFSQQIVAQYAQLTTPQVWATQVAMSADTPSGMLPTFRFVTGIEHQGQRQGFLVVTVKLQSLYQRLSFIYDQFDSPDILNSAGELLLSEHKPSGTRSTSSLHFSAQHPELWQKIQLNQQGFALSNQTWFSYIKVDLSSVLPDFKPLVLVLRINKAEIDKTYANARWALMSQAVTVLSLLSIIAAGFAAWNINHLKNSLDSKLARAAMDGMSAVVITDRQNRIIKVNNEFTRLSGYTFEDVKGKQPSIFASGLHKVEFYMQMWKALQDNGVWEGEVINKRKDGESITEILRIQSIRDEDNVIQFYVASFVDISHRKALENRLRELSEKDALTDLWNRRKFDQTISLECAKRRRYPDQAQSCLAIIDIDHFKRINDKFGHNEGDLVLRTVAKGIQDQLRESDFIARIGGEEFAIIFPYTSIEEAEQVLNRVRLHIASLHHQQVTLSGGVTDVCTSPDQSYKRADLALYESKTSGRNQISVLTAMEMHHFA

SEQ ID NO: 3 - DNA, 1386 bp, Vibrio cholerae
  source 1..1386: mol_type = genomic DNA
ttatgaccaggtacgaaagacaacctggttctttccattccgctttccttcgtacattaagctatcggcatcgtgcaaactgaatggccgagctgggtgtaggtaaaatgcacagcctaggctgatggtgagtgacagagagtgttgggcattcactacccactttttttctgcaactcgttggcaaattcgctcagctaactgctgcgactcttctgcatttttaccacgcgctacaatagcaaactcttcaccaccaatccttgcaaagtaggtatccgatgctaaagcttgtcgcacacacccaaccacgaaacagatggcattatctcctgcgccatgcccaaagcgatcgttaatggttttgaagtcatcaatatcaaaaaccatcaacgttaagctgcctgatcgtgtttgttccgcttcaagatgttcaaaaaacgaacggcgattagcaatgcccgtcaagctatccgttttcgctaaataggagagtttttgattggcttcttcaagttgctgtgttcgcaatcgaacggtacgccgtagctgaagagtataaataacgatactgagtaagagacctgaagcgagaatcggcattaagtaacgtggataaatcgtttcaatatgaacccatcgacttaaaatacggtttttctcattgctacttaattgtgcaaacccctgctctacttgctctaataaatccctattgcctttggcgaccgctggacgtaattcctctgaataaagaaacttcactggcgtaaaatctttcgcgccattggaaaccactatatagaaattggcgacctgagtatcggccacaaaaccatctaattctcgtcgctttgctgcagacatcatcaattcattgttggcgtactcaatcaacttaagttgaggatattctcgttgcatgaactcttgttcaaatcccccttttactacacctaatgagacgttaatggcccccgatagcagcgtatccaatttatcgcccaataacgtgcggtgtacgtagagttgtgtatcgattgtcagtaaaggttctgcaaaatcgagatacgctaatcttgaagcagaacggatcaaaccagcttgaacatcggatttgccaagcttcaccgcttctagggaatcattccaatccatcagttggaattcaatatcgacatgattcgcttcaccaaaagccaaccaaaaatcaatcaatatgccagaaggctgtccctgttcatccaaataagaatagggtttccatgcttttgagttggcaatagtcaaggtttggcgctctacagcctcactcattgatccgaataaaagcggccaagcaatcatgagaagcagaaacagtttggtcgaaaagcgatgatccat

SEQ ID NO: 4 - protein, 461 aa, Vibrio cholerae
MDHRFSTKLFLLLMIAWPLLFGSMSEAVERQTLTIANSKAWKPYSYLDEQGQPSGILIDFWLAFGEANHVDIEFQLMDWNDSLEAVKLGKSDVQAGLIRSASRLAYLDFAEPLLTIDTQLYVHRTLLGDKLDTLLSGAINVSLGVVKGGFEQEFMQREYPQLKLIEYANNELMMSAAKRRELDGFVADTQVANFYIVVSNGAKDFTPVKFLYSEELRPAVAKGNRDLLEQVEQGFAQLSSNEKNRILSRWVHIETIYPRYLMPILASGLLLSIVIYTLQLRRTVRLRTQQLEEANQKLSYLAKTDSLTGIANRRSFFEHLEAEQTRSGSLTLMVFDIDDFKTINDRFGHGAGDNAICFVVGCVRQALASDTYFARIGGEEFAIVARGKNAEESQQLAERICQRVAEKKWVVNAQHSLSLTISLGCAFYLHPARPFSLHDADSLMYEGKRNGKNQVVFRTWS

SEQ ID NO: 5 - DNA, 927 bp, Vibrio cholerae
  source 1..927: mol_type = genomic DNA
atgatagaacttaatagaattgaagagctttttgataaccaacagttctccttgcacgaactcgtgttgaacgaactgggagtctatgtcttcgtcaaaaatcgccgcggcgagtatctctatgctaaccctctgactctaaagttgtttgaagcggatgcacaatcgttgtttggcaagaccgatcacgatttttttcatgatgatcaactcagtgatatcttggcggccgatcaacaggtgtttgaaactcgtctctcggttatccatgaagaacgagccatcgccaaatccaatggtttggttcggatttatcgcgcagtcaaacaccctatcttgcaccgagtgacaggcgaagtgattgggctgattggagtttcaaccgatatcaccgatatcgtggaactgcgtgagcagctatatcagctcgccaataccgattctttaactcagctgtgtaatcggcgtaaattgtgggccgattttcgcgccgccttcgctcgcgcaaaacgtttaagacagccgttaagttgcatctctatcgatattgataatttcaaactgatcaatgaccaatttggtcacgataaaggtgatgaagtcctgtgttttctcgccaaactatttcagagcgtcatctctgaccatcatttttgtggtcgtgtgggaggtgaagagttcatcatcgttttggaaaatacgcacgtagagacggcttttcatttggctgaacagatccgccaacgttttgcagagcatccgttctttgaacaaaacgagcacatctacctctgtgcgggggtttccagcttgcatcatggtgatcatgacattgccgatatttatcgacgctccgatcaagcactgtataaagccaagcgtaatggtcgtaaccgttgctgtatctatcgccaatccacagaataa

SEQ ID NO: 6 - protein, 308 aa, Vibrio cholerae
MIELNRIEELFDNQQFSLHELVLNELGVYVFVKNRRGEYLYANPLTLKLFEADAQSLFGKTDHDFFHDDQLSDILAADQQVFETRLSVIHEERAIAKSNGLVRIYRAVKHPILHRVTGEVIGLIGVSTDITDIVELREQLYQLANTDSLTQLCNRRKLWADFRAAFARAKRLRQPLSCISIDIDNFKLINDQFGHDKGDEVLCFLAKLFQSVISDHHFCGRVGGEEFIIVLENTHVETAFHLAEQIRQRFAEHPFFEQNEHIYLCAGVSSLHHGDHDIADIYRRSDQALYKAKRNGRNRCCIYRQSTE

SEQ ID NO: 7 - DNA, 1374 bp, Vibrio cholerae
  source 1..1374: mol_type = genomic DNA
tcaaaagcgatagagtgggttttgcctacgcttagcggtatacatacgttcatcggccagtttgaacatttcatcaggtgtggcaaacgactggtcatacaaagcatatccgatacttacacgaacatggataagcttgtcgtcataaacgatgggcgtttcagaaatcctttttaaaatattgtcactgactttaagcacgtcttgttcacgatgaattcgtggaattaacacgagaaactcatcccccccaatccgcgccaccagatcggaaacccgcaggctcgatttaattctttccgcacaagccaccagcactttatcgcctgcgctatgtccatgggaatcgttgatagatttaaaacggtcaatatcaatgttcaacaaagcaaagttaccttcgctatgagagcgcttagcattttcaaagtagtgttcaatggtatagataaaatagcgccgattcggcaagtgggttaaagggtcatgtagcgcacgctcctccgcgacttgataaaggcgcatgataacgccaaagcctgccatcaataccaataacaccgagtatcccaacaagcgcactgcatttcgggtataccaagataactgctgtagtaaatcttgcttttcagcgaccgcaattcgccaacttccgtaagggaaatagacattctcttgtgcaaaagcgtgctcaaatactcgaggctctccaaaaaacacgtccccctcactgccacggctgtctaaaccacgaatcgcaacctgaaaatgctccccaaagctgtaaatactggttgctgaaagcaatgaatcccaatccatcaccacactcagtaccccccaataacgcgtatccttcggtgggtcgtagaatatcggttctcgaatcaccagcgcgcgcccaccttgaacgagatcgacaggtccagagacgaacgtctgtttgatttcacgtgctttttttattgactgccactgctgaggaacggtgcggtaatccaaaccgagtagtgcattggtttgaggaagcggatagctgaaagcgaccacatcattaggggcgatacctaatgagcgtaagtgatcgctattcctgatcaccgccgctgaaagcggctcccattgatagatattgaggtcgggatctagggttaacagggttgttaaaccttttacggtatagatatcacccaaaatctcagcttctaattgaaaacgtacgatggaaagatcttctttagcttgttgacgtaaaccctcttgtagatcacgtgtatggctaatatgaagggattcaataaccgcaatgcccaaaaagagtaaggcgagaaaataaattgagacatacttatatttgtgcgaggttaaccccat

SEQ ID NO: 8 - protein, 457 aa, Vibrio cholerae
MGLTSHKYKYVSIYFLALLFLGIAVIESLHISHTRDLQEGLRQQAKEDLSIVRFQLEAEILGDIYTVKGLTTLLTLDPDLNIYQWEPLSAAVIRNSDHLRSLGIAPNDVVAFSYPLPQTNALLGLDYRTVPQQWQSIKKAREIKQTFVSGPVDLVQGGRALVIREPIFYDPPKDTRYWGVLSVVMDWDSLLSATSIYSFGEHFQVAIRGLDSRGSEGDVFFGEPRVFEHAFAQENVYFPYGSWRIAVAEKQDLLQQLSWYTRNAVRLLGYSVLLVLMAGFGVIMRLYQVAEERALHDPLTHLPNRRYFIYTIEHYFENAKRSHSEGNFALLNIDIDRFKSINDSHGHSAGDKVLVACAERIKSSLRVSDLVARIGGDEFLVLIPRIHREQDVLKVSDNILKRISETPIVYDDKLIHVRVSIGYALYDQSFATPDEMFKLADERMYTAKRRQNPLYRF

SEQ ID NO: 9 - DNA, 1374 bp, Vibrio cholerae; identical to SEQ ID NO: 7
  source 1..1374: mol_type = genomic DNA

SEQ ID NO: 10 - protein, 457 aa, Vibrio cholerae; identical to SEQ ID NO: 8

SEQ ID NO: 11 - DNA, 1176 bp, Vibrio cholerae
  source 1..1176: mol_type = genomic DNA
atggatagctttgctggcaaccaattaaaagagatgacagagatgcgttttgctcgtaagcagcatattgtcctgatcagctctggtgttgctaccgctatttttcttgggtttgccctttactactattttaaccatcaacccctgtcatccggtttattgttattaagcggtattgtcaccttattgaatatgatttcgctgaatcgtcaccgcgaattacacactcaagccgatttaattctgtcattaattctgctcacttatgcgctggccttagtcagcaatgctcagcatgaattatcgcatctcttatggttatatccgctcatcaccactttagtcatgattaacccttttcggttaggcttggtttacagtgcagcgatatgcttagcgatgaccgcctctatcctttttaatccggcacaaactggctcgtaccctattgcacagacctattttttagtaagtctatttacgctgacgattatctgtaataccgcttctttctttttctcaaaagcgatcaattatattcataccctataccaagaaggtattgaagagttggcttatcttgatccgttaacgggcttagccaatcgttggagctttgaaacttgggccacagaaaagctcaaagaacaacagagttcgaataccattaccgcgcttgtttttctggatattgataatttcaaacgcattaatgacagttacggccatgatgttggcgatcaggtgttaaaacattttgcacaccgtctacgcaataatattcgtaataaagatcgagccaccaatcaacatgattattccattgctcgatttgctggtgatgagtttgtgctcttgttatatggtgtgcgaaatttgcgtgatctcgataatattctcaaccgtatctgtaatctcttcgtcgaccgctatcctgagacggatatgctcaacaacctcacggtgagtataggggcagctatttatcccaaagatgcgatcactctgccggaactaacccgctgcgcagataaagccatgtatgccgctaaacacggtggaaaaaatcagtaccgctattaccatgatgccgctttccctccggctgtagaaaccgtattaggcagtcagcccgttgaggctcctaacgtaactccactgaaaaaagcgcactaa

SEQ ID NO: 12 - protein, 391 aa, Vibrio cholerae
MDSFAGNQLKEMTEMRFARKQHIVLISSGVATAIFLGFALYYYFNHQPLSSGLLLLSGIVTLLNMISLNRHRELHTQADLILSLILLTYALALVSNAQHELSHLLWLYPLITTLVMINPFRLGLVYSAAICLAMTASILFNPAQTGSYPIAQTYFLVSLFTLTIICNTASFFFSKAINYIHTLYQEGIEELAYLDPLTGLANRWSFETWATEKLKEQQSSNTITALVFLDIDNFKRINDSYGHDVGDQVLKHFAHRLRNNIRNKDRATNQHDYSIARFAGDEFVLLLYGVRNLRDLDNILNRICNLFVDRYPETDMLNNLTVSIGAAIYPKDAITLPELTRCADKAMYAAKHGGKNQYRYYHDAAFPPAVETVLGSQPVEAPNVTPLKKAH

SEQ ID NO: 13 - DNA, 447 bp, Vibrio cholerae
  source 1..447: mol_type = genomic DNA
atgctagcgttacctgcggagtttgagcaattccattggatggtcgatatggttcagaatgtcgatatgggattgattgtgattaaccgagactacaacgtgcaagtgtggaatgggtttatgacccatcatagcggtaagcaagctcatgatgttattggtaaatctctgttcgagatttttccagagatccctgtggagtggtttaagttaaaaaccaaaccggtgtacgatctgggttgccgtagttttattacttggcagcagcgcccttatttgttccattgccgtaatgtgcgcccagtgactcagcaagccaaatttatgtatcaaaacgtcacgcttaacccaatgcgtacaccgacaggcgcgataaattcactcttcttatccattcaagatgcaacaagtgaagcccttgtttctcaacaagcttcttctcaataa

SEQ ID NO: 14 - protein, 148 aa, Vibrio cholerae
MLALPAEFEQFHWMVDMVQNVDMGLIVINRDYNVQVWNGFMTHHSGKQAHDVIGKSLFEIFPEIPVEWFKLKTKPVYDLGCRSFITWQQRPYLFHCRNVRPVTQQAKFMYQNVTLNPMRTPTGAINSLFLSIQDATSEALVSQQASSQ

SEQ ID NO: 15 - DNA, 1518 bp, Vibrio cholerae
  source 1..1518: mol_type = genomic DNA
ttagacaaaatttcgcacaacgtatcgatctcgtccgtgttctttcgcatgataaagtgccatatccgcctgatggaacaaagagagataagactccatctttggagaaatagcatacacaccaccaatgctcaccgttagatattggcagagtgcatcaaccggatttgcaatcgcgagctgctcgattttgcttctcatctgttgtgcatactgttctgcatcaaatgcacagtccgaagctaaaacaacacaaaactcttctcccccaaagcgcgccacgattttctcgccatggaactccaccgattggagcacatcagcaacggaacataaggcttcatcgccagccaaatgaccaaagctgtcattgaaacgtttgaaaaaatcgatatcgacaagaaacagcaccagataggcttgcggacgatcgctcaaataacttttaagctgcttttctaaatggcgacgattggaaatgcgggttagtggatcatgctcagactgccaacgtaacacttgttgactatcctccaattgtccgacgattcggttgatcgtagtggcaaattctttcatctccgatgagataaaagtactcgcatccggcatttttccgcccgatgttttaaattgttgcaacacttgactggcggtcgtgatcggtttgatcaaggcaatcaccacccataaattgactaagtacatcaccagtgaaaagaacagcaaagcaagaatttcttcggttcgaatgaagggaggatgcttaatgtgatggttaattttaaacaacacactggaattaccgctgtaatcgagttgcttgatgtatgaaacatccacttcgtcttgcggtaagggcgcatcatttttacaggttaagacttcaatatcgacaccagtggcttgctcaaccacattcgcaaactgggcgcggacttttttaataaagattaagaaacctttgttacaccctttcccatcactgtcacagacacgagccgtggcagctaaatagggctcatcctccaccaccatataacgaacggaagtcgagatttcatccacacttaaacgtgtcgcctgctgtaaaatacgtgaaaaatccggcaataagtgctcatagctagagctctgccccgttgctgcgtcatatttcttgccccaaaccaaattgccctcaggatcatagataaatacgccatcgaggaattgtgaactgaaagcgtgctctccaatattgctttgtgtgaactcaagggtgggttttgcaatgaagtctgccatttcatcccaagcggcataatctgccaaagaagcccccatcgccttacgttctaacgacaacaaggtttcaacccgctgcaactcggcctgttgtaactgcagcacttgcgcaacttcacgatcatgtgaccagaaatatttaaaggtcagataaaacattaaaaagcctaacaccaccgctaacgcattgagtgtcgttagccagcgtaggctaaagttatttaaattcat

SEQ ID NO: 16 - protein, 505 aa, Vibrio cholerae
MNLNNFSLRWLTTLNALAVVLGFLMFYLTFKYFWSHDREVAQVLQLQQAELQRVETLLSLERKAMGASLADYAAWDEMADFIAKPTLEFTQSNIGEHAFSSQFLDGVFIYDPEGNLVWGKKYDAATGQSSSYEHLLPDFSRILQQATRLSVDEISTSVRYMVVEDEPYLAATARVCDSDGKGCNKGFLIFIKKVRAQFANVVEQATGVDIEVLTCKNDAPLPQDEVDVSYIKQLDYSGNSSVLFKINHHIKHPPFIRTEEILALLFFSLVMYLVNLWVVIALIKPITTASQVLQQFKTSGGKMPDASTFISSEMKEFATTINRIVGQLEDSQQVLRWQSEHDPLTRISNRRHLEKQLKSYLSDRPQAYLVLFLVDIDFFKRFNDSFGHLAGDEALCSVADVLQSVEFHGEKIVARFGGEEFCVVLASDCAFDAEQYAQQMRSKIEQLAIANPVDALCQYLTVSIGGVYAISPKMESYLSLFHQADMALYHAKEHGRDRYVVRNFV

SEQ ID NO: 17 - DNA, 1740 bp, Vibrio cholerae
  source 1..1740: mol_type = genomic DNA
ttagtggtttggttgataaattgaggtctgattgcggccattcgctttggcttggtataaagcccgatccgctagctcaaccatttgctcaggtacatcctcaggccgaggaataagcgtcactatgcctaagctgacggtaatcctatcggcaaccttagaatgatcatgtggaatcgctaatccacgaactttctcatggattcgctctgcgaccagtattgctccggactgtggtgtattgggcagcaaaataccaaactcttctcccccgtagcgggcaacacaatcagaatggcgattggcgacttgagtaaaggcaatcgctatctgtttgagcgtctcatcgcccatcaaatggccataagcgtcgttgtaatctttgaaataatcgacatcacacagaatgatgcttaatggtttgccttcacgcacatgcaaatgccagagggtatgcagttgttcatcaaaacgacgacgattggcaacatgagtcaagctatctaaaaagcttaggcgttccagctcttggttggcggcttctaattgttcagcggcgagatagcgctccgacacatctcgcgccatgatcagcacgccattggtgcccgaagccggatctcgaaaaggcgatttcacaacatcaaaccagataaactcaccatctgagcgttcaattctgtcgatgtagcgcagagacttaccttggtgcaggacttggctatccgtatcggaaagacgcgcatagatgtgctcggggatcacatcttgcagccgtttaccaaccagatctgacacttccgcgatcccgagagcttccacaaacggctggttacaggcttggtagaccatgttttcattgaagataccaatcgaatcggggctagattctaagatgttttgtaaaatcgtatcgcgctgtgccaatgccacttcggtgtcacggcgtttttccatctcttctcttaattgacgctgcatgttgtaccagtcggtcacatcatgactgatgccaagtagcccaatattttcaccttgcggcgacatcaatacccgttggtaggtttctaacagacagctgcgcccatcaggcgtcacagtccagcaacgctgactcgtgcgccctttcataatgcctttaaaagtagcgctgccctcttcaatccggccttgccaaaactgatcaaacgctcggttggttgcgattaagtggccttcggtacttttaataaaaatcagctcggagagggaatcaagtgccgtgcgcgctatcgccagtgagtggcgctcttgttgaatgtcatggctgggacactcaaaaccaatcacattcactagccataatttcttcggccaacgacgtaagagcgaagctgagatctctagagtttgggtcaaattgcccggcacaggccaaagcagagggacggaacgcttttgctgtgcactgctggcgagcgctcgataaaaagcttgctgactctcttcactctgctcggcagaaaacagatagtgacgtcccaccaagcggatccccagtaacaaatacgcggcaagattggcacgtaaaacgcgatcctctcctaccaagagcatccctgacggtgcatggtgaagtaactgaatccactgttgaggttgaacatagcgctgccatcctgaaaaaagccataacccaccaccaagcacaagcccggcagcgaacaagaaacgtacaaattcagagagaaattcaggcat

SEQ ID NO: 18 - protein, 579 aa, Vibrio cholerae
MPEFLSEFVRFLFAAGLVLGGGLWLFSGWQRYVQPQQWIQLLHHAPSGMLLVGEDRVLRANLAAYLLLGIRLVGRHYLFSAEQSEESQQAFYRALASSAQQKRSVPLLWPVPGNLTQTLEISASLLRRWPKKLWLVNVIGFECPSHDIQQERHSLAIARTALDSLSELIFIKSTEGHLIATNRAFDQFWQGRIEEGSATFKGIMKGRTSQRCWTVTPDGRSCLLETYQRVLMSPQGENIGLLGISHDVTDWYNMQRQLREEMEKRRDTEVALAQRDTILQNILESSPDSIGIFNENMVYQACNQPFVEALGIAEVSDLVGKRLQDVIPEHIYARLSDTDSQVLHQGKSLRYIDRIERSDGEFIWFDVVKSPFRDPASGTNGVLIMARDVSERYLAAEQLEAANQELERLSFLDSLTHVANRRRFDEQLHTLWHLHVREGKPLSIILCDVDYFKDYNDAYGHLMGDETLKQIAIAFTQVANRHSDCVARYGGEEFGILLPNTPQSGAILVAERIHEKVRGLAIPHDHSKVADRITVSLGIVTLIPRPEDVPEQMVELADRALYQAKANGRNQTSIYQPNH

SEQ ID NO: 19 - DNA, 1584 bp, Vibrio cholerae
  source 1..1584: mol_type = genomic DNA
ttacataaagtcgaacatcctacctgaattgaaggcataattcgattctaccttgctgcattgctgcgcaatcgatacacgatttcgacctttcgatttactgagatagagctgatcatcaacactctgtaaaatttccggctcactgtactcacagttaatgctcgccccaatactgatggttaaggttaatggtgtctcggcattgagcatcacaggttctgcttcgaccactttacggatccgctctagataagtataaagcgccgtttcatcagtaacggatgacaagatggcaaactcatcaccgccgaaacgggcaaaaatatccgattcaaccaactcttttttgaccacatcaaccacatgcgttaaagcgtaatcccccgctaaatgcccatagctgtcgttgatttgcttaaagcggtcgatatcaaatgaaatcaaggtaaaggattgtttttcatctaacattttgcacaaatgctgactaaagaagcggcggttatagatgttggtcaaactgtcatgctccaccagataacgcagctctgcggtacgctcctcaatcatatctgtcagccgttgtttctcttccagttgcattcgcatgatgtagctaagcagcagagaaataataacgccacccaaccctaagcccagtagcacccactcttcactatggttaatcggctgatgcagttcaaactccagcacccaatcacggtttggcaacaccaatttgcgctctattttgggttcatcatccgctcgccacatcgggctttgataaagaaccggactgtcttccgaatcaaatccggtgtcaatcacgcgcatatcgagatcttgttccatgacgctgatttggaccagtttctcgaaataggtggataggcgcaccaccccgaccatcacaccaagtaagctgcgatcatcttctgaagaaaaaacagggtgatagaccaacatgccatctttgacgatcgacttatcaatcccatcttgtagcaggcgcactttatccgaaacattcggccgacgattaacgacaatatccgccagtattcgtttgaaacgttcacgctccgagtaaaagcctaacagtttacgattgtcataattgagtggataaatatccgataaaacgtatttcgcttggtcatccgtaccgaaaccgtatttgatctctcccgtttttggcaccgtgtacaaagtgaactcaggaaaacgttgctgcattcgcgcggtaaaagtttcagcctgaggcggctcaactttcactaaccattgtaaagcaatcaggctttgtgaacctttaagagtctcttctgcgaaagtgtgaaaacgcacccagtcatcgcttgtgcttgagcggaaaaagttggcggcagagccgataaaatggatatcaccatcgacaaactgttgcagtgccatagtttgcctatccgcaaggttttccagcagagtacgattatggcgcagctgtaatgagtatgcggtgtaaaccacaaacacagtcagaagcagagaaaacaacagtaccagcaagggcacaatcacgcgcacatgtttgagcat

SEQ ID NO: 20 - protein, 527 aa, Vibrio cholerae
MLKHVRVIVPLLVLLFSLLLTVFVVYTAYSLQLRHNRTLLENLADRQTMALQQFVDGDIHFIGSAANFFRSSTSDDWVRFHTFAEETLKGSQSLIALQWLVKVEPPQAETFTARMQQRFPEFTLYTVPKTGEIKYGFGTDDQAKYVLSDIYPLNYDNRKLLGFYSERERFKRILADIVVNRRPNVSDKVRLLQDGIDKSIVKDGMLVYHPVFSSEDDRSLLGVMVGVVRLSTYFEKLVQISVMEQDLDMRVIDTGFDSEDSPVLYQSPMWRADDEPKIERKLVLPNRDWVLEFELHQPINHSEEWVLLGLGLGGVIISLLLSYIMRMQLEEKQRLTDMIEERTAELRYLVEHDSLTNIYNRRFFSQHLCKMLDEKQSFTLISFDIDRFKQINDSYGHLAGDYALTHVVDVVKKELVESDIFARFGGDEFAILSSVTDETALYTYLERIRKVVEAEPVMLNAETPLTLTISIGASINCEYSEPEILQSVDDQLYLSKSKGRNRVSIAQQCSKVESNYAFNSGRMFDFM

SEQ ID NO: 21 - DNA, 1332 bp, Vibrio cholerae
  source 1..1332: mol_type = genomic DNA
tcagctcactaaactggtgtgatcgtgcttatcttggtgggcgcaatacaccgtattgccggattgatgttttgcggtgtacatcgcctcatcagcaatacgcaataattcaggtacttgggtcgcttgctctggatataaggcgacaccaatactcaccccaatctctaagctctcttggttaagttgaagcggcttttgtagtttttctagcatctgataagccttattgataacgccactgtgatcctgcagcagatctaggcataccacaaattcatccccccctaagcgcccacaaaaatccgattctcgtatcgaccctttgagccgttgagcgatttcttgcaagacaagatcacctacttcgtgccctttggtgtcattgatttctttaaatttatctaagtcaaaaaacagcaaagccagcttcatgtttgagcgcttcgctttaattaacgcgtgactaagctgctgtttaaaggcacggcggttcaaaatacctgtcaatgaatctctttctgacaagaaacgtaattccgctttttgacgctctaatttggcggtttttcttgccacttccgcttgtaactcatctttggtaacggttgtgctttgcagcgaagccttcatttgattgaaaaactgagttaattgaacaaactcttgttcattattttgagtggaaattcggctggcgagatcccctttcgccatttgttcaatcccttcttggagagttttacatccatgtcggaagcggcgtaacaccaccagcgcgataccacagacaaccgatgagaagagcagtaagtgcgccatggtggttaacaataaatagcgttgattattaatgctctcttccatgacttgacgctgaaaataggccaactcctcattcatgttttgcaccaaaatattgtatcgagagtgaagtagctcataagttccgatgccatcgaccaacttagtaatgcccgattcttcggccatgtagcgctcttgttctaatagcccggctaaactgttattcattctttggatgccggctaagtgttgcccaaagaccgtttccatctcgagctgcccagccaaaacctgctgcgcacgataaacctgctctaagctatgagcatcgttgtattgcagaaagacccagagctggctacgcaacatggcaatgctgttttggatttccaaaatcgtatccagctcagcattggtttgctgctgccgctgatctaagttcagtaatgagaaagcaataaaaccaactaacagcagtgatgcaataaacagtaacgtcattttgcggtttaatgagttgatcaa

SEQ ID NO: 22 - protein, 443 aa, Vibrio cholerae
MINSLNRKMTLLFIASLLLVGFIAFSLLNLDQRQQQTNAELDTILEIQNSIAMLRSQLWVFLQYNDAHSLEQVYRAQQVLAGQLEMETVFGQHLAGIQRMNNSLAGLLEQERYMAEESGITKLVDGIGTYELLHSRYNILVQNMNEELAYFQRQVMEESINNQRYLLLTTMAHLLLFSSVVCGIALVVLRRFRHGCKTLQEGIEQMAKGDLASRISTQNNEQEFVQLTQFFNQMKASLQSTTVTKDELQAEVARKTAKLERQKAELRFLSERDSLTGILNRRAFKQQLSHALIKAKRSNMKLALLFFDLDKFKEINDTKGHEVGDLVLQEIAQRLKGSIRESDFCGRLGGDEFVVCLDLLQDHSGVINKAYQMLEKLQKPLQLNQESLEIGVSIGVALYPEQATQVPELLRIADEAMYTAKHQSGNTVYCAHQDKHDHTSLVS

SEQ ID NO: 23 - DNA, 2022 bp, Vibrio cholerae
  source 1..2022: mol_type = genomic DNA
atgctacttaacgctttttcacgccgagtcttcctttggctaggttggctattgatttccaccagcagtttagccgctacatctacgacgtataaggtcgccaccgaagcggatgacgtggtgactcgtgtgctttttgattcgattgctcaccacttcaaccttgaaattgaatacgtcaactaccccagttttaacgatattctggtggcgatagagactggcaacgccgattttgctgccaacattacttacactgatttgcgtgctcaacgttttgatttttcaagaccaaccaacatcgagtacacctatctctacagttatggtggcctacgtttacccgagttgcgcctcgtgggtatcccgaaaggaaccacctacgggaccctactaaaagaacactatccctatatccagcaagttgagtatgaagggcatttagaagcgctcactttgctggaaagtggccgagtagacggagtggttgatgcgatcaatcagctcaaacctatgctactgaaagggcttgatgtacaactccttaacgaccaattaccgattcagcctgtttctattgtgacgcctaaaggcaaacactcagcgctattgggcaagattgaaaaatacgcgcattcggctcacgtacaacgtttattgcgtgaatcgatccaaaagtatcaattggacatccgtaagcaagctctgcgtcaatccgtggttgagagcggactcaacgtgcagcgtgtattgcgtgttaagctagagaacaacccgcaatatgcactttatcagccagacggttcggttcgtgggatcagtgctgatgttgtgtttcaggcctgtgagatgctactgctgaaatgcgaattggtcagtaatggtcaagaaacatgggagagcatgtttgatgatttacaggataaaagcatcgatattttggctcctataacggtttctcagcagcgtaaaaacctcgcttacttcagtgaaagctactaccacccacaagcgattttggtcaaacgtgaacactataaagacgatgtgtatagcaatgtgtctgagttggtggctgaacgtattggcgtcatcaaagacgatttttttgaagagctgttacagcagatgctgccgaacaagatcttgttcagctacgcaagtcaggaagagaaagttcaagccttactgaataaagaggtggactacatagtgctcaatagagccaattttaatctcttgcttcgcgagtcaacggagatgttaccgattgtagaagacaccatgattggcagtttctaccaatatgacattgcgataggttttgctaaaaatccacttggtgcaactctggcacctcttttctctcgggcaattaaaatgctcaataccgaacagatcatacatacctatgattatcagccaaattggcgagccacattacttgcggaaaagaaatatcagcgcagtactcaatggctttttgccatggctttcatcgttttgtttatggtggcgttttacctccatggcatatcacataccgataaccttactaagttgcgcaatcgtcgcgctttgtataaccgataccgccgcgggttatcgcctcgcctaagcttggtttatcttgacgtgaatacgtttaaatcaatcaacgatcagtatggacatgaagtgggtgacaaagtccttaagcagttggctcagcgcatcgaagcggtatggcgtgggcgcagctatcggattggtggggatgaatttattttaatcggtgaatgttctgctaagcggcttgaacatgtggttgcgcaatgtgaacgttttatgtttgtggatgcagagcgcgatgtcagttttgaagtgagtgtggcgattggtattgctaagaatcgtgagcggaccgaatcactcaatgaggtgatgcaccaagcggatattgcgatgtatcgcgctaaggcggaatcgacgcaatcgccatttcaggctgccagcaaggtaaaaggattacacatcgtttaa

SEQ ID NO: 24 - protein, 673 aa, Vibrio cholerae
MLLNAFSRRVFLWLGWLLISTSSLAATSTTYKVATEADDVVTRVLFDSIAHHFNLEIEYVNYPSFNDILVAIETGNADFAANITYTDLRAQRFDFSRPTNIEYTYLYSYGGLRLPELRLVGIPKGTTYGTLLKEHYPYIQQVEYEGHLEALTLLESGRVDGVVDAINQLKPMLLKGLDVQLLNDQLPIQPVSIVTPKGKHSALLGKIEKYAHSAHVQRLLRESIQKYQLDIRKQALRQSVVESGLNVQRVLRVKLENNPQYALYQPDGSVRGISADVVFQACEMLLLKCELVSNGQETWESMFDDLQDKSIDILAPITVSQQRKNLAYFSESYYHPQAILVKREHYKDDVYSNVSELVAERIGVIKDDFFEELLQQMLPNKILFSYASQEEKVQALLNKEVDYIVLNRANFNLLLRESTEMLPIVEDTMIGSFYQYDIAIGFAKNPLGATLAPLFSRAIKMLNTEQIIHTYDYQPNWRATLLAEKKYQRSTQWLFAMAFIVLFMVAFYLHGISHTDNLTKLRNRRALYNRYRRGLSPRLSLVYLDVNTFKSINDQYGHEVGDKVLKQLAQRIEAVWRGRSYRIGGDEFILIGECSAKRLEHVVAQCERFMFVDAERDVSFEVSVAIGIAKNRERTESLNEVMHQADIAMYRAKAESTQSPFQAASKVKGLHIV

SEQ ID NO: 25 - DNA, 1293 bp, Vibrio cholerae
  source 1..1293: mol_type = genomic DNA
ctatctgaactgatcctgcttgagttctttcgcactgggaagaggcaggatctcttcccccattcgataaatatgatagccatgtttgcctctgtatttgacccagtacatggctttatcggcttgtagcagcagtttttctaagtcaatgtgcagactgttcatatgactaatcccgatactgcaacccacttgcgcactctgctgacccaatccaatcggctcagaggaggattcgatcaactgagccgcaaaccgctcgatagattcggcaacaaattcatccagcggaatgtaaatagcaaactcatcaccaccgagccgtccgaccacaaaatcagaaaaatgtgtttgcgccaaggcataaaaacgtttggcgatttcacgtaatacctcatcgccagccgcatgccccaaggtatcattcacctgcttaaaaccatccaaatcaatcaatagcagcaccatagtggtgctagcacgctgtttgcggagcacgaacttctcacaacctaaacggttttttagtcccgtcagcgtgtcttgttcggcaatggtgcgatagtagctttcccaacgttcaatctgttgacgcagctcgcgttcacgcagtaaagcttgatgggaagcgtcgataaattcattgatgcttttggccaccaaaccaatctcgttgtggtgatcttctgcggctaccgccactttgcgatcatgatctggccgcacttcggataacgcctgtgaaagatccgtcaggggtttaccgaccaagcggcgaacgatccagataagcgcaataaaagtcacgagaaactggatcaaaaccacggctatctgatcaagaatctgattaatggcttgctgacgaatcacctgatgatcctcatgaatcatcagatagccaatcaaattaccatctacgggagaatctaatcggtagcggttcgcatcactccaataattctgctctttgtaggttgaggggatggtggtgcgctcaaagacaatgccatccacgctggctaacttaaccgcattgatctcttgatgaagcagcaacgcatccatcacctcggaggcaatatcgtaattattcacatacagtgcaatggccgccgagttactcaaggagagcgcaagcttctcttccagctcttgtttttgctgctcaacactctgtatgccgcgcggaataatgatggccaaaatgatcagcaaatacccaagtgcacacagtgaaatcatcttcagcaagcgattaaccagtggcgaagttcgcgtttgatcagtcat

SEQ ID NO: 26 - protein, 430 aa, Vibrio cholerae
MTDQTRTSPLVNRLLKMISLCALGYLLIILAIIIPRGIQSVEQQKQELEEKLALSLSNSAAIALYVNNYDIASEVMDALLLHQEINAVKLASVDGIVFERTTIPSTYKEQNYWSDANRYRLDSPVDGNLIGYLMIHEDHQVIRQQAINQILDQIAVVLIQFLVTFIALIWIVRRLVGKPLTDLSQALSEVRPDHDRKVAVAAEDHHNEIGLVAKSINEFIDASHQALLRERELRQQIERWESYYRTIAEQDTLTGLKNRLGCEKFVLRKQRASTTMVLLLIDLDGFKQVNDTLGHAAGDEVLREIAKRFYALAQTHFSDFVVGRLGGDEFAIYIPLDEFVAESIERFAAQLIESSSEPIGLGQQSAQVGCSIGISHMNSLHIDLEKLLLQADKAMYWVKYRGKHGYHIYRMGEEILPLPSAKELKQDQFR

SEQ ID NO: 27 - DNA, 1003 bp, Vibrio cholerae
  source 1..1003: mol_type = genomic DNA
agcgcatacgctcaagtagggcttgctcacgttgctccgctaagagtaagcgttcagaaagtgaagacatctcgcgcagtaaaggcgccattttcagcttgagctgttctagctccgtctgttctttgagcgcggtctggctacgagccactaaactgctcagctcgccattcatctcttggcggtgtgccatgtaactctggctttgctcaagattttgagtcgcgctttttaggttattgccaatcgagagattcacttgctcgagaaaggcttcggctgctttgcgctcagcatggcttccatcgacgactaaacgcagtacttcaagggtgagctcaagcagggtatgggtattgacgccaagcagaagcttggttcggatatcggtcagttgatcacccgattcaccattgaaatccaactcagtaatcaagtgttgtaaatcaacggcaagtcgatgcagcagttctcgatccgcttgttgagtaagctcattgagcgccaaattgggattggcacattgaattttgaccgcgcgttcataaatttccagcaaacgcaaagcttgctgagttttttccagcggctgtgcggcgctaaaactcagcagatctcgaagatcgcgtttgatcttggcgggtaagccggggacgcgcagtagcgtttcaccactgtgctgtagctggctatccagatgactcgtttgtttgtccatggccaatgactgttgtttcaacatgcgttccagtacggctaatttcgggatcagcgtactgatgtctttttgttgttctaatgcaaaacagagttcttctaaactttggtttagtcgagagctactgccgcggcaagtcgtagccaaggaagtgaccattcgtttaagaacttgctgctctcggttaaatttgaacgaagtatccctttgtgtcaaacgtacttgttctaactgagatttcagtttttgaagctctgcttggatatcttgttctagaacgcccat

SEQ ID NO: 28 - protein, 521 aa, Vibrio cholerae
MGVLEQDIQAELQKLKSQLEQVRLTQRDTSFKFNREQQVLKRMVTSLATTCRGSSSRLNQSLEELCFALEQQKDISTLIPKLAVLERMLKQQSLAMDKQTSHLDSQLQHSGETLLRVPGLPAKIKRDLRDLLSFSAAQPLEKTQQALRLLEIYERAVKIQCANPNLALNELTQQADRELLHRLAVDLQHLITELDFNGESGDQLTDIRTKLLLGVNTHTLLELTLEVLRLVVDGSHAERKAAEAFLEQVNLSIGNNLKSATQNLEQSQSYMAHRQEMNGELSSLVARSQTALKEQTELEQLKLKMAPLLREMSSLSERLLLAEQREQALLERMRYSKDQMEALSDLAQDYRRRLEDQALRAQLDPLTKVYNRSSFTERLEHEYRRWIRTQHNLRVVLFDIDKFKSINDSFGYTAGDKALSIIARTIKKELRDSDTVARFSGEEFILLLPERSDNESYQIIHQIQLNVSKLPFKFRDKSLTITLSAASIRFMDSDTPETVLDRLNLTLSEAKHIGPSQLAWK

SEQ ID NO: 29 - DNA, 563 bp, Vibrio cholerae
  source 1..563: mol_type = genomic DNA
atagcaaagatcagatggaagccctgtctgatttggcacaagattatcgtcgccgccttgaagatcaagcattgcgcgcacaactcgatcctctgaccaaagtgtacaaccgcagcagctttactgagcgacttgaacatgagtatcgccgctggatccgtacgcaacacaatttgcgggtagtgctgtttgatattgataaattcaaatcgatcaacgacagctttggctacaccgcaggcgataaggccttaagtatcattgctcgcaccatcaaaaaagaattacgagacagtgacaccgtggctcgcttctctggtgaagagttcattctgttactgcctgaacgctccgataatgagagttaccagattattcaccagatccagctcaacgtgtcgaaactaccgttcaagttccgcgataagagcctaaccatcacgctgtctgcggcgagtatccgcttcatggattcagatacccccgaaacggttcttgatcgtttaaatctgacgctaagtgaagccaaacatatcggtccaagtcagttagtttggaaataa

SEQ ID NO: 30 - protein, 434 aa, Vibrio cholerae
MLKQQSLAMDKQTSHLDSQLQHSGETLLRVPGLPAKIKRDLRDLLSFSAAQPLEKTQQALRLLEIYERAVKIQCANPNLALNELTQQADRELLHRLAVDLQHLITELDFNGESGDQLTDIRTKLLLGVNTHTLLELTLEVLRLVVDGSHAERKAAEAFLEQVNLSIGNNLKSATQNLEQSQSYMAHRQEMNGELSSLVARSQTALKEQTELEQLKMKMAPLLREMSSLSERLLLAEQREQALLERMRYSKDQMEALSDLAQDYRRRLEDQALRAQLDPLTKVYNRSSFTERLEHEYRRWIRTQHNLRVVLFDIDKFKSINDSFGYTAGDKALSIIARTIKKELRDSDTVARFSGEEFILLLPERSDNESYQIIHQIQLNVSKLPFKFRDKSLTITLSAASIRFMDSDTPETVLDRLNLTLSEAKHIGPSQLVWK

SEQ ID NO: 31 - protein, 339 aa, Vibrio cholerae
MMTTEDFKKSTANLKKVVPLMMKHHVAATPVNYALWYTYVDQAIPQLNAEMDSVLKNFGLCPPASGEHLYQQYIATKAETNINQLRANVEVLLGEISSSMSDTLSDTSSFANVIDKSFKDLERVEQDNLSIEEVMTVIRRLVSDSKDIRHSTNFLNNQLNAATLEISRLKEQLAKVQKDALFDSLSGLYNRRAFDGDMFTLIHAGQQVSLIMLDIDHFKALNDNYGHLFGDQIIRAIAKRLQSLCRDGVTAYRYGGEEFALIAPHKSLRIARQFAESVRRSIEKLTVKDRRSGQSVGSITASFGVVEKIEGDSLESLIGRADGLLYEAKNLGRNRVMPL

SEQ ID NO: 32 - DNA, 1020 bp, Vibrio cholerae
  source 1..1020: mol_type = genomic DNA
atgatgacaactgaagatttcaaaaaatccacggctaacttaaaaaaagtcgtacctttaatgatgaaacatcatgtcgcggccacccccgtgaactatgccttgtggtatacctacgtcgaccaagccattccgcaactgaatgcggaaatggactctgtattgaaaaattttgggctttgcccacccgcttctggtgaacatctttaccaacaatacattgcgaccaaagcagaaaccaatattaatcagttacgtgcgaatgttgaggtacttcttggtgaaattagcagttcaatgagtgatacgctcagtgacaccagttcctttgctaatgtgattgataaaagctttaaggatttagagcgcgtcgagcaagacaatctctcgattgaagaagtaatgacggtgatccgccgcttggtgagtgactctaaagatattcgacactcaaccaatttcctaaataatcaactgaacgcggcaacactagaaatctctcgtcttaaagagcagctggcgaaagttcagaaagatgctctgtttgacagtttatctggactctataaccgccgagcttttgatggcgatatgttcacgctgatccatgcaggtcaacaagtcagcctgatcatgctcgacatcgaccacttcaaagcccttaatgataactatggccacctgtttggtgaccaaattatccgtgcgatcgccaaacgtcttcaaagcctatgccgtgacggcgtgacagcttatcgttatggcggtgaagagtttgcactgattgctccgcacaaatcgctgcgtattgcacgccagtttgctgaatcggtgcgacgttcaatagaaaagctcaccgtaaaagatcggcgtagcggtcaatcggtcggtagcattaccgcttcgtttggtgtagtagaaaagattgaaggtgactctttggagtctcttatcggtcgagcggatggattgctgtatgaagcgaaaaatctgggccgcaatcgagtcatgccgctcttg

SEQ ID NO: 33 - DNA, 1020 bp, Vibrio cholerae
  source 1..1020: mol_type = genomic DNA
gtgatgacaactgaagatttcaaaaaatccacggctaacttaaaaaaagtcgtacctttaatgatgaaacatcatgtcgcggccacccccgtgaactatgccttgtggtatacctacgtcgaccaagccattccgcaactgaatgcggaaatggactctgtattgaaaaattttgggctttgcccacccgcttctggtgaacatctttaccaacaatacattgcgaccaaagcagaaaccaatattaatcagttacgtgcgaatgttgaggtacttcttggtgaaattagcagttcaatgagtgatacgctcagtgacaccagttcctttgctaatgtgattgataaaagctttaaggatttagagcgcgtcgagcaagacaatctctcgattgaagaagtaatgacggtgatccgccgcttggtgagtgactctaaagatattcgacactcaaccaatttcctaaataatcaactgaacgcggcaacactagaaatctctcgtcttaaagagcagctggcgaaagttcagaaagatgctctgtttgacagtttatctggactctataaccgccgagcttttgatggcgatatgttcacgctgatccatgcaggtcaacaagtcagcctgatcatgctcgacatcgaccacttcaaagcccttaatgataactatggccacctgtttggtgaccaaattatccgtgcgatcgccaaacgtcttcaaagcctatgccgtgacggcgtgacagcttatcgttatggcggtgaagagtttgcactgattgctccgcacaaatcgctgcgtattgcacgccagtttgctgaatcggtgcgacgttcaatagaaaagctcaccgtaaaagatcggcgtagcggtcaatcggtcggtagcattaccgcttcgtttggtgtagtagaaaagattgaaggtgactctttggagtctcttatcggtcgagcggatggattgctgtatgaagcgaaaaatctgggccgcaatcgagtcatgccgctctaa

SEQ ID NO: 34 - protein, 461 aa, Vibrio cholerae
MDHRFSTKLFLLLMIAWPLLFGSMSEAVERQTLTIANSKAWKPYSYLDEQGQPSGILIDFWLAFGEANHVDIEFQLMDWNDSLEAVKLGKSDVQAGLIRSASRLAYLDFAEPLLTIDTQLYVHRTLLGDKLDTLLSGAINVSLGVVKGGFEQEFMQREYPQLKLIEYANNELMMSAAKRRELDGFVADTQVANFYIVVSNGAKDFTPVKFLYSEELRPAVAKGNRDLLEQVEQGFAQLSSNEKNRILSRWVHIETIYPRYLMPILASGLLLSIVIYTLQLRRTVRLRTQQLEEANQKLSYLAKTDSLTDIANRRSFFEHLEAEQTRSGSLTLMVFDIDDFKTINDRFGHGAGDNAICFVVGCVRQALASDTYFARIGGEEFAIVARGKNAEESQQLAERICQRVAEKKWVVNAQHSLSLTISLGCAFYLHPARPFSLHDADSLMYEGKRNGKNQVVFRTWS

SEQ ID NO: 35 - DNA, 1386 bp, Vibrio cholerae
  source 1..1386: mol_type = genomic DNA
atggatcatcgcttttcgaccaaactgtttctgcttctcatgattgcttggccgcttttattcggatcaatgagtgaggctgtagagcgccaaaccttgactattgccaactcaaaagcatggaaaccctattcttatttggatgaacagggacagccttctggcatattgattgatttttggttggcttttggtgaagcgaatcatgtcgatattgaattccaactgatggattggaatgattccctagaagcggtgaagcttggcaaatccgatgttcaagctggtttgatccgttctgcttcaagattagcgtatctcgattttgcagaacctttactgacaatcgatacacaactctacgtacaccgcacgttattgggcgataaattggatacgctgctatcgggggccattaacgtctcattaggtgtagtaaaagggggatttgaacaagagttcatgcaacgagaatatcctcaacttaagttgattgagtacgccaacaatgaattgatgatgtctgcagcaaagcgacgagaattagatggttttgtggccgatactcaggtcgccaatttctatatagtggtttccaatggcgcgaaagattttacgccagtgaagtttctttattcagaggaattacgtccagcggtcgccaaaggcaatagggatttattagagcaagtagagcaggggtttgcacaattaagtagcaatgagaaaaaccgtattttaagtcgatgggttcatattgaaacgatttatccacgttacttaatgccgattctcgcttcaggtctcttactcagtatcgttatttatactcttcagctacggcgtaccgttcgattgcgaacacagcaacttgaagaagccaatcaaaaactctcctatttagcgaaaacggatagcttgacggacattgctaatcgccgttcgttttttgaacatcttgaagcggaacaaacacgatcaggcagcttaacgttgatggtttttgatattgatgacttcaaaaccattaacgatcgctttgggcatggcgcaggagataatgccatctgtttcgtggttgggtgtgtgcgacaagctttagcatcggatacctactttgcaaggattggtggtgaagagtttgctattgtagcgcgtggtaaaaatgcagaagagtcgcagcagttagctgagcgaatttgccaacgagttgcagaaaaaaagtgggtagtgaatgcccaacactctctgtcactcaccatcagcctaggctgtgcattttacctacacccagctcggccattcagtttgcacgatgccgatagcttaatgtacgaaggaaagcggaatggaaagaaccaggttgtctttcgtacctggtcataa

SEQ ID NO: 36 - protein, 420 aa, Vibrio cholerae
MDHRFSTKLFLLLMIAWPLLFGSMSEAVERQTLTIANSKAWKPYSYLDEQGQPSGILIDFWLAFGEANHVDIEFQLMDWNDSLEAVKLGKSDVQAGLIRSASRLAYLDFAEPLLTIDTQLYVHRTLLGDKLDTLLSGAINVSLGVVKGGFEQEFMQREYPQLKLIEYANNELMMSAAKRRELDGFVADTQVANFYIVVSNGAKDFTPVKFLYSEELRPAVAKGNRDLLEQVEQGFAQLSSNEKNRILSRWVHIETIYPRYLMPILASGLLLSIVIYTLQLRRTVRLRTQQLEEANQKLSYLAKTDSLTDIANRRSFFEHLEAEQTRSGSLTLMVFDIDDFKTINDRFGHGAGDNAICFVVGCVRQALASDTYFARIGGEEFAIVARGKNAEESQQLAERICQRVAEKKWVVNAQHSLSLT

SEQ ID NO: 37 - DNA, 1239 bp, Vibrio cholerae
  source 1..1239: mol_type = genomic DNA
ttagctagcgactttgacacaattgcgcccagcttgcttcgctttataaagtgccccatccgctgctttgagtgcctcaataggatggcggtacagctcagaatcacacacgccaatgctgatggtaatagtgacaatgtcactgttactttttcggctgcgtttttttgcaccttcagcatgacttttcgggcgctggttggtgtcacgaatcaccaactcgtaggactcaatatcctgccgtaaggcctcgatgaaaggcaaaacctcctttgccaattttcctttgtaaataatcgagaactcctcaccaccatagcggtaaactcgtgctttaccgttgatttcacgtaatcgagaggcaaccagtcttaatacatcgtcccccgtatcatgcccgtaagtatcgttaaacttcttgaaatggtcgacatcgagcatagcgagggtaaattttcgacctatatgttttaaatcctgatcaagcgcttgccgaccaggaatttgggtgagtgggtcgttaaatgccatctcatagcccgcggaaatgaggtaaaccagaataagcagcccagataaggtaaacatgatggtggaaatataaggcacatgaaacagcacaaacgcattcatgctcaatacaatcgaactataaaccacaacatcaagaatttgattgcgcgttaataccgagatagcagcaatacctgcgagtgcgacaagataggcaacaaccaccaagggtaagcgagaaatttgcggtacaacgaaaaatattccctcggtgaggctggaatggtctgtttcacctatgtgtagctgggtcagccaagcccaaaagatgaacagcaataaaatagccaagtaactgagaaaggatttgctgaataatccagcattcttgtaggcgtaaggtaaaaaacaggccacaggcaaaagcaagctcagcataatgagttcaagcatggtggaattgacggttaaaggcgtttgaagtcgaatttggatcaaccagtaagccagtaacatcgtcatcgctaccatggcgattctgctttgtttaaaaatgtgagcaacggttagcgcaatcaaaaagagaatgtaggggaggttgaccgccatgcctaagttagactttatcaccaataccacattgctcaagcctagccaaatggctaccagcagcaatagaggaaaaccgaaacggaaccaaggtgaagtaacaaagctagaagacat

SEQ ID NO: 38 - protein, 453 aa, Vibrio cholerae
MSSSFVTSPWFRFGFPLLLLVAIWLGLSNVVLVIKSNLGMAVNLPYILFLIALTVAHIFKQSRIAMVAMTMLLAYWLIQIRLQTPLTVNSTMLELIMLSLLLPVACFLPYAYKNAGLFSKSFLSYLAILLLFIFWAWLTQLHIGETDHSSLTEGIFFVVPQISRLPLVVVAYLVALAGIAAISVLTRNQILDVVVYSSIVLSMNAFVLFHVPYISTIMFTLSGLLILVYLISAGYEMAFNDPLTQIPGRQALDQDLKHIGRKFTLAMLDVDHFKKFNDTYGHDTGDDVLRLVASRLREINGKARVYRYGGEEFSIIYKGKLAKEVLPFIEALRQDIESYELVIRDTNQRPKSHAEGAKKRSRKSNSDIVTITISIGVCDSELYRHPIEALKAADGALYKAKQAGRNCVKVASISLGCAFYLHPARPFSLHDADSLMYEGKRNGKNQVVFRTWS

SEQ ID NO: 39 - DNA, 1884 bp, Vibrio cholerae
  source 1..1884: mol_type = genomic DNA
atgtacacctcagcccgtaaatatttcatacaatttgccattgttgcgtttgtacttggtttcattcctacactgtatttcatacatgctgctagccagcttgagactcaagcggtcagcagcgttgaaaaacagactcgcttacagcttgagttcagtcagcatgacttgttacgaatgctggaaagcacacaccaagccacccagctgttagctaaaaatgaccttttattcacggctgtcaccacaccaagcaaagaagcactcagtcaactcaaaacattgtgggatgtgacgttaagatcgcaagcgattttctcttcattcagattgctggatagacaaggaaaagaacaacttaaagcgatttacgatgggcaccaagtcacctttgttgaatctgctcaaacgacagatccgttcagccagcaaattgtggctcaatacgcccaactcacgacgcctcaagtttgggcaacgcaagtcgcgatgtcagcagatacgccttctggtatgctgccgacctttcgttttgtgacgggtattgagcatcaaggccaacggcaaggttttcttgtcgtgacggtgaagctacagtctctctatcaacgtctctcttttatttatgatcagtttgattcaccggatattttgaattcggcaggagaattactgctcagtgaacacaagccatccggtacacgttcaacctcttcactccacttttcagcccaacacccagagctttggcaaaaaatccaactcaaccaacaaggctttgctctatccaatcaaacctggtttagctatatcaaagtggatctcagttctgtcttacctgactttaaacctttggtattggtactgcgcatcaataaggcagaaatagataagacctacgcaaatgcgcgctgggcactgatgagtcaagcggtgacagtgttatcgctactctctatcattgcggctggatttgcggcatggaacatcaaccatttaaaaaatagccttgacagtaaattggctcgagcagcgatggatggcatgtcagcggtggtcattaccgaccgccagaatcgcatcatcaaagtaaacaacgaatttacccgcctaagtggttacacttttgaagatgtcaaaggtaagcagccgtccatttttgcttctggattacacaaagtcgaattctatatgcagatgtggaaagctctgcaagacaatggcgtatgggaaggtgaagtgatcaacaaacgcaaagatggcgaaagcatcaccgaaattctccgtattcaaagcatccgcgatgaagacaatgtcattcaattctacgttgcctcttttgtggatatttcacatcgcaaggcgctggagaatcgcctgcgtgagctgagcgaaaaagatgcgttaaccgatttgtggaatcgacgtaaattcgatcaaaccatctctttagagtgcgctaagcgtcgccgttatcccgatcaagcccagagctgccttgctatcattgatatcgaccactttaaacgcattaacgacaaattcggacacaacgaaggggacctagtgttacggaccgttgcgaaaggcatccaagatcagttacgggaatcggattttatcgcacggattggcggagaagagtttgccattattttcccctacacttccattgaagaagccgaacaagtacttaaccgcgtacgcctgcatatcgcttcattacaccatcaacaagtgaccctaagtggtggtgttaccgatgtttgcacatcacccgaccaaagctacaaaagagccgatctggctttatatgaatccaaaacatcgggacgcaaccaaatatcagtactcaccgccatggaaatgcatcactttgcgtga

SEQ ID NO: 40 - protein, 627 aa, Vibrio cholerae; identical to SEQ ID NO: 2

SEQ ID NO: 41 - DNA, 1884 bp, Vibrio cholerae
  source 1..1884: mol_type = genomic DNA
atggcaccgatcctttcacactcgatcccgatcccttctagcatgcaggcaaattggcagcagatgctcaacctgctggccgaagtgctgaaagtctcagccaccctgatcatgcgtttacgccatcacgatcttgatgtgttttgtaccagtgtcggcagtgacaatccataccaagtcggcatgaccgaacgattaggcacaggcttgtattgtgaaactgtggtcaatactcgccagatattgttagtcagtaacgccgacctcgacccattgtggaaggataacccagatctggaattgggcatgcgcgcttactgtggcgtaccattgcaatggccaaacggtgagctttttggatctttgtgtgtcaccgatcgtcaagctcgccagtttcttagtaccgatcagcaattgataaaaacctttgctgaatcgattgaagctcagcttaaaaccctttaccaacgcgaaacgttgttgcaaatgaaccaagatttgcacttcaaagttcgtcataaaatgcaaagcatcgcctcgctgaaccaatctctccatcaagagatcgataaacgccgtgccgcagaacagcagattgagtatcagcgcagtcacgaccttgggactggctttctgaatcgcacggcattggagcagcagctcgcgatgcagctggctcaattggcggaacacgaagagctcgctgtgattcatatcggttttgccaatgcccgccaattacaggcgcggctgggttaccacctttgggatgatgtgctaaagcagttacgtgagcgacttggtccggtgacggagggggaattactgaccgctcgccctaactcgaccaatttgacgctgatcttaaaagcccatccgctcgacacccaattaaatcagctttgccatcgtttaattcacgctgggcaagcgcaatttgtgacggaggggctgcccgttcacctcaacccttatattggtgtggcccttagccgtgaaacacgcgatccgcagcagctactgcgccatgccgtcagcagcatgttggcgtgtaaggactcgggatacaaagtgttttttcactctcccgcattagccgataaccatgcacggcaaaatcaattggaaaactatttactgcaagcggtgcgcaacaacgatctgctgctctacttccaacctaaagtcagcatgaaaacccagcgctgggtcggtgctgaggcattgttgcgttggaagcatccggtgttgggtgaattttccaatgaaaccttgattcatatggcagagcaaaatggtcttatctttgaagtggggcattttgttttgcaccaagctttaaaagccgccagtgattggttagcggtgtgcccaaccttttgtatcgcgatcaatgtctcttccgtacagctcaaaaacagtggctttgtcgagcagattcgagatctgctggcgctgtattgcttccctgcgcatcagttggaactggaaatcaccgaaagtggcctgatcgtcgatgagccgaccgcgagtgatattctcaaccgactacacacattaggcgtgacattatcactcgatgattttggtacgggttacgcttcgtttcagtatctaaaaaaattcccatttgatggcatcaagattgataaaagttttatggagcagatcgaacacagcgaaagcgatcaagaaatcgtgcgttctatgctgcatgtagcgaaaaaactgaacttaaacgtggtggtggaaggtattgagtcgacgcagcaagagcagttcattctggaacagggttgcgatgtcggccaaggctttttatatggcaaacctatgcccagtgaagtgtttaccctcaagctcgaaagccacgctctggcgtaa

SEQ ID NO: 42 - protein, 627 aa, Vibrio cholerae
MAPILSHSIPIPSSMQANWQQMLNLLAEVLKVSATLIMRLRHHDLDVFCTSVGSDNPYQVGMTERLGTGLYCETVVNTRQILLVSNADLDPLWKDNPDLELGMRAYCGVPLQWPNGELFGSLCVTDRQARQFLSTDQQLIKTFAESIEAQLKTLYQRETLLQMNQDLHFKVRHKMQSIASLNQSLHQEIDKRRAAEQQIEYQRSHDLGTGFLNRTALEQQLAMQLAQLAEHEELAVIHIGFANARQLQARLGYHLWDDVLKQLRERLGPVTEGELLTARPNSTNLTLILKAHPLDTQLNQLCHRLIHAGQAQFVTEGLPVHLNPYIGVALSRETRDPQQLLRHAVSSMLACKDSGYKVFFHSPALADNHARQNQLENYLLQAVRNNDLLLYFQPKVSMKTQRWVGAEALLRWKHPVLGEFSNETLIHMAEQNGLIFEVGHFVLHQALKAASDWLAVCPTFCIAINVSSVQLKNSGFVEQIRDLLALYCFPAHQLELEITESGLIVDEPTASDILNRLHTLGVTLSLDDFGTGYASFQYLKKFPFDGIKIDKSFMEQIEHSESDQEIVRSMLHVAKKLNLNVVVEGIESTQQEQFILEQGCDVGQGFLYGKPMPSEVFTLKLESHALA

SEQ ID NO: 43 - DNA, 981 bp, Vibrio cholerae
  source 1..981: mol_type = genomic DNA
ttagaaaagttcaacgtcatcagaaaatggccgttgcgcgctggcaattttaccgttctcacacagctgttcatagcagtgcacctgattccgaccatgctctttggcgtaatacaacgctttatcggcatggtcgagaatggtaggtaaatagtcacccggcctgagtgagcaaaaaccagcgctgaagctcagttcaccgattctcgggaagttatggcgtcggatctgttgacggaagccatccaactgttgcttgatttgtggctcattaccgcttgaaaaaataatcacgaactcttcaccaccaaagcgaaatagttgagaagacggtccgaaatagtgctgcatctgctgagcgaacataagcagaatttcatcaccaatcatgtgtccgaagtgatcattgatcgctttaaaatggtcaatatccaacatcgcgatccagagtttgtgattctcttctgtcgagggattgatggcaaaggtgtggcgcaatcggtcttctaacgttcgacgattgagtaatccggtcagcttatcgcgttcactctcatgcaaaatcaccgtgtaattacggtaaattttcgcaaatccgttgatcaacatgcgataaggttcaggatctttattgaggattaagcacagctctgcggaaaagtgttcttctatcggaatcgggcaaaagcattgatattggccattcgcttgttgggaaaacgccatttccgattgagagtgctggtaaccattgtcggcacatacttggtcgtattgccactggtactcctttttacctgcagcatttttggtaataattaaacgtgccaccataagggttgaacgtccaagatggtgaaataaggtcgccgtggagagcggtaacaattcagacaaggtcgccaaaatactgtaactgagtgccagcgaatttttctgctcagtaatttcaataaccgactcaagcactttgtcattcat

SEQ ID NO: 44 - protein, 326 aa, Vibrio cholerae
MNDKVLESVIEITEQKNSLALSYSILATLSELLPLSTATLFHHLGRSTLMVARLIITKNAAGKKEYQWQYDQVCADNGYQHSQSEMAFSQQANGQYQCFCPIPIEEHFSAELCLILNKDPEPYRMLINGFAKIYRNYTVILHESERDKLTGLLNRRTLEDRLRHTFAINPSTEENHKLWIAMLDIDHFKAINDHFGHMIGDEILLMFAQQMQHYFGPSSQLFRFGGEEFVIIFSSGNEPQIKQQLDGFRQQIRRHNFPRIGELSFSAGFCSLRPGDYLPTILDHADKALYYAKEHGRNQVHCYEQLCENGKIASAQRPFSDDVELF

SEQ ID NO: 45 - DNA, 927 bp, Vibrio cholerae; identical to SEQ ID NO: 5
  source 1..927: mol_type = genomic DNA

SEQ ID NO: 46 - protein, 308 aa, Vibrio cholerae; identical to SEQ ID NO: 6

SEQ ID NO: 47 - DNA, 1017 bp, Vibrio cholerae
  source 1..1017: mol_type = genomic DNA
atgacaactgaagatttcaaaaaatccacggctaacttaaaaaaagtcgtacctttaatgatgaaacatcatgtcgcggccacccccgtgaactatgccttgtggtatacctacgtcgaccaagccattccgcaactgaatgcggaaatggactctgtattgaaaaattttgggctttgcccacccgcttctggtgaacatctttaccaacaatacattgcgaccaaagcagaaaccaatattaatcagttacgtgcgaatgttgaggtacttcttggtgaaattagcagttcaatgagtgatacgctcagtgacaccagttcctttgctaatgtgattgataaaagctttaaggatttagagcgcgtcgagcaagacaatctctcgattgaagaagtaatgacggtgatccgccgcttggtgagtgactctaaagatattcgacactcaaccaatttcctaaataatcaactgaacgcggcaacactagaaatctctcgtcttaaagagcagctggcgaaagttcagaaagatgctctgtttgacagtttatctggactctataaccgccgagcttttgatggcgatatgttcacgctgatccatgcaggtcaacaagtcagcctgatcatgctcgacatcgaccacttcaaagcccttaatgataactatggccacctgtttggtgaccaaattatccgtgcgatcgccaaacgtcttcaaagcctatgccgtgacggcgtgacagcttatcgttatggcggtgaagagtttgcactgattgctccgcacaaatcgctgcgtattgcacgccagtttgctgaatcggtgcgacgttcaatagaaaagctcaccgtaaaagatcggcgtagcggtcaatcggtcggtagcattaccgcttcgtttggtgtagtagaaaagattgaaggtgactctttggagtctcttatcggtcgagcggatggattgctgtatgaagcgaaaaatctgggccgcaatcgagtcatgccgctctaa

SEQ ID NO: 48 - protein, 338 aa, Vibrio cholerae
MTTEDFKKSTANLKKVVPLMMKHHVAATPVNYALWYTYVDQAIPQLNAEMDSVLKNFGLCPPASGEHLYQQYIATKAETNINQLRANVEVLLGEISSSMSDTLSDTSSFANVIDKSFKDLERVEQDNLSIEEVMTVIRRLVSDSKDIRHSTNFLNNQLNAATLEISRLKEQLAKVQKDALFDSLSGLYNRRAFDGDMFTLIHAGQQVSLIMLDIDHFKALNDNYGHLFGDQIIRAIAKRLQSLCRDGVTAYRYGGEEFALIAPHKSLRIARQFAESVRRSIEKLTVKDRRSGQSVGSITASFGVVEKIEGDSLESLIGRADGLLYEAKNLGRNRVMPL

SEQ ID NO: 49 - DNA, 1374 bp, Vibrio cholerae; identical to SEQ ID NO: 7
  source 1..1374: mol_type = genomic DNA

SEQ ID NO: 50 - protein, 457 aa, Vibrio cholerae; identical to SEQ ID NO: 8

SEQ ID NO: 51 - DNA, 1176 bp, Vibrio cholerae; identical to SEQ ID NO: 11
  source 1..1176: mol_type = genomic DNA

SEQ ID NO: 52 - protein, 391 aa, Vibrio cholerae; identical to SEQ ID NO: 12

SEQ ID NO: 53 - DNA, 1911 bp, Vibrio cholerae
  source 1..1911: mol_type = genomic DNA
atgacgctatacaaacaactagtcgcagggatgattgcggtgtttattctgttgttgatttcggtttttactatcgaattcaacaccactcgcaacagtcttgaacaacaacaacgctctgaagtcaacaacaccataaatacggtgggtttggctttagcgccttatctggagaagaaagacaccattgcggtagagtcagtcatcaatgcgctgtttgatggcagtagttactcgatcgtacgtctgatttttctcgatgacggtacggaaatcctgcgctcataccctatccaacccaataatgtgccggcttggtttactcagttaaatctgtttgagcccatccatgatcggcgtgttgtaaccagtggttggatgcaattggcggaagtggaaatcgtcagccatcctggtgcggcttacgctcaactctggaaagcattaattcgtttaagtatcgcgtttttggcgatcttagtgattggtatgtttgccgtcgccttcattttgaagcgctctctaagaccactacaactcatcgtcaacaaaatggagcaggttgctaacaaccaatttggtgagcctctaccgcgccccaacactcgagatctgatttatgtagtagatggcatcaataagatgtctgaacaggtcgagaaagcgtttaaagcccaagccaaagaggcgcagcaactgcgtgaacgtgcttatcttgacccagtttctcatcttggcaaccgagcatactacatgagccaattgagtggctggctctctgaaagcggcatcggtggtgtagccattctacaagctgaattcatcaaagagctttatgaagagaagggctatgaagccggtgatggcatggtgcgcgaactggcggatcgccttaaaaactccatcaccatcaaggacatctctatcgctcgtatctccacttacgagttcggtatcatcatgcctaacatggatgaaactgagctcaaaatcgtggcagagagcatcatcacttgtgtggacgacattaaccctgatcctactggtatggcgaaagccaatttatcgcttggcgtggtaagcaataagcgtcaatccagcaccacaacgctcttgtccctgctggataatgcgttagctaaagcgaaatccaatcctgagctgaactacggctttattagcagtgatactgataaaatcatcttgggcaaacagcagtggaaaactctggtcgaagaggcaatccataacgactggtttactttccgctaccaagccgccaacagcagttggggaaaaacattccatcgcgaggtcttttctgcgtttgagaaagacggcgtgcgttacacggcaaaccaattcttgtttgcccttgaacagctcaatgctagccatatcttcgatcagtacgtgattgaacgtgtgattcaacagcttgaaaaaggcgaactgaccgatccactcgcgatcaacatcgcacaaggcagtatctctcaaccgagctttatccgttggatcagccaaaccttaagcaagcatctttctgtggccaacttactgcattttgagatcccagaaggctgtttcgtcaatgaaccgcattacactgcgctattttgtaacgcagtacgcaatgcaggggcggactttggggtagacaactacggacgtaacttccaatctctcgactacatcaacgagttccgtcctaaatacgtcaaactggattatctatttactcaccatttggatgatgaacgccagaaatttaccctgacctcaatctcgcgcaccgcgcataacttagggatcaccaccatcgcatcacgggttgaaacacagactcagctcgattttctttcagaacatttcatcgaagtcttccaaggcttcattgttgataagtaa

SEQ ID NO: 54 - protein, 636 aa, Vibrio cholerae
MTLYKQLVAGMIAVFILLLISVFTIEFNTTRNSLEQQQRSEVNNTINTVGLALAPYLEKKDTIAVESVINALFDGSSYSIVRLIFLDDGTEILRSYPIQPNNVPAWFTQLNLFEPIHDRRVVTSGWMQLAEVEIVSHPGAAYAQLWKALIRLSIAFLAILVIGMFAVAFILKRSLRPLQLIVNKMEQVANNQFGEPLPRPNTRDLIYVVDGINKMSEQVEKAFKAQAKEAQQLRERAYLDPVSHLGNRAYYMSQLSGWLSESGIGGVAILQAEFIKELYEEKGYEAGDGMVRELADRLKNSITIKDISIARISTYEFGIIMPNMDETELKIVAESIITCVDDINPDPTGMAKANLSLGVVSNKRQSSTTTLLSLLDNALAKAKSNPELNYGFISSDTDKIILGKQQWKTLVEEAIHNDWFTFRYQAANSSWGKTFHREVFSAFEKDGVRYTANQFLFALEQLNASHIFDQYVIERVIQQLEKGELTDPLAINIAQGSISQPSFIRWISQTLSKHLSVANLLHFEIPEGCFVNEPHYTALFCNAVRNAGADFGVDNYGRNFQSLDYINEFRPKYVKLDYLFTHHLDDERQKFTLTSISRTAHNLGITTIASRVETQTQLDFLSEHFIEVFQGFIVDK

SEQ ID NO: 55 - DNA, 1581 bp, Vibrio cholerae
  source 1..1581: mol_type = genomic DNA
ctactcaacacacacttggttacggccattggctttggcgcgatacaaagctttgtcagcgcggtagaacgtacgttgggtattttccccctcgcgatgcaaggtgataccgatactgaccgtcagtccccgttcgccaagtacgtcttgccatgggaaatcaaaaatacgttggcgataggtttcggcatgcatttgtgccatatcactggtgacgttttccaaaatcaccagaaattcctcgccaccgaaacgtacgcaggaggcaccacggaatttaaagtaactcgccagttcactggatacattgacaatcgctttatcccctaccaaatgactcaattcatcattgatcgatttaaagtggtcaatatcaacgactaagaaagcaaacggggtttcgtgcagcagcagatctttcagcttcacgtccaaccaacggcggttatgcagttttgtcagtggatcggtgaacacatcttgctgtagttgcaacaccgtattcttctggctttcggtggtttcttttagctcacgattttctaattccgacaaaatcagtttaagttgtagctcaaagcgcgataggcggcgtagctgaattgggcctaattcactgatggggatccgcttcatcaaatcgctttcgatgcgaaatgctttcttttcgtaaaccagtgcggttttgtacattccttcgagttcacacacttcgctgaacgcttcatagaggcgtttttcaaggaaaggggaatgaatgttttgtaagcgcttttcagtgctacccagcagcatggtggcaaaatgcgccttacctgctttagagaggcaatgcgctaactcgatgcgtagcatgcttgatagccaatccgatggcgtcagcgatgacgaatactgtgcattggcgagtgtcatcatcgccttttgcactttgccttgttgcagataaagcttggcttgatagagcatgatctgcccagtcagcagtttatcgctgaccagaatgctcaactcatcacactcttttatcagatcattggccgctgcataacgaccaaggctgatgtagcaagccagcatatacagcttgtaacgcaggcgcagtgagcggctagaaatcgcatgatctatgctgtcaattttttggtagtagcgtaacgcacggctgtgatcgccataagcatcacataaattgcccattccgagcactgcaagtacgtagtcatcaatcatgccatgctcaacggcgatgttggatatcgcaacgtattcagacagtgccgcgacatattcaccatggtcgagtaaacgctcactcaaactgtgtttgaccgagagcattaattccagatccgtcggtaactctaatagggaaagagcggcgcgcagctcttcaatactggtttgccactgtttcatttcgcggcggtattcggcgctgatgatgtagctttgtgcacgctcttgggcggtggttgccacgtgctgtctgacatggttccagaaaatgatcgcctcttcaccagcgacagcggccgcatccagtcccgcttctttgatcttattgagcagggtttccat

SEQ ID NO: 56 - protein, 526 aa, Vibrio cholerae
METLLNKIKEAGLDAAAVAGEEAIIFWNHVRQHVATTAQERAQSYIISAEYRREMKQWQTSIEELRAALSLLELPTDLELMLSVKHSLSERLLDHGEYVAALSEYVAISNIAVEHGMIDDYVLAVLGMGNLCDAYGDHSRALRYYQKIDSIDHAISSRSLRLRYKLYMLACYISLGRYAAANDLIKECDELSILVSDKLLTGQIMLYQAKLYLQQGKVQKAMMTLANAQYSSSLTPSDWLSSMLRIELAHCLSKAGKAHFATMLLGSTEKRLQNIHSPFLEKRLYEAFSEVCELEGMYKTALVYEKKAFRIESDLMKRIPISELGPIQLRRLSRFELQLKLILSELENRELKETTESQKNTVLQLQQDVFTDPLTKLHNRRWLDVKLKDLLLHETPFAFLVVDIDHFKSINDELSHLVGDKAIVNVSSELASYFKFRGASCVRFGGEEFLVILENVTSDMAQMHAETYRQRIFDFPWQDVLGERGLTVSIGITLHREGENTQRTFYRADKALYRAKANGRNQVCVE

SEQ ID NO: 57 - DNA, 1101 bp, Vibrio cholerae
  source 1..1101: mol_type = genomic DNA
tcacgatgaggggcttttttgtaggaatttcatttcatacatgtttttatctgccagatggatcaactggctcaaattggtgctgtcgagtggataagtactgaccccgacgctggtgttgagcttggctcgtaaatcgccacttaattcaaattcatggtcgaaacactgtttgatcatgcgctgcatcatcatctgctcggtcgaattgatgctgcttaggatgatggcaaattcatctccccccatccgaaacacacgataatcgaatgaaggaatcgagttgtttaagcgataagcaacctgtttgagtaccgcatcgcccatttgatggccgtaggtatcattaatttgtttaaaaccattcagatcgagcaaaaagagagagaatccaccgctgcggcggtggcgttctaattcggcgaacatggctgtgcggttttccagccctgttaatgggtccgttaaggccaagactctatggtgcgtggcctctttatgcaaaataaaactcaccagtcccacacagctaaacgtcaacaaaattaacgcaaactggatgcgactgaggtaattcagtttctctttttgctctacatacaaaggactttgcattccaaatgtgcggtttatgaactgaataaaaatctccagctcttgttgggcggcaacaataaaagtttgtaagctttctggatttttggccgcaagcagtagcggttcaagttgtttaaagcgcgcaaacgcggcttggaagaattcgcgagtgctgggcatgcctataatgccgtcggcttctgggctattgaggatcagatcaaaacggctccaagtcagctcatatttcaccatcacatcgcgctggttgctctccgactccaataggtagggggagagtgccagcatctcagtaaactctttattgagctggaataagaaccagatcgcttggttagtatgcgaagagtaagacttagataaatcgcgagtactgttgatcaaatacaaattggccaaaatcagaatcgccgacatgaagatcagcagtgttttggcatgtaagatcagcgggtggagcgttttctgagtttgtgtattcat

SEQ ID NO: 58 - protein, 366 aa, Vibrio cholerae
MNTQTQKTLHPLILHAKTLLIFMSAILILANLYLINSTRDLSKSYSSHTNQAIWFLFQLNKEFTEMLALSPYLLESESNQRDVMVKYELTWSRFDLILNSPEADGIIGMPSTREFFQAAFARFKQLEPLLLAAKNPESLQTFIVAAQQELEIFIQFINRTFGMQSPLYVEQKEKLNYLSRIQFALILLTFSCVGLVSFILHKEATHHRVLALTDPLTGLENRTAMFAELERHRRSGGFSLFLLDLNGFKQINDTYGHQMGDAVLKQVAYRLNNSIPSFDYRVFRMGGDEFAIILSSINSTEQMMMQRMIKQCFDHEFELSGDLRAKLNTSVGVSTYPLDSTNLSQLIHLADKNMYEMKFLQKSPSS

SEQ ID NO: 59 - DNA, 1824 bp, Vibrio cholerae
  source 1..1824: mol_type = genomic DNA
ttaggctacattcgtttcttttctccagcgttcaatcatcacactcggtaaatcaggtcgactgaagtaatacccttgaatttgctcacagcccatttgatagagtttatccagtgcttgttggttctctaccccctcagcgacgagatcgagtttaagctggttagcaagctgaataatcaaccacacgatactctcagaggtttggttggtaagtaggttacgcacaaatgcagcatcaatcttgatgcaatcaatcggataactgtgaatgtagttaaggctcgaataacctgtcccaaaatcatccaaggcaattttaaaacccaattcacgcaatatggtgagaatactgcatacttctgcggccttagagagtaaaaccgtttctgtcagctcaatagtgaactcgtcggcttgaaaaccataggctttaatggtttttaatagatgctcaaggtaacgattggaatgcgtcagctcatcggcggagcagttgatgcttaagcgaattttttggtcaataccttgttctaattcttgtttcgcgatgcaggccaattcgagaatacgttcgccaaattcgacaatcaggccagattgctctgctgcttcaatgaattccaatggcgttaccacaccgagcgtactgctattccaacgcgttaagatctcaaaatagtcccaatttctttgatgttttttcacgatcggttgcacgaccacatacagctcagtttgatggataggcttactcaattcactacgcagagcttcgatgatttgtgtacgccgatagtattgattgctgagtaagttgtcgtagaaacgaatgcgtgtgttatggttccgtttacactcttttaaagcgagacttgcattgaacagtaattgatcggcattgagcttttcaccactgtatttggtaataccaatactgacactgattttgagtcgacgatcttgatcgatataatcttgcgccagcttgttgagtatggtttggcagatcttcatcggctcacgatctgtggttaaaaaagcaaattcatcagcggcgattcgaaaggcgtatccttcttcggggacggcttgttttatcgcatccgcgacaaatttcagcacaagatctcccaaatagtgcccatgcagatcgtttatcgaacgaaattcatcaatatcaagaaaggccagagtgaaatgatgtctatcttcttgaacgagagccgtcagtttctcggctaaatcattacgattcattaaacccgttaagttgtcgtgagatatttcatgacgtaattgattgattaggctctgagagcgtacctccatctgtttacattccagatcatgagcgatcatctgagccaaaatctggtgaactaacacgagattagcaaagtcgtctaactgacgcgtaaaagtcgagatcaaaacgccgtagttttcgccatttgaaaaataaatcgggatacccagatacgcctcaatatggttctcaactaaataagcatcgttaggaaaaagttccgcgactttgcttgcaaataggcaataaggttgtctttgtaatccgacttgctcacaaggtgtgccttgtagttcgtaatacagctctaaactgctgggttcgacactggcacaacttaagttatgagctttgtagcgcattttatctagctcaatgaccattgagctgtggctattgaaggtgcggtggagaaactgagtgatttgtgagagcaactccaacccccccagctgactgaagtgatgtatggaatctaggctcagtttttctgttatcagttgagtcttggtcat

SEQ ID NO: 60 - protein, 607 aa, Vibrio cholerae
MTKTQLITEKLSLDSIHHFSQLGGLELLSQITQFLHRTFNSHSSMVIELDKMRYKAHNLSCASVEPSSLELYYELQGTPCEQVGLQRQPYCLFASKVAELFPNDAYLVENHIEAYLGIPIYFSNGENYGVLISTFTRQLDDFANLVLVHQILAQMIAHDLECKQMEVRSQSLINQLRHEISHDNLTGLMNRNDLAEKLTALVQEDRHHFTLAFLDIDEFRSINDLHGHYLGDLVLKFVADAIKQAVPEEGYAFRIAADEFAFLTTDREPMKICQTILNKLAQDYIDQDRRLKISVSIGITKYSGEKLNADQLLFNASLALKECKRNHNTRIRFYDNLLSNQYYRRTQIIEALRSELSKPIHQTELYVVVQPIVKKHQRNWDYFEILTRWNSSTLGVVTPLEFIEAAEQSGLIVEFGERILELACIAKQELEQGIDQKIRLSINCSADELTHSNRYLEHLLKTIKAYGFQADEFTIELTETVLLSKAAEVCSILTILRELGFKIALDDFGTGYSSLNYIHSYPIDCIKIDAAFVRNLLTNQTSESIVWLIIQLANQLKLDLVAEGVENQQALDKLYQMGCEQIQGYYFSRPDLPSVMIERWRKETNVA

SEQ ID NO: 61 - DNA, 1998 bp, Vibrio cholerae
  source 1..1998: mol_type = genomic DNA
gtggcaggtcacaccttactctcttccaacacgtttacgccgctagaagcgtatcctgaagccttttgggcatgggctgcgcagtttgatacttccgatggtttgatcccttttgccatcaatacctgtcgctggaactatttgccagtgatgggcggtgagtcgtttatttttatgctggataatcatcctcagcatcggacttatctgatcattcaagcggcatgcgtcgataaagtacacctgagcactcaatccggtgagttggattttttacagttaattgcagcgaaatggcaatgcttacgagcggaaattgaagcatcgaaagagtttaaaaatcgtgatttacgtgaggcgcagtaccttagtgaaattcgtcagcgagagcagtttattgacaacatgaagctggtgcatcaagtcgcgctcgagttgtccaaccccgccaatcttgatgagctacaccgcgcatcggtcgaggctatgcgacatcgtctcgggtttgatcgatccgcgctcttgttgcttgatatgaaaaagcgttgcttcagcggtacttatggtaccgatgagcacggtaatacgattgatgaacagcacacccagtatgatctgcaccaattagagcctcaatatctcgaagctttatccaatgaagagtgcactttgatggtggtggaagatgtgcctttgtacaccgtcggacaggtagtgggacaaggctggaatgccatgctgattttgcgtgatggtaatgacaccataggctggattgccatcgacaactatatcaatcggcagccgattaccgagtatcaaaagcagatgcttgagtcgtttggctcattgctcgcgcaaatttatattcgtaaaaagcaggaacaaaacgtacgtatgctgcatgccagcatggtcgaactgtctcgctgtatgacagtcagtgaagtgtgtaaatcggcagtcacctttgcgatcaaccgaatggggattgatcgcatggcggtgtttttgacggatgaagcttgctcttatattcaggggacgtgggggacggatattcaaggcaatattgtcgatgaatcctatttccgtggttcaacgcatgaaaatgacattgtcgaccttgccaaagtgtacccaaacgaagtggtgtttaaagagagtgttcccatctatcacgactgtaaaattgtcggttatggttggacggcgatgaccatgctcaccgacaaaggcaccccgattgcctttattgcggcggataatttgatccgacgttcccccttgacttcacaactgcgtgaagtgattcgtatgtttgcttcaaacctcaccgaagtcttgatgcgagccaaagcccaagaagcgatctcggtactcaatgaaacgctggagcttgaggtgcgtaatcgcactcgtgatttgcaaaaggccaacgaaaaactcgatttaatggcgaaattagatccgctgactcgtttagggaatcgccgtatgcttgagcaccaactggagcaaacttgcgaacagaccatcaaagaggtggtcaattatggcgtgatcttgcttgatattgaccatttcgggcttttcaacaactgctatggtcatcttgaaggcgatattgctctgatgcggattggtaatatcctcagtcgacatgcgcaatctgagcatgaactgttctgtcgtattggtggggaagagtttctgcttttagtcgccaatcgaagcgccgaggagattcacttactggctgaaaatattcgtaaaagtattgaagcagaatgcattgaacactgcgaaaatcccagtggtgagctactgaccgtatcgattggttatgctgcttctcgttataaaccgcgagagattcaatttgatcagctctatgcagaagcggataaagccttgtacagagcgaaaagccaaggacggaatcaggttattggcgttattgttgaaaatatcgactgcatacaggcagaaatgtag

SEQ ID NO: 62 - protein, 665 aa, Vibrio cholerae
MAGHTLLSSNTFTPLEAYPEAFWAWAAQFDTSDGLIPFAINTCRWNYLPVMGGESFIFMLDNHPQHRTYLIIQAACVDKVHLSTQSGELDFLQLIAAKWQCLRAEIEASKEFKNRDLREAQYLSEIRQREQFIDNMKLVHQVALELSNPANLDELHRASVEAMRHRLGFDRSALLLLDMKKRCFSGTYGTDEHGNTIDEQHTQYDLHQLEPQYLEALSNEECTLMVVEDVPLYTVGQVVGQGWNAMLILRDGNDTIGWIAIDNYINRQPITEYQKQMLESFGSLLAQIYIRKKQEQNVRMLHASMVELSRCMTVSEVCKSAVTFAINRMGIDRMAVFLTDEACSYIQGTWGTDIQGNIVDESYFRGSTHENDIVDLAKVYPNEVVFKESVPIYHDCKIVGYGWTAMTMLTDKGTPIAFIAADNLIRRSPLTSQLREVIRMFASNLTEVLMRAKAQEAISVLNETLELEVRNRTRDLQKANEKLDLMAKLDPLTRLGNRRMLEHQLEQTCEQTIKEVVNYGVILLDIDHFGLFNNCYGHLEGDIALMRIGNILSRHAQSEHELFCRIGGEEFLLLVANRSAEEIHLLAENIRKSIEAECIEHCENPSGELLTVSIGYAASRYKPREIQFDQLYAEADKALYRAKSQGRNQVIGVIVENIDCIQAEM

SEQ ID NO: 63 - DNA, 447 bp, Vibrio cholerae; identical to SEQ ID NO: 13
  source 1..447: mol_type = genomic DNA

SEQ ID NO: 64 - protein, 148 aa, Vibrio cholerae; identical to SEQ ID NO: 14

SEQ ID NO: 65 - DNA, 1080 bp, Vibrio cholerae
  source 1..1080: mol_type = genomic DNA
tcagcgatgaccatgagttgaacccaatagcgcatgacaatggtcaccattgagttcaatgacatgctcttcatcgaagctgacgcggtttttccccatttttttcgaatgatagagagcttggtctgcgcgtttgaaccactgctccggatcatcggtgcgaagtgcttcggctaaaccgacactgacggtgactttggcatggtatgggtagtgcgtttgttgaatccgacaaccaatatgactcatcacgagtgtagcgtcggttaacgacgtattttcaaacagcagtaaaaattcatcgccccctaatcgaaacaacagatctaactcacggcagtgagtattcattatttcaacaacttgggtaatgactttatctcctgtgtcgtgtccataaaggtcattaacagatttgaagtgatcgatatcgatcacggcgatcaccgccgattcattggcgagctggcggtggcgaagacattttttcaaaaaaccatccagttgatgacgattcaatgtgcccgttaatgcatgacgagtggaaagataaaaaagctcagtgtgcagcttacggatagcatctaccaccacatacatgatggcggcacaagcgctgatcgcaaggctaaagcgcaaggtgacttcggcggtttgatggggaattaaaacccatatgctggctggaatgataatggtgatggtcaataagttatctttctgggggagtagaaaagcaatcgcaatgagcacgggaaatagccagtagctggcgagggtgccgaaaatgtgaatagccatcaccacgatgactaccaccaatgccagtggaagcctaaaaccccatggtgttttcttttgataatagatagccgtaatttcaatgaggagcgtgcattggaatacgatgatcaacccgccaagaagaacgtagtcaatcagcaagtttttaacggcgagtggaaagaaaaccaaactagaaataaaaccaataaaaagcgacacccgacgttgatagtaagtgttcagtaactctgaaccggtaaaagcaggagagtgagtcgattttgtcatcgtcat

SEQ ID NO: 66 - protein, 359 aa, Vibrio cholerae
MTMTKSTHSPAFTGSELLNTYYQRRVSLFIGFISSLVFFPLAVKNLLIDYVLLGGLIIVFQCTLLIEITAIYYQKKTPWGFRLPLALVVVIVVMAIHIFGTLASYWLFPVLIAIAFLLPQKDNLLTITIIIPASIWVLIPHQTAEVTLRFSLAISACAAIMYVVVDAIRKLHTELFYLSTRHALTGTLNRHQLDGFLKKCLRHRQLANESAVIAVIDIDHFKSVNDLYGHDTGDKVITQVVEIMNTHCRELDLLFRLGGDEFLLLFENTSLTDATLVMSHIGCRIQQTHYPYHAKVTVSVGLAEALRTDDPEQWFKRADQALYHSKKMGKNRVSFDEEHVIELNGDHCHALLGSTHGHR

SEQ ID NO: 67 - DNA, 1386 bp, Vibrio cholerae; identical to SEQ ID NO: 35
  source 1..1386: mol_type = genomic DNA

SEQ ID NO: 68 - DNA, 981 bp, Vibrio cholerae
  source 1..981: mol_type = genomic DNA
atgaatgacaaagtgcttgagtcggttattgaaattactgagcagaaaaattcgctggcactcagttacagtattttggcgaccttgtctgaattgttaccgctctccacggcgaccttatttcaccatcttggacgttcaacccttatggtggcacgtttaattattaccaaaaatgctgcaggtaaaaaggagtaccagtggcaatacgaccaagtatgtgccgacaatggttaccagcactctcaatcggaaatggcgttttcccaacaagcgaatggccaatatcaatgcttttgcccgattccgatagaagaacacttttccgcagagctgtgcttaatcctcaataaagatcctgaaccttatcgcatgttgatcaacggatttgcgaaaatttaccgtaattacacggtgattttgcatgagagtgaacgcgataagctgaccggattactcaatcgtcgaacgttagaagaccgattgcgccacacctttgccatcaatccctcgacagaagagaatcacaaactctggatcgcgatgttggatattgaccattttaaagcgatcaatgatcacttcggacacatgattggtgatgaaattctgcttatgttcgctcagcagatgcagcactatttcggaccgtcttctcaactatttcgctttggtggtgaagagttcgtgattattttttcaagcggtaatgagccacaaatcaagcaacagttggatggcttccgtcaacagatccgacgccataacttcccgagaatcggtgaactgagcttcagcgctggtttttgctcactcaggccgggtgactatttacctaccattctcgaccatgccgataaagcgttgtattacgccaaagagcatggtcggaatcaggtgcactgctatgaacagctgtgtgagaacggtaaaattgccagcgcgcaacggccattttctgatgacgttgaacttttctaa

SEQ ID NO: 69 - protein, 326 aa, Vibrio cholerae; identical to SEQ ID NO: 44

SEQ ID NO: 70 - DNA, 1311 bp, Vibrio cholerae
  source 1..1311: mol_type = genomic DNA
gtgagaatgacttggaactttcaccagtactacacaaaccgaaatgatggcttgatgggcaagctagttcttacagacgaggagaagaacaatctaaaggcattgcgtaagatcatccgcttaagaacacgagatgtatttgaagaagctaagggtattgccaaggctgtgaaaaaaagtgctcttacgtttgaaattattcaggaaaaggtgtcaacgacccaaattaagcacctttctgacagcgaacaacgagaagtggctaagcttatttacgagatggatgatgatgctcgtgatgagtttttgggattgacacctcgcttttggactcagggaagctttcagtatgacacgctgaatcgcccgtttcagcctggtcaagaaatggatattgatgatggaacctatatgccaatgcctatttttgagtcagagcctaagattggtcattctttactaattcttcttgttgacgcgtcacttaagtcacttgtagctgaaaatcatggctggaaatttgaagctaagcagacttgtgggaggattaagattgaggcagagaaaacacatattgatgtaccaatgtatgcaatccctaaagatgagttccagaaaaagcaaatagctttagaagcaaatagatcatttgttaaaggtgccatttttgaatcatatgttgcagattcaattactgacgatagtgaaacttatgaattagattcagaaaacgtaaaccttgctcttcgtgaaggtgatcggaagtggatcaatagcgaccccaaaatagttgaagattggttcaacgatagttgtatacgtattggtaaacatcttcgtaaggtttgtcgctttatgaaagcgtggagagatgcgcagtgggatgttggaggtccgtcatcgattagtcttatggctgcaacggtaaatattcttgatagcgttgctcatgatgctagtgatctcggagaaacaatgaagataattgctaagcatttacctagtgagtttgctaggggagtagagagccctgacagtaccgatgaaaagccactcttcccaccctcttataagcatggccctcgggagatggacattatgagcaaactagagcgtttgccagagattctgtcatctgctgagtcagctgactctaagtcagaggccttgaaaaagattaatatggcgtttgggaatcgtgttactaatagcgagcttattgttttggcaaaggctttaccggctttcgctcaagaacctagttcagcctcgaaacctgaaaaaatcagcagcacaatggtaagtggctga

SEQ ID NO: 71 - DNA, 2201 bp, Homo sapiens
  source 1..2201: mol_type = genomic DNA
gcgacttcccagcctggggttccccttcgggtcgcagactcttgtgtgcccgccagtagtgcttggtttccaacagctgctgctggctcttcctcttgcggccttttcctgaaacggattcttctttcggggaacagaaagcgccagccatgcagccttggcacggaaaggccatgcagagagcttccgaggccggagccactgcccccaaggcttccgcacggaatgccaggggcgccccgatggatcccaccgagtctccggctgcccccgaggccgccctgcctaaggcgggaaagttcggccccgccaggaagtcgggatcccggcagaaaaagagcgccccggacacccaggagaggccgcccgtccgcgcaactggggcccgcgccaaaaaggcccctcagcgcgcccaggacacgcagccgtctgacgccaccagcgcccctggggcagaggggctggagcctcctgcggctcgggagccggctctttccagggctggttcttgccgccagaggggcgcgcgctgctccacgaagccaagacctccgcccgggccctgggacgtgcccagccccggcctgccggtctcggcccccattctcgtacggagggatgcggcgcctggggcctcgaagctccgggcggttttggagaagttgaagctcagccgcgatgatatctccacggcggcggggatggtgaaaggggttgtggaccacctgctgctcagactgaagtgcgactccgcgttcagaggcgtcgggctgctgaacaccgggagctactatgagcacgtgaagatttctgcacctaatgaatttgatgtcatgtttaaactggaagtccccagaattcaactagaagaatattccaacactcgtgcatattactttgtgaaatttaaaagaaatccgaaagaaaatcctctgagtcagtttttagaaggtgaaatattatcagcttctaagatgctgtcaaagtttaggaaaatcattaaggaagaaattaacgacattaaagatacagatgtcatcatgaagaggaaaagaggagggagccctgctgtaacacttcttattagtgaaaaaatatctgtggatataaccctggctttggaatcaaaaagtagctggcctgctagcacccaagaaggcctgcgcattcaaaactggctttcagcaaaagttaggaagcaactacgactaaagccattttaccttgtacccaagcatgcaaaggaaggaaatggtttccaagaagaaacatggcggctatccttctctcacatcgaaaaggaaattttgaacaatcatggaaaatctaaaacgtgctgtgaaaacaaagaagagaaatgttgcaggaaagattgtttaaaactaatgaaataccttttagaacagctgaaagaaaggtttaaagacaaaaaacatctggataaattctcttcttatcatgtgaaaactgccttctttcacatggagtctcgctctgtcgcccaggctggagtccagtggcatgatcttggctcactgcaagctctgcttcctgggttcatgccattctcctgcctcagccttccgagtagctgggactacaggtgcccgccaccacatccggctaattttttgtatttttagtaaagatggggtttcaccatgttagccaggatggtctcgatctccttaccttgtgatccgcccgccttggcctcccaaagtgctgggattacaggtgtgagccaccacgcctggctgaaatacataatcttaaaagaaaacataagatactttattttaatatacgtgactaaatgtaaaacctaacttattttctgttatctatttatttttactttcagtaacactttttttattttaggtagcattcagcctagaggcaactgctgtttgttaaatatttcctgttcatatattttgcacattttcttatgggttagttttcttctcattgttttgggaagttcttaatatatttggggtatttatctttcattcgttgtctgtgtaacaaataacttctgccatatgggttgtctgcacattttttggtgtcttttagtaaacaaggtttttttgttttgtattgttttgtttattgtaaagatttttaaattttaatggagttgatttcttttctcattcaagcttttgagaataaattggagttgaattttt

SEQ ID NO: 72 - protein, 497 aa, Homo sapiens
MQPWHGKAMQRASEAGATAPKASARNARGAPMDPTESPAAPEAALPKAGKFGPARKSGSRQKKSAPDTQERPPVRATGARAKKAPQRAQDTQPSDATSAPGAEGLEPPAAREPALSRAGSCRQRGARCSTKPRPPPGPWDVPSPGLPVSAPILVRRDAAPGASKLRAVLEKLKLSRDDISTAAGMVKGVVDHLLLRLKCDSAFRGVGLLNTGSYYEHVKISAPNEFDVMFKLEVPRIQLEEYSNTRAYYFVKFKRNPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHMESRSVAQAGVQWHDLGSLQALLPGFMPFSCLSLPSSWDYRCPPPHPANFLYF

SEQ ID NO: 73 - DNA, 1071 bp, Peptoclostridium difficile
  source 1..1071: mol_type = genomic DNA
atggagaattttctagataataaaaatatgctatatgcattaaaaatgatatctcctggaactccacttagattaggtctaaacaatgtactaagagctaagactggtggattaattgtaattgcaacaaacgaagatgtaatgaaaatagtagatggaggatttgctataaatgcagaatattcaccatcatatctatatgaattagctaaaatggatggagctatagttttaagtggtgatgtaaagaaaatattatttgctaatgcacaacttatacctgactattttatagaaacatcagagacaggaacaagacatagaacagcagaaagagtagcaaaacaaactggtgctatagtcataggaatttcacaaagaagaaatgttataacagtttatagaggaaatgagaagtatgtagtcgaagatatatctaagatatttactaaggcaaatcaggctatacaaactctggaaaaatataagacagtattggaccaagctgtaacaaatttaaatgccttagagtttaatgatttggtaactatttatgatgttgcattagtcatgcaaaagatggaaatggtaatgagagttacaagtataattgaaaaatatgtgatagaattgggtgatgaaggaactttagtaagtatgcaattagaagaattaatgggtacaaccagaatagaccagaaattaatattcaaagattataataaagaaaacacagaaataaaagaacttatgaaaaaggtcaaaaatttaaattcagaagaactaatagaattggttaatatggcaaaactattagggtatagtggtttttcagaaagtatggatatgcctataaaaacaagaggttataggattcttagcaaaatacatagactaccaacagcaataatagaaaacttagtaaattattttgaaaactttcaacaaattttagatgcatctattgaagaattagatgaggttgaaggaataggtgaaataagagcaacatatataaaaaatggactcataaaaatgaaacaattagtcttattagatagacacatatga

SEQ ID NO: 74 - DNA, 1083 bp, Bacillus subtilis
  source 1..1083: mol_type = genomic DNA
atggaaaaagagaaaaaaggggcgaaacacgagttagacctgtcatctatattgcagtttgttgctccgggtacaccgctcagagcggggatggaaaacgtcttgagagcaaatacaggcggtctgattgttgttggatataatgataaagtaaaagaagtggtggacggcggctttcacataaacacggctttttctccggcgcatttatatgagctggctaaaatggatggagcgatcattttaagtgattctggtcaaaagatcctatacgcgaatactcagctgatgccggatgccacaatttcttcatcagaaacaggaatgcggcacagaactgccgaaagagtagctaagcaaactggctgtcttgtaatcgccatttctgaaagaagaaatgtcataacgttatatcaggaaaacatgaagtatacactaaaagacataggatttattttaaccaaggcgaaccaagccattcaaacacttgaaaaatataagacaatcctcgataaaacgattaatgcactgaacgcgttagagtttgaggaacttgttaccttcagtgatgtcttgtctgtcatgcatcgttatgaaatggtactgagaatcaaaaacgaaattaatatgtatatcaaagagctggggacagaagggcatctgatcaaactgcaagtcattgaattgattacggatatggaagaagaggccgctttatttattaaggactatgtaaaagaaaagattaaagatccgtttgttctcttgaaggagctgcaggatatgtccagttatgatctgctggatgattccattgtgtataagcttctcggttaccctgcttctactaatcttgatgattatgtattgccgagaggatacaggctgttaaataagataccgcgtcttccgatgccgattgttgaaaatgttgtagaagcatttggagtcctgccaaggattattgaggcgagtgcagaagaattagatgaagtagagggaatcggtgaagtacgagcccaaaaaatcaaaaaaggattaaaacgcctgcaagagaagcattatttagacagacaactgtga

SEQ ID NO: 75 - protein, 360 aa, Bacillus subtilis
MEKEKKGAKHELDLSSILQFVAPGTPLRAGMENVLRANTGGLIVVGYNDKVKEVVDGGFHINTAFSPAHLYELAKMDGAIILSDSGQKILYANTQLMPDATISSSETGMRHRTAERVAKQTGCLVIAISERRNVITLYQENMKYTLKDIGFILTKANQAIQTLEKYKTILDKTINALNALEFEELVTFSDVLSVMHRYEMVLRIKNEINMYIKELGTEGHLIKLQVIELITDMEEEAALFIKDYVKEKIKDPFVLLKELQDMSSYDLLDDSIVYKLLGYPASTNLDDYVLPRGYRLLNKIPRLPMPIVENVVEAFGVLPRIIEASAEELDEVEGIGEVRAQKIKKGLKRLQEKHYLDRQL

SEQ ID NO: 76 - protein, 454 aa, Pelobacter propionicus
MRRILVVEDDRFFRDLFYDLLVGQGYDVDRASSGEEGLDRLSTYAFDLVVTDLVMPGVDGMDILARARENDPSADVIMVTGNANLESAIFALKHGARDYFVKPINPDEFLHSVAQCLEQRRILDENEELKSMLNLYQISQAIAGCLDMERLQHLIFDAFTREIGTSRGMCLFATETGLELCEVKGVETAVAERCIASVLERLSEDHPDECNSLRISFQGGGDDSGIEAAILIPLRGKGSQRGVVVAFNEPGLGLPELGARKKNILFLLEQSLLALENASSYSLAKDMLFIDDLSGLYNQRYLEVALEREMKRIGRFSSQLAVLFLDMDSFKQVNDTHGHLVGSRVLKEMGTLLRLSVRDVDVVIRYGGDEYTAILVETSPAIAANVAERIRSMVASHVFLADEGYDIRLTCSIGYSCCPEDALTKEELLEMADQAMYTGKGRGKNCVVRFTKTS

SEQ ID NO: 77 - protein, 462 aa, Geobacter uraniireducens
MERILVVEDDSFFREVFADLLIEDGFHVDVAASGEQALVMVQNREYQLVVTDLVMPDITGLDILSKVKQLDPTIDVIMVTGHANMETAIFALKNGARDYLVKPINHDEFKHAVALCFEQRRLLDENQELKGLINLYHVSQTIANCLDLERIHTLLVDSLAKEFAVSRGLGYFLDGADNLELKALKGVSEASAGRLGELILSRYNVQGEDSRSFVLLHDFMQPDADFGLGTDGDMKEAMLFFVRSRTVLQGIVILFSEPGTSFPADIQFKNINFLLDQSSLALENAVRYNNAKNLLYIDELTGLFNYRYLDVALEREIRRAERYGSHISVIFLDIDLFKRVNDMYGHLVGSRALNEVGILLKKSVRDVDTVIRYGGDEYTIILIETGIDGAAAVAERIRRSIEAHGFMAADGLNLKLTASLGYACYPEDAKTKTELLELADQAMYRGKADGKNRVFYVSAKNN

SEQ ID NO: 78 - protein, 460 aa, Geobacter daltonii
MERILVVEDDSFFREVFADLLRDDGFAVDVACSGEKALEMLRSSEYALVVTDLVMPDITGLDLLSKVKQFDPSIDVILVTGHANTETAVFALKNGARDYLVKPINSEEFKHAVALCFEQRRLLDENQELKGLLNLFQISQTIANSLDFDRIHTILVDSLAKEFGLSRLTGYFQNDDGTLELKEIKGFDEETASSLGELIFDIFDVREEDNRSFVLLNDLEQRSRFFAEHSVTEAMLFFVRAKTALLGIIIVFNESQSVFPAHLDFKNINFLLDQASLALENASRYNNAKNLLYIDELTGLFNYRYLDVALEREVRRAERYSSNISIIFLDIDLFKRINDQYGHLVGSKALAEVGLLLKKSVRDVDTVIRYGGDEYTIILIETGIDGASVVAERIRSTIEGHVFIQSEGLDIKLTASLGCASYPEDACTKLELLELADQAMYRSKACGKNMVFHISAYKKQ

SEQ ID NO: 79 - protein, 4 aa, unidentified
  REGION 1..4: note = source = /note="Description of Unknown: 'DEAD' box family motif peptide"
DEAD

SEQ ID NO: 80 - protein, 8 aa, synthetic construct
  REGION 1..8: note = source = /note="Description of Artificial Sequence: Synthetic peptide"
SIINFEKL

SEQ ID NO: 81 - DNA, 36 bp, synthetic construct
  misc_feature 1..36: note = source = /note="Description of Artificial Sequence: Synthetic primer"
  source 1..36: mol_type = other DNA
ataggtaccccaccgtgatgacaactgaagatttca

SEQ ID NO: 82 - DNA, 29 bp, synthetic construct
  misc_feature 1..29: note = source = /note="Description of Artificial Sequence: Synthetic primer"
  source 1..29: mol_type = other DNA
atactcgagttagagcggcatgactcgat

SEQ ID NO: 83 - DNA, 40 bp, synthetic construct
  misc_feature 1..40: note = source = /note="Description of Artificial Sequence: Synthetic primer"
  source 1..40: mol_type = other DNA
tgacagcttatcgttatgccgctgaagagtttgcactgat

SEQ ID NO: 84 - protein, 5 aa, Vibrio cholerae
GGEEF

SEQ ID NO: 85 - protein, 5 aa, synthetic construct
  REGION 1..5: note = source = /note="Description of Artificial Sequence: Synthetic peptide"
AAEEF

SEQ ID NO: 86 - protein, 15 aa, synthetic construct
  REGION 1..15: note = source = /note="Description of Artificial Sequence: Synthetic peptide"
VNGSRYYFDTDTAIA

SEQ ID NO: 87 - DNA, 31 bp, synthetic construct
  misc_feature 1..31: note = source = /note="Description of Artificial Sequence: Synthetic primer"
  source 1..31: mol_type = other DNA
ataggtaccccaccatgaatgacaaagtgct

SEQ ID NO: 88 - DNA, 34 bp, synthetic construct
  misc_feature 1..34: note = source = /note="Description of Artificial Sequence: Synthetic primer"
  source 1..34: mol_type = other DNA
atactcgagttagaaaagttcaacgtcatcagaa

SEQ ID NO: 89 - DNA, 50 bp, synthetic construct
  misc_feature 1..50: note = source = /note="Description of Artificial Sequence: Synthetic primer"
  source 1..50: mol_type = other DNA
gtcttctcaactatttcgctttgctgctgaagagttcgtgattatttttt

SEQ ID NO: 90 - DNA, 20 bp, synthetic construct
  misc_feature 1..20: note = source = /note="Description of Artificial Sequence: Synthetic primer"
  source 1..20: mol_type = other DNA
gctactacgaggacggcctg

SEQ ID NO: 91 - DNA, 21 bp, synthetic construct
  misc_feature 1..21: note = source = /note="Description of Artificial Sequence: Synthetic primer"
  source 1..21: mol_type = other DNA
ctcatcgatgatcagcttgcc

SEQ ID NO: 92 - protein, 9 aa, synthetic construct
  REGION 1..9: note = source = /note="Description of Artificial Sequence: Synthetic peptide"
AMQMLKETI

SEQ ID NO: 93 - protein, 26 aa, Pseudomonas aeruginosa
LTDPLTGLPNRAALSERLEQEVARRH

SEQ ID NO: 94 - protein, 14 aa, Pseudomonas aeruginosa
LLAVLDIDHFKRIN

SEQ ID NO: 95 - protein, 25 aa, Pseudomonas aeruginosa
DFGHLAGDKVLKIIAGELRKRLRQA

SEQ ID NO: 96 - protein, 17 aa, Pseudomonas aeruginosa
IARFGGEEFVVLLPATS

SEQ ID NO: 97 - protein, 19 aa, Pseudomonas aeruginosa
EAGRQLLERLRAAIAACPF

SEQ ID NO: 98 - protein, 19 aa, Pseudomonas aeruginosa
PLSITCSAGITAFEGNEAG

SEQ ID NO: 99 - protein, 4 aa, Pseudomonas aeruginosa
AVFE

SEQ ID NO: 100 - protein, 19 aa, Pseudomonas aeruginosa
ADQALYRAKRAGRDRLEVA

SEQ ID NO: 101 - protein, 26 aa, Pseudomonas aeruginosa
YIDEPTGLYNRLRLQEDVSLRLQRDG

SEQ ID NO: 102 - protein, 14 aa, Pseudomonas aeruginosa
TVIAADLLPLALLN

SEQ ID NO: 103 - protein, 25 aa, Pseudomonas aeruginosa
TLGYPFSNDLMLEARDRIRAELPDF

SEQ ID NO: 104 - protein, 17 aa, Pseudomonas aeruginosa
LYKISPTRFGLLLPRQQ

SEQ ID NO: 105 - protein, 19 aa, Pseudomonas aeruginosa
EETESVCLRLLRAFESPVV

SEQ ID NO: 106 - protein, 15 aa, Pseudomonas aeruginosa
KANVGLGVLPLADDT

SEQ ID NO: 107 - protein, 4 aa, Pseudomonas aeruginosa
DWLR

SEQ ID NO: 108 - protein, 19 aa, Pseudomonas aeruginosa
VVSAADDARDRGVGWARYN

SEQ ID NO: 109 - protein, 26 aa, Pseudomonas aeruginosa
SQDPVTGLYNRSHFLDLMDAAVQQAV

SEQ ID NO: 110 - protein, 14 aa, Pseudomonas aeruginosa
TLAYIHLNGYPSLQ

SEQ ID NO: 111 - protein, 25 aa, Pseudomonas aeruginosa
DHGLSGIDLLLGQLAGLMREQFGEE

SEQ ID NO: 112 - protein, 17 aa, Pseudomonas aeruginosa
LARFGDSIFAALFKGKT

SEQ ID NO: 113 - protein, 19 aa, Pseudomonas aeruginosa
EQAQAALQRLLKKVENHLF

SEQ ID NO: 114 - protein, 15 aa, Pseudomonas aeruginosa
QATLSIGVAGLDEKT

SEQ ID NO: 115 - protein, 4 aa, Pseudomonas aeruginosa
DVMN

SEQ ID NO: 116 - protein, 19 aa, Pseudomonas aeruginosa
AHRCADDAARKGGSQIKQY

SEQ ID NO: 117 - protein, 26 aa, Salmonella enterica
MRFPVSELPNKAFLMALLEQVITRQQ

SEQ ID NO: 118 - protein, 12 aa, Salmonella enterica
ALIIVTCETLRD

SEQ ID NO: 119 - protein, 27 aa, Salmonella enterica
AGVLQETQREILLLTLVEKLKSVLAPR

SEQ ID NO: 120 - protein, 17 aa, Salmonella enterica
LTQVSGYDFAIIAHGVK

SEQ ID NO: 121 - protein, 19 aa, Salmonella enterica
WHAITLGQQILTIINERLP

SEQ ID NO: 122 - protein, 17 aa, Salmonella enterica
QLRPSCSIGIAMYYGDL

SEQ ID NO: 123 - protein, 23 aa, Salmonella enterica
LYGRAVSAAFTARRKGKNQIQFF

SEQ ID NO: 124 - protein, 26 aa, Vibrio cholerae
SHDLGTGFLNRTALEQQLAMQLAQLA

SEQ ID NO: 125 - protein, 14 aa, Vibrio cholerae
AVIHIGFANARQLQ

SEQ ID NO: 126 - protein, 25 aa, Vibrio cholerae
RLGYHLWDDVLKQLRERLGPVTEGE

SEQ ID NO: 127 - protein, 17 aa, Vibrio cholerae
TARPNSTNLTLILKAHP

SEQ ID NO: 128 - protein, 10 aa, Vibrio cholerae
TQLNQLCHRL

SEQ ID NO: 129 - protein, 9 aa, Vibrio cholerae
AGQAQFVTE

SEQ ID NO: 130 - protein, 15 aa, Vibrio cholerae
HLNPYIGVALSRETR

SEQ ID NO: 131 - protein, 6 aa, Vibrio cholerae
PQQLLR

SEQ ID NO: 132 - protein, 19 aa, Vibrio cholerae
AVSSMLACKDSGYKVFFHS

SEQ ID NO: 133 - protein, 26 aa, Xylella fastidiosa
QKASVTELQTRTHIVEQLEVALSSGA

SEQ ID NO: 134 - protein, 14 aa, Xylella fastidiosa
ALFFIEVSSALNLR

SEQ ID NO: 135 - protein, 25 aa, Xylella fastidiosa
RYGYTAFERLMNQVEYHLAQEAHPY

SEQ ID NO: 136 - protein, 17 aa, Xylella fastidiosa
LARISDHSFLLLAIDLA

SEQ ID NO: 137 - protein, 10 aa, Xylella fastidiosa
EHQALASHLR

SEQ ID NO: 138 - protein, 9 aa, Xylella fastidiosa
LATLPLPIQ

SEQ ID NO: 139 - protein, 15 aa, Xylella fastidiosa
HLRSAIGYAPLNQGF

SEQ ID NO: 140 - protein, 4 aa, Xylella fastidiosa
DAVE

SEQ ID NO: 141 - protein, 19 aa, Xylella fastidiosa
TERATLEARQNNEGIYAYV

SEQ ID NO: 142 - protein, 26 aa, Xylella fastidiosa
QRDPITGLLNRPAFIIALEDAVARAG

SEQ ID NO: 143 - protein, 12 aa, Xylella fastidiosa
GLLLVEPNHYTR

SEQ ID NO: 144 - protein, 27 aa, Xylella fastidiosa
LQEIGLDSADTLITSLANFFSEMIDSK

SEQ ID NO: 145 - protein, 27 aa, Xylella fastidiosa
TARFCETRFALLLEGDYTHTMTLAERI

SEQ ID NO: 146 - protein, 9 aa, Xylella fastidiosa
IDIAQHIFL

SEQ ID NO: 147 - protein, 15 aa, Xylella fastidiosa
TVTVSIGGVQIGERI

SEQ ID NO: 148 - protein, 4 aa, Xylella fastidiosa
QVLH

SEQ ID NO: 149 - protein, 19 aa, Xylella fastidiosa
AAESVQIATQLGGNTAIIY

SEQ ID NO: 150 - protein, 26 aa, Escherichia coli
MRFPVSDLPNKALLMEMLEQVVARKQ

SEQ ID NO: 151 - protein, 12 aa, Escherichia coli
ALMIITCETLRD

SEQ ID NO: 152 - protein, 27 aa, Escherichia coli
AGVLKEAQREILLLTLVEKLKSVLSPR

SEQ ID NO: 153 - protein, 17 aa, Escherichia coli
LAQISGYDFAVIANGVQ

SEQ ID NO: 154 - protein, 19 aa, Escherichia coli
WHAITLGQQVLTIMSERLP

SEQ ID NO: 155 - protein, 17 aa, Escherichia coli
QLRPHCSIGVAMFYGDL

SEQ ID NO: 156 - protein, 23 aa, Escherichia coli
LYSRAISAAFTARHKGKNQIQFF

SEQ ID NO: 157 - protein, 26 aa, Escherichia coli
MRFPVSDLPNKALLMEMLEQVVARKQ

SEQ ID NO: 158 - protein, 12 aa, Escherichia coli
ALMIITCETLRD

SEQ ID NO: 159 - protein, 27 aa, Escherichia coli
AGVLKEAQREILLLTLVEKLKSVLSPR

SEQ ID NO: 160 - protein, 17 aa, Escherichia coli
LAQISGYDFAVIANGVQ

SEQ ID NO: 161 - protein, 19 aa, Escherichia coli
WHAITLGQQVLTIMSERLP

SEQ ID NO: 162 - protein, 17 aa, Escherichia coli
QLRPHCSIGVAMFYGDL

SEQ ID NO: 163 - protein, 23 aa, Escherichia coli
LYSRAISAAFTARHKGKNQIQFF

SEQ ID NO: 164 - protein, 26 aa, Escherichia coli
MRFPVSDLPNKALLMEMLEQVVARKQ

SEQ ID NO: 165 - protein, 12 aa, Escherichia coli
ALMIITCETLRD

SEQ ID NO: 166 - protein, 27 aa, Escherichia coli
AGVLKEAQREILLLTLVEKLKSVLSPR

SEQ ID NO: 167 - protein, 17 aa, Escherichia coli
LAQISGYDFAVIANGVQ

SEQ ID NO: 168 - protein, 19 aa, Escherichia coli
WHAITLGQQVLTIMSERLP

SEQ ID NO: 169 - protein, 17 aa, Escherichia coli
QLRPHCSIGVAMFYGDL

SEQ ID NO: 170 - protein, 23 aa, Escherichia coli
LYSRAISAAFTARHKGKNQIQFF